(19) United States
(12) Patent Application Publication
(10) Pub. No.: US 2004/0106653 A1
(43) Pub. Date: Jun. 3, 2004

(54) Aminoalcohol derivatives

(75) Inventors: Minoru Sakurai, Osaka (JP); Hitoshi Hamashima, Osaka (JP); Kouji Hattori, Osaka (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: FUJISAWA PHARMACEUTICAL CO., LTD., Osaka-shi (JP)

(21) Appl. No.: 10/716,513

(22) Filed: Nov. 20, 2003

(30) Foreign Application Priority Data
Nov. 21, 2002 (AU) 2002952839

(51) Int. Cl. A61K 31/455; A61K 31/401; A61K 31/195; C07D 213/60; C 07D 2 7/34

(52) U.S. Cl. 514/355; 514/423; 514/553; 514/567; 514/534; 546/315; 548/530; 560/37

(57) ABSTRACT

The present invention relates to a compound formula [I]:

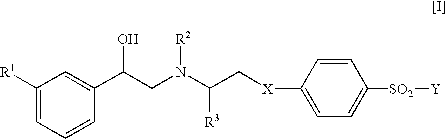

wherein

R¹ is hydrogen or halogen,

R² is hydrogen or an amino protective group,

R³ is hydrogen or lower alkyl,

X is bond, —CH₂— or —O—, and

Y is

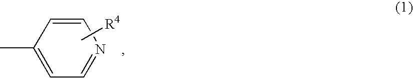

in which R⁴ is lower alkoxycarbonyl,

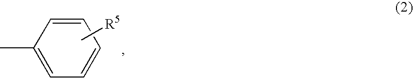

in which R⁵ is carboxy(lower)alkyl, etc.,

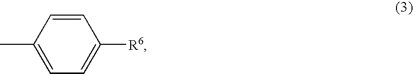

in which R⁶ is hydroxy, etc., and so on,

or a salt thereof. The compound [I] of the present invention and pharmaceutically acceptable salts thereof are useful for the prophylactic and/or the therapeutic treatment of pollakiurea or urinary incontinence.

### TECHNICAL FIELD

This invention relates to new aminoalcohol derivatives and salts thereof which are beta-3 (β₃) adrenergic receptor agonists and useful as a medicament.

### BACKGROUND OF THE INVENTION

International Publication No. WO 02/094770, published Nov. 28, 2002, describes aminoalcohol derivatives useful as 3 adrenergic receptor agonists.

### DISCLOSURE OF INVENTION

This invention relates to new aminoalcohol derivatives which are 3 adrenergic receptor agonists and salts thereof.

More particularly, it relates to new aminoalcohol derivatives and salts thereof which have gut sympathomimetic, anti-ulcerous, anti-pancreatitis, lipolytic, anti-urinary incontinence, anti-pollakiuria activities, anti-diabetes and anti-obesity, to processes for the preparation thereof, to a pharmaceutical composition comprising the same and to a method of using the same therapeutically in the treatment and/or prevention of gastro-intestinal disorders caused by smooth muscle contractions in a human being or an animal.

One object of this invention is to provide new and useful aminoalcohol derivatives and salts thereof which have gut sympathomimetic, anti-ulcerous, lipolytic, anti-urinary incontinence, anti-pollakiuria activities, anti-diabetes and anti-obesity.

Another object of this invention is to provide processes for the preparation of said aminoalcohol derivatives and salts thereof.

A further object of this invention is to provide a pharmaceutical composition comprising, as an active ingredient, said aminoacohol derivatives and salts thereof.

Still further object of this invention is to provide a therapeutical method for the treatment and/or prevention of aforesaid diseases in a human being or an animal, using said aminoalcohol derivatives and salts thereof.

The object aminoalcohol derivatives of this invention are new and can be represented by compound of the following formula [I]:

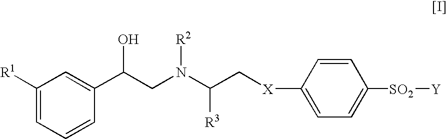

wherein

R¹ is hydrogen or halogen,

R² is hydrogen or an amino protective group,

R³ is hydrogen or lower alkyl,

X is bond, —CH₂— or —O—, and

Y is

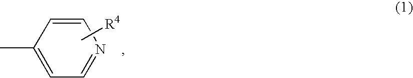

in which R⁴ is lower alkoxycarbonyl,

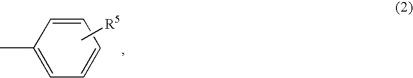

in which R⁵ is carboxy(lower)alkyl, (lower alkoxy)-carbonyl(lower)alkyl, lower alkanoyl, mono(or di or tri)halo(lower)alkylsulfonyloxy, carboxyphenoxy, (lower alkoxy)carbonylphenoxy, carboxypyridyloxy, (lower alkanoyl)pyridyl, carboxypyrrolidinyl(lower)alkyl, (lower alkoxy)carbonylpyrrolidinyl(lower)alkyl, carboxyphenyl or (lower alkyl)phenyl,

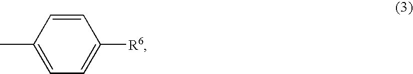

in which R⁶ is —OH, —COOH, —COOC₂H₅,

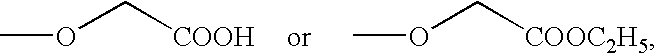

provided that

(i) when R⁶ is —OH, then X is —CH₂—,

(ii) when R is —COOH, then R¹ is —H, or

(iii) when R⁶ is —COOC₂H₅,

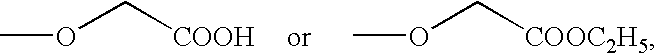

then X is —O—,

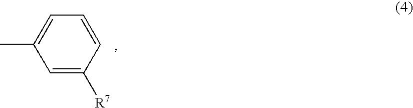

in which

R⁷ is —OH, —COOH, —COOC₂H₅,

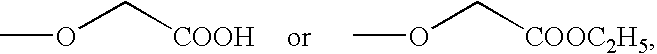

and

X is —CH₂—,

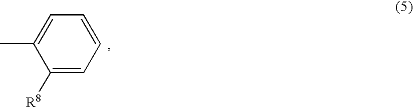

in which R⁸ is —OH, —COOH, —COOC₂H₅,

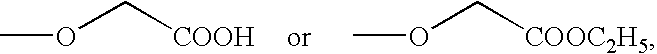

provided that

when R⁸ is —OH,

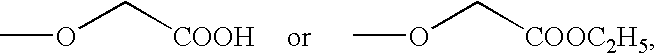

then R³ is —CH₃,

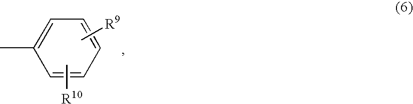

in which

R⁹ is hydroxy, cyclo(lower)alkyl, mono(or di or tri)halo(lower)alkyl, hydroxy(lower)alkoxy, lower alkoxy(lower)alkoxy, carboxy(lower)alkoxy, lower alkoxycarbonyl(lower)alkoxy, phenoxy, nitro, amino, lower alkylamino, [lower alkoxy(lower)alkyl]amino, [hydroxy(lower)alkyl]amino, [lower alkoxycarbonyl]amino, lower alkanoylamino, [hydroxy(lower)alkanoyl]amino, benzoylamino, (lower alkylsulfonyl)amino, lower alkylthio or phenyl, and

R¹⁰ is carboxy, lower alkanoyl, lower alkoxycarbonyl, carbamoyl, lower alkylcarbamoyl, carboxy(lower)alkyl, (lower alkoxycarbonyl)(lower)alkyl, carboxy(lower)-alkenyl, (lower alkoxycarbonyl)(lower)alkenyl or phenyl optionally substituted with carboxy or lower alkoxycarbonyl,

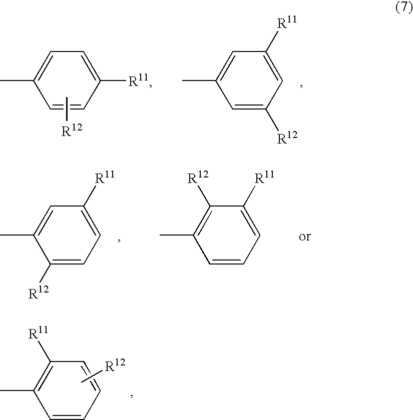

in which

R¹¹ is halogen or lower alkyl, and

R¹² is carboxy, lower alkanoyl, lower alkoxycarbonyl, carbamoyl, lower alkylcarbamoyl, carboxy(lower)alkyl, (lower alkoxycarbonyl)(lower)alkyl, carboxy(lower)-alkenyl or (lower alkoxycarbonyl)(lower)alkenyl,

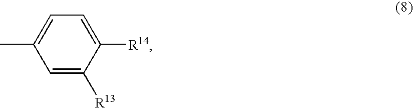

in which

R¹³ is —Cl or —CH₃,

R¹⁴ is —COOH or —COOC₂H₅, and

X is —CH₂—,

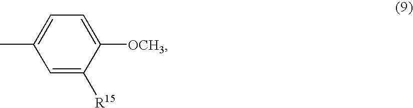

in which

R¹⁵ is —COOH or —COOC₂H₅, and

X is —CH₂—, or

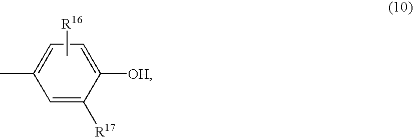

in which

R¹⁶ is lower alkyl or lower alkoxy, and

R¹⁷ is carboxy or lower alkoxycarbonyl, or a prodrug thereof or a pharmaceutially acceptable salt thereof.

According to this invention, the object compounds can be prepared by processes which are illustrated in the following schemes.

Process 1

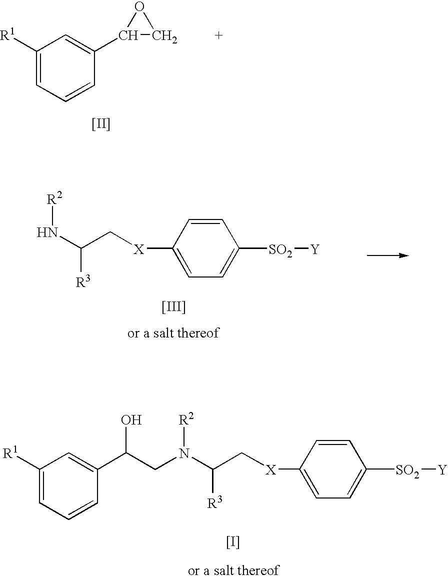

Process 2

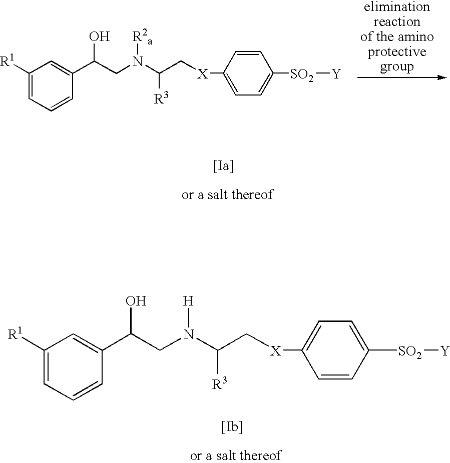

Process 3

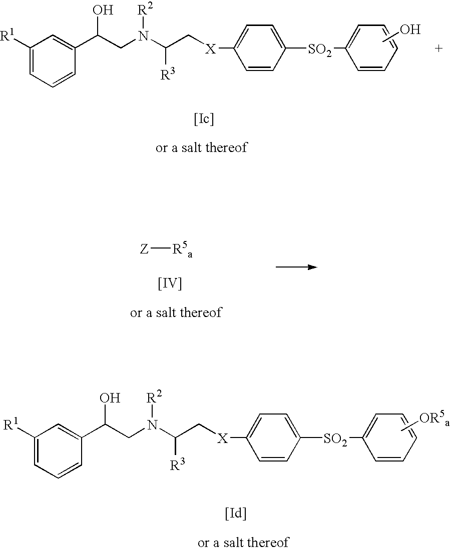

wherein

R¹, R², R³, X and Y are each as defined above,

Rₐ² is an amino protective group, and

Rₐ⁵ is lower alkyl optionally substituted with carboxy or lower alkoxycarbonyl; phenyl substituted with lower alkanoyl, carboxy or lower alkoxycarbonyl; or pyridyl optionally substituted with lower alkanoyl, carboxy or lower alkoxycarbonyl, and

Z is halogen.

As to the starting compounds [II], [III], [Ia], [Ic] and [IV], some of them are novel and can be prepared by the procedures described in the Preparations and Examples mentioned later or a conventional manner.

In the above and subsequent description of the present specification, suitable examples of the various definition to be included within the scope of the invention are explained in detail in the following.

The term “lower” is intended to mean a group having 1 to 6, preferably 1 to 4, carbon atom(s), unless otherwise indicated.

Suitable “lower alkyl” and “lower alkyl” moiety in the term of “mono(or di or tri)halo(lower)alkylsulfonyloxy” may include straight or branched one having 1 to 6 carbon atom(s), such as methyl, ethyl, propyl, isopropyl, butyl, isobutyl, sec-butyl, tert-butyl, pentyl, 1-methylpentyl, tert-pentyl, neo-pentyl, hexyl, isohexyl and the like, in which more preferable one is C₁-C₄ alkyl, and the most preferable one is methyl, ethyl, propyl or isobutyl.

Suitable “cyclo(lower)alkyl” may include cyclopropyl, cyclobutyl, cyclopentyl, cyclohexyl, cycloheptyl and the like, in which the preferred one is cyclo(C₃-C₆)alkyl and the most preferred one is cyclohexyl.

The term “lower alkenyl” means one having one or two double bond(s) in the straight or branched lower alkyl group as defined above.

Suitable “lower alkenyl” moiety in the terms of “carboxy(lower)alkenyl” and “(lower alkoxycarbonyl)(lower)-alkenyl” may include one having 2 to 6 carbon atoms such as vinyl, 1-propenyl, 2-propenyl, 1,3-butadienyl, 1-methylvinyl and the like, in which the preferred one is vinyl.

Suitable “lower alkoxy” and “lower alkoxy” moiety in the term of “lower alkoxycarbonyl” may include methoxy, ethoxy, propoxy, isopropoxy, butoxy, iso-butoxy, t-butoxy, pentyloxy, t-pentyloxy, hexyloxy and the like, in which preferable one is C₁-C₄ alkoxy, and the most preferable one is methoxy or ethoxy.

Suitable “lower alkanoyl” may include formyl, acetyl, propanoyl, butanoyl, 2-methylpropanoyl, pentanoyl, 2,2-dimethylpropanoyl, hexanoyl and the like, in which preferable one is C₂-C₄ alkanoyl, and the most preferable one is formyl.

Suitable “halogen” may be fluoro, chloro, bromo and iodo, in which preferable one is fluoro or chloro.

Suitable “mono(or di or tri)halo(lower)alkyl” and “mono(or di or tri)halo(lower)alkyl” moiety in the term of “mono(or di or tri)halo(lower)alkylsulfonyloxy” may include chloromethyl, dichloromethyl, trichloromethyl, bromomethyl, dibromomethyl, tribromomethyl, fluoromethyl, difluoromethyl, trifluoromethyl, 1 or 2-chloroethyl, 1 or 2-bromoethyl, 1 or 2-fluoroethyl, 1,1-difluoroethyl, 2,2-difluoroethyl and the like, in which more preferable one is mono(or di or tri)halo(C₁-C₄)alkyl, and the most preferable one is trifluoromethyl.

Suitable example of “amino protective group” moiety may be common amino protective group such as substituted or unsubstituted lower alkanoyl [e.g. formyl, acetyl, propionyl, trifluoroacetyl, etc.], phthaloyl, lower alkoxycarbonyl [e.g. tert-butoxycarbonyl, tert-amyloxycarbonyl, etc.], substituted or unsubstituted aralkyloxycarbonyl [e.g. benzyloxycarbonyl, p-nitrobenzyloxycarbonyl, etc.], substituted or unsubstituted arenesulfonyl [e.g. benzenesulfonyl, tosyl, etc.], nitrophenylsulfenyl, ar(lower)alkyl [e.g. trityl, benzyl, etc.], and the like, in which preferable one is benzyl or tert-butoxycarbonyl.

Suitable salts of the object aminoalcohol derivative [I] are pharmaceutically acceptable salts and include conventional non-toxic salts such as an inorganic acid addition salt [e.g. hydrochloride, hydrobromide, sulfate, phosphate, etc.], an organic acid addition salt [e.g. formate, acetate, trifluoroacetate, oxalate, maleate, fumarate, tartrate, citrate, methanesulfonate, benzenesulfonate, toluenesulfonate, etc., an alkali metal salt [e.g. sodium salt, potassium salt, etc.] or the like.

The Processes 1 to 3 for preparing the object compounds of the present invention are explained in detail in the following.

Process 1

The object compound [I] or a salt thereof can be prepared by reacting a compound [II] with a compound [III] or a salt thereof.

Suitable salt of the compound [III] may be the same as those exemplified for the compound [I].

The reaction is preferably carried out in the presence of a base such as an alkali metal carbonate [e.g. sodium carbonate, potassium carbonate, etc.], an alkaline earth metal carbonate [e.g. magnesium carbonate, calcium carbonate, etc.], an alkali metal bicarbonate [e.g. sodium bicarbonate, potassium bicarbonate, etc.], tri(lower)alkylamine [e.g. trimethylamine, triethylamine, etc.], picoline or the like.

The reaction is usually carried out in a conventional solvent, such as an alcohol [e.g. methanol, ethanol, propanol, isopropanol, etc.], diethyl ether, tetrahydrofuran, dioxane, or any other organic solvent which does not adversely influence the reaction.

The reaction temperature is not critical, and the reaction can be carried out under cooling to heating.

Process 2

The object compound [Ib] or a salt thereof can be prepared by subjecting a compound [Ia] or a salt thereof to elimination reaction of the amino protective group.

Suitable salts of the compounds [Ia] and [Ib] may be the same as those exemplified for the compound [I].

This reaction can be carried out in a similar manner to that of Example 7 or 25 mentioned below.

Process 3

The object compound [Id] or a salt thereof can be prepared by reacting a compound [Ic] or a salt thereof with a compound [IV] or a salt thereof.

Suitable salts of the compounds [Ic] and [IV] may be the same as those exemplified for the compound [I].

This reaction can be carried out in a similar manner to that of Example 22 or 24.

The compounds obtained by the above processes can be isolated and purified by a conventional method such as pulverization, recrystallization, column chromatography, reprecipitation, or the like, and converted to the desired salt in conventional manners, if necessary.

It is to be noted that the compound [I] and the other compounds may include one or more stereoisomers due to asymmetric carbon atoms, and all of such isomers and mixture thereof are included within the scope of this invention.

It is further to be noted that isomerization or rearrangement of the object compound [I] may occur due to the effect of the light, acid base or the like, and the compound obtained as the result of said isomerization or rearrangement if also included within the scope of the present invention.

It is also to be noted that the solvating form of the compound [I] (e.g. hydrate, etc.) and any form of the crystal of the compound [I] are included within the scope of the present invention.

The object compound [I] or a salt thereof possesses gut sympathomimetic, anti-ulcerous, anti-pancreatitis, lipolytic, anti-urinary incontinence and anti-pollakiuria activities, and are useful for the treatment and/or prevention of gastro-intestinal disorders caused by smooth muscle contractions in human beings or animals, and more parcitularly for the treatment and/or prevention of spasm or hyperanakinesia in case of irritable bowel syndrome, gastritis, gastric ulcer, duodenal ulcer, enteritis, cholecystopathy, cholantitis, urinary calculus and the like; for the treatment and/or prevention of ulcer such as gastric ulcer, duodenal ulcer, peptic ulcer, ulcer causes by non steroidal anti-inflammatory drags, or the like; for the treatment and/or prevention of dysuria such as pollakiuria, urinary incontinence or the like in case of nervous pollakiuria, neurogenic bladder dysfunction, nocturia, unstable bladder, cystospasm, chronic cystitis, chronic prostatitis, prostatic hypertrophy or the like; for the treatment and/or prevention of overactive bladder disorder, stress incontinence, urge incontinence, mixted incontinence, functional incontinence, overflow incontinence; for the treatment and/or prevention of pancreatitis, obesity, diabetes, glycosuria, hyperlipidemia, hypertension, atherosclerosis, glaucoma, melancholia, depression or the like; for the treatment and/or prevention of diseases as the result of insulin resistance (e.g. hypertension, hyperinsulinemia, etc.); for the treatment and/or prevention of neurogenetic inflammation; and for reducing a wasting condition, and the like.

Additionally, β₃ adrenergic receptor agonists are known to lower triglyceride and cholesterol levels and to raise high density lipoprotein levels in mammals (U.S. Pat. No. 5,451,677). Accordingly, the object compound [I] in useful in the treatment and/or prevention of conditions such as hyper-triglyceridaemia, hypercholesterolaemia and in lowering high density lipoprotein levels as well as in the treatment of atherosclerotic and cardiovascular diseases and relates conditions.

Moreover, the object compound [I] is useful for inhibiting uterine contractions, preventing premature labor, and treating and preventing dysmenorrhea.

For therapeutic purpose, the compound (I) and a pharmaceutically acceptable salt thereof of the present invention can be used in a form of pharmaceutical preparation containing one of said compounds, as an active ingredient, in admixture with a pharmaceutically acceptable carrier such as an organic or inorganic solid or liquid excipient suitable for oral, parenteral, external including topical, internal, intravenous, intramuscular, inhalant, nasal, intraarticular, intraspinal, transtracheal or transocular administration. The pharmaceutical preparations may be solid, semi-solid or solutions such as capsules, tablets, pellets, dragees, powders, granules, suppositories, ointments, creams, lotions, inhalants, injections, cataplasms, gels, tapes, eye drops, solution, syrups, aerosols, suspension, emulsion, or the like. If desired, there may be included in these preparations, auxiliary substances, stabilizing agents, wetting or emulsifying agents, buffers and other commonly used additives.

While the dosage of the compound (I) will vary depending upon the age and condition of a patient, an average single dose of about 0.1 mg, 1 mg, 10 mg, 50 mg, 100 mg, 250 mg, 500 mg and 1000 mg of the compound (I) may be effective for treating diseases such as pollakiurea, urinary incontinence and the like. In general, amounts between 0.1 mg/body and about 1,000 mg/body may be administered per day.

In order to show the usefulness of the compound [I] for the prophylactic and therapeutic treatment of abovementioned disease in human being or animals, the pharmacological test data of a representative compound thereof are shown in the following.

Test

Effect on the increase in intravesical pressure induced by carbachol in anesthetized dog

Test Compound

(1) [[4-[[4-[(2R)-2-[[(2R)-2-(3-Chlorophenyl)-2-hydroxyethyl]amino]propyl]phenyl]sulfonyl]benzoyl]-(methyl)amino]acetic acid hydrochloride (the object compound of Example 25 mentioned below)

Test Method

Female Beagle dogs weighing 8.0-15.0 kg were fasted for 24 hours and maintained under halothane anesthesia. A 12F Foley catheter was lubricated with water soluble jelly, inserted into the urethral orifice and advanced approximately 10 cm until the balloon tip was placed well inside the bladder. The balloon was then inflated with 5 ml of room air and catheter slowly withdrawn just part the first resistance that is felt at the bladder neck. Urine was completely drained out through the catheter, and 30 ml of biological saline was infused. The catheter was connected to pressure transducer, and intravesical pressure was continuously recorded. Intravenous administration of test compound (I) inhibited carbachol (1.8 μg/kg)-induced increase in intravesical pressure (IVP).

Test Results

| | % inhibition of carbachol-induced |
|---|---|
| Treatment | increase in IVP |
| Test Compound (1) | 80.8% |
| (0.032 mg/kg) | |

Preferred embodiments of the object compound [I] are as follows:

R¹ is hydrogen or chloride,

R² is hydrogen or benzyl,

R³ is hydrogen or methyl,

X is bond, —CH₂— or —O—, and

Y is

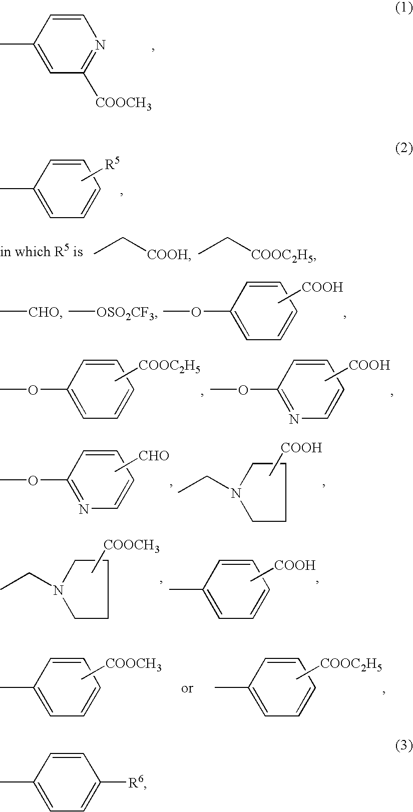

in which R⁶ is —OH, —COOH, —COOC₂H₅,

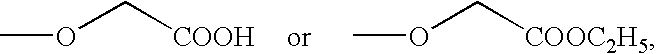

provided that

(i) when R⁶ is —OH, then X is —CH₂—,

(ii) when R⁶ is —COOH, then R¹ is —H, or

(iii) when R⁶ is —COOC₂H₅,

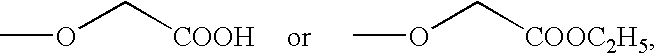

then X is —O—,

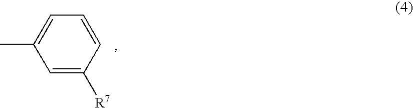

in which R⁷ is —OH, —COOH, —COOC₂H₅,

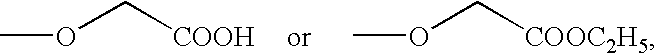

and X is —CH₂—,

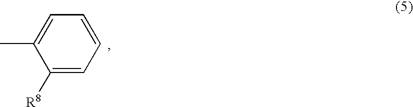

in which R⁸ is —OH, —COOH, —COOC₂H₅,

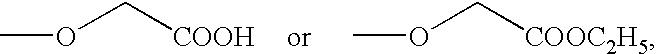

provided that

when R⁸ is —OH,

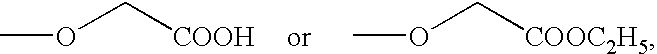

then R³ is —CH₃,

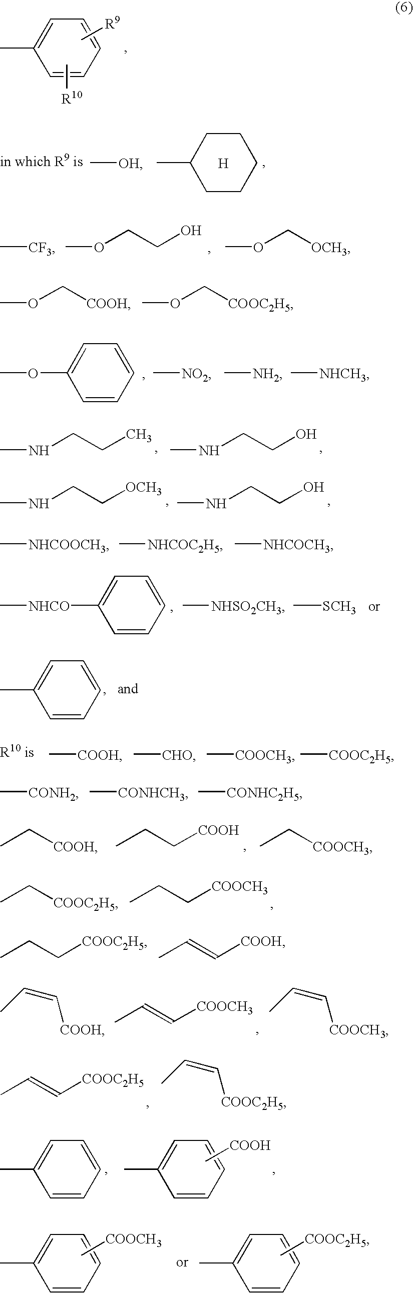

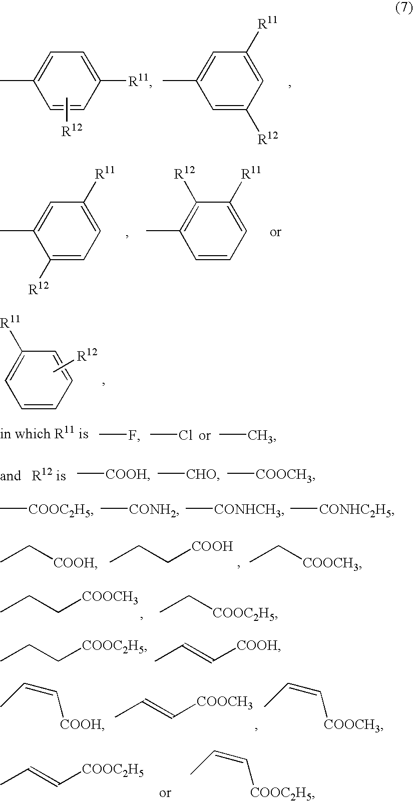

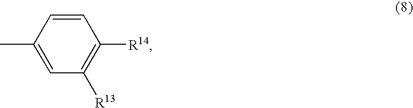

in which

R¹³ is —Cl or —CH₃,

R¹⁴ is —COOH or —COOC₂H₅, and

X is —CH₂—,

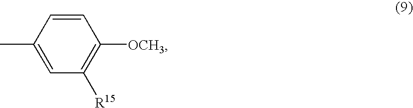

in which

R¹⁵ is —COOH or —COOC₂H₅, and

X is —CH₂—, or

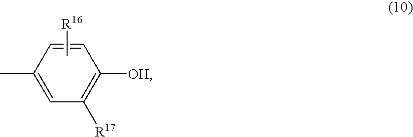

in which

R¹⁶ is —CH₃ or —OCH₃, and

R¹⁷ is —COOH, —COOCH₃ or —COOC₂H₅.

More preferred embodiments of the object compound [I] are as follows:

Y is

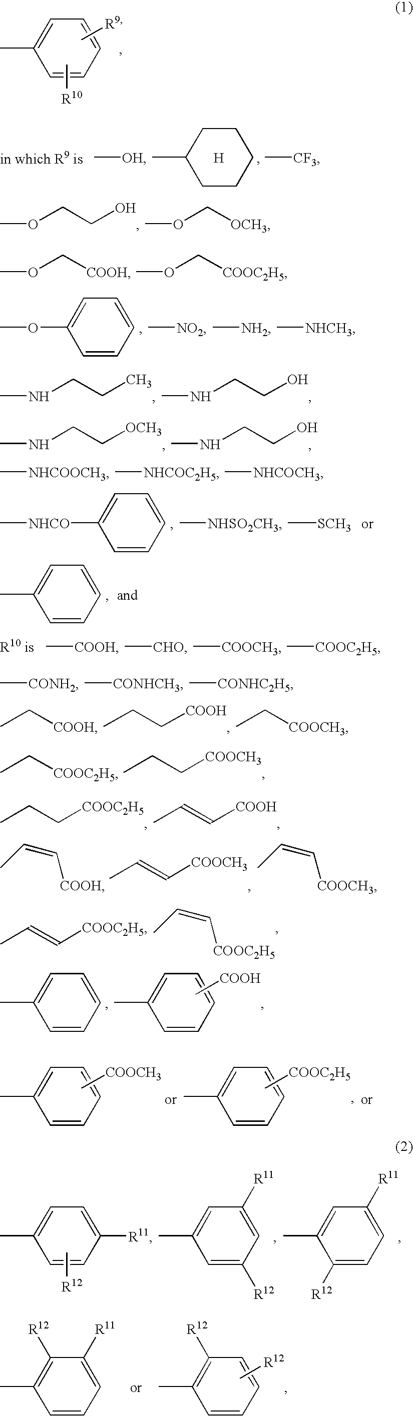

in which

R¹¹ is —F, —Cl or —CH₃, and

R¹² is —COOH, —CHO, —COOCH₃, —COOC₂H₅, —CONH₂, —CONHCH₃, —CONHC₂H₅,

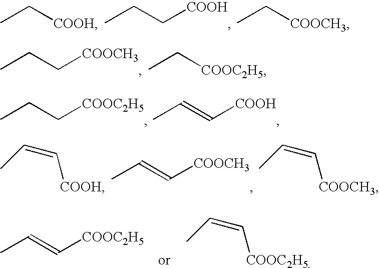

And, more preferred embodiments of the object compound [I] are as follows:

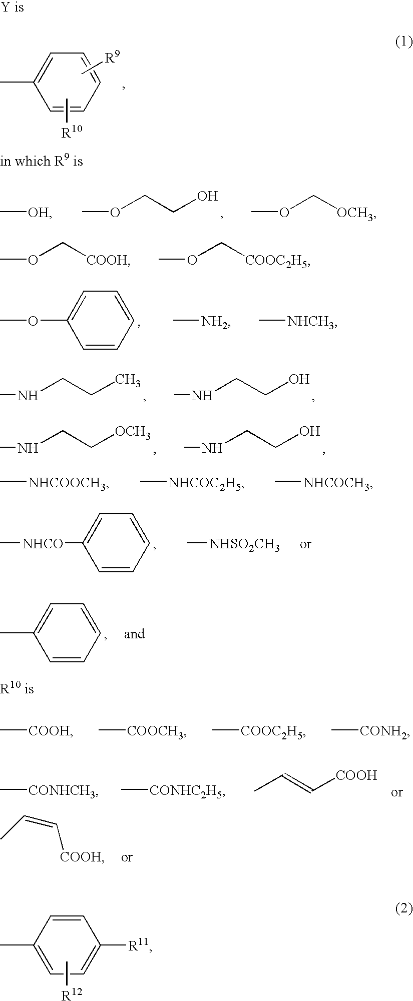

in which

R¹¹ is —CH₃, and

R¹² is —COOH, —COOCH₃, —COOC₂H₅. —CONH₂, —CONHCH₃, —CONHC₂H₅,

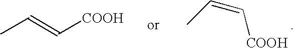

The following Preparations and Examples are given for the purpose of illustrating this invention.

Preparation 1

Under nitrogen, to a mixture of 2-phenylethanamine (40 g) and triethylamine (59.8 ml) in tetrahydrofuran (250 ml) was added trifluoromethanesulfonic anhydride (51.3 ml) dropwise under ice-water cooling, and the mixture was stirred at the same temperature for 1 hour. The resulting mixture was poured into saturated aqueous sodium bicarbonate and the aqueous mixture was extracted with ethyl acetate. The organic layer was dried over anhydrous magnesium sulfate, evaporated under reduced pressure and dried in vacuo to give 2,2,2-trifluoro-N-(2-phenylethyl)acetamide (67.73 g).

NMR (CHCl₃, δ): 2.89 (2H, t, J=7 Hz), 3.64 (2H, q, J=7 Hz), 7.20-7.40 (5H, m)

Preparation 2

The following compound was obtained according to a similar manner to that of Preparation 48.

(R)-[2-[(Trifluoroacetyl)amino]propyl]benzenesulfonyl chloride

NMR (DMSO-d₆, δ): 2.83 (2H, t, J=7 Hz), 3.40 (2H, q, J=7 Hz), 7.10-7.20 (2H, m), 7.40-7.60 (2H, m)

Preparation 3

Under nitrogen atmosphere, to a suspension of zinc powder (2.29 g) in 1,2-dichloroethane (10 ml) was added dichlorodimethylsilane (4.3 ml). The mixture was heated to 55° C. whereupon a solution of 4-[2-[(trifluoroacetyl)amino]-ethyl]benzenesulfonyl chloride (3.15 g) and 1,3-dimethyl-2-imidazolidinone (3.3 ml) in 1,2-dichloroethane (10 ml) was added dropwise while keeping the temperature below 75° C. The mixture was stirred at 70° C. for 1.5 hours and allowed to cool to room temperature. Methanol (5 ml) was added to the mixture and the mixture was stirred at room temperature for 30 minutes. The mixture was partitioned between ethyl acetate and water. The organic layer was separated, washed successively with water and brine, dried over magnesium sulfate, and filtered. The filtrate was concentrated, and the residue was purified by column chromatography (silica gel, hexane/ethyl acetate) to give 2,2,2-trifluoro-N-[2-(4-mercaptophenyl)ethyl]acetamide (1.48 g) as a white powder.

NMR (CDCl₃, δ): 2.84 (2H, t, J=7 Hz), 3.44 (1H, s), 3.59 (2H, q, J=7 Hz), 6.27 (1H, br s), 7.06 (2H, d, J=8 Hz), 7.25 (2H, d, J=8 Hz)

(+)ESI-MS (m/z): 272 (M+Na)⁺

Preparation 4

Under nitrogen at room temperature, to a solution of 2,2,2-trifluoro-N-[2-(4-mercaptophenyl)ethyl]acetamide (1.0 g) in N,N-dimethylformamide (20 ml) were added 4-chloro-2-pyridinecarboxylic acid (695 mg) and potassium carbonate (1.22 g), and the mixture was stirred at 100° C. for 26 hours. The mixture was cooled to room temperature, and iodoethane (0.355 ml) was added. After being stirred at the same temperature for 12 hours, the resulting mixture was poured into saturated aqueous sodium bicarbonate and the aqueous mixture was extracted with ethyl acetate. The organic layer was washed successively with water two times and brine, dried over anhydrous magnesium sulfate and evaporated under reduced pressure. The residue was purified by column chromatography on silica gel (hexane/ethyl acetate=3:1 to 1:2) to give ethyl 4-[[2-[2-[(trifluoroacetyl)amino]ethyl]-phenyl]thio]-2-pyridinecarboxylate (713 mg).

NMR (CDCl₃, δ): 1.41 (3H, t, J=7.1 Hz), 2.97 (2H, t, J=7.1 Hz), 3.6-3.7 (2H, m), 4.43 (2H, q, J=7.1 Hz), 7.0-7.05 (1H, m), 7.31 (2H, d, J=8.1 Hz), 7.53 (2H, d, J=8.1 Hz), 7.76 (1H, d, J=1.9 Hz), 8.44 (1H, d, J=5.4 Hz)

(+)ESI-MS (m/z): 399 (M+H)⁺

Preparation 5

To a solution of ethyl 4-[[4-[2-[(trifluoroacetyl)-amino]ethyl]phenyl]thio]-2-pyridinecarboxylate (631 mg) in a mixture of ethanol (6.3 ml) and methanol (10 ml) was added 1N sodium hydroxide at room temperature, and the mixture was stirred at the same temperature overnight. To the resulting mixture was added 1N hydrochloric acid (6.3 ml) and the mixture was evaporated under reduced pressure. Under nitrogen, the mixture of the obtained product and a reagent of 10-20% hydrogen chloride in methanol (20 ml) was refluxed for 24 hours. After evaporation, to a mixture of the residue in a mixture of tetrahydrofuran (5 ml) and water (5 ml) was added a solution of di-tert-butyl dicarbonate (691 mg) in tetrahydrofuran (3 ml) with adjusting pH to around 8 by 5N sodium hydroxide at room temperature. After being stirred at the same temperature for 1.5 hours, to the resulting mixture was added ethyl acetate followed by separation. The organic layer was washed with brine, dried over anhydrous magnesium sulfate and evaporated under reduced pressure. The residue was purified by column chromatography on silica gel (hexane/ethyl acetate=1:1 to 1:5) to give methyl 4-[[4-[2-[(tert-butoxycarbonyl)amino]-ethyl]phenyl]thio]-2-pyridinecarboxylate (470 mg).

NMR (CDCl₃, δ): 1.44 (9H, s), 2.87 (2H, t, J=7.0 Hz), 3.35-3.5 (2H, m), 3.97 (3H, s), 7.0-7.05 (1H, m), 7.32 (2H, d, J=8.1 Hz), 7.50 (2H, d, J=8.1 Hz), 7.82 (1H, d, J=1.9 Hz), 8.44 (1H, d, J=5.2 Hz)

(+)ESI-MS (m/z): 411 (M+Na)⁺

Preparation 6

Under nitrogen at 5° C., to a solution of methyl 4-[[4-[2-[(tert-butoxycarbonyl)amino]ethyl]phenyl]thio]-2-pyridinecarboxylate (461 mg) in dichloromethane (10 ml) was added m-chloroperoxybenzoic acid (655 mg), and the mixture was stirred at room temperature for 3.5 hours. The resulting mixture was poured into aqueous sodium thiosulfate and the aqueous mixture was extracted with ethyl acetate. The organic layer was washed successively with saturated aqueous sodium bicarbonate two times and brine, dried over anhydrous magnesium sulfate, evaporated under reduced pressure and dried in vacuo to give methyl 4-[[4-[2-[(tert-butoxycarbonyl)amino]ethyl]phenyl]sulfonyl]-2-pyridinecarboxylate (514 mg).

NMR (CDCl₃, δ): 1.39 (9H, s), 2.88 (2H, d, J=6.9 Hz), 3.3-3.45 (2H, m), 4.04 (3H, s), 7.40 (2H, d, J=8.3 Hz), 7.85-8.0 (3H, m), 8.54 (1H, m), 8.95 (1H, d, J=5.1Hs)

(+)ESI-MS (m/z): 443 (M+Na)⁺

Preparation 7

Under nitrogen at room temperature, a solution of methyl 4-([4-[2-[(tert-butoxycarbonyl)amino]ethyl]phenyl]-sulfonyl]-2-pyridinecarboxylate (500 mg) and hydrogen chloride (4N in ethyl acetate, 4 ml) in ethyl acetate (4 ml) was stirred for 3 hours. The resulting mixture was evaporated under reduced pressure. The residue was dissolved into a mixture of saturated aqueous sodium bicarbonate and chloroform. After separation, the organic layer was dried over anhydrous magnesium sulfate, evaporated under reduced pressure and dried in vacuo to give methyl 4-[[4-(2-aminoethyl)phenyl]sulfonyl]-2-pyridinecarboxylte (346 mg).

NMR (DMSO-d₆, δ): 2.6-2.85 (4H, m), 3.8-3.9 (3H, m), 7.05-7.2 (2H, m), 7.35-7.5 (2H, m), 7.75-8.2 (3H, m)

(+)ESI-MS (m/z): 321 (M+H)⁺

Preparation 8

The following compound was obtained according to a similar manner to that of Preparation 44.

N-[2-[4-[(3,4-Dihydroxyphenyl)sulfonyl]phenyl]ethyl]-2,2,2-trifluoroacetamide

NMR (DMSO-d₆, δ): 2.86 (2H, t, J=7.0 Hz), 3.2-3.5 (2H, m), 6.89 (1H, d, J=8.4 Hz), 7.2-7.3 (2H, m), 7.42 (2H, d, J=8.3 Hz), 7.78 (2H, d, J=8.3 Hz)

(+)ESI-MS (m/z): 412 (M+Na)⁺

Preparation 9

Under nitrogen at 5° C., to a solution of N-[2-[4-[(3,4-dihydroxyphenyl)sulfonyl]phenyl]ethyl]-2,2,2-trifluoroacetamide (8.68 g) in N,N-dimethylformamide (86 ml) were added potassium carbonate (3.39 g) and benzyl bromide (2.92 ml), and the mixture was stirred at room temperature for 36 hours. The resulting mixture was poured into 1N hydrochloric acid and the aqueous mixture was extracted with ethyl acetate. The organic layer was washed successively with water two times and brine, dried over anhydrous magnesium sulfate and evaporated under reduced pressure. The residue was purified by column chromatography on silica gel (hexane/ethyl acetate=2:1 to 4:3) to give N-[2-[4-[[4-(benzyloxy)-3-hydroxyphenyl]sulfonyl]phenyl]ethyl]-2,2,2-trifluoroacetamide (4.38 g).

NMR (CDCl₃, δ): 2.93 (2H, t, J=7.1 Hz), 3.5-3.7 (2H, m), 5.15 (2H, s), 6.95-7.1 (1H, m), 7.2-7.6 (9H, m), 7.8-7.9 (2H, m)

(+)ESI-MS (m/z): 502 (M+Na)⁺

Preparation 10

Under nitrogen at 5° C., to a solution of N-[2-[4-[[4-(benzyloxy)-3-hydroxyphenyl]sulfonyl]phenyl]ethyl]-2,2,2-trifluoroacetamide (1.68 g) and 2,6-lutidine (0.527 ml) in dichloromethane (50 ml) was added trifluoromethanesulfonic anhydride (0.648 ml), and the mixture was stirred at the same temperature for 1 hour. The resulting mixture was poured into aqueous ammonia and the aqueous mixture was extracted with ethyl acetate. The organic layer was washed successively with 1N hydrochloric acid, water, saturated aqueous sodium bicarbonate and brine, dried over anhydrous magnesium sulfate and evaporated under reduced pressure. The residue was purified by column chromatography on silica gel (hexane/ethyl acetate=2:1 to 4:3) to give 2-(benzyloxy)-5-[[4-[2-[(trifluoroacetyl)amino]ethyl]phenyl]-sulfonyl]phenyl trifluoromethanesulfonate (1.59 g).

NMR (CDCl₃, δ): 2.9-3.0 (2H, m), 3.55-3.7 (2H, m), 5.23 (2H, s), 7.15 (1H, d, J=8.7 Hz), 7.3-7.45 (7H, m), 7.75-7.9 (4H, m)

(+)ESI-MS (m/z): 634 (M+Na)⁺

Preparation 11

Under nitrogen at room temperature, to a solution of 2-(benzyloxy)-5-[[4-[2-[(trifluoroacetyl)amino]ethyl]phenyl]-sulfonyl]phenyl trifluoromethanesulfonate (1.58 g) in N,N-dimethylformamdie (12 ml) were added palladium(II)acetate (29 mg), 1,3-bis(diphenylphosphino)propane (53 mg), ethanol (6 ml) and triethylamine (1.08 ml), and under carbon monoxide (1 atm), the mixture was stirred at 60° C. for 2 hours. The resulting mixture was poured into water and the aqueous mixture was extracted with ethyl acetate. The organic layer was washed successively with water two times and brine, dried over anhydrous magnesium sulfate and evaporated under reduced pressure. The residue was purified by column chromatography on silica gel (hexane/ethyl acetate=2:1 to 4:3) to give ethyl 2-(benzyloxy)-5-[[4-[2-[(trifluoroacetyl)amino]ethyl]phenyl]sulfonyl]benzoate (959 mg).

NMR (CDCl₃, δ): 1.34 (3H, t, J=7.1 Hz), 2.85-3.0 (2H, m) 3.5-3.65 (2H, m), 4.37 (2H, q, J=7.1 Hz), 5.22 (2H, s), 7.10 (1H, d, J=8.9 Hz), 7.25-7.5 (5H, m), 7.85-7.9 (2H, m), 7.99 (1H, dd, J=2.5, 8.7 Hz), 8.33 (1H, d, J=2.5 Hz)

(+)ESI-MS (m/z): 558 (M+Na)⁺

Preparation 12

Under nitrogen at 5° C., to a solution of ethyl 2-(benzyloxy)-5-[[4-[2-[(trifluoroacetyl)amino]ethyl]phenyl]-sulfonyl]benzoate (957 mg) in N,N-dimethylformamide (15 ml) was added sodium hydride (60% in oil, 78.6 mg), and the mixture was stirred at room temperature for 30 minutes. The mixture was cooled to 5° C., and benzyl bromide (0.234 ml) was added. After being stirred at room temperature overnight, the resulting mixture was poured into water and the aqueous mixture was extracted with ethyl acetate. The organic layer was washed successively with water two times and brine, dried over anhydrous magnesium sulfate and evaporated under reduced pressure. The residue was purified by column chromatography on silica gel (hexane/ethyl acetate=2:1) to give ethyl 2-(benzyloxy)-5-[[4-[2-[benzyl(trifluoroacetyl)-amino]ethyl]phenyl]sulfonyl]benzoate (965 mg).

NMR (CDCl₃, δ): 1.33 (3H, t, J=7.1 Hz), 2.75-2.95 (2H, m), 3.4-3.55 (2H, m), 4.36 (2H, q, J=7.1 Hz), 4.45-4.70 (2H, m), 5.20 (2H, s), 7.07 (1H, d, J=8.9 Hz), 7.1-7.5 (12H, m), 7.8-8.0 (3H, m), 8.32 (1H, d, J=2.4 Hz)

(+)ESI-MS (m/z): 648 (M+Na)⁺

Preparation 13

A mixture of ethyl 2-(benzyloxy)-5-[[4-[2-[benzyl(trifluoroacetyl)amino]ethyl]phenyl]sulfonyl]benzoate (963 mg) and 10% palladium on activated carbon (50% wet, 100 mg) in ethanol (15 ml) was stirred at room temperature in the presence of hydrogen at an atmospheric pressure for 3 hours. After filtration, the filtrate was evaporated under reduced pressure followed by dryness in vacuo to give ethyl 5-[[4-[2-[benzyl(trifluoroacetyl)amino]ethyl]phenyl]-sulfonyl]-2-hydroxybenzoate (848 mg).

NMR (CDCl₃, δ): 1.45 (3H, t, J=7.1 Hz), 2.75-2.95 (2H, m), 3.4-3.55 (2H, m), 4.4-4.7 (3H, m), 7.05 (1H, d, J=8.9 Hz), 7.1-7.45 (7H, m), 7.8-7.95 (3H, m), 8.47 (1H, d, J=2.4 Hz)

(+)ESI-MS (m/z): 558 (M+Na)⁺

Preparation 14

Under nitrogen, the mixture of ethyl 5-[[4-[2-[benzyl(trifluoroacetyl)amino]ethyl]phenyl]sulfonyl]-2-hydroxybenzoate (845 mg) and hydrogen chloride (7N in ethanol, 6 ml) in ethanol (3 ml) was refluxed for 2.5 days. The resulting mixture was evaporated under reduced pressure. The residue was dissolved into a mixture of saturated aqueous sodium bicarbonate and chloroform/methanol (10:1). After separation, the organic layer was washed with brine, dried over anhydrous magnesium sulfate, evaporated under reduced pressure and dried in vacuo to give ethyl 5-[[4-[2-(benzylamino)ethyl]phenyl]sulfonyl]-2-hydroxybenzoate (630 mg).

NMR (DMSO-d₆, δ): 1.33 (3H, t, J=7.1 Hz), 2.65-2.9 (4H, m), 3.71 (2H, s), 4.35 (2H, q, J=7.1 Hz), 7.09 (1H, d, J=8.8 Hz), 7.15-7.3 (5H, m), 7.44 (2H, d, J=8.3 Hz), 7.82 (2H, d, J=8.3 Hz), 7.95 (1H, dd, J=2.5, 8.8 Hz), 8.20 (1H, d, J=2.5 Hz)

(+)ESI-MS (m/z): 440 (M+H)⁺

Preparation 15

Under nitrogen at room temperature, to a solution of N-[2-[4-[[4-(benzyloxy)-3-hydroxyphenyl]sulfonyl]phenyl]-ethyl]-2,2,2-trifluoroacetamide (1.0 g) in N,N-dimethylformamide (10 ml) were added potassium carbonate (346 mg) and chloromethyl methyl ether (0.339 ml), and the mixture was stirred at the same temperature overnight. The resulting mixture was poured into water and the aqueous mixture was extracted with ethyl acetate. The organic layer was washed successively with water two times and brine, dried over anhydrous magnesium sulfate, evaporated under reduced pressure and dried in vacuo to give N-[2-[4-[[4-(benzyloxy)-3-(methoxymethoxy)phenyl]sulfonyl]phenyl]ethyl]-2,2,2-trifluoroacetamide (1.1 g).

NMR (CDCl₃, δ): 2.85-3.0 (2H, m), 3.45-3.7 (5H, m), 5.15-5.3 (4H, m), 6.97 (1H, d, J=8.6 Hz), 7.2-7.9 (11H, m)

(+)ESI-MS (m/z): 546 (M+Na)⁺

Preparation 16

The following compounds were obtained according to a similar manner to that of Preparation 13.

(1) 2,2,2-Trifluoro-N-[2-[4-[[4-hydroxy-3-(methoxymethoxy)phenyl]sulfonyl]phenyl]ethyl]acetamide

NMR (CDCl₃, δ): 2.85-3.0 (3H, m), 3.45-3.65 (5H, m), 5.2 (2H, m), 7.02 (1H, d, J=8.4 Hz), 7.31 (2H, d, J=8.2 Hz), 7.45-7.65 (2H, m), 7.87 (2H, d, J=8.2 Hz)

(−)ESI-MS (m/z): 432 (M−H)⁻

(2) Ethyl(R)-4′-[[4-[2-(trifluoroacetyl)amino]propyl]-phenyl]sulfonyl]-1,1′-biphenyl-3-carboxylate

NMR (CDCl₃, δ): 1.22 (3H, d, J=6.5 Hz), 1.41 (3H, q, J=7.1 Hz), 2.75-3.1 (2H, m), 4.15-4.5 (3H, m), 7.2-7.4 (2H, m), 7.54 (1H, t, J=7.7 Hz), 7.65-8.15 (5H, m), 8.24 (1H, s)

(+)ESI-MS (m/z): 542 (M+Na)⁺

(3) Ethyl 5-[[4-[3-[benzyl(trifluoroacetyl)amino]propyl]-phenyl]sulfonyl]-2-hydroxybenzoate

NMR (CDCl₃, δ): 1.45 (3H, t, J=7 Hz), 1.68-2.02 (2H, m), 2.60 (2H, t, J=7 Hz), 3.30 (2H, t, J=7 Hz), 4.46 (2H, q, J=7 Hz), 4.57, 4.61 (2H, a pair of s), 6.95-7.45 (9H, m), 7.83 (2H, m), 7.92 (1H, dd, J=9, 2 Hz), 8.48 (1H, d, J=2 Hz), 11.41 (1H, s, OH)

(+)ESI-MS (m/z): 572 (M+Na)⁺

(4) 2,2,2-Trifluoro-N-[3-[4-[[4-hydroxy-3-(methoxymethoxy)phenyl]sulfonyl]phenyl]propyl]acetamide

NMR (CDCl₃, δ): 1.92 (2H, quintet, J=7 Hz), 2.72 (2H, t, J=7 Hz), 3.38 (2H, q, J=7 Hz), 3.52 (3H, s), 5.24 (2H, s), 6.33 (1H, br s), 6.43 (1H, s, OH), 7.03 (1H, d, J=9 Hz), 7.29 (2H, d, J=8 Hz), 7.55 (1H, dd, J=9, 2 Hz), 7.66 (1H, d, J=2 Hz), 7.83 (2H, d, J=8 Hz)

(+)ESI-MS (m/z): 470 (M+Na)⁺

(5) Methyl 5-[[4-2-benzyl(trifluoroacetyl)amino]ethoxy]-phenyl]sulfonyl]-2-hydroxybenzoate

NMR (CDCl₃, δ): 3.60-3.85 (2H, m), 4.00 (3H, s), 4.04-4.25 (2H, m), 4.77, 4.81 (total 2H, a pair of s), 6.92 (2H, d, J=9 Hz), 7.06 (1H, d, J=9 Hz), 7.12-7.50 (5H, m), 7.85 (2H, d, J=8 Hz), 7.93 (1H, dd, J=9, 2 Hz), 8.46 (1H, d, J=2 Hz), 11.25 (1H, br s, OH)

(+)ESI-MS (m/z): 560 (M+Na)⁺

Preparation 17

The following compounds were obtained according to a similar manner to that of Preparation 10.

(1) 2-(Methoxymethoxy)-4-[[4-[2-[(trifluoroacetyl)amino]-ethyl]phenyl]sulfonyl]phenyl trifluoromethanesulfonate

NMR (CDCl₃, δ): 2.9-3.05 (2H, m), 3.5 (3H, m), 3.55-3.7 (2H, m), 5.30 (2H, s), 7.3-7.45 (3H, m), 7.60 (1H, dd, J=2.0, 8.5 Hz), 7.8-7.95 (3H, m)

(+)ESI-MS (m/z): 588 (M+Na)⁺

(2) 2-Methyl-4-[[4-[3-[(trifluoroacetyl)amino]propyl]-phenyl]sulfonyl]phenyl trifluoromethanesulfonate

NMR (CDCl₃, δ): 1.8-2.1 (2H, m), 2.43 (3H, s), 2.65-2.8 (2H, m), 3.35-3.5 (2H, m), 7.3-7.4 (3H, m), 7.8-7.95 (4H, m)

(+)ESI-MS (m/z): 556 (M+Na)⁺

(3) tert-Butyl [4-[[(trifluoromethyl)sulfonyl]oxy]phenyl]-acetate

NMR (CDCl₃, δ): 1.44 (9H, s), 3.55 (2H, s), 7.2-7.4 (4H, m)

(+)ESI-MS (m/z): 363 (M+Na)⁺

(4) (R)-2-Chloro-4-[[4-[2-(trifluoroacetyl)amino]propyl]-phenyl]sulfonyl]phenyl trifluoromethanesulfonate

NMR (CDCl₃, δ): 1.24 (3H, d, J=6.8 Hz), 2.87 (1H, dd, J=7.3, 13.5 Hz), 3.00 (1H, dd, J=6.2, 13.5 Hz), 4.28 (1H, heptuplet, J=7.0 Hz), 6.13 (1H, d, J=7.6 Hz), 7.38 (2H, d, J=8.4 Hz), 7.49 (1H, d, J=8.7 Hz), 7.87-7.92 (3H, m), 8.09 (1H, d, J=2.2 Hz)

(+) APCI-MS (m/z): 576 (M+Na)⁺

(5) (R)-3-[[4-[2-[(2,2,2-Trifluoroacetyl)amino]propyl]-phenyl]sulfonyl]phenyl trifluoromethanesulfonate

NMR (CDCl₃, δ): 1.22 (3H, d, J=6.8 Hz), 2.8-3.05 (2H, m), 4.15-4.4 (1H, m), 7.36 (2H, d, J=8.3 Hz), 7.45-7.5 (1H, m), 7.63 (1H, t, J=8.2 Hz), 7.8-8.0 (4H, m)

(+)ESI-MS (m/z): 542 (M+Na)⁺

(6) 2-Benzyloxy-5-[[4-[3-[(trifluoroacetyl)amino]propyl]-phenyl]sulfonyl]phenyl trifluoromethanesulfonate

NMR (CDCl₃, δ): 1.93 (2H, quintet, J=7 Hz), 2.73 (2H, t, J=7 Hz), 3.39 (2H, q, J=7 Hz), 5.23 (2H, s), 6.29 (1H, br s), 7.08-7.50 (9H, m), 7.75-7.93 (3H, m)

(+)ESI-MS (m/z): 648 (M+Na)⁺

(7) 2-Methoxymethoxy-4-[[4-[3-[(trifluoroacetyl)amino]-propyl]phenyl]sulfonyl]phenyl trifluoromethnesulfonate

NMR (CDCl₃, δ): 1.94 (2H, quintet, J=7 Hz), 2.75 (2H, t, J=7 Hz), 3.40 (2H, q, J=7 Hz), 3.51 (3H, s), 5.30 (2H, s), 6.31 (1H, br s), 6.95 (1H, d, J=8 Hz), 7.33 (2H, d, J=8 Hz), 7.60 (1H, dd, J=8, 2 Hz), 7.86 (1H, d, J=2 Hz), 7.88 (2H, d, J=8 Hz)

(+)ESI-MS (m/z): 602 (M+H)⁺

(8) 2-Chloro-4-[[4-[3-[(trifluoroacetyl)amino]propyl]-phenyl]sulfonyl]phenyl trifluoromethanesulfonate

NMR (CDCl₃, δ): 1.95 (2H, quintet, J=7 Hz), 2.76 (2H, t, J=7 Hz), 3.40 (2H, q, J=7 Hz), 6.31 (1H, br s), 7.38 (2H, d, J=8 Hz), 7.49 (1H, d, J=9 Hz), 7.88 (2H, d, J=8 Hz), 7.91 (1H, dd, J=9, 2 Hz), 8.09 (1H, d, J=2 Hz)

(+)ESI-MS (m/z): 576 (M+H)⁺

Preparation 18

The following compounds were obtained according to a similar manner to that of Preparation 11.

(1) Ethyl 2-(methoxymethoxy)-4-[[4-[2-[(trifluoroacetyl)amino]ethyl]phenyl]sulfonyl]benzoate

NMR (CDCl₃, δ): 1.36 (3H, t, J=7.1 Hz), 2.96 (2H, t, J=7.1 Hz), 3.50 (3H, s), 3.55-3.7 (2H, m), 4.36 (2H, q, J=7.1 Hz), 5.28 (2H, s), 7.35 (2H, d, J=8.3 Hz), 7.57 (1H, dd, J=1.5, 8.1 Hz), 7.75 (1H, d, J=1.5 Hz), 7.80 (1H, d, J=8.1 Hz), 7.85-7.95 (2H, m)

(+)ESI-MS (m/z): 512 (M+Na)⁺

(2) Ethyl 2-methyl-4-[[4-[3-[(trifluoroacetyl)amino]-propyl]phenyl]sulfonyl]benzoate

NMR (CDCl₃, δ): 1.30 (3H, t, J=7.1 Hz), 1.7-1.9 (2H, m), 2.55 (3H, m), 2.6-2.75 (2H, m), 3.1-3.25 (2H, m), 4.31 (2H, d, J=7.1 Hz), 7.55-7.65 (2H, m), 7.8-8.0 (5H, m)

(+)ESI-MS (m/z): 480 (M+Na)⁺

(3) Ethyl 2-benzyloxy-5-[[4-[3-[(trifluoroacetyl)amino]-propyl]phenyl]sulfonyl]benzoate

NMR (CDCl₃, δ): 1.34 (3H, t, J=7 Hz), 1.91 (2H, quintet, J=7 Hz), 2.71 (2H, t, J=7 Hz), 3.37 (2H, q, J=7 Hz), 4.36 (2H, q, J=7 Hz), 5.21 (2H, s), 6.39 (1H, br s), 7.08 (1H, d, J=9 Hz), 7.20-7.55 (7H, m), 7.84 (2H, d, J=8 Hz), 7.98 (1H, dd, J=9, 2 Hz), 8.33 (1H, d, J=2 Hz)

(+)ESI-MS (m/z): 572 (M+Na)⁺

(4) Ethyl 2-methoxymethoxy-4-[[4-[3-[(trifluoroacetyl)-amino]propyl]phenyl]sulfonyl]benzoate

NMR (CDCl₃, δ): 1.36 (3H, t, J=7 Hz), 1.92 (2H, quintet, J=7 Hz), 2.73 (2H, t, J=7 Hz), 3.38 (2H, q, J=7 Hz), 3.50 (3H, s), 4.36 (2H, q, J=7 Hz), 5.28 (2H, s), 6.37 (1H, br s), 7.33 (2H, d, J=8 Hz), 7.55 (1H, dd, J=8, 2 Hz), 7.75 (1H, d, J=2 Hz), 7.79 (1H, d, J=2 Hz), 7.86 (2H, d, J=8 Hz)

(+)ESI-MS (m/z): 526 (M+Na)⁺

(5) Ethyl 2-chloro-4-[[4-[3-[(trifluoroacetyl)amino]-propyl]phenyl]sulfonyl]benzoate

NMR (CDCl₃, δ): 1.39 (3H, t, J=7 Hz), 1.93 (2H, quintet, J=7 Hz), 2.74 (2H, t, J=7 Hz), 3.39 (2H, q, J=7 Hz), 4.41 (2H, q, J=7 Hz), 6.31 (1H, br s), 7.35 (2H, d, J=8 Hz), 7.75-7.94 (4H, m), 8.00 (1H, d, J=2 Hz)

(+)ESI-MS (m/z): 500 (M+Na)⁺

Preparation 19

The following compounds were obtained according to a similar manner to that of Preparation 12.

(1) Ethyl 4-[[4-[2-[benzyl(trifluoroacetyl)amino]ethyl]-phenyl]sulfonyl]-2-(methoxymethoxy)benzoate

NMR (CDCl₃, δ): 1.35 (3H, t, J=7.2 Hz), 2.75-2.95 (2H, m), 3.4-3.55 (5H, m), 4.36 (2H, q, J=7.2 Hz), 4.45-4.7 (2H, m), 5.27 (2H, s), 7.1-7.4 (7H, m), 7.45-7.55 (1H, m), 7.7-7.9 (4H, m)

(+)ESI-MS (m/z): 602 (M+Na)⁺

(2) Ethyl 4′-[[4-[2-[benzyl(trifluoroacetyl)amino]ethyl]-phenyl]sulfonyl]-2′-(methoxymethoxy)1,1′-biphenyl-3-carboxylate

NMR (CDCl₃, δ): 1.38 (3H, t, J=7.1 Hz), 2.8-2.95 (2H, m) 3.37 (3H, s), 3.45-3.55 (2H, m), 4.36 (2H, q, J=7.1 Hz), 4.5-4.7 (2H, m), 5.36 (2H, s), 7.15-7.5 (9H, m), 7.6-7.65 (2H, m), 7.75 (1H, m), 7.85-7.90 (2H, m), 8.05-8.1 (1H, m), 8.13 (1H, m)

(+)ESI-MS (m/z): 678 (M+Na)⁺

(3) Ethyl 4-[[4-[3-[benzyl(trifluoroacetyl)amino]propyl]-phenyl]sulfonyl]-2-(methoxymethoxy)benzoate

NMR (CDCl₃, δ): 1.36 (3H, t, J=7 Hz), 1.72-2.00 (2H, m), 2.59-2.66 (2H, a pair of t, J=7 Hz), 3.31, 3.33 (2H, a pair of t, J=7 Hz), 3.50 (3H, s), 4.36 (2H, q, J=7 Hz), 4.57, 4.61 (2H, a pair of s), 5.28 (2H, s), 7.10-7.42 (7H, m), 7.55 (1H, dd, J=8, 2 Hz), 7.70-7.95 (4H, m)

(4) Ethyl 2-benzyloxy-5-[[4-[3-[benzyl(trifluoroacetyl)-amino]propyl]phenyl]sulfonyl]benzoate

NMR (CDCl₃, δ): 1.34 (3H, t, J=7 Hz), 1.65-2.00 (2H, m), 2.58 (2H, t, J=7 Hz), 3.30 (2H, m), 4.36 (2H, q, J=7 Hz), 4.56, 4.61 (2H, a pair of s), 5.21 (2H, s) 7.00-7.50 (13H, m), 7.81 (2H, m), 7.97 (1H, dd, J=9, 2 Hz), 8.34 (1H, d, J=2 Hz)

(+)ESI-MS (m/z): 662 (M+Na)⁺

Preparation 20

The following compounds were obtained according to a similar manner to that of Preparation 14.

(1) Ethyl 4-[[4-[2-(benzylamino)ethyl]phenyl]sulfonyl]-2-hydroxybenzoate

NMR (CDCl₃, δ): 1.30 (3H, t, J=7.1 Hz), 2.65-2.9 (4H, m) 3.68 (2H, s), 4.33 (2H, q, J=7.1 Hz), 7.1-7.3 (5H, m), 7.35-7.05 (4H, m), 7.8-7.9 (3H, m)

(+)ESI-MS (m/z): 440 (M+H)⁺

(2) Ethyl(R)-3-[4-[[4-(2-aminopropyl)phenyl]sulfonyl]-phenoxy]benzoate

NMR (CDCl₃, δ): 1.12 (3H, d, J=6.2 Hz), 1.38 (3H, t, J=7.2 Hz), 2.5-2.7 (2H, m), 3.1-3.2 (1H, m), 4.37 (2H, q, J=7.2 Hz), 6.95-7.1 (2H, m), 7.2-7.4 (3H, m), 7.47 (1H, t, J=8.0 Hz), 7.7 (1H, m), 7.8-8.0 (5H, m)

(+)ESI-MS (m/z): 440 (M+H)⁺

(3) Ethyl 4-[[4-(3-aminopropyl)phenyl]sulfonyl]-2-methylbenzoate

NMR (CDCl₃₁ 5): 1.38 (3H, t, J=7.1 Hz), 1.6-1.85 (2H, m) 2.62 (3H, s), 2.65-2.8 (4H, m), 4.36 (2H, q, J=7.1 Hz), 7.33 (2H, d, J=8.3 Hz), 7.7-7.9 (4H, m), 7.96 (1H, d, J=8.1 Hz)

(+)ESI-MS (m/z): 362 (M+H)⁺

(4) (R)-Ethyl 3-[3-[[4-(2-aminopropyl)phenyl]sulfonyl]-phenoxy]benzoate

NMR (CDCl₃, δ): 1.12 (3H, d, J=6.4 Hz), 1.39 (3H, t, J=7.2 Hz), 2.55-2.8 (2H, m), 3.1-3.3 (1H, m), 4.38 (2H, q, J=7.2 Hz), 7.1-7.7 (9H, m), 7.8-7.9 (3H, m)

(+)ESI-MS (m/z): 440 (M+H)⁺

(5) Ethyl(R)-4′-[[4-(2-aminopropyl)phenyl]sulfonyl]-1,1′-biphenyl-3-carboxylate

NMR (CDCl₃, δ): 1.12 (3H, d, J=6.2 Hz), 1.41 (3H, t, J=7.2 Hz), 2.5-2.8 (2H, m), 3.1-3.3 (1H, m), 4.41 (2H, q, J=7.2 Hz), 7.35 (2H, d, J=8.3 Hz), 7.54 (1H, t, J=7.8 Hz), 7.7-8.15 (8H, m), 8.24 (1H, m)

(+)ESI-MS (m/z): 424 (M+H)⁺

(6) Ethyl(R)-3′-[[4-(2-aminopropyl)phenyl]sulfonyl]-1,1′-biphenyl-3-carboxylate

NMR (CDCl₃, δ): 1.11 (3H, d, J=6.3 Hz), 1.41 (3H, t, J=7.2 Hz), 2.5-2.8 (2H, m), 3.1-3.25 (1H, m), 4.43 (2H, q, J=7.2 Hz), 7.34 (2H, d, J=8.3 Hz), 7.4-8.3 (10H, m)

(+)ESI-MS (m/z): 424 (M+H)⁺

(7) Ethyl 4′-[[4-[2-(benzylamino)ethyl]phenyl]sulfonyl]-2′-hydroxy-1,1′-biphenyl-3-carboxylate

NMR (DMSO-d₆, δ): 1.31 (3H, t, J=7.1 Hz), 2.65-2.8 (4H, m), 3.69 (2H, s), 4.33 (2H, q, J=7.1 Hz), 7.15-7.65 (11H, m), 7.75-8.0 (4H, m), 8.1-8.15 (1H, m)

(+)ESI-MS (m/z): 516 (M+H)⁺

(8) Ethyl(R)-4-[[4-[(2-aminopropyl)oxy]phenyl]-sulfonyl]benzoate

NMR (CDCl₃, δ): 1.17 (3H, d, J=6.5 Hz), 1.39 (3H, t, J=7.2 Hz), 3.25-3.45 (1H, m), 3.65-3.8 (1H, m), 3.85-3.95 (1H, m), 4.39 (2H, q, J=7.2 Hz), 6.95-7.0 (2H, m), 7.8-8.0 (4H, m), 8.1-8.2 (2H, m)

(+)ESI-MS (m/z): 364 (M+H)⁺

(9) Ethyl 5-[[4-[3-(benzylamino)propyl]phenyl]sulfonyl]-2-hydroxybenzoate

NMR (DMSO-d₆, δ): 1.32 (3H, t, J=7 Hz), 1.78 (2H, quintet, J=7 Hz), 2.61 (2H, t, J=7 Hz), 2.69 (2H, t, J=7 Hz), 3.82 (2H, s), 4.33 (2H, q, J=7 Hz), 7.07 (1H, d, J=9 Hz), 7.20-7.42 (5H, m), 7.42 (2H, d, J=8 Hz), 7.82 (2H, d, J=8 Hz), 7.91 (1H, dd, J=9, 2 Hz), 8.19 (1H, d, J=2 Hz)

(+) APCI-MS (m/z): 454 (M+H)⁺

(10) Ethyl 4-[[4-[3-(benzylamino)propyl]phenyl]sulfonyl]-2-hydroxybenzoate

NMR (DMSO-d₆, δ): 1.30 (3H, t, J=7 Hz), 1.82 (2H, quintet, J=7 Hz), 2.65 (2H, t, J=7 Hz), 2.72 (2H, t, J=7 Hz), 3.87 (2H, s), 4.33 (2H, q, J=7 Hz), 7.10-7.55 (9H, m), 7.87 (2H, d, J=8 Hz), 7.88 (1H, d, J=8 Hz)

(11) Ethyl 5-[[4-[2-(benzylamino)ethoxy]phenyl]sulfonyl]-2-hydroxybenzoate

NMR (CDCl₃, δ): 1.45 (3H, t, J=7 Hz), 3.03 (2H, t, J=5 Hz), 3.87 (2H, s), 4.13 (2H, t, J=5 Hz), 4.45 (2H, q, J=7 Hz), 6.92 (2H, d, J=9 Hz), 7.04 (1H, d, J=9 Hz), 7.15-7.47 (5H, m), 7.84 (2H, d, J=9 Hz), 7.90 (1H, dd, J=9, 2 Hz), 8.46 (1H, d, J=2 Hz)

(+)ESI-MS (m/z): 456 (M+H)⁺

(12) Ethyl 4-[[4-(3-aminopropyl)phenyl]sulfonyl]-2-chlorobenzoate

NMR (DMSO-d₆, δ): 1.30 (3H, t, J=7 Hz), 1.84 (2H, quintet, J=7 Hz), 2.60-2.88 (4H, m), 4.35 (2H, q, J=7 Hz), 7.51 (2H, d, J=8 Hz), 7.85-8.10 (4H, m), 8.14 (1H, s)

(+)ESI-MS (m/z): 382 (M+H)⁺

(13) Ethyl 5-[[4-(3-aminopropyl)phenyl]sulfonyl]-2-methoxybenzoate

NMR (DMSO-d₆, δ): 1.29 (3H, t, J=7 Hz), 1.67 (2H, quintet, J=7 Hz), 2.35-2.80 (4H, m), 3.90 (3H, s), 4.28 (2H, q, J=7 Hz), 7.36 (1H, d, J=9 Hz), 7.44 (2H, d, J=8 Hz), 7.86 (2H, d, J=8 Hz), 8.09 (1H, dd, J=9, 2 Hz), 8.12 (1H, d, J=2 Hz)

(+)ESI-MS (m/z): 378 (M+H)⁺

Preparation 21

To a solution of 2,2,2-trifluoro-N-[(1R)-1-methyl-2-phenylethyl]acetamide (3.75 g) in acetic acid (32 ml)-water (6.5 ml)-sulfuric acid (0.97 ml) were added iodine (1.65 g) and periodic acid dihydrate (740 mg) at room temperature, and the mixture was heated to 60-80° C. for 5 hours. After being allowed to cool to room temperature, the mixture was partitioned between hexane/ethyl acetate and water. The organic layer was separated, washed successively with water, sodium sulfite solution, water, and brine, dried over magnesium sulfate, and filtered. The filtrate was concentrated and the residue was recrystallized from diisopropyl ether (44 ml) to give 2,2,2-trifluoro-N-[(1R)-2-(4-iodophenyl)-1-methylethyl]acetamide (2.15 g) as a colorless needle.

NMR (CDCl₃, δ): 1.21 (3H, d, J=7 Hz), 2.74 (1H, dd, J=14, 7 Hz), 2.85 (1H, dd, J=14, 6 Hz), 4.26 (1H, m), 6.04 (1H, br s), 6.92 (2H, d, J=8 Hz), 7.65 (2H, d, J=8 Hz)

(+)ESI-MS (m/z): 380 (M+Na)⁺

Preparation 22

Under nitrogen at room temperature, to a mixture of bis(dibenzylideneacetone)palladium(0) (403 mg) and bis(2-diphenylphosphinophenyl)ether (407 mg) was added toluene (90 ml). After being stirred at the same temperature for 15 minutes, (R)-2,2,2-trifluoro-N-[2-(4-iodophenyl)-1-methylethyl]acetamide (5 g), potassium tert-butoxide (1.89 g) and 4-methoxybenzenethiol (1.89 ml) were added, and the mixture was stirred at 80° C. for 3 hours. The resulting mixture was poured into water and the aqueous mixture was extracted with ethyl acetate. The organic layer was washed successively with water and brine, dried over anhydrous magnesium sulfate and evaporated under reduced pressure. The residue was purified by column chromatography on silica gel (hexane/ethyl acetate=10:1 to 5:1) to give (R)-2,2,2-trifluoro-N-[2-[4-[(4-methoxyphenyl)thio]phenyl]-1-methylethyl]acetamide (4.39 g).

NMR (DMSO-d₆, δ): 1.14 (3H, d, J=6.7 Hz), 2.73 (2H, d, J=7.1 Hz), 3.77 (3H, s), 3.9-4.1 (1H, m), 6.9-7.2 (6H, m), 7.3-7.4 (2H, m)

(+)ESI-MS (m/z): 392 (M+H)⁺

Preparation 23

Under nitrogen at 5° C., to a solution of (R)-2,2,2-trifluoro-N-[2-[4-[(4-methoxyphenyl)thio]phenyl]-1-methylethyl]acetamide (4.38 g) in dichloromethane (88 ml) was added boron tribromide (1M in dichloromethane, 35.6 ml) dropwise, and the mixture was stirred at room temperature overnight. The resulting mixture was evaporated under reduced pressure. The residue was dissolved into a mixture of saturated aqueous sodium bicarbonate and ethyl acetate. After separation, the organic layer was washed successively with water and brine, dried over anhydrous magnesium sulfate, evaporated under reduced pressure and dried in vacuo to give (R)-2,2,2-trifluoro-N-[2-[4-[(4-hydroxyphenyl)thio]phenyl]-1-methylethyl]acetamide (3.97 g).

NMR (CDCl₃, δ): 1.20 (3H, d, J=6.6 Hz), 2.65-2.9 (2H, m), 4.1-4.35 (1H, m), 6.75-6.9 (2H, m), 6.95-7.15 (4H, m), 7.3-7.4 (2H, m)

(+)ESI-MS (m/z): 378 (M+Na)⁺

Preparation 24

A mixture of (R)-2,2,2-trifluoro-N-[2-[4-[(4-hydroxyphenyl)thio]phenyl]-1-methylethyl]acetamide (500 mg), 3-ethoxycarbonylphenylboronic acid (546 mg), copper(II) acetate (256 mg), powdered molecular sieves 4 Å (500 mg) and pyridine (0.569 ml) in dichloromethane (15 ml) was stirred at room temperature for 4 days. After the resulting mixture was filtered with celite, the filtrate was poured into 0.1N hydrochloric acid and the aqueous mixture was extracted with ethyl acetate. The organic layer was washed successively with saturated aqueous sodium bicarbonate and brine, dried over anhydrous magnesium sulfate and evaporated under reduced pressure. The residue was purified by column chromatography on silica gel (hexane/ethyl acetate=5:1) to give ethyl(R)-3-[4-[[4-[2-[(trifluoroacetyl)amino]propyl]-phenyl]thio]phenoxy]benzoate (463 mg).

NMR (CDCl₃, δ): 1.22 (3H, d, J=6.6 Hz), 1.39 (3H, t, J=6.9 Hz), 2.7-2.95 (2H, m), 4.15-4.45 (3H, m), 6.9-7.85 (12H, m)

(+)ESI-MS (m/z): 526 (M+Na)⁺

Preparation 25

The following compounds were obtained according to a similar manner to that of Preparation 6.

(1) Ethyl(R)-3-[4-[[4-[2-[(trifluoroacetyl)amino]propyl]-phenyl]sulfonyl]phenoxy]benzoate

NMR (CDCl₃, δ): 1.22 (3H, d, J=6.6 Hz), 1.37 (3H, t, J=7.1 Hz), 2.75-3.05 (2H, m), 4.15-4.45 (3H, m), 6.95-7.1 (2H, m), 7.2-7.4 (3H, m), 7.47 (1H, t, J=8.0 Hz), 7.7 (1H, m), 7.85-7.95 (5H, m)

(+)ESI-MS (m/z): 558 (M+Na)⁺

(2) tert-Butyl benzyl[2-[4-[[4-(2-formylphenoxy)phenyl]-sulfonyl]phenyl]ethyl]carbamate

NMR (CDCl₃, δ): 1.41 (9H, s), 2.7-2.9 (2H, m), 3.25-3.5 (2H, m), 4.25-4.5 (2H, m), 6.95-7.4 (10H, m), 7.5-7.65 (1H, m), 7.75-8.0 (6H, m), 10.31 (1H, s)

(+)ESI-MS (m/z): 594 (M+Na)⁺

(3) tert-Butyl benzyl[2-[4-[[3-(2-formylphenoxy)phenyl]-sulfonyl]phenyl]ethyl]carbamate

NMR (CDCl₃, δ): 1.40 (9H, s), 2.7-2.9 (2H, m), 3.25-3.5 (2H, m), 4.25-4.5 (2H, m), 6.85-8.0 (17H, m), 10.40 (1H, s)

(+)ESI-MS (m/z): 594 (M+H)⁺

(4) tert-Butyl [4-[[4-[2-[benzyl(tert-butoxycarbonyl)-amino]ethyl]phenyl]sulfonyl]phenyl]acetate

NMR (CDCl₃, δ): 1.39 (9H, br s), 1.42 (9H, s), 2.7-2.9 (2H, m), 3.25-3.5 (2H, m), 3.56 (2H, s), 4.25-4.45 (2H, m), 7.1-7.35 (9H, m), 7.8-7.95 (4H, m)

(+)ESI-MS (m/z): 588 (M+Na)⁺

(5) Ethyl(R)-3-[3-[[4-[2-[(trifluoroacetyl)amino]propyl]-phenyl]sulfonyl]phenoxy]benzoate

NMR (CDCl₃, δ): 1.21 (3H, d, J=6.6 Hz), 1.39 (3H, t, J=7.2 Hz), 2.8-3.05 (2H, m), 4.2-4.45 (3H, m), 7.1-7.7 (9H, m), 7.8-7.9 (3H, m)

(+)ESI-MS (m/z): 558 (M+Na)⁺

(6) tert-Butyl benzyl[2-[4-[(3-hydroxyphenyl)sulfonyl]-phenyl]ethyl]carbamate

NMR (CDCl₃, δ): 1.38 (9H, br s), 2.7-2.9 (2H, m), 3.25-3.5 (2H, m), 4.37 (2H, br s), 6.95-7.05 (1H, m), 7.15-7.5 (10H, m), 7.75-7.85 (2H, m)

(+)ESI-MS (m/z): 490 (M+Na)⁺

(7) Ethyl 4-[3-[[4-[2-[benzyl(tert-butoxycarbonyl)amino]-ethyl]phenyl]sulfonyl]phenoxy]benzoate

NMR (CDCl₃, δ): 1.3-1.45 (12H, m), 2.7-2.9 (2H, m), 3.3-3.5 (2H, m), 4.3-4.5 (4H, m), 6.95-7.05 (2H, m), 7.1-7.75 (13H, m), 7.82 (2H, d, J=8.2 Hz), 8.0-8.1 (2H, m)

(+)ESI-MS (m/z): 638 (M+Na)⁺

(8) Ethyl 3-[3-[[4-[2-[benzyl(tert-butoxycarbonyl)amino]-ethyl]phenyl]sulfonyl]phenoxy]benzoate

NMR (CDCl₃, δ): 1.3-1.5 (12H, m), 2.7-2.95 (2H, m), 3.3-3.5 (2H, m), 4.25-4.5 (4H, m), 7.1-7.7 (14H, m), 7.75-7.9 (3H, m)

(+)ESI-MS (m/z): 638 (M+Na)⁺

(9) tert-Butyl benzyl[2-[4-[(4-hydroxyphenyl)sulfonyl]-phenyl]ethyl]carbamate

(+)ESI-MS (m/z): 490 (M+Na)⁺

(10) 2,2,2-Trifluoro-N-[3-[4-[(3-methoxyphenyl)sulfonyl]-phenyl]propyl]acetamide

NMR (CDCl₃, δ): 1.92 (2H, quintet, J=7 Hz), 2.72 (2H, t, J=7 Hz), 3.39 (2H, q, J=7 Hz), 3.84 (3H, s), 6.31 (1H, br s), 7.00-7.16 (1H, m), 7.20-7.58 (5H, m), 7.86 (2H, d, J=8 Hz)

(+)ESI-MS (m/z): 424 (M+Na)⁺

(11) tert-Butyl benzyl[2-[4-[(4-hydroxyphenyl)sulfonyl]-phenoxy]ethyl]carbamate

NMR (CDCl₃, δ): 1.45 (9H, s), 3.58 (2H, br s), 4.08 (2H, br s), 4.53 (2H, s), 6.86 (2H, d, J=8 Hz), 6.89 (2H, d, J=8 Hz), 7.10-7.42 (5H, m), 7.64-7.90 (4H, m)

(+)ESI-MS (m/z): 506 (M+Na)⁺

(12) Methyl 2-benzyloxy-5-[[4-[2-[benzyl(trifluoroacetyl)-amino]ethoxy]phenyl]sulfonyl]benzoate

NMR (CDCl₃, δ): 3.60-3.85 (2H, m), 3.91 (3H, s), 4.03-4.23 (2H, m), 4.77, 4.81 (total 2H, a pair of s), 5.23 (2H, s), 6.91 (2H, d, J=9 Hz), 7.07 (1H, d, J=9 Hz), 7.14-7.52 (10H, m), 7.85 (2H, d, J=8 Hz), 7.96 (1H, dd, J=9, 2 Hz), 8.35 (1H, d, J=2 Hz)

(+)ESI-MS (m/z): 650 (M+Na)⁺

Preparation 26

Under nitrogen at room temperature, to a solution of 4-fluorobenzaldehyde (3.0 g) in N,N-dimethylformamide (60 ml) was added 4-methoxybenzenethiol (3.3 ml) and potassium carbonate (3.7 g), and the mixture was stirred at 120° C. for 6 hours. The resulting mixture was poured into water and the aqueous mixture was extracted with ethyl acetate. The organic layer was washed successively with water and brine, dried over anhydrous magnesium sulfate and evaporated under reduced pressure. The residue was purified by column chromatgraphy on silica gel (hexane:ethyl acetate=10:1) to give 4-[(4-methoxyphenyl)thio]benzaldehyde (4.9 g).

NMR (CDCl₃, δ): 3.86 (3H, s), 6.95-7.0 (2H, m), 7.1-7.2 (2H, m), 7.45-7.5 (2H, m), 7.65-7.7 (2H, m), 9.89 (1H, s)

(+) APCI-MS (m/z): 245 (M+H)⁺

Preparation 27

Under nitrogen at room temperature, to a solution of 4-[(4-methoxyphenyl)thio]benzaldehyde (5.1 g) in methanol (51 ml) were added nitromethane (1.7 ml), acetic acid (0.60 ml) and butylamine (1.0 ml), and the mixture was stirred at the same temperature overnight to give precipitates. Water (51 ml) was poured into the resulting mixture and the mixture was the mixture was stirred for 30 minutes. The deposits were collected by filtration and the filter cake was washed with water followed by air-drying to give 1-methoxy-4-[[4-(2-nitroethenyl)phenyl]thio]benzene (5.4 g).

NMR (CDCl₃, δ): 3.86 (3H, s), 6.9-7.15 (4H, m), 7.3-7.6 (5H, m), 7.85-7.95 (1H, m)

(+)ESI-MS (m/z): 310 (M+Na)⁺

Preparation 28

Under nitrogen at 5° C., to a suspension of lithium aluminum hydride (3.2 g) in tetrahydrofuran (80 ml) was added dropwise 1-methoxy-4-[[4-(2-nitroethenyl)phenyl]thio]-benzene (4.8 g) in tetrahydrofuran (50 ml), and the mixture was refluxed for 6.5 hours. The resulting mixture was cooled to 5° C., and to this one was added sodium fluoride (14 g) followed by water (4.5 ml) dropwise carefully. The mixture was vigorously stirred at room temperature for 30 minutes. The precipitates were removed by filtration, and the filter cake was washed with a mixture of ethyl acetate and ethanol (95:5). The filtrate was evaporated under reduced pressure. The residue was dissolved into ethyl acetate (40 ml) and cooled to 5° C. To this one was added 4N hydrogen chloride in 1,4-dioxane (8.4 ml) and the mixture was stirred at room temperature for 30 minutes to deposit the corresponding salt followed by collection by filtration. The filter cake was washed with ethyl acetate and dissolved into a mixture of ethyl acetate and 1N sodium hydroxide. After separation, the organic layer was dried over anhydrous magnesium sulfate, evaporated under reduced pressure and dried to give 2-[4-[(4-methoxyphenyl)thio]phenyl]ethylamine (2.0 g).

NMR (CDCl₃, δ): 2.69 (2H, t, J=6.8 Hz), 2.93 (2H, t, J=6.8 Hz), 3.81 (3H, s), 6.85-6.95 (2H, m), 7.05-7.2 (4H, m), 7.35-7.45 (2H, m)

(+) APCI-MS (m/z): 260 (M+H)⁺

Preparation 29

Under nitrogen at room temperature, to a solution of 2-[4-[(4-methoxyphenyl)thio]phenyl]ethylamine (2.0 g) in dichloromethane (20 ml) was added benzaldehyde (0.78 ml), and the mixture was stirred at the same temperature for 20 minutes. To this one was added toluene and evaporated under reduced pressure. Under nitrogen at 5° C., to a solution of the residue in tetrahydrofuran (20 ml) was added sodium borohydride (0.32 g) followed by methanol (10 ml) dropwise and the mixture was stirred at room temperature for 40 minutes. The resulting mixture was poured into a mixture of ethyl acetate and water, and stirred for 10 minutes. After separation, the organic layer was washed with brine, dried over anhydrous magnesium sulfate and evaporated under reduced pressure. The residue was purified by column chromatography on silica gel (chloroform:methanol=100:1 to 20:1) to give N-benzyl-N-[2-[4-[(4-methoxyphenyl)thio]-phenyl]ethyl]amine (2.0 g).

NMR (CDCl₃, δ): 2.7-2.9 (4H, m), 3.81 (2H, s), 3.83 (3H, s), 6.85-6.95 (2H, m), 7.05-7.45 (11H, m)

(+) APCI-MS (m/z): 350 (M+H)⁺

Preparation 30

The following compounds were obtained according to a similar manner to that of Preparation 23.

(1) 4-[[4-[2-(Benzylamino)ethyl]phenyl]thio]phenol

NMR (DMSO-d₆, δ): 2.65-2.75 (4H, m), 3.71 (2H, s), 6.75-6.85 (2H, m), 6.95-7.35 (11H, m)

(+) APCI-MS (m/z): 336 (M+H)⁺

(2) 3-[[4-[2-(Benzylamino)ethyl]phenyl]thio]phenol

NMR (DMSO-d₆, δ): 2.7-2.85 (4H, m), 3.74 (2H, s), 7.55-7.75 (3H, m), 7.05-7.4 (10H, m)

(+) APCI-MS (m/z): 336 (M+H)⁺

(3) 2,2,2-Trifluoro-N-[3-[4-[(4-hydroxy-3-methylphenyl)sulfonyl]phenyl]propyl]acetamide

NMR (CDCl₃, δ): 1.8-2.0 (2H, m), 2.24 (3H, s), 2.6-2.75 (2H, m), 3.3-3.45 (2H, m), 6.83 (1H, d, J=8.3 Hz), 7.25-7.3 (2H, m), 7.6-7.7 (2H, m), 7.75-7.9 (2H, m)

(+)ESI-MS (m/z): 424 (M+Na)⁺

(4) (R)-2,2,2-Trifluoro-N-[2-[4-[(3-hydroxyphenyl)thio]-phenyl]-1-methylethyl]acetamide

NMR (CDCl₃, δ): 1.30 (3H, d, J=6.7 Hz), 2.65-2.95 (2H, m), 4.15-4.4 (1H, m), 6.3 (1H, m), 6.6-6.65 (1H, m), 6.8-6.85 (1H, m), 7.05-7.2 (3H, m), 7.35-7.45 (2H, m)

(+)ESI-MS (m/z): 378 (M+Na)⁺

(5) (R)-N-[2-[4-[(3-Chloro-4-hydroxyphenyl)sulfonyl]-phenyl]-1-methylethyl]-2,2,2-trifluoroacetamide

(+) APCI-MS (m/z): 444 (M+Na)⁺

(6) 3-[[4-[3-(Benzylamino)propyl]phenyl]sulfonyl]phenol

NMR (DMSO-d₆, δ): 1.75 (2H, quintet, J=7 Hz), 2.55 (2H, t, J=7 Hz), 2.66 (2H, t, J=7 Hz), 3.76 (2H, s), 6.95-7.11 (1H, m), 7.11-7.55 (10H, m), 7.81 (2H, d, J=8 Hz)

(+)ESI-MS (m/z): 382 (M+H)⁺

(7) 2-[[4-[(2R)-2-(Benzylamino)propyl]phenyl]sulfonyl]-phenol

NMR (DMSO-d₆, δ): 0.95 (3H, d, J=7 Hz), 2.40-3.00 (3H, m), 3.76 (1H, d, J=14 Hz), 3.80 (1H, d, J=14 Hz), 6.88 (1H, d, J=8 Hz), 7.00 (1H, t, J=8 Hz), 7.05-7.35 (5H, m), 7.37 (2H, d, J=8 Hz), 7.48 (1H, t, J=8 Hz), 7.80 (2H, d, J=8 Hz), 7.89 (1H, d, J=8 Hz)

(+)ESI-MS (m/z): 382 (M+H)⁺

(8) N-[3-[4-[(3-Chloro-4-hydroxyphenyl)sulfonyl]phenyl]-propyl]-2,2,2-trifluroacetamide

NMR (CDCl₃, δ): 1.92 (2H, quintet, J=7 Hz), 2.72 (2H, t, J=7 Hz), 3.38 (2H, q, J=7 Hz), 6.15 (1H, s, OH), 6.33 (1H, br s), 7.10 (1H, d, J=9 Hz), 7.32 (2H, d, J=8 Hz), 7.75 (1H, dd, J=9, 2 Hz), 7.83 (2H, d, J=8 Hz), 7.93 (1H, d, J=2 Hz)

(+)ESI-MS (m/z): 444 (M+Na)⁺

Preparation 31

Under nitrogen at room temperature, to a solution of 4-[[4-[2-(benzylamino)ethyl]phenyl]thio]phenol (794 mg) in tetrahydrofuran (8 ml) was added di-tert-butyl dicarbonate (775 mg) in tetrahydrofuran (2 ml), and the mixture was stirred at the same temperature for 9.5 hours. The resulting mixture was evaporated under reduced pressure. The residue was purified by column chromatography on silica gel (hexane:ethyl acetate=10:1 to 2:1) to give tert-butyl benzyl[2-[4-[(4-hydroxyphenyl)thio]phenyl]ethyl]carbamate (849 mg).

NMR (CDCl₃, δ): 1.45 (9H, s), 2.6-2.85 (2H, m), 3.25-3.45 (2H, m), 4.3-4.45 (2H, m), 6.75-6.85 (2H, m), 6.9-7.4 (11H, m)

(+)ESI-MS (m/z): 458 (M+Na)⁺

Preparation 32

Under nitrogen at room temperature, to a solution of tert-butyl benzyl[2-[4-[(4-hydroxyphenyl)thio]phenyl]-ethyl]carbamate (1.8 g) in N,N-dimethylformamide (20 ml) were added potassium carbonate (628 mg) and 2-fluorobenzaldehyde (0.497 ml), and the mixture was stirred at 130° C. for 1.5 hours. The resulting mixture was poured into water and the aqueous mixture was extracted with ethyl acetate. The organic layer was washed successively with water two times and brine, dried over anhydrous magnesium sulfate and evaporated under reduced pressure. The residue was purified by column chromatography on silica gel (hexane/ethyl acetate=10:1 to 5:1) to give tert-butyl benzyl[2-[4-[[4-(2-formylphenoxy)phenyl]thio]phenyl]ethyl]-carbamate (1.76 g).

NMR (CDCl₃, δ): 1.46 (9H, s), 2.6-2.9 (2H, m), 3.25-3.5 (2H, m), 4.25-4.45 (2H, m), 6.9-7.4 (15H, m), 7.45-7.6 (1H, m), 7.9-8.0 (1H, m), 10.47 (1H, s)

(+)ESI-MS (m/z): 562 (M+Na)⁺

Preparation 33

To a solution of tert-butyl benzyl[2-[4-[[4-(2-formylphenoxy)phenyl]sulfonyl]phenyl]ethyl]carbamate (1.17 g) in acetonitrile (18 ml) were added sodium dihydrogenphosphate (51.6 mg) and 30% hydrogen peroxide (0.232 ml) at room temperature. After the mixture was cooled to 5° C., sodium chlorite (333 mg) in water (18 ml) was added dropwise and the mixture was stirred at room temperature for 2.5 days. To the resulting mixture was added sodium sulfite, and the mixture was stirred for 10 minutes, followed by being adjusted pH to around 2.5 with 1N hydrochloric acid to give deposits. The precipitates were collected and washed with water followed by dryness in vacuo to give 2-[4-[[4-[2-[benzyl(tert-butoxycarbonyl)amino]-ethyl]phenyl]sulfonyl]phenoxy]benzoic acid (1.0 g).

NMR (DMSO-d₆, δ): 1.0-1.4 (9H, m), 2.7-2.9 (2H, m), 3.1-3.45 (2H, m), 4.25-4.5 (2H, m), 6.8-7.5 (10H, m), 7.55-8.0 (7H, m)

(−)ESI-MS (m/z): 586 (M−H)⁻

Preparation 34

Under nitrogen at room temperature, to a solution of 2-[4-[[4-[2-[benzyl(tert-butoxycarbonyl)amino]ethyl]-phenyl]sulfonyl]phenoxy]benzoic acid (1.0 g) in N,N-dimethylformamide (10 ml) were added potassium carbonate (282 mg) and iodoethane (0.15 ml), and the mixture was stirred at the same temperature for 2.5 hours. The resulting mixture was poured into water and the aqueous mixture was extracted with ethyl acetate. The organic layer was washed successively with water two times and brine, dried over anhydrous magnesium sulfate and evaporated under reduced pressure. The residue was purified by column chromatography on silica gel (hexane/ethyl acetate=3:1 to 12:5) to give ethyl 2-[4-[[4-[2-[benzyl(tert-butoxycarbonyl)amino]ethyl]phenyl]sulfonyl]-phenoxy]benzoate (783 mg).

NMR (CDCl₃, δ): 1.06 (3H, t, J=7.1 Hz), 1.41 (9H, s), 2.7-2.9 (2H, m), 3.25-3.5 (2H, m), 4.16 (2H, q, J=7.1 Hz), 4.25-4.5 (2H, m), 6.85-7.4 (11H, m), 7.5-7.6 (1H, m), 7.75-8.0 (5H, m)

(+)ESI-MS (m/z): 638 (M+Na)⁺

Preparation 35

The following compounds were obtained according to a similar manner to that of Preparation 7.

(1) Ethyl 2-[4-[[4-[2-(benzylamino)ethyl]phenyl]sulfonyl]-phenoxy]benzoate

NMR (CDCl₃, δ): 1.06 (3H, t, J=7.1 Hz), 2.8-2.95 (4H, m) 3.79 (2H, s), 4.16 (2H, q, J=7.1 Hz), 6.85-7.1 (3H, m), 7.2-7.4 (8H, m), 7.5-7.6 (1H, m), 7.75-9.9 (5H, m)

(+)ESI-MS (m/z): 516 (M+H)⁺

(2) Ethyl 2-[3-[[4-[2-(benzylamino)ethyl]phenyl]-sulfonyl]phenoxy]benzoate

NMR (CDCl₃, δ): 1.04 (3H, t, J=7.2 Hz), 2.8-2.95 (4H, m) 3.79 (2H, s), 4.05-4.2 (2H, m), 6.95-7.1 (2H, m), 7.2-7.65 (12H, m), 7.75-7.85 (2H, m), 7.9-8.0 (1H, m)

(+)ESI-MS (m/z): 516 (M+H)⁺

(3) 3-[[4-[2-(Benzylamino)ethyl]phenyl]sulfonyl]phenol

NMR (CDCl₃, δ): 2.7-3.0 (4H, m), 3.81 (2H, s), 6.9-7.0 (1H, m), 7.1-7.5 (10H, m), 7.75-7.85 (2H, m)

(−) APCI-MS (m/z): 366 (M−H)⁻

(4) Ethyl 4-[3-[[4-[2-(benzylamino)ethyl]phenyl]-sulfonyl]phenoxy]benzoate

NMR (CDCl₃, δ): 1.40 (3H, t, J=7.1 Hz), 2.8-2.95 (4H, m) 3.79 (2H, s), 4.38 (2H, q, J=7.1 Hz), 6.95-7.05 (2H, m), 7.15-7.4 (8H, m), 7.48 (1H, t, J=8.0 Hz), 7.55-7.75 (2H, m), 7.84 (2H, d, J=8.4 Hz), 8.0-8.1 (2H, m)

(+)ESI-MS (m/z): 516 (M+H)⁺

(5) Ethyl 3-[3-[[4-[2-(benzylamino)ethyl]phenyl]-sulfonyl]phenoxy]benzoate

NMR (CDCl₃, δ): 1.38 (3H, t, J=7.1 Hz), 2.8-2.95 (4H, m) 3.79 (2H, s), 4.37 (2H, q, J=7.1 Hz), 7.1-7.7 (14H, m), 7.8-7.9 (3H, m)

(+)ESI-MS (m/z): 516 (M+H)⁺

(6) (R)-1-Phenoxy-2-propanamine

NMR (DMSO-d₆, δ): 1.05 (3H, d, J=6.4 Hz), 3.05-3.2 (1H, m), 3.65-3.8 (2H, m), 6.85-7.0 (3H, m), 7.25-7.4 (2H, m)

(+)ESI-MS (m/z): 152 (M+H)⁺

(7) 4-[[4-[2-(Benzylamino)ethyl]phenyl]sulfonyl]phenol

(+)ESI-MS (m/z): 368 (M+H)⁺

(8) 4-[[4-[2-(Benzylamino)ethoxy]phenyl]sulfonyl]phenol

NMR (DMSO-d₆, δ): 2.85 (2H, t, J=6 Hz), 3.57 (2H, s), 4.10 (2H, t, J=6 Hz), 6.90 (2H, d, J=8 Hz), 7.09 (2H, d, J=8 Hz), 7.15-7.40 (5H, m), 7.72 (2H, d, J=8 Hz) 7.79 (2H, d, J=8 Hz)

(+)ESI-MS (m/z): 384 (M+H)⁺

Preparation 36

The following compound was obtained according to a similar manner to that of Preparation 26. 4-[(3-Methoxyphenyl)thio]benzaldehyde

NMR (CDCl₃, δ): 3.81 (3H, s), 6.9-7.0 (1H, m), 7.05-7.15 (2H, m), 7.25-7.4 (3H, m), 7.7-7.8 (2H, m), 9.92 (1H, s)

(+) APCI-MS (m/z): 245 (M+H)⁺

Preparation 37

The following compound was obtained according to a similar manner to that of Preparation 27. 1-Methoxy-3-[[4-(2-nitroethenyl)phenyl]thio]benzene

NMR (CDCl₃, δ): 3.80 (3H, s), 6.85-7.15 (3H, m), 7.2-7.55 (6H, m), 7.9-8.0 (1H, m)

(+)ESI-MS (m/z): 310 (M+Na)⁺

Preparation 38

The following compound was obtained according to a similar manner to that of Preparation 28. 2-[4-[(3-Methoxyphenyl)thio]phenyl]ethylamine

NMR (CDCl₃, δ): 2.74 (2H, t, J=6.9 Hz), 2.97 (2H, t, J=6.9 Hz), 3.75 (3H, s), 6.7-6.9 (3H, m), 7.1-7.4 (5H, m)

(+)ESI-MS (m/z): 260 (M+H)⁺

Preparation 39

The following compounds were obtained according to a similar manner to that of Preparation 29.

(1) N-Benzyl-N-[2-[4-[(3-methoxyphenyl)thio]phenyl]ethyl]-amine

NMR (CDCl₃, δ): 2.75-3.0 (4H, m), 3.78 (3H, s), 3.80 (2H, s), 6.7-6.95 (3H, m), 7.1-7.4 (10H, m)

(+) APCI-MS (m/z): 350 (M+H)⁺

(2) N-Benzyl-N-[3-[4-[(3-methoxyphenyl)sulfonyl]phenyl]-propyl]amine

NMR (CDCl₃, δ): 1.81 (2H, quintet, J=7 Hz), 2.52-2.80 (4H, m), 3.77 (2H, s), 3.84 (3H, s), 7.00-7.12 (1H, m), 7.15-7.55 (10H, m), 7.83 (2H, d, J=8 Hz)

(+)ESI-MS (m/z): 396 (M+H)⁺

(3) N-Benzyl-N-[(1R)-2-[4-[(2-methoxyphenyl)sulfonyl]-phenyl]-1-methylethyl]amine

NMR (CDCl₃, δ): 1.07 (3H, d, J=6 Hz), 2.68 (1H, dd, J=13, 6 Hz), 2.82 (1H, dd, J=13, 7 Hz), 2.94 (1H, m), 3.72 (1H, d, J=13 Hz), 3.73 (3H, s), 3.83 (1H, d, J=13 Hz), 6.89 (1H, d, J=8 Hz), 7.10-7.43 (7H, m), 7.14 (1H, t, J=8 Hz), 7.54 (1H, t, J=8 Hz), 7.88 (2H, d, J=8 Hz), 8.15 (1H, d, J=8 Hz)

(+)ESI-MS (m/z): 396 (M+H)⁺

Preparation 40

The following compound was obtained according to a similar manner to that of Preparation 31.

tert-Butyl benzyl[2-[4-[(3-hydroxyphenyl)thio]phenyl]ethyl]carbamate

NMR (CDCl₃, δ): 1.45 (9H, br s), 2.7-2.85 (2H, m), 3.3-3.5 (2H, m), 4.37 (2H, s), 6.55-6.7 (2H, m), 6.75-6.85 (1H, m), 7.05-7.4 (10H, m)

(+)ESI-MS (m/z): 458 (M+Na)⁺

Preparation 41

The following compound was obtained according to a similar manner to that of Preparation 32.

tert-Butyl benzyl[2-[4-[[3-(2-formylphenoxy)phenyl]thio]phenyl]ethyl]carbamate

NMR (CDCl₃, δ): 1.47 (9H, s), 2.65-2.9 (2H, m), 3.25-3.5 (2H, m), 4.25-4.5 (2H, m), 6.8-7.6 (16H, m), 7.85-7.95 (1H, m), 10.45 (1H, s)

(+)ESI-MS (m/z): 562 (M+H)⁺

Preparation 42

The following compound was obtained according to a similar manner to that of Preparation 33. 2-[3-[[4-[2-[Benzyl(tert-butoxycarbonyl)amino]ethyl]-phenyl]sulfonyl]phenoxy]benzoic acid

NMR (CDCl₃, δ): 1.0-1.4 (9H, m), 2.7-2.95 (2H, m), 3.2-3.5 (2H, m), 4.25-4.45 (2H, m), 6.8-8.0 (17H, m)

(−)ESI-MS (m/z): 586 (M−H)⁻

Preparation 43

The following compound was obtained according to a similar manner to that of Preparation 34.

Ethyl. 2-[3-[[4-[2-[benzyl(tert-butoxycarbonyl)amino]-ethyl]phenyl]sulfonyl]phenoxy]benzoate

NMR (CDCl₃, δ): 1.06 (3H, t, J=7.1 Hz), 1.42 (9H, s), 2.7-2.9 (2H, m), 3.25-3.5 (2H, m), 4.15 (2H, q, J=7.1 Hz), 4.25-4.5 (2H, m), 7.0-7.1 (2H, m), 7.1-7.6 (12H, m), 7.75-7.85 (2H, m), 7.9-8.0 (1H, m)

(+)ESI-MS (m/z): 638 (M+Na)⁺

Preparation 44

Under nitrogen at room temperature, to a solution of (R)-2,2,2-trifluoro-N-(1-methyl-2-phenylethyl)acetamide (1.5 g) and methyl 5-(chlorosulfonyl)-2-hydroxybenzoate (2.18 g) in 1,2-dichloroethane (15 ml) was added aluminum chloride (3.03 g), and the mixture was stirred at 60-65° C. for 4.5 hours. After the resulting mixture was cooled to room temperature, chloroform and water were added, followed by being stirred for 30 minutes. After separation, the organic layer was dried over anhydrous magnesium sulfate and evaporated under reduced pressure. The residue was purified by column chromatography on silica gel (chloroform/ethyl acetate=20:1) to give methyl(R)-2-hydroxy-5-[[4-[2-[(trifluoroacetyl)amino]propyl]phenyl]sulfonyl]benzoate (2.12 g).

NMR (CDCl₃, δ): 1.21 (3H, d, J=6.7 Hz), 2.75-3.05 (2H, m), 3.98 (3H, s), 4.15-4.4 (1H, m), 7.07 (1H, d, J=8.8 Hz), 7.32 (2H, d, J=8.3 Hz), 7.87 (2H, d, J=8.3 Hz), 7.95 (1H, dd, J=2.4, 8.9 Hz), 8.48 (1H, d, J=2.4 Hz)

(+)ESI-MS (m/z): 468 (M+Na)⁺

Preparation 45

Under nitrogen at room temperature, a mixture of methyl (R)-2-hydroxy-5-[[4-[2-[(trifluoroacetyl)amino]propyl]-phenyl]sulfonyl]benzoate (2.1 g) and 7N hydrogen chloride in ethanol (40 ml) was refluxed for 12 hours. The resulting mixture was evaporated under reduced pressure followed by dryness in vacuo to give ethyl(R)-5-[[4-(2-aminopropyl)-phenyl]sulfonyl]-2-hydroxybenzoate hydrochloride (1.97 g).

NMR (DMSO-d₆, δ): 1.11 (3H, d, J=6.5 Hz), 1.34 (3H, t, J=7.1 Hz), 2.8-3.55 (3H, m), 4.37 (2H, q, J=7.1 Hz), 7.22 (1H, d, J=8.7 Hz), 7.51 (2H, d, J=8.3 Hz), 7.85-8.3 (3H, m)

(+)ESI-MS (m/z): 364 (M−HCl+H)⁺

Preparation 46

Ethyl(R)-5-[[4-(2-aminopropyl)phenyl]sulfonyl]-2-hydroxybenzoate hydrochloride (1.96 g) was dissolved into a mixture of chloroform/methanol (4:1) and water, and sodium bicarbonate (412 mg) was added. After separation, the organic layer was dried over anhydrous magnesium sulfate and evaporated under reduced pressure. Under nitrogen, a mixture of the residue and (R)-2-(3-chlorophenyl)oxirane (758 mg) in ethanol (34 ml) was stirred at 70° C. for 19.5 hours. The resulting mixture was evaporated under reduced pressure. The residue was purified by column chromatography on silica gel (chloroform/methanol=20:1) to give ethyl 5-[[4-[(2R)-2-[[(2R)-2-(3-chlorophenyl)-2-hydroxyethyl]amino]-propyl]phenyl]sulfonyl]-2-hydroxybenzoate (810 mg).

NMR (CDCl₃, δ): 1.05 (3H, d, J=6.1 Hz), 1.45 (3H, t, J=7.2 Hz), 2.55-3.0 (5H, m), 4.35-4.6 (3H, m), 7.06 (1H, d, J=8.9 Hz), 7.1-7.35 (6H, m), 7.8-8.0 (3H, m), 8.50 (1H, d, J=2.3 Hz)

(+)ESI-MS (m/z): 518, 520 (M+H)⁺

Preparation 47

Under nitrogen at room temperature, to a solution of 3-phenyl-1-propylamine (100 g) in methanol (500 ml) was added ethyl trifluoroacetate (106 ml) dropwise, and the mixture was stirred at the same temperature for 4 hours. The resulting mixture was evaporated under reduced pressure and dried in vacuo to give 2,2,2-trifluoro-N-(3-phenylpropyl)-acetamide (171 g).

NMR (CDCl₃, δ): 1.85-2.0 (2H, m), 2.69 (2H, t, J=7.4 Hz), 3.3-3.5 (2H, m), 7.15-7.4 (5H, m)

(+)ESI-MS (m/z): 254 (M+Na)⁺

Preparation 48

Under nitrogen at 5° C., to a solution of 2,2,2-trifluoro-N-(3-phenylpropyl)acetamide (100 g) in chloroform (800 ml) was added chlorosulfonic acid (144 ml) dropwise, and the mixture was stirred at the same temperature for 1 hour and at room temperature for 36 hours. The resulting mixture was carefully poured into a stirred mixture of water and chloroform under ice-water cooling. After separation, the organic layer was washed with water, dried over anhydrous magnesium sulfate and evaporated under reduced pressure. The residue was purified by column chromatography on silica gel (hexane/ethyl acetate=4:1 to 2:1) to give 4-[3-[(trifluoroacetyl)amino]propyl]benzenesulfonyl chloride (109 g).

NMR (CDCl₃, δ): 1.9-2.1 (2H, m), 2.81 (2H, t, J=7.4 Hz), 3.35-3.55 (2H, m), 7.4-7.5 (2H, m), 7.95-8.05 (2H, m)

Preparation 49

The following compounds were obtained according to a similar manner to that of Preparation 44.

(1) 2,2,2-Trifluoro-N-[3-[4-[(4-methoxy-3-methylphenyl)sulfonyl]phenyl]propyl]acetamide

NMR (CDCl₃, δ): 1.8-2.0 (2H, m), 2.21 (3H, s), 2.6-2.75 (2H, m), 3.3-3.45 (2H, m), 3.86 (3H, s), 6.87 (1H, d, J=8.6 Hz), 7.25-7.3 (2H, m), 7.65 (1H, m), 7.75-7.9 (3H, m)

(+)ESI-MS (m/z): 438 (M+Na)⁺

(2) (R)-N-[2-[4-[(3-Chloro-4-methoxyphenyl)sulfonyl]-phenyl]-1-methylethyl]-2,2,2-trifluoroacetamide

(+) APCI-MS (m/z): 458 (M+Na)⁺

(3) (R)-4-[[4-[[2-[(Trifluoroacetyl)amino]propyl]oxy]-phenyl]sulfonyl]benzoic acid

NMR (DMSO-d₆, δ): 1.1-1.3 (3H, m), 3.9-4.4 (3H, m), 7.1-7.3 (2H, m), 7.85-8.2 (6H, m)

(−)ESI-MS (m/z): 430 (M−H)⁻

(4) N-[3-[4-[(3,4-Dihydroxyphenyl)sulfonyl]phenyl]propyl]-2,2,2-trifluoroacetamide

NMR (DMSO-d₆, δ): 1.78 (2H, quintet, J=7 Hz), 2.65 (2H, t, J=7 Hz), 3.18 (2H, t, J=7 Hz), 6.88 (1H, d, J=8 Hz), 7.22 (1H, s), 7.24 (1H, d, J=8 Hz), 7.43 (2H, d, J=8 Hz), 7.76 (2H, d, J=8 Hz)

(−)ESI-MS (m/z): 402 (M−H)⁻

(5) Methyl 5-[[4-[[(2R)-2-(formylamino)propyl]oxy]-phenyl]sulfonyl]-2-hydroxybenzoate

NMR (CDCl₃, δ): 1.33, 1.35 (total 3H, J=7 Hz, a pair of d), 3.90-4.25 (2H, m), 4.00, 3.99 (total 3H, a pair of s), 4.49 (1H, m), 5.76 (1H, br d, J=6 Hz), 6.80-7.15 (3H, m), 7.86 (2H, d, J=9 Hz), 7.92, 8.11 (total 1H, J=9, 2 Hz, a pair of dd), 8.16, 8.23 (total 1H, a pair of br s), 8.46, 8.50 (total 1H, J=2 Hz, a pair of d), 11.25, 11.29 (total 1H, a pair of s, OH)

(+)ESI-MS (m/z): 416 (M+Na)⁺

(6) N-[3-[4-[(3-Chloro-4-methoxyphenyl)sulfonyl]phenyl]-propyl]-2,2,2-trifluoroacetamide

NMR (CDCl₃, δ): 1.92 (2H, quintet, J=7 Hz), 2.72 (2H, t, J=7 Hz), 3.38 (2H, q, J=7 Hz), 3.94 (3H, s), 6.36 (1H, br s), 7.00 (1H, d, J=9 Hz), 7.31 (2H, d, J=8 Hz), 7.83 (2H, d, J=8 Hz), 7.83 (1H, dd, J=9, 2 Hz), 7.91 (1H, d, J=2 Hz)

(+)ESI-MS (m/z): 458 (M+Na)⁺

(7) Methyl 2-hydroxy-5-[[4-[3-[(trifluoroacetyl)amino]-propyl]phenyl]sulfonyl]benzoate

NMR (CDCl₃, δ): 1.92 (2H, quintet, J=7 Hz), 2.72 (2H, t, J=7 Hz), 3.38 (2H, q, J=7 Hz), 4.00 (3H, s), 6.33 (1H, br s), 7.07 (1H, d, J=9 Hz), 7.31 (2H, d, J=8 Hz), 7.85 (1H, d, J=8 Hz), 7.95 (1H, dd, J=9 and 2 Hz), 8.48 (1H, d, J=2 Hz), 11.28 (1H, s, OH)

(+)ESI-MS (m/z): 468 (M+Na)⁺

Preparation 50

Under nitrogen at room temperature, to a solution of methyl(4-hydroxyphenyl)acetate (10 g) in N,N-dimethylformamide (50 ml) were added potassium carbonate (9.3 g) and benzyl bromide (8.0 ml), and the mixture was stirred at 60° C. for 1 hour. The resulting mixture was poured into water and the aqueous mixture was extracted with hexane/ethyl acetate (1:1). The organic layer was washed successively with water and brine, dried over anhydrous magnesium sulfate, evaporated under reduced pressure and dried in vacuo to give methyl [4-(benzyloxy)phenyl]acetate (16 g).

NMR (CDCl₃, δ): 3.56 (2H, s), 3.68 (3H, s), 5.05 (2H, s), 6.9-7.0 (2H, m), 7.1-7.5 (7H, m)

(+)ESI-MS (m/z): 279 (M+Na)⁺

Preparation 51

To a solution of methyl [4-(benzyloxy)phenyl]acetate (16 g) in methanol (160 ml) was added 1N sodium hydroxide (68.5 ml) at room temperature, and the mixture was stirred at the same temperature for 2 hours. After removal of methanol under reduced pressure, the residue was dissolved into a mixture of water and ethyl acetate. The aqueous layer was adjusted to pH 2-3 with 6N hydrochloric acid to give deposits. The precipitates were collected and washed with water followed by dryness in vacuo to give [4-(benzyloxy)phenyl]acetic acid (11 g).

NMR (DMSO-d₆, δ): 3.48 (2H, s), 5.08 (2H, s), 6.9-7.0 (2H, m), 7.15-7.2 (2H, m), 7.25-7.5 (5H, m)

(−)ESI-MS (m/z): 241 (M−H)

Preparation 52

Under nitrogen, to a suspension of [4-(benzyloxy)-phenyl]acetic acid (10.8 g) in dichloromethane (300 ml) were added concentrated sulfuric acid (0.5 ml) and the excess amount of isobutene in dryice-acetone bath, and the mixture was raised to room temperature slowly followed by being stirred at the same temperature for 3.5 days. The resulting mixture was poured into saturated aqueous sodium bicarbonate and the aqueous mixture was extracted with ethyl acetate. The organic layer was washed successively with saturated aqueous sodium bicarbonate two times and brine, dried over anhydrous magnesium sulfate and evaporated under reduced pressure. The residue was purified by column chromatography on silica gel (hexane/ethyl acetate=10:1) to give tert-butyl [4-(benzyloxy)phenyl]acetate (11.3 g).

NMR (CDCl₃, δ): 1.43 (9H, s), 3.46 (2H, s), 5.05 (2H, s), 6.9-6.95 (2H, m), 7.15-7.5 (7H, m)

(+)ESI-MS (m/z): 321 (M+Na)⁺

Preparation 53

A mixture of tert-butyl [4-(benzyloxy)phenyl]acetate (11.3 g) and 10% palladium on activated carbon (50% wet, 550 mg) in methanol (110 ml) was stirred at room temperature in the presence of hydrogen at an atmospheric pressure for 5.5 hours. After filtration, the filtrate was evaporated under reduced pressure and dried in vacuo to give tert-butyl(4-hydroxyphenyl)acetate (8.56 g).

NMR (CDCl₃, δ): 1.44 (9H, s), 3.45 (2H, s), 6.7-6.9 (2H, m), 7.05-7.15 (2H, m)

(+)ESI-MS (m/z): 231 (M+Na)⁺

Preparation 54

Under nitrogen at room temperature, to a solution of tert-butyl benzyl[2-[4-[(triisopropylsilyl)thio]phenyl]-ethyl]carbamate (210 mg) in toluene (3 ml) were added tert-butyl [4-[[(trifluoromethyl)sulfonyl]oxy]phenyl]acetate (157 mg), bis(dibenzylideneacetone)palladium(0) (24.2 mg)bis(2-diphenylphosphinophenyl)ether (22.6 mg) and cesium fluoride (70.2 mg), and the mixture was stirred at 80° C. for 17 hours. The resulting mixture was poured into water and the aqueous mixture was extracted with ethyl acetate. The organic layer was washed with brine, dried over anhydrous magnesium sulfate and evaporated under reduced pressure. The residue was purified by column chromatography on silica gel (hexane/ethyl acetate=10:1) to give tert-butyl [4-[[4-[2-[benzyl(tert-butoxycarbonyl)amino]ethyl]phenyl]thio]phenyl]-acetate (136 mg).

NMR (CDCl₃, δ): 1.43 (9H, s), 1.46 (9H, s), 2.65-2.9 (2H, m), 3.25-3.5 (4H, m), 4.3-4.45 (2H, m), 6.95-7.4 (13H, m)

(+)ESI-MS (m/z): 556 (M+Na)⁺

Preparation 55

Under nitrogen at room temperature, to a solution of tert-butyl [4-[[4-[2-[benzyl(tert-butoxycarbonyl)amino]-ethyl]phenyl]sulfonyl]phenyl]acetate (725 mg) in dichloromethane (5 ml) was added trifluoroacetic acid (1 ml), and the mixture was stirred at the same temperature for 4 hours. The resulting mixture was evaporated under reduced pressure. Under nitrogen at room temperature, to the residue in ethanol (10 ml) was added 4N hydrogen chloride in 1,4-dioxane (2 ml), and the mixture was stirred at the same temperature overnight. The resulting mixture was evaporated under reduced pressure. The residue was dissolved into a mixture of saturated aqueous sodium bicarbonate and ethyl acetate. After separation, the organic layer was dried over anhydrous magnesium sulfate, evaporated under reduced pressure and dried in vacuo to give ethyl [4-[[4-[2-(benzylamino)ethyl]phenyl]sulfonyl]phenyl]acetate (573 mg).

NMR (CDCl₃, δ): 1.24 (3H, t, J=7.1 Hz), 2.75-2.95 (4H, m), 3.65 (2H, s), 3.79 (2H, s), 4.14 (2H, q, J=7.1 Hz), 7.15-7.5 (9H, m), 7.8-7.95 (4H, m)

(+)ESI-MS (m/z): 438 (M+H)⁺

Preparation 56

Under nitrogen at room temperature, to a mixture of bis(dibenzylideneacetone)palladium(0) (13.1 mg) and bis(2-diphenylphosphinophenyl)ether (13.3 mg) was added toluene (2 ml). After being stirred at the same temperature for 15 minutes, tert-butyl benzyl[2-(4-iodophenyl)ethyl]carbamate (200 mg) in toluene (2 ml), potassium tert-butoxide (61.6 mg) and triisopropylsilanethiol (0.108 ml) were added, and the mixture was stirred at 80° C. for 1 hour. The resulting mixture was poured into saturated aqueous sodium bicarbonate and the aqueous mixture was extracted with ethyl acetate. The organic layer was washed successively with water and brine, dried over anhydrous magnesium sulfate and evaporated under reduced pressure. The residue was purified by column chromatography on silica gel (hexane/ethyl acetate=20:1) to give tert-butyl benzyl[2-4-[(triisopropylsilyl)thio]-phenyl]ethyl carbamate (210 mg).

NMR (CDCl₃, δ): 1.07 (18H, d, J=6.3 Hz), 1.1-1.3 (3H, m) 1.4-1.6 (9H, m), 2.65-2.85 (2H, m), 3.2-3.45 (2H, m), 4.2-4.35 (2H, m), 6.9-7.45 (9H, m)

Preparation 57

Under nitrogen, a mixture of formic acid (0.828 ml) and acetic anhydride (2.07 ml) was stirred at 5° C. for 30 minutes. To this one was added (R)-1-phenoxy-2-propanamine (1.66 g) in dichloromethane (5 ml), and the mixture was stirred at room temperature for 2 hours. The resulting mixture was poured into water and the aqueous mixture was extracted with ethyl acetate. The organic layer was washed successively with saturated aqueous sodium bicarbonate, water and brine, dried over anhydrous magnesium sulfate and evaporated under reduced pressure. The residue was purified by column chromatography on silica gel (hexane/ethyl acetate=1:1 to 1:2) to give (R)-1-methyl-2-phenoxyethylformamide (147 g).

NMR (CDCl₃, δ): 1.3-1.4 (3H, m), 3.8-4.1 (2H, m), 4.35-4.5 (1H, m), 6.8-7.05 (3H, m), 7.2-7.4 (2H, m), 8.17 (1H, s)

(+)ESI-MS (m/z): 202 (M+Na)⁺

Preparation 58

A mixture of 4-mercaptophenol (16.2 g) in dimethyl sulfoxide (15 ml) was stirred at 800° C. for 5 hours. The resulting mixture was poured into a mixture of water and the aqueous mixture was extracted with hexane/ethyl acetate (1:1). After separation, the organic layer was washed successively with water two times and brine, dried over anhydrous magnesium sulfate, evaporated under reduced pressure and dried in vacuo to give di(4-hydroxyphenyl)-disulfide (16.54 g).

(−)ESI-MS (m/z): 249 (M−H)-

Preparation 59

Under nitrogen at room temperature, to a solution of N-benzylethanolamine (50 g) in methanol (250 ml) was added ethyl trifluoroacetate (59 ml) dropwise, and the mixture was stirred at 45°C for 2 hours. The resulting mixture was evaporated under reduced pressure. The residue was dissolved into a mixture of 1N hydrochloric acid and hexane/ethyl acetate (1:1). After separation, the organic layer was washed successively with water, saturated aqueous sodium bicarbonate and brine, dried over anhydrous magnesium sulfate, evaporated under reduced pressure and dried in vacuo to give N-benzyl-2,2,2-trifluoro-N-(2-hydroxyethyl)-acetamide (64 g).

(+)ESI-MS (m/z): 270 (M+Na)⁺

Preparation 60

To a solution of (R)-2-chloro-4-[[4-[2-[(trifluoroacetyl)amino]propyl]phenyl]sulfonyl]phenyl trifluoromethanesulfonate (1.0 g) and 3-ethoxycarbonylphenylboronic acid (455 mg) in 1,2-dimethoxyethane (10 ml) were added tetrakis(triphenylphosphine)palladium(0) (104 mg) and 2M sodium carbonate (1.90 ml) at room temperature, and the mixture was stirred at 80° C. for 4 hours. The resulting mixture was poured into water and the aqueous mixture was extracted with ethyl acetate. The organic layer was washed with brine, dried over anhydrous magnesium sulfate and evaporated under reduced pressure. The residue was purified by column chromatography on silica gel (hexane/ethyl acetate=3:1 to 2:1) to give ethyl(R)-2′-chloro-4′-[[4-[2-[(trifluoroacetyl)amino]propyl]phenyl]sulfonyl]-1,1′-biphenyl-3-carboxylate (783 mg).

NMR (CDCl₃, δ): 1.23 (3H, d, J=6.7 Hz), 1.39 (3H, t, J=7.1 Hz), 2.8-3.1 (2H, m), 4.2-4.5 (3H, m), 7.38 (2H, d, J=8.3 Hz), 7.4-7.6 (3H, m), 7.8-8.2 (6H, m)

(+)ESI-MS (m/z): 576 (M+Na)⁺

Preparation 61

Under nitrogen at room temperature, to a solution of (R)-1-phenoxy-2-propanamine (1.4 g) in methanol (7 ml) was added ethyl trifluoroacetate (1.32 ml) dropwise, and the mixture was stirred at the same temperature overnight. The resulting mixture was evaporated under reduced pressure and dried in vacuo to give (R)-2,2,2-trifluoro-N-(1-methyl-2-phenoxyethyl)acetamide (2.13 g).

NMR (CDCl₃, δ): 1.41 (3H, d, J=6.9 Hz), 3.9-4.1 (2H, m), 4.3-4.55 (1H, m), 6.85-7.05 (3H, m), 7.2-7.4 (2H, m)

(+)ESI-MS (m/z): 270 (M+Na)⁺

Preparation 62

To a solution of 2,2,2-trifluoro-N-[3-[4-[(3-methoxyphenyl)sulfonyl]phenyl]propyl]acetamide (6.13 g) in 1,4-dioxane (61 ml) was added 1N sodium hydroxide solution (23 ml), and the mixture was stirred at room temperature for 12 hours. After being concentrated, the mixture was partitioned between ethyl acetate and water. The organic layer was washed with brine, dried over magnesium sulfate, and filtered. The filtrate was concentrated to give 3-[4-[(3-methoxyphenyl)sulfonyl]phenyl]propylamine (3.46 g) as a pale yellow oil.

NMR (CDCl₃, δ): 1.76 (2H, quintet, J=7 Hz), 2.60-2.82 (4H, m), 3.84 (3H, s), 7.01-7.13 (1H, m), 7.20-7.55 (5H, m), 7.85 (2H, d, J=8 Hz)

(+)ESI-MS (m/z): 306 (M+H)⁺

Preparation 63

Under nitrogen atmosphere, to an ice-cooled solution of 4-iodophenol (15.40 g), triphenylphosphine (22.03 g), and tert-butyl benzyl(2-hydroxyethyl)carbamate (21.05 g) in tetrahydrofuran (123 ml) was added diethyl azodicarboxylate (14.58 g) in tetrahydrofuran (31 ml) for 25 minutes, and the mixture was stirred at room temperature for 2 hours. After being concentrated, the mixture was treated with hexane/ethyl acetate (5/1, 180 ml). The precipitate formed was filtered off, the filtrate was concentrated, and the residue was purified by column chromatography (silica gel, hexane/ethyl acetate) to give tert-butyl benzyl[2-(4-iodophenoxy)ethyl]carbamate (7.17 g) as a colorless oil.

NMR (CDCl₃, δ): 1.45 (9H, s), 3.58 (2H, br s), 4.07 (2H, br s), 4.55 (2H, s), 6.62 (2H, d, J=8 Hz), 7.10-7.40 (5H, m), 7.53 (2H, d, J=8 Hz)

(+)ESI-MS (m/z): 476 (M+Na)⁺

Preparation 64

To a solution of N-[2-[4-[[4-[2-[benzyl(2,2,2-trifluoroacetyl)amino]ethoxy]phenyl]dithio]phenoxy]ethyl]-N-benzyl-2,2,2-trifluoroacetamide (359 mg) in ethanol/tetrahydrofuran (2/1, 5.4 ml) was added triphenylphosphine (142 mg), and the mixture was stirred at room temperature for 6 hours. The mixture was partitioned between ethyl acetate and water. The organic layer was separated, washed successively with water and brine, dried over magnesium sulfate, and filtered. The filtrate was concentrated to give N-benzyl-2,2,2-trifluoro-N-[2-(4-mercaptophenoxy)ethyl]acetamide (525 mg) as a colorless oil.

NMR (CDCl₃, δ): 3.37 (1H, s), 3.55-3.85 (2H, m), 4.00-4.20 (2H, m), 4.80, 4.84 (total 2H, a pair of s), 6.75 (2H, d, J=9 Hz), 7.05-7.85 (7H, m)

(−) APCI-MS (m/z): 354 (M−H)⁻

Preparation 65.

To a solution of methyl 5-iodosalicylate (5.56 g) in N,N-dimethylformamide (56 ml) were added powdered potassium carbonate (3.04 g) and benzyl bromide (2.6 ml), and the mixture was stirred at room temperature for 45 hours. The mixture was partitioned between hexane/ethyl acetate (1/2) and water. The organic layer was separated, washed successively with water and brine, dried over magnesium sulfate, and filtered. The solvent was evaporated to give methyl 2-benzyloxy-5-iodobenzoate (8.07 g) as a pale yellow oil.

NMR (CDCl₃, δ): 3.90 (3H, s), 5.17 (2H, s), 6.78 (1H, d, J=9 Hz), 7.26-7.52 (5H, m), 7.69 (1H, dd, J=9, 2 Hz), 8.10 (1H, d, J=2 Hz)

(+)ESI-MS (m/z): 391 (M+Na)⁺

Preparation 66

Chlorosulfonic acid (10 ml) was cooled in an ice bath whereupon methyl salicylate (7.60 g) was added dropwise over 20 minutes. The mixture was heated to 40° C. for 30 minutes, allowed to cool to room temperature, and poured onto crashed ice. The precipitate formed was collected, washed with water, and dried in vacuo to give methyl 5-chlorosulfonyl-2-hydroxybenzoate (7.89 g) as a white powder.

NMR (CDCl₃, δ): 4.04 (3H, s), 7.18 (1H, d, J=9 Hz), 8.09 (1H, dd, J=9, 2 Hz), 8.57 (1H, d, J=2 Hz), 11.55 (1H, s, OH)

Preparation 67

Methyl 5-[[4-[[(2R)-2-(formylamino)propyl]oxy]phenyl]-sulfonyl]-2-hydroxybenzoate (1.60 g) and hydrogen chloride in methanol (10-20%, 16 ml) were mixed and stirred at room temperature for 12 hours. The solvent was evaporated to give methyl 5-[[4-[[(2R)-2-aminopropyl]oxy]phenyl]sulfonyl]-2-hydroxybenzoate hydrochloride (1.67 g) as a white solid.

NMR (DMSO-d₆, δ): 1.28 (3H, d, J=7 Hz), 3.35-3.75 (1H, m), 3.89 (3H, s), 3.92-4.32 (2H, m), 7.18 (1H, d, J=9 Hz), 7.19 (2H, d, J=9 Hz), 7.90 (2H, d, J=9 Hz), 7.97 (1H, dd, J=9, 2 Hz), 8.21 (1H, d, J=2 Hz), 11.27 (1H, s, OH)

(+)ESI-MS (m/z): 366 (free, M+H)⁺

Preparation 68

To a solution of methyl 2-hydroxy-5-[[4-[3-[(trifluoroacetyl)amino]propyl]phenyl]sulfonyl]benzoate (4.43 g) in N,N-dimethylformamide (35 ml) were added powdered potassium carbonate (2.73 g) and iodomethane (0.93 ml), and the mixture was stirred at 50° C. for 2 hours. After being allowed to cool to room temperature, the mixture was partitioned between hexane/ethyl acetate (1/2) and water. The organic layer was separated, washed successively with water and brine, dried over magnesium sulfate, and filtered. The filtrate was concentrated to give methyl 2-methoxy-5-[[4-[3-[(trifluoroacetyl)amino]propyl]phenyl]sulfonyl]-benzoate (4.81 g) as a pale yellow solid.

NMR (CDCl₃, δ): 1.92 (2H, quintet, J=7 Hz), 2.68 (2H, t, J=7 Hz), 3.38 (2H, q, J=7 Hz), 3.86 (3H, s), 3.95 (3H, s), 6.40 (1H, br s), 7.06 (1H, d, J=9 Hz), 7.31 (2H, d, J=8 Hz), 7.85 (2H, d, J=8 Hz), 8.03 (1H, dd, J=9, 2 Hz), 8.34 (1H, d, J=2 Hz)

(−)ESI-MS (m/z): 458 (M−H)⁻

Preparation 69

The following compounds were obtained according to a similar manner to that of Preparation 22.

(1) (R)-2,2,2-Trifluoro-N-[2-[4-[(3-methoxyphenyl)thio]-phenyl]-1-methylethyl]acetamide

NMR (CDCl₃, δ): 1.22 (3H, d, J=6.7 Hz), 2.7-2.95 (2H, m), 3.75 (3H, s), 4.2-4.35 (1H, m), 6.7-6.95 (3H, m), 7.05-7.35 (5H, m)

(+)ESI-MS (m/z): 392 (M+Na)⁺

(2) 2,2,2-Trifluoro-N-[3-[4-[(3-methoxyphenyl)thio]-phenyl]propyl]acetamide

NMR (CDCl₃, δ): 1.92 (2H, quintet, J=7 Hz), 2.67 (2H, t, J=7 Hz), 3.40 (2H, q, J=7 Hz), 3.76 (3H, s), 6.25 (1H, br s), 6.65-6.95 (3H, m), 7.13 (2H, d, J=8 Hz), 7.20 (1H, t, J=8 Hz), 7.32 (2H, d, J=8 Hz)

(+)ESI-MS (m/z): 392 (M+Na)⁺

(3) tert-Butyl benzyl[2-[4-[(4-hydroxyphenyl)thio]-phenoxy]ethyl]carbamate

NMR (CDCl₃, δ): 1.45 (9H, s), 3.58 (2H, br s), 4.07 (2H, br s), 4.55 (2H, s), 5.20 (1H, br s, OH), 6.77 (4H, d, J=8 Hz), 7.10-7.42 (9H, m)

(+)ESI-MS (m/z): 474 (M+Na)⁺

(4) Methyl 2-benzyloxy-5-[[4-(2-[benzyl(trifluoroacetyl)-amino]ethoxy]phenyl]thio]benzoate

NMR (CDCl₃, δ): 3.60-3.83 (2H, m), 3.88 (3H, s), 4.02-4.22 (2H, m), 4.81, 4.85 (total 2H, a pair of s), 5.16 (2H, s), 6.80 (2H, d, J=9 Hz), 6.93 (1H, d, J=9 Hz), 7.15-7.55 (13H, m), 7.80 (1H, d, J=2 Hz)

(+)ESI-MS (m/z): 618 (M+Na)⁺

Preparation 70

The following compounds were obtained according to a similar manner to that of Preparation 24.

(1) Ethyl(R)-3-[3-[[4-[2-[(trifluoroacetyl)amino]propyl]-phenyl]thio]phenoxy]benzoate

NMR (CDCl₃, δ): 1.21 (3H, d, J=6.6 Hz), 1.39 (3H, t, J=7.3 Hz), 2.7-2.95 (2H, m), 4.2-4.45 (3H, m), 6.75-7.85 (12H, m)

(+)ESI-MS (m/z): 526 (M+Na)⁺

(2) Ethyl 4-[3-[[4-[2-[benzyl(tert-butoxycarbonyl)amino]-ethyl]phenyl]thio]phenoxy]benzoate

NMR (CDCl₃, δ): 1.38 (3H, t, J=7.2 Hz), 1.4-1.55 (9H, m), 2.7-2.9 (2H, m), 3.3-3.5 (2H, m), 4.3-4.5 (4H, m), 6.8-7.4 (15H, m), 7.95-8.0 (2H, m)

(+)ESI-MS (m/z): 606 (M+Na)⁺

(3) Ethyl 3-[3-[[4-[2-[benzyl(tert-butoxycarbonyl)amino]-ethyl]phenyl]thio]phenoxy]benzoate

NMR (CDCl₃, δ): 1.39 (3H, t, J=7.2 Hz), 1.4-1.55 (9H, m), 2.65-2.85 (2H, m), 3.25-3.5 (2H, m), 4.3-4.5 (4H, m), 6.75-7.4 (15H, m), 7.64 (1H, m), 7.76 (1H, m)

(+)ESI-MS (m/z): 606 (M+Na)⁺

Preparation 71

The following compound was obtained according to a similar manner to that of Preparation 48. (R)-4-[2-[(Trifluoroacetyl)amino]propyl]benzenesulfonyl chloride

NMR (CDCl₃, δ): 1.27 (3H, d, J=6.7 Hz), 2.92 (1H, dd, J=7.3, 13.6 Hz), 3.07 (1H, dd, J=6.1, 13.6 Hz), 4.32 (1H, h, J=7.0 Hz), 6.19 (1H, br), 7.44 (2H, d, J=8.5 Hz), 8.00 (2H, d, J=8.5 Hz)

Preparation 72

The following compounds were obtained according to a similar manner to that of Preparation 60.

(1) Ethyl(R)-3′-[[4-[2-[(trifluoroacetyl)amino]propyl]-phenyl]sulfonyl]-1,1′-biphenyl-3-carboxylate

NMR (CDCl₃, δ): 1.21 (3H, d, J=6.7 Hz), 1.42 (3H, t, J=7.1 Hz), 2.75-3.05 (2H, m), 4.15-4.35 (1H, m), 4.43 (2H, q, J=7.1 Hz), 7.33 (2H, d, J=8.3 Hz), 7.45-8.3 (10H, m)

(+)ESI-MS (m/z): 542 (M+Na)⁺

(2) Ethyl 2′-(methoxymethoxy)-4′-[[4-[2-[(trifluoroacetyl)-amino]ethyl]phenyl]sulfonyl]-1,1′-biphenyl-3-carboxylate

NMR (CDCl₃, δ): 1.26 (3H, t, J=7.1 Hz), 2.97 (2H, t, J=7.1 Hz), 3.93 (3H, s), 2.6-2.65 (2H, m), 4.38 (2H, q, J=7.1 Hz), 5.18 (2H, s), 7.36 (2H, d, J=8.4 Hz), 7.45-7.55 (2H, m), 7.6-7.7 (2H, m), 7.76 (1H, m), 7.96 (2H, d, J=8.4 Hz), 8.05 (1H, d, J=7.8 Hz), 8.15 (1H, m)

(+)ESI-MS (m/z): 588 (M+Na)⁺

Preparation 73

The following compound was obtained according to a similar manner to that of Preparation 21.

2,2,2-Trifluoro-N-[3-(4-iodophenyl)propyl]acetamide

NMR (CDCl₃, δ): 1.90 (2H, quintet, J=7 Hz), 2.62 (2H, t, J=7 Hz), 3.38 (2H, q, J=7 Hz), 6.26 (1H, br s), 6.93 (2H, d, J=8 Hz), 7.62 (2H, d, J=8 Hz)

(+)ESI-MS (m/z): 380 (M+Na)⁺

Preparation 74

The following compound was obtained according to a similar manner to that of Preparation 62.

(1R)-2-[4-[(2-Methoxyphenyl)sulfonyl]phenyl]-1-methylethylamine

NMR (CDCl₃, δ): 1.12 (3H, d, J=6 Hz), 2.62 (1H, dd, J=13, 8 Hz), 2.75 (1H, dd, J=13, 6 Hz), 3.08-3.34 (1H, m), 3.77 (3H, s), 6.91 (1H, d, J=8 Hz), 7.10 (1H, t, J=8 Hz), 7.30 (2H, d, J=8 Hz), 7.44-7.64 (1H, m), 7.90 (2H, d, J=8 Hz), 8.15 (1H, d, J=8 Hz)

(+)ESI-MS (m/z): 306 (M+H)⁺

Preparation 75

The following compound was obtained according to a similar manner to that of Preparation 9.

N-[3-[4-[[4-(Benzyloxy)-3-hydroxyphenyl]sulfonyl]-phenyl]propyl]-2,2,2-trifluoroacetamide

NMR (CDCl₃, δ): 1.89 (2H, quintet, J=7 Hz), 2.69 (2H, t, J=7 Hz), 3.36 (2H, q, J=7 Hz), 5.14 (2H, s), 5.93 (1H, s, OH), 6.60 (1H, br s), 6.97 (1H, d, J=8 Hz) 7.15-7.60 (9H, m), 7.80 (2H, d, J=8 Hz)

(−)ESI-MS (m/z): 492 (M−H)⁻

Preparation 76

The following compound was obtained according to a similar manner to that of Preparation 15.

N-[3-[4-[[4-Benzyloxy-3-(methoxymethoxy)phenyl]-sulfonyl]phenyl]propyl]-2,2,2-trifluoroacetamide

NMR (CDCl₃, δ): 1.95 (2H, quintet, J=7 Hz), 2.71 (2H, t, J=7 Hz), 3.37 (2H, q, J=7 Hz), 3.50 (3H, s), 5.17 (2H, s), 5.24 (2H, s), 6.34 (1H, br s), 6.96 (1H, d, J=9 Hz), 7.16-7.50 (7H, m), 7.54 (1H, dd, J=9, 2 Hz), 7.67 (1H, d, J=2 Hz), 7.83 (2H, d, J=8 Hz)

(+)ESI-MS (m/z): 560 (M+Na)⁺

Preparation 77

The following compound was obtained according to a similar manner to that of Preparation 63.

N-[2-[4-[[4-[2-[Benzyl(2,2,2-trifluoroacetyl)amino]-ethoxy]phenyl]dithio]phenoxy]ethyl]-N-benzyl-2,2,2-trifluoroacetamide

NMR (CDCl₃, δ): 3.55-3.85 (4H, m), 4.00-4.25 (4H, m), 4.80, 4.84 (total 4H, a pair of s), 6.79 (4H, d, J=8 Hz), 7.10-7.50 (14H, m)

(+)ESI-MS (m/z): 731 (M+Na)⁺

### EXAMPLE 1

Under nitrogen at room temperature, to a solution of methyl 4-[[4-(2-aminoethyl)phenyl]sulfonyl]-2-pyridinecarboxylate (335 mg) in dimethylsulfoxide (5 ml) was added N,O-bis(trimethylsilyl)acetamide (0.127 ml), and the mixture was stirred at the same temperature for 1 hour. To this one was added (R)-2-(3-chlorophenyl)oxirane (194 mg) and the mixture was stirred at 80° C. for 20 hours. The resulting mixture was cooled to room temperature and 10% aqueous acetic acid was added. After being stirred for 20 minutes, the mixture was poured into saturated aqueous sodium bicarbonate and the aqueous mixture was extracted with chloroform. The organic layer was washed successively with water and brine, dried over anhydrous magnesium sulfate and evaporated under reduced pressure. The residue was purified by column chromatography on silica gel (chloroform/methanol=20:1 to 15:1) to give methyl(R)-4-[[4-[2-[[2-(3-chlorophenyl)-2-hydroxyethyl]amino]ethyl]-phenyl]sulfonyl]-2-pyridinecarboxylate (158 mg).

NMR (CDCl₃, δ): 2.6-3.1 (6H, m), 4.03 (3H, s), 4.6-4.7 (1H, m), 7.15-8.05 (8H, m), 8.45-8.75 (2H, m), 8.95 (1H, d, J=5.0 Hz)

(+)ESI-MS (m/z): 475, 477 (M+H)⁺

### EXAMPLE 2

To a suspension of methyl(R)-4-[[4-[2-[[2-(3-chlorophenyl)-2-hydroxyethyl]amino]ethyl]phenyl]sulfonyl]-2-pyridinecarboxylate (155 mg) in a mixture of ethanol (3 ml) and tetrahydrofuran (1.5 ml) was added 1N sodium hydroxide (0.326 ml) at room temperature, and the mixture was stirred at the same temperature for 3.5 hours. The resulting mixture was evaporated under reduced pressure. The residue was purified by reversed phase chromatography to give sodium (R)-4-[[4-[2-[[2-(3-chlorophenyl)-2-hydroxyethyl]amino]-ethyl]phenyl]sulfonyl]-2-pyridinecarboxylate (3.9 mg).

NMR (DMSO-d₆, δ): 2.55-2.85 (6H, m), 4.5-4.65 (1H, m), 7.2-7.35 (4H, m), 7.48 (2H, d, J=8.3 Hz), 7.75-7.8 (1H, m), 7.87 (2H, d, J=8.3 Hz), 8.15 (1H, br s), 8.72 (1H, d, J=5.0 Hz)

(−)ESI-MS (m/z): 459, 461 (M−Na)⁻

### EXAMPLE 3

The following compounds were obtained according to a similar manner to that of Example 6.

(1) Ethyl 5-[[4-[2-[benzyl[(2R)-2-(3-chlorophenyl)-2-hydroxyethyl]amino]ethyl]phenyl]sulfonyl]-2-hydroxybenzoate

NMR (CDCl₃, δ): 1.45 (3H, t, J=7 Hz), 2.45-3.00 (6H, m), 3.54 (1H, d, J=13 Hz), 3.63 (1H, br s, OH), 3.90 (1H, d, J=13 Hz), 4.45 (2H, q, J=7 Hz), 4.60 (1H, dd, J=10, 4 Hz), 7.05 (1H, d, J=9 Hz), 7.05-7.40 (11H, m), 7.80 (2H, d, J=8 Hz), 7.92 (1H, dd, J=9, 2 Hz), 8.49 (1H, d, J=2 Hz), 11.40 (1H, s, OH)

(+)ESI-MS (m/z): 594 (M+H)⁺

(2) Ethyl 3-[4-[[4-[(2R)-2-[[(2R)-2-(3-chlorophenyl)-2-hydroxyethyl]amino]propyl]phenyl]sulfonyl]phenoxy]-benzoate

NMR (CDCl₃, δ): 1.06 (3H, d, J=6.2 Hz), 1.37 (3H, t, J=7.1 Hz), 2.6-3.0 (5H, m), 4.37 (2H, q, J=7.1 Hz), 4.55 (1H, dd, J=3.8, 8.5 Hz), 6.95-7.1 (2H, m), 7.1-7.55 (8H, m), 7.7 (1H, m), 7.8-7.95 (5H, m)

(+)ESI-MS (m/z): 594, 596 (M+H)⁺

(3) Ethyl(R)-2-[4-[[4-[2-[benzyl[2-(3-chlorophenyl)-2-hydroxyethyl]amino]ethyl]phenyl]sulfonyl]phenoxy]-benzoate

NMR (CDCl₃, δ): 1.06 (3H, t, J=7.1 Hz), 2.5-2.95 (6H, m), 3.55 (1H, d, J=1-3.4 Hz), 3.91 (1H, d, J=13.4 Hz), 4.16 (2H, q, J=7.1 Hz), 4.62 (1H, dd, J=3.5, 9.8 Hz), 6.85-7.35 (15H, m), 7.5-7.6 (1H, m), 7.7-8.0 (5H, m)

(+)ESI-MS (m/z): 670, 672 (M+H)⁺

(4) Ethyl(R)-2-[3-[[4-[2-[benzyl[2-(3-chlorophenyl)-2-hydroxyethyl]amino]ethyl]phenyl]sulfonyl]phenoxy]-benzoate

NMR (CDCl₃, δ): 1.06 (3H, t, J=7.1 Hz), 2.5-2.95 (6H, m), 3.56 (1H, d, J=13.4 Hz), 3.92 (1H, d, J=13.4 Hz), 4.15 (2H, d, J=7.1 Hz), 4.62 (1H, dd, J=3.7, 9.8 Hz), 6.95-7.6 (18H, m), 7.75-7.85 (2H, m), 7.9-8.0 (1H, m)

(+)ESI-MS (m/z): 670 (M+H)⁺

(5) Ethyl(R)-[4-[[4-[2-[benzyl[2-(3-chlorophenyl)-2-hydroxyethyl]amino]ethyl]phenyl]sulfonyl]phenyl]acetate

NMR (CDCl₃, δ): 1.25 (3H, t, J=7.3 Hz), 2.52.95 (6H, m), 3.55 (1H, d, J=13.4 Hz), 3.64 (2H, s), 3.90 (1H, d, J=13.4 Hz), 4.12 (2H, t, J=7.3 Hz), 4.61 (1H, dd, J=3.7, 9.8 Hz), 7.1-7.35 (11H, m), 7.41 (2H, d, J=8.3 Hz), 7.75-7.95 (4H, m)

(+)ESI-MS (m/z): 592, 594 (M+H)⁺

(6) (R)-4-[[4-[2-[Benzyl[2-(3-chlorophenyl)-2-hydroxyethyl]amino]ethyl]phenyl]sulfonylphenol

NMR (CDCl₃, δ): 2.5-2.95 (6H, m), 3.5-3.95 (2H, m), 4.55-4.65 (1H, m), 6.85-6.95 (2H, m), 7.1-7.4 (11H, m), 7.75-7.9 (4H, m)

(+)ESI-MS (m/z): 522, 524 (M+H)⁺

(7) Ethyl 3-[3-[[4-[(2R)-2-[[(2R)-2-(3-chlorophenyl)-2-hydroxyethyl]amino]propyl]phenyl]sulfonyl]phenoxy]-benzoate

NMR (CDCl₃, δ): 1.07 (3H, d, J=6.2 Hz), 1.38 (3H, t, J=7.2 Hz), 2.6-3.0 (5H, m), 4.37 (2H, q, J=7.2 Hz), 4.5-4.6 (1H, m), 7.1-7.7 (13H, m), 7.8-7.9 (3H, m)

(+) APCI-MS (m/z): 594 (M+H)⁺

(8) Ethyl 4′-[[4-[(2R)-2-[[(2R)-2-(3-chlorophenyl)-2-hydroxyethyl]amino]propyl]phenyl]sulfonyl]-1,1′-biphenyl-3-carboxylate

NMR (CDCl₃, δ): 1.06 (3H, d, J=6.1 Hz), 1.44 (3H, t, J=7.1 Hz), 2.6-3.0 (5H, m), 4.41 (2H, q, J=7.1 Hz), 7.1-7.35 (6H, m), 7.55 (1H, t, J=7.7 Hz), 7.65-8.1 (8H, m), 8.2-8.25 (1H, m)

(+)ESI-MS (m/z): 578, 580 (M+H)⁺

(9) Ethyl 3′-[[4-[(2R)-2-[[(2R)-2-(3-chlorophenyl)-2-hydroxyethyl]amino]propyl]phenyl]sulfonyl]-1,1′-biphenyl-3-carboxylate

NMR (CDCl₃, δ): 1.05 (3H, d, J=6.1 Hz), 1.42 (3H, t, J=7.2 Hz), 2.55-3.0 (5H, m), 4.42 (2H, q, J=7.2 Hz), 4.45-4.55 (1H, m), 7.1-7.35 (6H, m), 7.45-7.65 (2H, m), 7.7-8.3 (8H, m)

(+)ESI-MS (m/z): 578 (M+H)⁺

(10) (R)-3-[[4-[2-[Benzyl[2-(3-chlorophenyl)-2-hydroxyethyl]amino]ethyl]phenyl]sulfonyl]phenol

NMR (CDCl₃, δ): 2.45-3.0 (6H, m), 3.5-4.0 (2H, m), 4.45-4.55 (1H, m), 6.9-7.45 (14H, m), 7.5-7.55 (1H, m), 7.8-7.9 (2H, m)

(+) APCI-MS (m/z): 522, 524 (M+H)⁺

(11) Ethyl(R)-3-[3-[[4-[2-[benzyl[2-(3-chlorophenyl)-2-hydroxyethyl]amino]ethyl]phenyl]sulfonyl]phenoxy]-benzoate

NMR (CDCl₃, δ): 1.38 (3H, t, J=7.1 Hz), 2.5-2.95 (6H, m) 3.55 (1H, d, J=13.4 Hz), 3.91 (1H, d, J=13.4 Hz), 4.37 (2H, q, J=7.1 Hz), 7.1-7.5 (15H, m), 7.55-7.7 (3H, m), 7.75-7.9 (3H, m)

(+)ESI-MS (m/z): 670, 672′ (M+H)⁺

(12) Ethyl 5-[(4-[3-[[(2R)-2-(3-chlorophenyl)-2-hydroxyethyl]amino]propyl]phenyl]sulfonyl]-2-methoxybenzoate

NMR (CDCl₃, δ): 1.38 (3H, t, J=7 Hz), 1.82 (2H, quintet, J=7 Hz), 2.55-3.00 (6H, m), 3.93 (3H, s), 4.36 (2H, q, J=7 Hz), 4.69 (1H, dd, J=9, 4 Hz), 7.04 (1H, d, J=9 Hz), 7.10-7.45 (6H, m), 7.83 (2H, d, J=8 Hz), 8.02 (1H, dd, J=9, 2 Hz), 8.32 (1H, d, J=2 Hz)

(+)ESI-MS (m/z): 532 (M+H)⁺

(13) Ethyl(R)-4-[3-[[4-[2-[benzyl[2-(3-chlorophenyl)-2-hydroxyethyl]amino]ethyl]phenyl]sulfonyl]phenoxy]-benzoate

NMR (CDCl₃, δ): 1.39 (3H, t, J=7.1 Hz), 2.55-2.95 (6H, m), 3.55 (1H, d, J=13.4 Hz), 3.91 (1H, d, J=13.4 Hz) 4.38 (2H, q, J=7.1 Hz), 4.61 (1H, dd, J=3.6, 9.8 Hz), 6.95-7.05 (2H, m), 7.1-7.35 (12H, m), 7.4-7.75 (3H, m), 7.80 (2H, d, J=8.2 Hz), 8.0-8.1 (2H, m)

(+)ESI-MS (m/z): 670, 672 (M+H)⁺

(14) Ethyl 4′-[[4-[2-[benzyl[2-(3-chlorophenyl)-2-hydroxyethyl]amino]ethyl]phenyl]sulfonyl]-2′-hydroxy-1,1′-biphenyl-3-carboxylate

NMR (CDCl₃, δ): 1.38 (3H, t, J=7.1 Hz), 2.45-3.0 (6H, m), 3.54 (1H, d, J=13.4 Hz), 3.92 (1H, d, J=13.4 Hz), 4.38 (2H, q, J=7.1 Hz), 4.53 (1H, dd, J=3.8, 9.9 Hz), 7.0-7.7 (16H, m), 7.90 (2H, d, J=8.3 Hz), 8.0-8.2 (2H, m)

(+)ESI-MS (m/z): 670, 672 (M+H)⁺

(15) Ethyl 4-[[4-[[(2R)-2-[[(2R)-2-(3-chlorophenyl)-2-hydroxyethyl]amino]propyl]oxy]phenyl]sulfonyl]benzoate

NMR (CDCl₃, δ): 1.19 (3H, d, J=6.5 Hz), 1.39 (3H, t, J=7.1 Hz), 2.71 (1H, dd, J=9.0, 12.2 Hz), 2.97 (1H, dd, J=3.7, 12.2 Hz), 3.05-3.2 (1H, m), 3.8-4.0 (2H, m), 4.39 (2H, q, J=7.1 Hz), 4.63 (1H, dd, J=3.6, 8.9 Hz), 6.9-7.0 (2H, m), 7.15-7.4 (4H, m), 7.8-8.0 (4H, m), 8.1-8.2 (2H, m)

(+)ESI-MS (m/z): 518, 520 (M+H)⁺

(16) 3-[[4-[3-[Benzyl[(2R)-2-(3-chlorophenyl)-2-hydroxyethyl]amino]propyl]phenyl]sulfonyl]phenol

NMR (CDCl₃, δ): 1.81 (2H, quintet, J=7 Hz), 2.35-2.80 (6H, m), 3.48 (1H, d, J=13 Hz), 3.86 (1H, d, J=13 Hz), 4.59 (1H, dd, J=10, 4 Hz), 6.90-7.60 (15H, m), 7.80 (2H, d, J=8 Hz)

(+)ESI-MS (m/z): 536 (M+H)⁺

(17) 4-[[4-[2-[Benzyl[(2R)-2-(3-chlorophenyl)-2-hydroxyethyl]amino]ethoxy]phenyl]sulfonyl]phenol

NMR (CDCl₃, δ): 2.65 (1H, dd, J=13, 10 Hz), 2.82-3.22 (2H, m), 2.85 (1H, dd, J=13, 4 Hz), 3.69 (1H, d, J=13 Hz), 3.86-4.18 (2H, m), 3.94 (1H, d, J=13 Hz), 4.64 (1H, dd, J=10, 3 Hz), 6.85 (2H, d, J=8 Hz), 6.91 (2H, d, J=8 Hz), 7.05-7.40 (9H, m), 7.76 (2H, d, J=8 Hz), 7.81 (2H, d, J=8 Hz)

(+)ESI-MS (m/z): 538 (M+H)⁺

(18) 2-[[4-[(2R)-2-[Benzyl[(2R)-2-(3-chlorophenyl)-2-hydroxyethyl]amino]propyl]phenyl]sulfonyl]phenol

NMR (CDCl₃, δ): 1.03 (3H, d, J=6 Hz), 2.40-2.90 (4H, m), 3.00-3.25 (1H, m), 3.47 (1H, d, J=13 Hz), 3.56. (1H, br s, OH), 3.80 (1H, d, J=13 Hz), 4.56 (1H, dd, J=10, 4 Hz), 6.85-7.55 (14H, m), 7.66 (1H, t, J=8 Hz), 7.77 (2H, d, J=8 Hz), 9.23 (1H, br s)

(−)ESI-MS (m/z): 534 (M−H)⁻

(19) Ethyl 5-[[4-[3-[benzyl[(2R)-2-(3-chlorophenyl)-2-hydroxyethyl]amino]propyl]phenyl]sulfonyl]-2-hydroxybenzoate

NMR (CDCl₃, δ): 1.45 (3H, t, J=7 Hz), 1.80 (2H, quintet, J=7 Hz), 2.32-2.80 (6H, m), 3.48 (1H, d, J=13 Hz), 3.87 (1H, d, J=13 Hz), 3.90 (1H, br s, OH), 4.46 (2H, q, J=7 Hz), 4.60 (1H, dd, J=10, 4 Hz), 7.05 (1H, d, J=9 Hz), 7.05-7.45 (11H, m), 7.80 (2H, d, J=8 Hz) 7.93 (1H, dd, J=9, 2 Hz), 8.49 (1H, d, J=2 Hz), 11.40 (1H, s, OH)

(20) Ethyl 4-[[4-[3-[benzyl[(2R)-2-(3-chlorophenyl)-2-hydroxyethyl]amino]propyl]phenyl]sulfonyl]-2-hydroxybenzoate

NMR (CDCl₃, δ): 1.41 (3H, t, J=7 Hz), 1.80 (2H, quintet, J=7 Hz), 2.32-2.80 (6H, m), 3.48 (1H, d, J=13 Hz), 3.87 (1H, d, J=13 Hz), 3.88 (1H, br s, OH), 4.43 (2H, q, J=7 Hz), 4.60 (1H, dd, J=10, 4 Hz), 7.05-7.45 (11H, m), 7.41 (1H, dd, J=8, 2 Hz), 7.51 (1H, d, J=2 Hz), 7.82 (2H, d, J=8 Hz), 7.96 (1H, d, J=8 Hz), 11.01 (1H, s, OH)

(+)ESI-MS (m/z): 608 (M+H)⁺

(21) Ethyl 5-[[4-[2-[benzyl[(2R)-2-(3-chlorophenyl)-2-hydroxyethyl]amino]ethoxy]phenyl]sulfonyl]-2-hydroxybenzoate

NMR (CDCl₃, δ): 1.49 (3H, t, J=7 Hz), 2.64 (1H, dd, J=13, 10 Hz), 2.83-3.20 (2H, m), 2.85 (1H, dd, J=13, 4 Hz), 3.69 (1H, d, J=13 Hz), 3.90-4.10 (2H, m), 3.94 (1H, d, J=13 Hz), 4.46 (2H, q, J=7 Hz), 4.64 (1H, dd, J=10, 4 Hz), 6.93 (2H, d, J=9 Hz), 7.05 (1H, d, J=9 Hz), 7.10-7.38 (9H, m), 7.85 (2H, d, J=9 Hz), 7.92 (1H, dd, J=9, 2 Hz), 8.47 (1H, d, J=2 Hz), 11.38 (1H, s, OH)

(+)ESI-MS (m/z): 610 (M+H)⁺

(22) Ethyl 5-[[4-[[(2R)-2-[[(2R)-2-(3-chlorophenyl)-2-hydroxyethyl]amino]propyl]oxy]phenyl]sulfonyl]-2-hydroxybenzoate

NMR (CDCl₃, δ): 1.19 (3H, d, J=6 Hz), 1.45 (3H, t, J=7 Hz), 2.70 (1H, dd, J=12, 9 Hz), 2.97 (1H, dd, J=12, 4 Hz), 3.00-3.25 (1H, m), 3.72-4.00 (2H, m), 4.45 (2H, q, J=7 Hz), 4.63 (1H, dd, J=9, 4 Hz), 6.96 (2H, d, J=9 Hz), 7.05 (1H, d, J=9 Hz), 7.12-7.45 (4H, m), 7.86 (2H, d, J=9 Hz), 7.91 (1H, dd, J=9, 2 Hz) 8.46 (1H, d, J=2 Hz)

(−)ESI-MS (m/z): 532 (M−H)⁻

(23). Ethyl 2-chloro-4-[[4-[3-[[(2R)-2-(3-chlorophenyl)-2-hydroxyethyl]amino]propyl]phenyl]sulfonyl]benzoate

NMR (CDCl₃, δ): 1.39 (3H, t, J=7 Hz), 1.94 (2H, quintet, J=7 Hz), 2.60-3.10 (6H, m), 4.40 (2H, q, J=7 Hz), 4.89 (1H, dd, J=9, 4 Hz), 7.10-7.45 (6H, m), 7.70-7.97 (4H, m), 7.99 (1H, s)

(+)ESI-MS (m/z): 536 (M+H)⁺

### EXAMPLE 4

The following compound was obtained according to a similar manner to that of Example 23.

Ethyl 5-[[4-[2-[[(2R)-2-(3-chlorophenyl)-2-hydroxyethyl]amino]ethyl]phenyl]sulfonyl]-2-hydroxybenzoate hydrochloride

NMR (DMSO-d₆, δ): 1.34 (3H, t, J=7 Hz), 2.92-3.32 (6H, m), 4.37 (2H, q, J=7 Hz), 4.98 (1H, m), 6.33 (1H, br s, OH), 7.19 (1H, d, J=9 Hz), 7.25-7.60 (6H, m), 7.91 (2H, d, J=8 Hz), 8.00 (1H, dd, J=9, 2 Hz), 8.23 (1H, d, J=2 Hz)

(+)ESI-MS (m/z): 504 (free, M+H)⁺

### EXAMPLE 5

The following compounds were obtained according to a similar manner to that of Example 8.

(1) Sodium [5-[[4-[3-[[(2R)-2-(3-chlorophenyl)-2-hydroxyethyl]amino]propyl]phenyl]sulfonyl]-2-hydroxybenzoate

NMR (DMSO-d₆, δ): 2.50-2.85 (6H, m), 4.60 (1H, m), 5.39 (1H, br s, OH), 6.72 (1H, d, J=9 Hz), 7.12-7.50 (6H, m), 7.65 (1H, dd, J=9, 2 Hz), 7.73 (2H, d, J=8 Hz), 8.13 (1H, d, J=2 Hz), 18.20 (1H, br s, OH)

(−)ESI-MS (m/z): 474 (free, M−H)⁻

(2) Sodium (R)-2-[4-[[4-[2-[[2-(3-chlorophenyl)-2-hydroxyethyl]amino]ethyl]phenyl]sulfonyl]phenoxy]-benzoate

NMR (DMSO-d₆, δ): 2.65-2.85 (6H, m), 4.5-4.65 (2H, m), 6.8-6.95 (3H, m), 7.1-7.6 (9H, m), 7.75-7.9 (4H, m)

(−)ESI-MS (m/z): 550, 552 (M−Na)⁻

(3) Sodium 3-[4-[[4-[(2R)-2-[[(2R)-2-(3-chlorophenyl)-2-hydroxyethyl]amino]propyl]phenyl]sulfonyl]phenoxy]-benzoate

NMR (DMSO-d₆, δ): 0.90 (3H, d, J=5.9 Hz), 2.4-2.95 (5H, m), 4.45-4.55 (1H, m), 6.95-7.5 (11H, m), 7.65-7.95 (5H, m)

(−)ESI-MS (m/z): 564, 566 (M−Na)⁻

(4) Sodium (R)-2-[3-[[4-(2-[[2-(3-chlorophenyl)-2-hydroxyethyl]amino]ethyl)phenyl]sulfonyl]phenoxy]-benzoate

NMR (DMSO-d₆, δ): 2.55-2.85 (6H, m), 4.55-4.7 (1H, m), 6.85-7.6 (14H, m), 7.80 (2H, d, J=8.2 Hz)

(−)ESI-MS (m/z): 550, 552 (M−Na)⁻

(5) Sodium 5-[[4-[(2R)-2-[[(2R)-2-(3-chlorophenyl)-2-hydroxyethyl]amino]propyl]phenyl]sulfonyl]-2-hydroxybenzoate

NMR (DMSO-d₆, δ): 1.06 (3H, d, J=6.2 Hz), 2.6-3.3 (5H, m), 4.8-4.95 (1H, m), 6.74 (1H, d, J=8.8 Hz), 7.25-7.55 (6H, m), 7.68 (1H, dd, J=2.6, 8.6 Hz), 7.82 (2H, d, J=8.3 Hz), 8.15 (1H, m)

(−)ESI-MS (m/z): 488, 490 (M−Na)

(6) Sodium 3-[3-[[4-[(2R)-2-[[(2R)-2-(3-chlorophenyl)-2-hydroxyethyl]amino]propyl]phenyl]sulfonyl]phenoxy]-benzoate

NMR (DMSO-d₆, δ): 1.04 (3H, d, J=6.1 Hz), 2.4-2.9 (5H, m), 4.5-4.6 (1H, m), 7.0-7.05 (1H, m), 7.2-7.9 (15H, m)

(−)ESI-MS (m/z): 564, 566 (M−Na)⁻

(7) Sodium 4′-[[4-[(2R)-2-[[(2R)-2-(3-chlorophenyl)-2-hydroxyethyl]amino]propyl]phenyl]sulfonyl]-1,1′-biphenyl-3-carboxylate

NMR (DMSO-d₆, δ): 0.92 (3H, d, J=5.9 Hz), 2.4-2.95 (5H, m), 4.55-4.65 (1H, m), 7.2-7.55 (7H, m), 7.75-8.1 (8H, m), 8.2 (1H, m)

(−)ESI-MS (m/z): 548, 550 (M−Na)⁻

(8) Sodium 3′-[[4-[(2R)-2-[[(2R)-2-(3-chlorophenyl)-2-hydroxyethyl]amino]propyl]phenyl]sulfonyl]-1,1′-biphenyl-3-carboxylate

NMR (DMSO-d₆, δ): 0.89 (3H, d, J=5.9 Hz), 2.5-2.9 (5H, m), 4.5-4.9 (1H, m), 7.15-7.45 (7H, m), 7.55-7.75 (2H, m), 7.85-8.0 (5H, m), 8.1-8.15 (2H, m)

(−)ESI-MS (m/z): 548, 550 (M−Na)⁻

(9) Sodium (R)-3′-[[4-[2-[[2-(3-chlorophenyl)-2-hydroxyethyl]amino]ethyl]phenyl]sulfonyl]-1,1′-biphenyl-4-carboxylate

NMR (DMSO-d₆, δ): 2.55-2.9 (6H, m), 4.55-4.65 (1H, m), 7.2-7.5 (6H, m), 7.6-7.8 (3H, m), 7.85-8.1 (6H, m) 8.18 (1H, m)

(−)ESI-MS (m/z): 535 (M−Na)⁻

(10) Sodium (R)-3′-[[4-[2-[[2-(3-chlorophenyl)-2-hydroxyethyl]amino]ethyl]phenyl]sulfonyl]-1,1′-biphenyl-3-carboxylate

NMR (DMSO-d₆, δ): 2.5-2.8 (6H, m), 4.5-4.6 (1H, m), 7.2-7.5 (7H, m), 7.6-7.8 (2H, m), 7.85-8.0 (5H, m) 8.1-8.15 (2H, m)

(−)ESI-MS (m/z): 534 (M−Na)⁻

(11) Sodium (R)-3′-([4-(2-[[2-(3-chlorophenyl)-2-hydroxyethyl]amino]ethyl)phenyl]sulfonyl]-1,1′-biphenyl-2-carboxylate

NMR (DMSO-d₆, δ): 2.5-2.9 (6H, m), 4.55-4.7 (1H, m), 7.15-8.0 (16H, m)

(−)ESI-MS (m/z): 534, 536 (M−Na)⁻

(12) Sodium (R)-4-[3-[[4-[2-[[2-(3-chlorophenyl)-2-hydroxyethyl]amino]ethyl]phenyl]sulfonyl]phenoxy]-benzoate

NMR (DMSO-d₆, δ): 2.5-2.9 (6H, m), 4.45-4.6 (1H, m), 6.85-7.0 (2H, m), 7.15-7.5 (8H, m), 7.5-7.7 (2H, m), 7.7-8.0 (4H, m)

(−)ESI-MS (m/z): 550, 552 (M−Na)⁻

(13) Sodium (R)-3-[3-[[4-[2-[[2-(3-chlorophenyl)-2-hydroxyethyl]amino]ethyl]phenyl]sulfonyl]phenoxy]-benzoate

NMR (DMSO-d₆, δ): 2.55-2.85 (6H, m), 4.55-4.7 (1H, m), 7.0-7.1 (1H, m), 7.2-7.5 (10H, m), 7.55-7.9 (5H, m)

(−)ESI-MS (m/z): 550, 552 (M−Na)⁻

(14) Sodium (R)-4′-[[4-[2-[[2-(3-chlorophenyl)-2-hydroxyethyl]amino]ethyl]phenyl]sulfonyl]-2′-hydroxy-1,1′-biphenyl-3-carboxylate

NMR (DMSO-d₆, δ): 2.4-3.0 (6H, m), 4.2-4.4 (1H, m), 7.2-7.65 (11H, m), 7.75-7.9 (3H, m), 8.07 (1H, m)

(−)ESI-MS (m/z): 550, 552 (M−Na)

(15) Sodium [3-[[4-[3-[[(2R)-2-(3-chlorophenyl)-2-hydroxyethyl]amino]propyl]phenyl]sulfonyl]phenoxy]-acetate

NMR (DMSO-d₆, δ): 1.67 (2H, quintet, J=7 Hz), 2.40-2.80 (6H, m), 4.17 (2H, s), 4.60 (1H, m), 5.51 (1H, br s, OH), 6.92-7.60 (1H, m), 7.82 (2H, d, J=8 Hz)

(−)ESI-MS (m/z): 502 (free, M−H)⁻

(16) Sodium 3-[[4-[3-[[(2R)-2-(3-chlorphenyl)-2-hydroxyethyl]amino]propyl]phenyl]sulfonyl]benzoate

NMR (DMSO-d₆, δ): 1.66 (2H, quintet, J=7 Hz), 2.40-2.80 (6H, m), 4.60 (1H, m), 5.44 (1H, br s, OH), 7.15-7.60 (7H, m), 7.72-7.92 (3H, m), 8.07 (1H, d, J=8 Hz), 8.30 (1H, s)

(−)ESI-MS (m/z): 472 (free, M−H)⁻

(17) Sodium [4-[[4-[2-[[(2R)-2-(3-chlorophenyl)-2-hydroxyethyl]amino]ethoxy]phenyl]sulfonyl]phenoxy]-acetate

NMR (DMSO-d₆, δ): 2.55-3.00 (4H, m), 4.08 (2H, m), 4.20 (2H, s), 4.63 (1H, m), 5.50 (1H, br s, OH), 6.93 (2H, d, J=8 Hz), 7.08 (2H, d, J=8 Hz), 7.15-7.45 (4H, m), 7.75 (2H, d, J=8 Hz), 7.80 (2H, d, J=8 Hz)

(+)ESI-MS (m/z): 504 (free, M+H)⁺

(18) Sodium 4-[[4-[2-[[(2R)-2-(3-chlorophenyl)-2-hydroxyethyl]amino]ethoxy]phenyl]sulfonyl]benzoate

NMR (DMSO-d₆, δ): 2.58-3.00 (4H, m), 4.08 (2H, m), 4.63 (1H, m), 5.47 (1H, br s, OH), 7.11 (2H, d, J=8 Hz), 7.20-7.45 (4H, m), 7.79 (2H, d, J=8 Hz), 7.84 (2H, d, J=8 Hz), 7.98 (2H, d, J=8 Hz)

(+)ESI-MS (m/z): 474 (free, M+H)⁺

(19) Sodium [2-[[4-[(2R)-2-[[(2R)-2-(3-chlorophenyl)-2-hydroxyethyl]amino]propyl]phenyl]sulfonyl]phenoxy]-acetate

NMR (DMSO-d₆, δ): 0.93 (3H, d, J=6 Hz), 2.40-3.10 (5H, m), 4.03 (2H, s), 4.54 (1H, m), 6.04 (1H, br s, OH), 6.82-7.62 (9H, m), 7.78-8.05 (3H, m)

(−)ESI-MS (m/z): 502 (free, M−H)⁻

(20) Sodium 2-[[4-[(2R)-2-[[(2R)-2-(3-chlorophenyl)-2-hydroxyethyl]amino]propyl[phenyl]sulfonyl]benzoate

NMR (DMSO-d₆, δ): 0.74 (3H, d, J=6 Hz), 2.50-3.20 (5H, m), 4.72 (1H, m), 7.10-7.60 (9H, m), 7.80-8.15 (3H, m)

(−)ESI-MS (m/z): 472 (free, M−H)⁻

(21) Sodium 4′-[[4-[2-[[(2R)-2-(3-chlorophenyl)-2-hydroxyethyl]amino]ethoxy]phenyl]sulfonyl]-1,1′-biphenyl-3-carboxylate

NMR (DMSO-d₆, δ): 2.58-3.02 (4H, m), 4.10 (2H, m), 4.64 (1H, m), 5.56 (1H, br s, OH), 7.05-7.75 (8H, m), 7.75-8.10 (7H, m), 8.20 (1H, s)

(−)ESI-MS (m/z): 550 (free, M−H)⁻

(22) Sodium 4′-[[4-[2-[[(2R)-2-(3-chlorophenyl)-2-hydroxyethyl)amino]ethoxy]phenyl]sulfonyl]-1,1′-biphenyl-4-carboxylate

NMR (DMSO-d₆, δ): 2.60-3.05 (4H, m), 4.12 (2H, m), 4.66 (1H, m), 5.58 (1H, br s, OH), 7.15 (2H, d, J=8 Hz), 7.17-7.50 (4H, m), 7.63 (2H, d, J=8 Hz), 7.80-8.18 (8H, m)

(+)ESI-MS (m/z): 550 (free, M+H)⁺

(23) Sodium 4′-[[4-[3-[[(2R)-2-(3-chlorophenyl)-2-hydroxyethyl]amino]propyl]phenyl]sulfonyl]-1,1′-biphenyl-3-carboxylate

NMR (DMSO-d₆, δ): 1.67 (2H, quintet, J=7 Hz), 2.40-2.80 (6H, m), 4.60 (1H, m), 5.48 (1H, br s, OH), 7.10-8.28 (16H, m)

(+)ESI-MS (m/z): 550 (free, M+H)⁺

(24) Sodium 4′-[[4-[3-[[(2R)-2-(3-chlorophenyl)-2-hydroxyethyl]amino]propyl]phenyl]sulfonyl]-1,1′-biphenyl-4-carboxylate

NMR (DMSO-d₆, δ): 1.67 (2H, quintet, J=7 Hz), 2.40-2.80 (6H, m), 4.61 (1H, m), 5.53 (1H, br s, OH), 7.05-8.20 (16H, m)

(+)ESI-MS (m/z): 550 (free, M+H)⁺

(25) Sodium 3-[4-[[4-[3-[[(2R)-2-(3-chlorophenyl)-2-hydroxyethyl]amino]propyl]phenyl]sulfonyl]phenoxy]-benzoate

NMR (DMSO-d₆, δ): 1.67 (2H, quintet, J=7 Hz), 2.40-2.80 (6H, m), 4.60 (1H, m), 5.51 (1H, br s, OH), 6.95-8.00 (16H, m)

(+)ESI-MS (m/z): 566 (free, M+H)⁺

(26) Sodium 3′-[[4-[3-[[(2R)-2-(3-chlorophenyl)-2-hydroxyethyl]amino]propyl]phenyl]sulfonyl]-1,1′-biphenyl-3-carboxylate

NMR (DMSO-d₆, δ): 1.65 (2H, quintet, J=7 Hz), 2.40-2.80 (6H, m), 4.61 (1H, m), 5.68 (1H, br s, OH), 7.10-8.30 (1H, m)

(+)ESI-MS (m/z): 550 (free, M+H)⁺

(27) Sodium 3-[3-[[4-[3-[[(2R)-2-(3-chlorophenyl)-2-hydroxyethyl]amino]propyl]phenyl]sulfonyl]phenoxy]-benzoate

NMR (DMSO-d₆, δ): 1.65 (2H, quintet, J=7 Hz), 2.40-2.80 (6H, m), 4.61 (1H, m), 6.90-8.05 (16H, m)

(+)ESI-MS (m/z): 566 (free, M+H)⁺

(28) Sodium 5-[[4-[3-[[(2R)-2-(3-chlorophenyl)-2-hydroxyethyl]amino]propyl]phenyl]sulfonyl]-2-hydroxybenzoate

NMR (DMSO-d₆, δ): 1.64 (2H, quintet, J=7 Hz), 2.40-2.90 (6H, m), 4.63 (1H, m), 6.73 (1H, d, J=9 Hz), 7.10-7.50 (6H, m), 7.66 (1H, dd, J=9, 2 Hz), 7.75 (2H, d, J=8 Hz), 8.14 (1H, d, J=2 Hz)

(+)ESI-MS (m/z): 490 (free, M+H)⁺

(29) Sodium 4-[[4-[3-[[(2R)-2-(3-chlorophenyl)-2-hydroxyethyl]amino]propyl]phenyl]sulfonyl]-2-hydroxybenzoate

NMR (DMSO-d₆, δ): 1.77 (2H, quintet, J=7 Hz), 2.50-2.90 (6H, m), 4.72 (1H, m), 7.00-7.55 (8H, m), 7.83 (2H, d, J=8 Hz), 7.84 (1H, d, J=8 Hz)

(+)ESI-MS (m/z): 490 (free, M+H)⁺

(30) Sodium 5-[[4-[2-[[(2R)-2-(3-chlorophenyl)-2-hydroxyethyl]amino]ethoxy]phenyl]sulfonyl]-2-hydroxybenzoate

NMR (DMSO-d₆, δ): 2.55-3.05 (4H, m), 4.08 (2H, m), 4.64 (1H, m), 5.45 (1H, br s, OH), 6.72 (1H, d, J=9 Hz), 7.09 (2H, d, J=9 Hz), 7.15-7.45 (4H, m), 7.64 (1H, dd, J=9, 2 Hz), 7.77 (2H, d, J=9 Hz), 8.12 (1H, d, J=2 Hz)

(−)ESI-MS (m/z): 490 (free, M−H)⁻

(31) Sodium 5-[[4-[[(2R)-2-[[(2R)-2-(3-chlorophenyl)-2-hydroxyethyl]amino]propyl]oxy]phenyl]sulfonyl]-2-hydroxybenzoate

NMR (DMSO-d₆, δ): 1.21 (3H, d, J=6 Hz), 2.75-3.55 (3H, m), 4.09 (2H, m), 4.80 (1H, m), 5.91 (1H, br s, OH), 6.70 (1H, d, J=9 Hz), 7.11 (2H, d, J=9 Hz), 7.22-7.50 (4H, m), 7.63 (1H, dd, J=9, 2 Hz), 7.80 (2H, d, J=9 Hz), 8.09 (1H, d, J=2 Hz)

(−)ESI-MS (m/z): 504 (free, M−H)⁻

(32) Sodium 2-chloro-4-[[4-[3-[[(2R)-2-(3-chlorophenyl)-2-hydroxyethyl]amino]propyl]phenyl]sulfonyl]benzoate

NMR (DMSO-d₆, δ): 1.69 (2H, quintet, J=7 Hz), 2.32-2.82 (6H, m), 4.63 (1H, m), 5.55 (1H, br s, OH), 7.17-7.55 (7H, m), 7.60-7.86 (2H, m), 7.86 (2H, d, J=8 Hz)

(+)ESI-MS (m/z): 508 (free, M+H)⁺

(33) Sodium 5-[[4-[3-[[(2R)-2-(3-chlorophenyl)-2-hydroxyethyl]amino]propyl]phenyl]sulfonyl]-2-methoxybenzoate

NMR (DMSO-d₆, δ): 1.66 (2H, quintet, J=7 Hz), 2.32-2.75 (6H, m), 3.73 (3H, s), 4.56 (1H, m), 5.47 (1H, br s, OH), 7.02 (1H, d, J=9 Hz), 7.15-7.48 (6H, m), 7.55 (1H, d, J=2 Hz), 7.69 (1H, dd, J=9, 2 Hz), 7.76 (2H, d, J=8 Hz)

(+)ESI-MS (m/z): 504 (free, M+H)⁺

### EXAMPLE 6

Under nitrogen, a mixture of ethyl 4-[[4-[2-(benzylamino)ethyl]phenyl]sulfonyl]-2-hydroxybenzoate (215 mg) and (R)-2-(3-chlorophenyl)oxirane (90.7 mg) in ethanol (10 ml) was refluxed for 48 hours. The resulting mixture was evaporated under reduced pressure. The residue was purified by column chromatography on silica gel (hexane/ethyl acetate=3:1 to 3:2) to give ethyl(R)-4-[[4-[2-[benzyl[2-(3-chlorophenyl)-2-hydroxyethyl]amino]ethyl]-phenyl]sulfonyl]-2-hydroxybenzoate (208 mg).

NMR (CDCl₃, δ): 1.40 (3H, t, J=7.1 Hz), 2.55-2.9 (6H, m), 3.55 (1H, d, J=13.4 Hz), 3.96 (1H, d, J=13.4 Hz), 4.42 (2H, q, J=7.1 Hz), 4.6-4.65 (1H, m), 7.15-7.35 (11H, m), 7.4-7.45 (1H, m), 7.5 (1H, m), 7.82 (2H, d, J=8.4 Hz), 7.95 (1H, d, J=8.3 Hz)

(+)ESI-MS (m/z): 594, 596 (M+H)⁺

### EXAMPLE 7

To a solution of ethyl(R)-4-[[4-[2-[benzyl[2-(3-chlorophenyl)-2-hydroxyethyl]amino]ethyl]phenyl]sulfonyl]-2-hydroxybenzoate (204 mg) in ethyl acetate (3 ml) was added 4N hydrogen chloride in ethyl acetate (0.5 ml) at room temperature, and the mixture was evaporated under reduced pressure. A mixture of the residue and 10% palladium on activated carbon (50% wet, 10 mg) in a mixture of ethanol (1.5 ml) and chlorobenzene (3.5 ml) was stirred at room temperature in the presence of hydrogen at an atmospheric pressure for 2 hours. After filtration, the filtrate was evaporated under reduced pressure. The residue was dissolved into a mixture of saturated aqueous sodium bicarbonate and ethyl acetate which contained a little of methanol. After separation, the organic layer was dried over anhydrous magnesium sulfate and evaporated under reduced pressure. The residue was purified by column chromatography on silica gel (chloroform/methanol=20:1 to 15:1) to give ethyl(R)-4-[[4-[2-[[2-(3-chlorophenyl)-2-hydroxyethyl]amino]ethyl]phenyl]sulfonyl]-2-hydroxybenzoate (149 mg).

NMR (CDCl₃, δ): 1.41 (3H, t, J=7.2 Hz), 2.65-3.0 (6H, m) 4.43 (2H, q, J=7.2 Hz), 4.6-4.65 (1H, m), 7.15-7.45 (7H, m), 7.52 (1H, m), 7.85-7.9 (2H, m), 7.97 (1H, d, J=8.4 Hz)

(+)ESI-MS (m/z): 504, 506 (M+H)⁺

### EXAMPLE 8

To a suspension of ethyl(R)-4-[[4-[2-[[2-(3-chlorophenyl)-2-hydroxyethyl]amino]ethyl]phenyl]sulfonyl]-2-hydroxybenzoate (145 mg) in methanol (3 ml) was added 1N sodiumhydroxide (0.72 ml) at room temperature, and the mixture was stirred at the same temperature for 4 days. To the resulting mixture was added 1N hydrochloric acid (0.43 ml), and the mixture was evaporated under reduced pressure. The residue was purified by reversed phase chromatography to give sodium (R)-4-[[4-[2-[[2-(3-chlorophenyl)-2-hydroxyethyl]amino]ethyl]phenyl]sulfonyl]-2-hydroxybenzoate (110 mg).

NMR (DMSO-d₆, δ): 2.85-3.2 (6H, m), 4.75-4.9 (1H, m), 7.0-7.1 (2H, m), 7.25-7.55 (6H, m), 7.75-7.9 (3H, m)

(−)ESI-MS (m/z): 474, 476 (M−Na)

### EXAMPLE 9

The following compounds were obtained according to a similar manner to that of Example 7.

(1) Ethyl(R)-2-[4-[[4-(2-[[2-(3-chlorophenyl)-2-hydroxyethyl]amino]ethyl]phenyl]sulfonyl]phenoxy]-benzoate

NMR (CDCl₃, δ): 1.08 (3H, t, J=7.1 Hz), 2.6-3.0 (6H, m), 4.17 (2H, q, J=7.1 Hz), 4.64 (1H, dd, J=3.6, 8.7 Hz), 6.85-7.0 (2H, m), 7.05-7.4 (8H, m), 7.5-7.6 (1H, m), 7.8-8.0 (5H, m)

(+)ESI-MS (m/z): 580, 582 (M+H)⁺

(2) Ethyl(R)-2-[3-[[4-[2-[[2-(3-chlorophenyl)-2-hydroxyethyl)amino]ethyl]phenyl]sulfonyl]phenoxy]-benzoate

NMR (CDCl₃, δ): 1.07 (3H, t, J=7.1 Hz), 2.6-3.0 (6H, m), 4.15 (2H, q, J=7.1 Hz), 4.64 (1H, dd, J=3.6, 8.8 Hz), 7.0-7.1 (2H, m), 7.15-7.65 (11H, m), 7.8-7.9 (2H, m), 7.95-8.0 (1H, m)

(+)ESI-MS (m/z): 580 (M+H)⁺

(3) Ethyl(R)-[4-[[4-[2-[[2-(3-chlorophenyl)-2-hydroxyethyl]amino]ethyl]phenyl]sulfonyl]phenyl]acetate

NMR (CDCl₃, δ): 1.24 (3H, t, J=7.1 Hz), 2.6-3.0 (6H, m) 3.65 (2H, s), 4.14 (2H, q, J=7.1 Hz), 4.63 (1H, dd, J=3.7, 8.8 Hz), 7.15-7.35 (6H, m), 7.42 (2H, d, J=8.3 Hz), 7.8-7.95 (4H, m)

(+)ESI-MS (m/z): 502, 504 (M+H)⁺

(4) Methyl(R)-3′-[[4-[2-[[2-(3-chlorophenyl)-2-hydroxyethyl]amino]ethyl]phenyl]sulfonyl]-1,1′-biphenyl-4-carboxylate

NMR (CDCl₃, δ): 2.6-3.0 (6H, m), 3.95 (3H, s), 4.62 (1H, dd, J=3.6, 8.7 Hz), 7.1-7.4 (6H, m), 7.55-7.7 (3H, m), 7.75-8.0 (4H, m), 8.1-8.2 (3H, m)

(+)ESI-MS (m/z): 550, 552 (M+H)⁺

(5) Ethyl(R)-3′-[[4-[2-[[2-(3-chlorophenyl)-2-hydroxyethyl]amino]ethyl]phenyl]sulfonyl]-1,1′-biphenyl-3-carboxylate

NMR (CDCl₃, δ): 1.42 (3H, t, J=7.2 Hz), 2.6-3.0 (6H, m), 4.42 (2H, q, J=7.2 Hz), 4.63 (1H, dd, J=3.6, 8.7 Hz) 7.1-7.4 (6H, m), 7.5-7.7 (2H, m), 7.7-8.0 (5H, m), 8.05-8.3 (3H, m)

(+)ESI-MS (m/z): 564, 566 (M+H)⁺

(6) Ethyl(R)-3′-[[4-[2-[[2-(3-chlorophenyl)-2-hydroxyethyl]amino]ethyl]phenyl]sulfonyl]-1,1′-biphenyl-2-carboxylate

NMR (CDCl₃, δ): 0.87 (3H, t, J=7.1 Hz), 2.6-2.7 (1H, m), 2.8-3.0 (5H, m), 3.96 (2H, q, J=7.1 Hz), 4.64 (1H, dd, J=3.5, 8.9 Hz), 7.15-7.35 (7H, m), 7.45-7.6 (4H, m), 7.85-8.0 (5H, m)

(+)ESI-MS (m/z): 564 (M+H)⁺

(7) Ethyl(R)-4-[3-[[4-[2-[[2-(3-chlorophenyl)-2-hydroxyethyl]amino]ethyl]phenyl]sulfonyl]phenoxy]-benzoate

NMR (CDCl₃, δ): 1.40 (3H, t, J=7.1 Hz), 2.6-3.05 (6H, m), 4.38 (2H, q, J=7.1 Hz), 4.64 (1H, dd, J=3.7, 8.8 Hz), 6.95-7.05 (2H, m), 7.15-7.35 (7H, m), 7.4-7.75 (3H, m), 7.8-7.9 (2H, m), 8.0-8.1 (2H, m)

(−)ESI-MS (m/z): 578, 580 (M−H)⁻

(8) Ethyl(R)-3-[3-[[4-[2-[[2-(3-chlorophenyl)-2-hydroxyethyl]amino]ethyl]phenyl]sulfonyl]phenoxy]-benzoate

NMR (CDCl₃, δ): 1.38 (3H, t, J=7.1 Hz), 2.6-3.0 (6H, m), 4.37 (2H, q, J=7.1 Hz), 4.64 (1H, dd, J=3.7, 8.8 Hz), 7.1-7.7 (13H, m), 7.8-7.9 (3H, m)

(+)ESI-MS (m/z): 580, 582 (M+H)⁺

(9) Ethyl(R)-4′-[[4-[2-[[2-(3-chlorophenyl)-2-hydroxyethyl]amino]ethyl]phenyl]sulfonyl]-2′-hydroxy-1,1′-biphenyl-3-carboxylate

NMR (CDCl₃, δ): 1.37 (3H, t, J=7.1 Hz), 2.6-3.0 (6H, m), 4.38 (2H, q, J=7.1 Hz), 4.65 (1H, dd, J=3.6, 8.8 Hz), 7.1-7.7 (10H, m), 7.90 (2H, d, J=8.3 Hz), 8.0-8.1 (1H, m), 8.16 (1H, m)

(+)ESI-MS (m/z): 580, 582 (M+H)⁺

(10) Ethyl [2-[[4-[(2R)-2-[[(2R)-2-(3-chlorophenyl)-2-hydroxyethyl]amino]propyl]phenyl]sulfonyl]phenoxy]-acetate

NMR (CDCl₃, δ): 1.07 (3H, d, J=6 Hz), 1.24 (3H, t, J=7 Hz), 2.50-3.05 (5H, m), 4.18 (2H, q, J=7 Hz), 4.52 (1H, dd, J=9, 4 Hz), 4.59 (2H, s), 6.80 (1H, d, J=8 Hz), 7.02-7.40 (7H, m), 7.52 (1H, t, J=8 Hz), 7.99 (2H, d, J=8 Hz), 8.20 (1H, d, J=8 Hz)

(+)ESI-MS (m/z): 532 (free, M+H)⁺

(11) Ethyl 2-[[4-[(2R)-2-[[(2R)-2-(3-chlorophenyl)-2-hydroxyethyl]amino]propyl]phenyl]sulfonyl]benzoate

NMR (CDCl₃, δ): 1.06 (3H, d, J=6 Hz), 1.38 (3H, t, J=7 Hz), 2.50-3.05 (5H, m), 4.42 (2H, q, J=7 Hz), 4.53 (1H, dd, J=9, 4 Hz), 7.00-8.20 (12H, m)

(+)ESI-MS (m/z): 502 (M+H)⁺

### EXAMPLE 10

Under nitrogen, a mixture of ethyl 4-[[4-(3-aminopropyl)phenyl]sulfonyl]-2-methylbenzoate (3.42 g) and (R)-2-(3-chlorophenyl)oxirane (731 mg) in ethanol (34 ml) was refluxed for 24 hours. The resulting mixture was evaporated under reduced pressure. The residue was purified by column chromatography on silica gel (chloroform/methanol=20:1 to 40:3) to give ethyl(R)-4-[[4-[3-[[2-(3-chlorophenyl)-2-hydroxyethyl]amino]propyl]phenyl]sulfonyl]-2-methylbenzoate (1.44 g).

NMR (CDCl₃, δ): 1.38 (3H, t, J=7.1 Hz), 1.7-1.9 (2H, m), 2.55-2.9 (7H, m), 4.36 (2H, q, J=7.1 Hz), 4.64 (1H, dd, J=3.6, 8.7 Hz), 7.15-7.4 (6H, m), 7.7-8.0 (5H, m)

(+)ESI-MS (m/z): 516, 518 (M+H)⁺

### EXAMPLE 11

To a suspension of ethyl(R)-4-[[4-[3-[[2-(3-chlorophenyl)-2-hydroxyethyl]amino]propyl]phenyl]sulfonyl]-2-methylbenzoate (1.42 g) in ethanol (14 ml) was added 1N sodiumhydroxide (2.75 ml) at room temperature, and the mixture was stirred at 60° C. for 1.3 hours. The resulting mixture was evaporated under reduced pressure and dried in vacuo to give sodium (R)-4-[[4-[3-[[2-(3-chlorophenyl)-2-hydroxyethyl]amino]propyl]phenyl]sulfonyl]-2-methylbenzoate (1.42 g).

NMR (DMSO-d₆, δ): 1.55-1.75 (2H, m), 2.35-2.7 (9H, m), 4.55-4.65 (1H, m), 7.2-7.65 (9H, m), 7.81 (2H, d, J=8.2 Hz)

(−)ESI-MS (m/z): 486, 488 (M−Na)⁻

### EXAMPLE 12

The following compound was obtained according to a similar manner to that of Example 11.

Sodium (R)-[4-[[4-[2-[[2-(3-chlorophenyl)-2-hydroxyethyl]amino]ethyl]phenyl]sulfonyl]phenyl]acetate

NMR (DMSO-d₆, δ): 2.55-2.8 (6H, m), 3.23 (2H, s), 4.55-4.65 (1H, m), 7.2-7.45 (8H, m), 7.7-7.85 (4H, m)

(+)ESI-MS (m/z): 472, 474 (M−Na)

### EXAMPLE 13

A mixture of (R)-4-[[4-[2-[benzyl[2-(3-chlorophenyl)-2-hydroxyethyl]amino]ethyl]phenyl]sulfonyl]phenol (1.31 g), triethylamine (3.3 ml) and 10% palladium on activated carbon (0.50% wet, 0.65 g) in a mixture of methanol (13 ml) and chlorobenzene (13 ml) was stirred at room temperature in the presence of hydrogen at an atmospheric pressure for 5 hours. After filtration, the filtrate was evaporated under reduced pressure. The residue was dissolved into a mixture of ethyl acetate and saturated aqueous sodium hydrogencarbonate. After separation, the organic layer was washed with brine, dried over anhydrous magnesium sulfate and evaporated under reduced pressure. The residue was purified by column chromatography on silica gel (chloroform:methanol=20:1 to 8:1) to give (R)-4-[[4-[2-[[2-(3-chlorophenyl)-2-hydroxyethyl]amino]ethyl]phenyl]sulfonyl]phenol (789 mg).

NMR (DMSO-d₆, δ): 2.55-2.85 (6H, m), 4.55-4.6 (1H, m), 6.9-6.95 (2H, m), 7.2-7.8 (4H, m)

(+)ESI-MS (m/z): 432, 434 (M+H)⁺

### EXAMPLE 14

Under nitrogen at room temperature, to a solution of (R)-4-[[4-[2-[[2-(3-chlorophenyl)-2-hydroxyethyl]amino]-ethyl]phenyl]sulfonyl]phenol (1.0 g) in tetrahydrofuran (8 ml) was added di-tert-butyl dicarbonate (0.56 g) in tetrahydrofuran (2 ml), and the mixture was stirred at the same temperature for 12 hours. The resulting mixture was poured into water and the aqueous mixture was extracted with ethyl acetate. The organic layer was washed with brine, dried over anhydrous magnesium sulfate and evaporated under reduced pressure. The residue was purified by column chromatography on silica gel (hexane:ethyl acetate=2:1 to 1:1) to give tert-butyl(R)-[2-(3-chlorophenyl)-2-hydroxyethyl][2-[4-[(4-hydroxyphenyl)sulfonyl]phenyl]-ethyl]carbamate (1.1 g).

NMR (CDCl₃, δ): 1.2-1.5 (9H, m), 2.6-2.95 (2H, m), 3.15-3.6 (4H, m), 4.8-4.95 (1H, m), 6.8-6.95 (2H, m), 7.15-7.45 (6H, m), 7.7-7.9 (2H, m)

(+)ESI-MS (m/z): 554, 556 (M+Na)⁺

### EXAMPLE 15

A mixture of (R)-3-[[4-[2-[benzyl[2-(3-chlorophenyl)-2-hydroxyethyl]amino]ethyl]phenyl]sulfonyl]phenol (202 mg) and 10% palladium on activated carbon (50% wet, 100 mg) in a mixture of methanol (2 ml) and chlorobenzene (2 ml) was stirred at room temperature in the presence of hydrogen at an atmospheric pressure for 2 hours. After filtration, the filtrate was evaporated under reduced pressure. The residue was dissolved into a mixture of saturated aqueous sodium bicarbonate and ethyl acetate. After separation, the organic layer was dried over magnesium sulfate and evaporated under reduced pressure. The residue was purified by column chromatography on silica gel (chloroform:methanol=20:1 to 8:1) followed by treatment with 4N hydrogen chloride in 1,4-dioxane and dryness to give (R)-3-[[4-[2-[[2-(3-chlorophenyl)-2-hydroxyethyl]amino]ethyl]phenyl]-sulfonyl]phenol hydrochloride (90 mg).

NMR (DMSO-d₆, δ): 2.9-3.5 (6H, m), 4.85-5.0 (1H, m), 7.0-7.1 (1H, m), 7.2-7.6 (9H, m), 7.85-7.95 (2H, m)

(+) APCI-MS (m/z): 432, 434 (M−HCl+H)⁺

### EXAMPLE 16

Under nitrogen, to a solution of (R)-3-[[4-[2-[benzyl[2-(3-chlorophenyl)-2-hydroxyethyl]amino]ethyl]-phenyl]sulfonyl]phenol (3.55 g) and 2,6-lutidine (1.09 ml) in dichloromethane (35 ml) was added trifluoromethanesulfonic anhydride (1.26 ml) in dryice-acetone bath, and the mixture was stirred at the same temperature for 1 hour. The resulting mixture was poured into 1N hydrochloric acid and the aqueous mixture was extracted with ethyl acetate. The organic layer was washed successively with water, saturated aqueous sodium bicarbonate and brine, dried over anhydrous magnesium sulfate and evaporated under reduced pressure. The residue was purified by column chromatography on silica gel (hexane/ethyl acetate=5:1 to 2:1) to give (R)-3-[[4-[2-[benzyl[2-(3-chlorophenyl)-2-hydroxyethyl]amino]ethyl]-phenyl]sulfonyl]phenyl trifluoromethanesulfonate (3.95 g).

NMR (CDCl₃, δ): 2.5-2.9 (6H, m), 3.55 (1H, d, J=13.4 Hz) 3.90 (1H, d, J=13.4 Hz), 4.60 (1H, dd, J=3.7, 9.9 Hz), 7.1-7.35 (11H, m), 7.4-7.7 (2H, m), 7.8-8.0 (4H, m)

(+)ESI-MS (m/z): 654 (M+H)⁺

### EXAMPLE 17

To a solution of (R)-3-[[4-[2-[benzyl[2-(3-chlorophenyl)-2-hydroxyethyl]amino]ethyl]phenyl]sulfonyl]-phenyl trifluoromethanesulfonate (480 mg) and 2-carboxyphenylboronic acid (480 mg) in 1,2-dimethoxyethane (7 ml) were added tetrakis(triphenylphosphine)palladium(0) (42.4 mg) and 2M sodium carbonate (1.14 ml) at room temperature, and the mixture was stirred at 80° C. for 10 hours. The resulting mixture was poured into pH 4 phosphate buffer and the aqueous mixture was extracted with chloroform. The organic layer was dried over anhydrous magnesium sulfate and evaporated under reduced pressure. The residue was purified by column chromatography on silica gel (chloroform/methanol=30:1 to 20:1) to give (R)-3′-[[4-[2-[benzyl[2-(3-chlorophenyl)-2-hydroxyethyl]amino]ethyl]phenyl]sulfonyl]-1,1′-biphenyl-2-carboxylic acid (354 mg).

NMR (DMSO-d₆, δ): 2.55-2.8 (6H, m), 3.58 (1H, d, J=13.9 Hz), 3.73 (1H, d, J=13.9 Hz), 4.6-4.75 (1H, m), 6.95-8.0 (21H, m)

(−)ESI-MS (m/z): 624 (M−H)

### EXAMPLE 18

To a solution of methyl 4′-[[4-[2-[(tert-butoxycarbonyl)[(2R)-2-(3-chlorophenyl)-2-hydroxyethyl]amino]ethyl]phenyl]sulfonyl]-1,1′-biphenyl-3-carboxylate (125 mg) in 1,4-dioxane (1.3 ml) was added 1N sodium hydroxide solution (0.48 ml), and the mixture was stirred at 50° C. for 19 hours. After the solution was made acidic with 1N hydrochloric acid, the mixture was extracted with chloroform-methanol. The organic layer was washed with brine, dried over magnesium sulfate, and filtered. The filtrate was concentrated to give 4′-[[4-[2-[(tert-butoxycarbonyl)[(2R)-2-(3-chlorophenyl)-2-hydroxyethyl]-amino]ethyl]phenyl]sulfonyl]-1,1′-biphenyl-3-carboxylic acid (104 mg) as a white amorphous.

NMR (DMSO-d₆, δ): 1.07, 1.19 (total 9H, a pair of s), 2.70-2.95 (2H, m), 2.95-3.45 (4H, m), 4.71 (1H, m), 5.58 (1H, br s, OH), 7.10-7.53 (6H, m), 7.64 (1H, t, J=8 Hz), 7.82-8.12 (8H, s), 8.20 (1H, s)

(−)ESI-MS (m/z): 634 (M−H)⁻

### EXAMPLE 19

4′[[4-[2-[(tert-Butoxycarbonyl)[(2R)-2-(3-chlorophenyl)-2-hydroxyethyl]amino]ethyl]phenyl]sulfonyl]-1,1′-biphenyl-3-carboxylic acid (91 mg) and 4N hydrogen chloride in 1,4-dioxane (0.92 ml) were mixed and stirred at room temperature for 15.5 hours. The solvent was evaporated and the residual powder was treated with ethanol (0.92 ml)-1N sodium hydroxide solution (0.35 ml). The solvent was evaporated to give sodium 4′-[[4-[2-[[(2R)-2-(3-chlorophenyl)-2-hydroxyethyl]amino]ethyl]phenyl]sulfonyl]-1,1′-biphenyl-3-carboxylate (54 mg) as a white powder.

NMR (DMSO-d₆, δ): 2.50-2.90 (6H, m), 4.60 (1H, m), 5.48 (1H, br s, OH), 7.10-7.55 (7H, m), 7.55-7.72 (1H, m), 7.72-8.10 (7H, m), 8.20 (1H, s)

(−)ESI-MS (m/z): 534 (free, M−H)⁻

### EXAMPLE 20

Ethyl 3-[4-[[4-[2-[(tert-butoxycarbonyl)[(2R)-2-(3-chlorophenyl)-2-hydroxyethyl]amino]ethyl]phenyl]sulfonyl]-phenoxy]benzoate (48 mg) and 4N hydrogen chloride in 1,4-dioxane (1 ml) were mixed and stirred at room temperature for 6.5 hours. The solvent was evaporated and the residual powder was treated with ethanol (1 ml)-1N sodium hydroxide solution (0.16 ml). After the mixture was heated to reflux for 9 hours, the solvent was evaporated to give sodium 3-[4-[[4-[2-[[(2R)-2-(3-chlorophenyl)-2-hydroxyethyl]amino]-ethyl]phenyl]sulfonyl]phenoxy]benzoate (46 mg) as a white powder.

NMR (DMSO-d₆, δ): 2.50-2.90 (6H, m), 4.60 (1H, m), 5.50 (1H, br s, OH), 6.95-7.16 (3H, m), 7.16-7.60 (8H, m), 7.65-8.00 (5H, m)

(+)ESI-MS (m/z): 552 (free, M+H)⁺

### EXAMPLE 21

Under nitrogen atmosphere, a mixture of 4-[[4-[2-[(tert-butoxycarbonyl)[(2R)-2-(3-chlorophenyl)-2-hydroxyethyl]amino]ethyl]phenyl]sulfonyl]phenyl trifluoromethanesulfonate (265 mg), palladium(II)acetate (5 mg), 2-[bis(tert-butyl)phosphino]biphenyl (12 mg), and powdered potassium phosphate (177 mg) in toluene (2.6 ml) was heated to 100° C. for 10 hours. After being allowed to cool to room temperature, the mixture was concentrated and the residue was purified by column chromatography (silica gel, hexane/ethyl acetate) to give ethyl 4-[4-[[4-[2-[(tert-butoxycarbonyl)[(2R)-2-(3-chlorophenyl)-2-hydroxyethyl]-amino]ethyl]phenyl]sulfonyl]phenoxy]benzoate (93 mg) as a white amorphous.

NMR (CDCl₃, δ): 1.36 (9H, br s), 1.40 (3H, t, J=7 Hz), 2.60-3.05 (2H, m), 3.05-3.60 (4H, m), 4.27 (1H, br s, OH), 4.38 (2H, q, J=7 Hz), 4.86 (1H, m), 6.90-7.45 (10H, m), 7.86 (2H, d, J=8 Hz), 7.90 (2H, d, J=8 Hz), 8.07 (2H, d, J=8 Hz)

(+)ESI-MS (m/z): 702 (M+Na)⁺

### EXAMPLE 22

To a solution of 3-[[4-[3-[benzyl[(2R)-2-(3-chlorophenyl)-2-hydroxyethyl]amino]propyl]phenyl]sulfonyl]-phenol (282 mg) in N,N-dimethylformamide (2.3 ml) were added powdered potassium carbonate (88 mg) and ethyl bromoacetate (0.07 ml), and the mixture was stirred at 60° C. for 1.5 hours. After being allowed to cool to room temperature, the mixture was partitioned between hexane/ethyl acetate (1/2) and water. The organic layer was separated, washed successively with water and brine, dried over magnesium sulfate, and filtered. The filtrate was concentrated and the residue was purified by column chromatography (silica gel, hexane/ethyl acetate) to give ethyl [3-[[4-[3-[benzyl[(2R)-2-(3-chlorophenyl)-2-hydroxyethyl]amino]propyl]phenyl]sulfonyl]phenoxy]acetate (270 mg) as a colorless oil.

NMR (CDCl₃, δ): 1.29 (3H, t, J=7 Hz), 1.80 (2H, quintet, J=7 Hz), 2.35-2.80 (6H, m), 3.48 (1H, d, J=13 Hz), 3.87 (1H, d, J=13 Hz), 4.26 (2H, q, J=7 Hz), 4.61 (1H, dd, J=10, 4 Hz), 4.65 (2H, s), 7.00-7.62 (15H, m), 7.80 (2H, d, J=8 Hz)

(+)ESI-MS (m/z): 622 (M+H)⁺

### EXAMPLE 23

To a solution of ethyl [3-[[4-[3-[benzyl[(2R)-2-(3-chlorophenyl)-2-hydroxyethyl]amino]propyl]phenyl]sulfonyl]-phenoxy]acetate (252 mg) in ethyl acetate (2.5 ml) was added 4N hydrogen chloride/ethyl acetate (0.5 ml). After the solvent was evaporated, the residue was dissolved in chlorobenzene (3.5 ml)-ethanol (1.5 ml), and the solution was hydrogenated (1 atm) over 10% palladium on carbon (12 mg) at room temperature for 3.5 hours. After the catalyst was filtered off, the filtrate was concentrated to give ethyl [3-[[4-[3-[[(2R)-2-(3-chlorophenyl)-2-hydroxyethyl]-amino]propyl]phenyl]sulfonyl]phenoxy]acetate hydrochloride (221 mg) as a white powder.

NMR (DMSO-d₆, δ): 1.19 (3H, t, J=7 Hz), 1.96 (2H, quintet, J=7 Hz), 2.73 (2H, t, J=7 Hz), 2.80-3.25 (4H, m), 4.15 (2H, q, J=7 Hz), 4.92 (2H, s), 4.95 (1H, m), 6.29 (1H, br s, OH), 7.15-7.62 (10H, m), 7.92 (2H, d, J=8 Hz)

(+)ESI-MS (m/z): 532 (free, M+H)⁺

### EXAMPLE 24

To a solution of 3-[[4-[3-[benzyl[(2R)-2-(3-chlorophenyl)-2-hydroxyethyl]amino]propyl]phenyl]sulfonyl]-phenol (287 mg) in dimethyl sulfoxide (1.5 ml) were added powdered potassium carbonate (115 mg) and 2-fluorobenzaldehyde (79 mg), and the mixture was stirred at 100° C. for 4 hours. After being allowed to cool to room temperature, the mixture was partitioned between hexane/ethyl acetate (1/2) and water. The organic layer was separated, washed successively with water and brine, dried over magnesium sulfate, and filtered. The filtrate was concentrated and the residue was purified by column chromatography (silica gel, hexane/ethyl acetate) to give 2-[3-[[4-[3-[benzyl[(2R)-2-(3-chlorophenyl)-2-hydroxyethyl]-amino]propyl]phenyl]sulfonyl]phenoxy]benzaldehyde (166 mg) as a colorless oil.

NMR (CDCl₃, δ): 1.81 (2H, quintet, J=7 Hz), 2.35-2.80 (6H, m), 3.49 (1H, d, J=13 Hz), 3.88 (1H, d, J=13 Hz), 4.61 (1H, dd, J=10, 4 Hz), 6.80-8.10 (21H, m), 10.40 (1H, s)

(+)ESI-MS (m/z): 640 (M+H)⁺

### EXAMPLE 25

2-[3-[[4-[3-[(tert-Butoxycarbonyl)[(2R)-2-(3-chlorophenyl)-2-hydroxyethyl]amino]propyl]phenyl]sulfonyl]-phenoxy]benzoic acid (171 mg) and 4N hydrogen chloride in 1,4-dioxane (1.7 ml) were mixed and stirred at room temperature for 15 hours. The solvent was evaporated to give 2-[3-[[4-[3-[[(2R)-2-(3-chlorophenyl)-2-hydroxyethyl]-amino]propyl]phenyl]sulfonyl]phenoxy]benzoic acid hydrochloride (163 mg) as a white amorphous.

NMR (DMSO-d₆, δ): 1.82-2.12 (2H, m), 2.74 (2H, t, J=7 Hz), 2.83-3.30 (4H, m), 4.96 (1H, m), 6.31 (1H, br s, OH), 7.08-7.98 (16H, m)

(−)ESI-MS (m/z): 564 (free, M-H)

### EXAMPLE 26

To a suspension of 3-[[4-[2-([(2R)-2-(3-chlorophenyl)-2-hydroxyethyl]amino]ethyl]phenyl]sulfonyl]phenol hydrochloride (324 mg) in tetrahydrofuran (3.2 ml) were added 1N sodium hydroxide solution (0.7 ml) and di-tert-butyl dicarbonate (169 mg), and the mixture was stirred at room temperature for 1 hour. The mixture was partitioned between ethyl acetate and water. The organic layer was separated, washed successively with water and brine, dried over magnesium sulfate, and filtered. The filtrate was concentrated and the residue was purified by column chromatography (silica gel, hexane/ethyl acetate) to give tert-butyl [(2R)-2-(3-chlorophenyl)-2-hydroxyethyl][2-[4-[(3-hydroxyphenyl)sulfonyl]phenyl]ethyl]carbamate (318 mg) as a white amorphous.

NMR (CDCl₃, δ): 1.33 (9H, s), 2.45-3.00 (2H, m) 3.00-3.65 (4H, m), 4.55 (1H, br s, OH), 4.71 (1H, m), 6.50-8.00 (12H, m)

(+)ESI-MS (m/z): 554 (M+Na)⁺

### EXAMPLE 27

The following compounds were obtained according to a similar manner to that of Example 16.

(1) 4-[[4-[2-[(tert-Butoxycarbonyl)[(2R)-2-(3-chlorophenyl)-2-hydroxyethyl]amino]ethyl]phenyl]-sulfonyl]phenyl trifluoromethanesulfonate

NMR (DMSO-d₆, δ): 1.31 (9H, br s), 2.60-3.05 (2H, m), 3.05-3.60 (4H, m), 4.24 (1H, br s, OH), 4.87 (1H, m), 7.05-7.48 (8H, m), 7.87 (2H, d, J=8 Hz), 8.03 (2H, d, J=9 Hz)

(+) APCI-MS (m/z): 564 (M−Boc+H)⁺

(2) 3-[[4-[3-[Benzyl[(2R)-2-(3-chlorophenyl)-2-hydroxyethyl]amino]propyl]phenyl]sulfonyl]phenyl trifluoromethanesulfonate

NMR (CDCl₃, δ): 1.87 (2H, quintet, J=7 Hz), 2.43-2.90 (6H, m), 3.62 (1H, d, J=13 Hz), 3.92 (1H, d, J=13H) 4.66 (1H, dd, J=10, 4 Hz), 7.05-8.00 (17H, m)

(+)ESI-MS (m/z): 668 (M+H)⁺

(3) 4-[[4-[2-[Benzyl[(2R)-2-(3-chlorophenyl)-2-hydroxyethyl]amino]ethoxy]phenyl]sulfonyl]phenyl trifluoromethanesulfonate

NMR (CDCl₃, δ): 2.65 (1H, dd, J=13, 10 Hz), 2.84-3.22 (2H, m), 2.86 (1H, dd, J=13, 4 Hz), 3.69 (1H, d, J=13 Hz), 3.95 (1H, d, J=13 Hz), 3.97-4.09 (2H, m), 4.65 (1H, dd, J=10, 4 Hz), 6.95 (2H, d, J=8 Hz), 7.10-7.38 (9H, m), 7.39 (2H, d, J=8 Hz), 7.87 (2H, d, J=8 Hz), 8.02 (2H, d, J=8 Hz)

(+)ESI-MS (m/z): 670 (M+H)⁺

(4) 2-[[4-[(2R)-[Benzyl[(2R)-2-(3-chlorophenyl)-2-hydroxyethyl]amino]propyl]phenyl]sulfonyl]phenyl trifluoromethanesulfonate

NMR (CDCl₃, δ): 1.03 (3H, d, J=6 Hz), 2.35-2.95 (4H, m), 3.00-3.26 (1H, m), 3.51 (1H, d, J=13 Hz), 3.84 (1H, d, J=13 Hz), 4.53 (1H, dd, J=10, 4 Hz), 6.85-7.95 (16H, m), 8.29 (1H, d, J=8 Hz)

(+)ESI-MS (m/z): 668 (M+H)⁺

(5) 4-[[4-[3-[Benzyl[(2R)-2-(3-chlorophenyl)-2-hydroxyethyl]amino]propyl]phenyl]sulfonyl]phenyl trifluoromethanesulfonate

NMR (CDCl₃, δ): 1.81 (2H, quintet, J=7 Hz), 2.38-2.80 (6H, m), 3.49 (1H, d, J=13 Hz), 3.88 (1H, d, J=13 Hz), 4.61 (1H, dd, J=10, 4 Hz), 7.05-7.50 (13H, m), 7.82 (2H, d, J=8 Hz), 8.03 (2H, d, J=8 Hz)

(+)ESI-MS (m/z): 668 (M+H)⁺

### EXAMPLE 28

The following compound was obtained according to a similar manner to that of Example 11.

Sodium 4-[[4-[[(2R)-2-[[(2R)-2-(3-chlorophenyl)-2-hydroxyethyl]amino]propyl]oxy]phenyl]sulfonyl]benzoate

NMR (DMSO-d₆, δ): 1.0-1.1 (3H, m), 2.65-2.75 (2H, m), 2.9-3.05 (1H, m), 3.75-3.9 (2H, m), 4.55-4.65 (1H, m), 7.05-7.15 (2H, m), 7.2-7.4 (4H, m), 7.75-7.9 (4H, m), 7.95-8.0 (2H, m)

(−)ESI-MS (m/z): 488 (M−Na)

### EXAMPLE 29

The following compound was obtained according to a similar manner to that of Example 18. 4′-[[4-[2-[(tert-Butoxycarbonyl)[(2R)-2-(3-chlorophenyl)-2-hydroxyethyl]amino]ethyl]phenyl]sulfonyl]-1,1′-biphenyl-4-carboxylic acid

NMR (DMSO-d₆, δ): 1.07, 1.19 (total 9H, a pair of s), 2.70-2.95 (2H, m), 2.95-3.45 (4H, m), 4.72 (1H, m) 5.59 (1H, br s, OH), 7.10-7.52 (6H, m), 7.75-8.12 (10H, m)

(−)ESI-MS (m/z): 634 (M−H)

### EXAMPLE 30

The following compound was obtained according to a similar manner to that of Example 19.

Sodium 4′-[[4-[2-[[(2R)-2-(3-chlorophenyl)-2-hydroxyethyl]amino]ethyl]phenyl]sulfonyl]-1,1′-biphenyl-4-carboxylate

NMR (DMSO-d₆, δ): 2.50-2.90 (6H, m), 4.60 (1H, m), 5.49 (1H, br s, OH), 7.10-7.72 (8H, m), 7.72-8.10 (8H, m)

(−)ESI-MS (m/z): 534 (free, M-H)

### EXAMPLE 31

The following compounds were obtained according to a similar manner to that of Example 20.

(1) Sodium 3-[4-[[4-[2-[[(2R)-2-(3-chlorophenyl)-2-hydroxyethyl]amino]ethyl]phenyl]sulfonyl]phenoxy]-benzoate

NMR (DMSO-d₆, δ): 2.50-2.90 (6H, m), 4.63 (1H, m), 7.00-7.20 (3H, m), 7.20-7.55 (8H, m), 7.65-8.00 (5H, m)

(−)ESI-MS (m/z): 550 (free, M−H)⁻

(2) Sodium 4-[4-[[4-[2-[[(2R)-2-(3-chlorophenyl)-2-hydroxyethyl]amino]ethyl]phenyl]sulfonyl]phenoxy]-benzoate

NMR (DMSO-d₆, δ): 2.50-2.90 (6H, m), 4.61 (1H, m), 6.31 (1H, br s, OH), 6.90-8.10 (16H, m)

(−)ESI-MS (m/z): 550 (free, M−H)⁻

(3) Sodium 2-[3-[[4-[3-[[(2R)-2-(3-chlorophenyl)-2-hydroxyethyl]amino]propyl]phenyl]sulfonyl]phenoxy]-nicotinate

NMR (DMSO-d₆, δ): 1.67 (2H, quintet, J=7 Hz), 2.30-2.80 (6H, m), 4.61 (1H, m), 5.54 (1H, br s, OH), 7.00-8.10 (15H, m)

(+)ESI-MS (m/z): 567 (free, M+H)⁺

### EXAMPLE 32

The following compounds were obtained according to a similar manner to that of Example 21.

(1) Methyl 3-[4-[[4-[2-[(tert-butoxycarbonyl)[(2R)-2-(3-chlorophenyl)-2-hydroxyethyl]amino]ethyl]phenyl]-sulfonyl]phenoxy]benzoate

NMR (CDCl₃, δ): 1.36 (9H, br s), 2.60-3.05 (2H, m), 3.05-3.60 (4H, m), 3.91 (3H, s), 4.31 (1H, br s, OH), 4.86 (1H, m), 7.00 (2H, d, J=9 Hz), 7.10-7.40 (7H, m), 7.48 (1H, t, J=8 Hz), 7.67 (1H, s), 7.75-7.98 (5H, m)

(+)ESI-MS (m/z): 688 (M+Na)⁺

(2) Ethyl 3-[4-[[4-[3-[benzyl[(2R)-2-(3-chlorophenyl)-2-hydroxyethyl]amino]propyl]phenyl]sulfonyl]phenoxy]-benzoate

NMR (CDCl₃, δ): 1.41 (3H, t, J=7 Hz), 1.81 (2H, quintet, J=7 Hz), 2.37-2.80 (6H, m), 3.49 (1H, d, J=13 Hz), 3.87 (1H, d, J=13 Hz), 4.37 (2H, q, J=7 Hz), 4.61 (1H, dd, J=10, 4 Hz), 7.01 (2H, d, J=8 Hz), 7.05-7.70 (15H, m), 7.81 (2H, d, J=8 Hz), 7.89 (2H, d, J=8 Hz)

(+)ESI-MS (m/z): 684 (M+H)⁺

### EXAMPLE 33

The following compounds were obtained according to a similar manner to that of Example 22.

(1) Ethyl [2-[[4-[(2R)-2-[benzyl[(2R)-2-(3-chlorophenyl)-2-hydroxyethyl]amino]propyl]phenyl]sulfonyl]phenoxy]-acetate

NMR (CDCl₃, δ): 1.02 (3H, d, J=6 Hz), 1.24 (3H, t, J=7 Hz), 2.40-2.90 (4H, m), 2.98-3.22 (1H, m), 3.48 (1H, d, J=13 Hz), 3.82 (1H, d, J=13 Hz), 4.19 (2H, q, J=7 Hz), 4.52 (1H, dd, J=10, 4 Hz), 4.59 (2H, s), 6.81 (1H, d, J=8 Hz), 6.92-7.40 (12H, m), 7.51 (1H, t, J=8 Hz), 7.94 (2H, d, J=8 Hz), 8.17 (1H, d, J=8 Hz)

(+)ESI-MS (m/z): 644 (M+Na)⁺

(2) Ethyl [4-[[4-[2-[benzyl[(2R)-2-(3-chlorophenyl)-2-hydroxyethyl]amino]ethoxy]phenyl]sulfonyl]phenoxy]-acetate

NMR (CDCl₃, δ): 1.29 (3H, t, J=7 Hz) 2.64 (1H, dd, J=13, 10Hz), 2.80-3.22 (2H, m), 2.85 (1H, dd, J=13, 4 Hz), 3.69 (1H, d, J=13 Hz), 3.94-4.10 (2H, m), 4.01 (1H, d, J=13 Hz), 4.26 (2H, q, J=7 Hz), 4.64 (1H, dd, J=10, 3 Hz), 4.65 (2H, s), 6.91 (2H, d, J=8 Hz), 6.95 (2H, d, J=8 Hz), 7.06-7.40 (9H, m), 7.83 (2H, d, J=8 Hz), 7.86 (2H, d, J=8 Hz)

(+)ESI-MS (m/z): 624 (M+H)⁺

### EXAMPLE 34

The following compounds were obtained according to a similar manner to that of Example 23.

(1) Ethyl 4-[[4-[2-[[(2R)-2-(3-chlorophenyl)-2-hydroxyethyl]amino]ethoxy]phenyl]sulfonyl]benzoate hydrochloride

NMR (DMSO-d₆, δ): 1.32 (3H, t, J=7 Hz), 2.95-3.55 (4H, m), 4.34 (2H, q, J=7 Hz), 4.40 (2H, m), 5.02 (1H, m), 6.32 (1H, br s, OH), 7.20 (2H, d, J=8 Hz), 7.30-7.50 (4H, m), 7.95 (2H, d, J=8 Hz), 8.06 (2H, d, J=8 Hz), 8.14 (2H, d, J=8 Hz)

(+)ESI-MS (m/z): 534 (free, M+H)⁺

(2) Ethyl [4-[[4-[2-[[(2R)-2-(3-chlorophenyl)-2-hydroxyethyl]amino]ethoxy]phenyl]sulfonyl]phenoxy]-acetate hydrochloride

NMR (DMSO-d₆, δ) 1.20 (3H, t, J=7 Hz), 2.95-3.50 (4H, m), 4.16 (2H, q, J=7 Hz), 4.39 (2H, m), 4.90 (2H, s), 5.01 (1H, m), 6.32 (1H, br s, OH), 7.11 (2H, d, J=8 Hz), 7.17 (2H, d, J=8 Hz), 7.30-7.50 (4H, m), 7.84 (2H, d, J=8 Hz), 7.89 (2H, d, J=8 Hz)

(+)ESI-MS (m/z): 534 (free, M+H)⁺

(3) Ethyl 3-[[4-[3-[[(2R)-2-(3-chlorophenyl)-2-hydroxyethyl]amino]propyl]phenyl]sulfonyl]benzoate hydrochloride

NMR (DMSO-d₆, δ): 1.34 (3H, t, J=7 Hz), 1.96 (2H, quintet, J=7 Hz), 2.74 (2H, t, J=7 Hz), 2.80-3.25 (4H, m), 4.36 (2H, q, J=7 Hz), 4.96 (1H, m), 6.30 (1H, br s, OH), 7.26-7.60 (6H, m), 7.80 (1H, t, J=8 Hz), 7.95 (2H, d, J=8 Hz), 8.23 (1H, d, J=8 Hz), 8.23 (1H, d, J=8 Hz), 8.39 (1H, s)

(+)ESI-MS (m/z): 502 (free, M+H)⁺

(4) Ethyl 4′-[[4-[2-[[(2R)-2-(3-chlorophenyl)-2-hydroxyethyl]amino]ethoxy]phenyl]sulfonyl]-1,1′-biphenyl-3-carboxylate hydrochloride

NMR (DMSO-d₆, δ): 1.34 (3H, t, J=7 Hz), 2.95-3.55 (4H, m), 4.35 (2H, q, J=7 Hz), 4.40 (2H, m), 5.00 (1H, m), 6.33 (1H, br s, OH), 7.20 (2H, d, J=8 Hz), 7.30-7.53 (5H, m), 7.67 (1H, t, J=8 Hz), 7.85-8.13 (7H, m), 8.20 (1H, s)

(+)ESI-MS (m/z): 580 (free, M+H)⁺

(5) Methyl 4′-[[4-[2-[[(2R)-2-(3-chlorophenyl)-2-hydroxyethyl]amino]ethoxy]phenyl]sulfonyl]-1,1′-biphenyl-4-carboxylate hydrochloride

NMR (DMSO-d₆, δ): 2.98-3.50 (4H, m), 3.88 (3H, s), 4.40 (2H, m), 5.01 (1H, m), 6.32. (1H, br s, OH), 7.20 (2H, d, J=8 Hz), 7.28-7.50 (4H, m), 7.80-8.15 (10H, m)

(+)ESI-MS (m/z): 566 (free, M+H)⁺

(6) Ethyl 4′-[[4-[3-[[(2R)-2-(3-chlorophenyl)-2-hydroxyethyl]amino]propyl]phenyl]sulfonyl]-1,1′-biphenyl-3-carboxylate hydrochloride

NMR (DMSO-d₆, δ): 1.34 (3H, t, J=7 Hz), 1.97 (2H, quintet, J=7 Hz), 2.74 (2H, t, J=7 Hz), 2.82-3.25 (4H, m), 4.35 (2H, q, J=7 Hz), 4.95 (1H, m), 6.29 (1H, br s, OH), 7.20-8.28 (16H, m)

(+)ESI-MS (m/z): 578 (free, M+H)⁺

(7) Methyl 4′-[[4-[3-[[(2R)-2-(3-chlorophenyl)-2-hydroxyethyl]amino]propyl]phenyl]sulfonyl]-1,1′-biphenyl-4-carboxylate hydrochloride

NMR (DMSO-d₆, δ): 1.97 (2H, quintet, J=7 Hz), 2.74 (2H, t, J=7 Hz), 2.82-3.22 (4H, m), 3.88 (3H, s), 4.97 (1H, m), 6.29 (1H, br s, OH) 7.20-7.60 (6H, m), 7.80-8.15 (10H, m)

(+)ESI-MS (m/z): 564 (free, M+H)⁺

(8) Ethyl 3-[4-[[4-[3-[[(2R)-2-(3-chlorophenyl)-hydroxyethyl]amino]propyl]phenyl]sulfonyl]phenoxy]-benzoate hydrochloride

NMR (DMSO-d₆, δ): 1.30 (3H, t, J=7 Hz), 1.96 (2H, quintet, J=7 Hz), 2.73 (2H, t, J=7 Hz), 2.80-3.30 (4H, m), 4.28 (2H, q, J=7 Hz), 4.94 (1H, m), 6.30 (1H, br s, OH), 7.16 (2H, d, J=8 Hz), 7.22-8.05 (14H, m)

(+)ESI-MS (m/z): 594 (free, M+H)⁺

(9) Ethyl 3′-[[4-[3-[[(2R)-2-(3-chlorophenyl)-2-hydroxyethyl]amino]propyl]phenyl]sulfonyl]-1,1′-biphenyl-3-carboxylate hydrochloride

NMR (DMSO-d₆, δ): 1.35 (3H, t, J=7 Hz), 1.97 (2H, quintet, J=7 Hz), 2.73 (2H, t, J=7 Hz), 2.79-3.30 (4H, m), 4.37 (2H, q, J=7 Hz), 4.95 (1H, m), 6.30 (1H, br s, OH), 7.25-8.30 (16H, m)

(+)ESI-MS (m/z): 578 (free, M+H)⁺

(10) Ethyl 3-[3-[[4-[3-[[(2R)-2-(3-chlorophenyl)-2-hydroxyethyl]amino]propyl]phenyl]sulfonyl]phenoxy]-benzoate hydrochloride

NMR (DMSO-d₆, δ): 1.30 (3H, t, J=7 Hz), 1.97 (2H, quintet, J=7 Hz), 2.74 (2H, t, J=7 Hz), 2.80-3.30 (4H, m), 4.31 (2H, q, J=7 Hz), 4.95 (1H, m), 6.30 (1H, br s, OH), 7.20-8.00 (16H, m)

(+)ESI-MS (m/z): 594 (free, M+H)⁺

(11) Sodium 2-[3-[[4-[3-[[(2R)-2-(3-chlorophenyl)-2-hydroxyethyl]amino]propyl]phenyl]sulfonyl]phenoxy]-benzoate

NMR (DMSO-d₆, δ): 1.66 (2H, quintet, J=7 Hz), 2.35-2.80 (6H, m), 4.60 (1H, m), 5.54 (1H, br s, OH), 6.80-7.95 (16H, m)

(−)ESI-MS (m/z): 564 (free, M−H)⁻

(12) 3-[[4-[3-[[(2R)-2-(3-Chlorophenyl)-2-hydroxyethyl]amino]propyl]phenyl]sulfonyl]phenol hydrochloride

NMR (DMSO-d₆, δ): 1.97 (2H, quintet, J=7 Hz), 2.73 (2H, t, J=7 Hz), 2.75-3.30 (4H, m), 4.96 (1H, m), 6.30 (1H, br s, OH), 6.95-7.60 (10H, m), 7.86 (2H, d, J=8 Hz), 8.75 (1H, br s), 9.03 (1H, br s), 10.32 (1H, s, OH)

(+)ESI-MS (m/z): 446 (free, M+H)⁺

(13) Ethyl 5-[[4-[3-[[(2R)-2-(3-chlorophenyl)-2-hydroxyethyl]amino]propyl]phenyl]sulfonyl]-2-hydroxybenzoate hydrochloride

NMR (DMSO-d₆, δ): 1.34 (3H, t, J=7 Hz), 2.00 (2H, quintet, J=7 Hz), 2.60-3.25 (6H, m), 4.37 (2H, q, J=7 Hz), 4.96 (1H, m), 6.28 (1H, br s, OH) 7.19 (1H, d, J=9 Hz), 7.25-7.60 (6H, m), 7.88 (2H, d, J=8 Hz), 8.00 (1H, dd, J=9, 2 Hz), 8.23 (1H, d, J=2 Hz)

(+)ESI-MS (m/z): 518 (free, M+H)⁺

(14) Ethyl 4-[[4-[3-[[(2R)-2-(3-chlorophenyl)-2-hydroxyethyl]amino]propyl]phenyl]sulfonyl]-2-hydroxybenzoate hydrochloride

NMR (DMSO-d₆, δ): 1.30 (3H, t, J=7 Hz), 1.97 (2H, quintet, J=7 Hz), 2.60-3.30 (6H, m), 4.33 (2H, q, J=7 Hz), 4.96 (1H, m), 6.29 (1H, br s, OH), 7.20-7.62 (8H, m), 7.77-8.03 (3H, m)

(+)ESI-MS (m/z): 518 (free, M+H)⁺

(15) Ethyl 5-[[4-[2-[[(2R)-2-(3-chlorophenyl)-2-hydroxyethyl]amino]ethoxy]phenyl]sulfonyl]-2-hydroxybenzoate hydrochloride

NMR (DMSO-d₆, δ): 1.34 (3H, t, J=7 Hz), 2.95-3.55 (4H, m), 4.37 (2H, q, J=7 Hz), 4.38 (2H, m), 5.01 (1H, m), 6.33 (1H, br s, OH), 7.18 (2H, d, J=9 Hz), 7.25-7.55 (5H, m), 7.91 (2H, d, J=9 Hz), 7.98 (1H, dd, J=9, 2 Hz), 8.21 (12H, d, J=2 Hz), 11.27 (1H, br s, OH)

(+)ESI-MS (m/z): 520 (free, M+H)⁺

### EXAMPLE 35

The following compounds were obtained according to a similar manner to that of Example 24.

(1) tert-Butyl [(2R)-2-(3-chlorophenyl)-2-hydroxyethyl][3-[4-[[3-(2-formylphenoxy)phenyl]sulfonyl]phenyl]propyl]-carbamate

NMR (CDCl₃, δ): 1.44 (9H, s), 1.60-1.95 (2H, m), 2.61 (2H, t, J=7 Hz), 2.90-3.60 (4H, m), 4.47 (1H, br s, OH), 4.89 (1H, m), 6.91 (1H, d, J=8 Hz), 7.10-8.02 (15H, m), 10.39 (1H, s)

(+)ESI-MS (m/z): 672 (M+Na)⁺

(2) tert-Butyl [(2R)-2-(3-chlorophenyl)-2-hydroxyethyl][2-[4-[[3-[(3-formyl-2-pyridyl)oxy]phenyl]sulfonyl]-phenyl]ethyl]carbamate

NMR (CDCl₃, δ): 1.36 (9H, s), 2.60-3.02 (2H, m), 3.02-3.60 (4H, m), 4.29 (1H, br s, OH), 4.87 (1H, m), 7.05-7.65 (9H, m), 7.70-8.00 (4H, m), 8.20-8.40 (2H, m)

(−)ESI-MS (m/z): 635 (M−H)⁻

(3) tert-Butyl [(2R)-2-(3-chlorophenyl)-2-hydroxyethyl][2-[4-[[4-[(3-formyl-2-pyridyl)oxy]phenyl]sulfonyl]-phenyl]ethyl]carbamate

NMR (CDCl₃, δ): 1.36 (9H, s), 2.60-3.00 (2H, m), 3.05-3.60 (4H, m), 4.30 (1H, br s, OH), 4.88 (1H, m), 7.10-7.45 (9H, m), 7.89 (2H, d, J=8 Hz), 8.00 (2H, d, J=8 Hz), 8.20-8.40 (2H, m)

(−)ESI-MS (m/z): 635 (M−H)⁻

(4) tert-Butyl [(2R)-2-(3-chlorophenyl)-2-hydroxyethyl][3-[4-[[3-[(3-formyl-2-pyridyl)oxy]phenyl]sulfonyl]-phenyl]propyl]carbamade

NMR (CDCl₃, δ): 1.44 (9H, s), 1.76 (2H, quintet, J=7 Hz) 2.61 (2H, m), 2.85-3.55 (4H, m), 4.48 (1H, br s, OH), 4.88 (1H, m), 7.05-7.65 (9H, m), 7.70-8.00 (4H, m), 8.20-8.36 (2H, m), 10.51 (1H, s)

(−)ESI-MS (m/z): 649 (M−H)⁻

### EXAMPLE 36

The following compounds were obtained according to a similar manner to that of Example 25.

(1) 2-[3-[[4-[2-[[(2R)-2-(3-Chlorophenyl)-2-hydroxyethyl]amino]ethyl]phenyl]sulfonyl]phenoxy]-nicotinic acid dihydrochloride

NMR (DMSO-d₆, δ): 2.90-3.40 (6H, m), 4.99 (1H, m) 6.34 (1H, br s, OH), 7.20-7.90 (11H, m), 7.97 (2H, d, J=8 Hz), 8.20-8.40 (2H, m)

(+)ESI-MS (m/z): 553 (free, M+H)⁺

(2) 2-[4-[[4-[2-[[(2R)-2-(3-Chlorophenyl)-2-hydroxyethyl]amino]ethyl]phenyl]sulfonyl]phenoxy]-nicotinic acid dihydrochloride

NMR (DMSO-d₆, δ): 2.90-3.40 (6H, m), 4.99 (1H, m), 6.34 (1H, br s, OH), 7.22-7.62 (9H, m), 7.96 (2H, d, J=8 Hz), 7.99 (2H, d, J=8 Hz), 8.23-8.40 (2H, m)

(−)ESI-MS (m/z): 551 (free, M−H)⁻

### EXAMPLE 37

The following compound was obtained according to a similar manner to that of Example 26.

tert-Butyl [(2R)-2-(3-chlorophenyl)-2-hydroxyethyl][3-[4-[(3-hydroxyphenyl)sulfonyl]phenyl]propyl]carbamate

NMR (CDCl₃, δ): 1.43 (9H, s), 1.78 (2H, quintet, J=7 Hz) 2.60 (2H, t, J=7 Hz), 2.85-3.50 (4H, m), 4.58 (1H, br s, OH), 4.86 (1H, m), 6.84 (1H, br s, OH), 6.92-7.52 (10H, m), 7.81 (2H, d, J=8 Hz)

(+)ESI-MS (m/z): 568 (M+Na)⁺

### EXAMPLE 38

The following compound was obtained according to a similar manner to that of Preparation 24 starting from the object compound of Example 14.

Ethyl 3-[4-[[4-[2-[(tert-butoxycarbonyl)[(2R)-2-(3-chlorophenyl)-2-hydroxyethyl]amino]ethyl]phenyl]sulfonyl]-phenoxy]benzoate

NMR (CDCl₃, δ): 1.37 (9H, br s), 1.38 (3H, t, J=7 Hz), 2.60-3.05 (2H, m), 3.05-3.60 (4H, d), 4.33 (1H, br s, OH), 4.37 (2H, q, J=7 Hz), 4.87 (1H, m), 7.00 (2H, d, J=9 Hz), 7.10-7.42 (7H, m), 7.47 (1H, t, J=8 Hz), 7.69 (1H, s), 7.74-7.96 (5H, m)

(+)ESI-MS (m/z): 702 (M+Na)⁺

### EXAMPLE 39

The following compound was obtained according to a similar manner to that of Preparation 24 starting from the object compound of Example 3-(16).

Ethyl 3-[3-[[4-[3-[benzyl[(2R)-2-(3-chlorophenyl)-2-hydroxyethyl]amino]propyl]phenyl]sulfonyl]phenoxy]benzoate

NMR (CDCl₃, δ): 1.38 (3H, t, J=7 Hz), 1.81 (2H, quintet, J=7 Hz), 2.35-2.83 (6H, m), 3.49 (1H, d, J=13 Hz), 3.87 (1H, d, J=13 Hz), 3.91 (1H, br s, OH), 4.37 (2H, q, J=7 Hz), 4.61 (1H, dd, J=10 and 4 Hz), 7.05-7.95 (21H, m)

(+)ESI-MS (m/z): 684 (M+H)⁺

### EXAMPLE 40

The following compounds were obtained according to a similar manner to that of Preparation 60 starting from the object compound of Example 16.

(1) Methyl(R)-3′-[[4-[2-[benyl[2-(3-chlorophenyl)-2-hydroxyethyl]amino]ethyl]phenyl]sulfonyl]-1,1′-biphenyl-4-carboxylate

NMR (CDCl₃, δ): 2.5-2.9 (6H, m), 3.53 (1H, d, J=13.4 Hz) 3.90 (1H, d, J=13.4 Hz), 3.95 (3H, s), 4.59 (1H, dd, J=3.7, 9.8 Hz), 7.1-7.35 (11H, m), 7.55-7.7 (3H, m), 7.75-8.0 (4H, m), 8.1-8.2 (3H, m)

(+)ESI-MS (m/z): 640, 642 (M+H)⁺

(2) Ethyl(R)-3′-[[4-[2-[benzyl[2-(3-chlorophenyl)-2-hydroxyethyl]amino]ethyl]phenyl]sulfonyl]-1,1′-biphenyl-3-carboxylate

NMR (CDCl₃, δ): 1.42 (3H, t, J=7.2 Hz), 2.5-2.9 (6H, m), 3.54 (1H, d, J=13.4 Hz), 3.89 (1H, d, J=13.4 Hz), 4.42 (2H, q, J=7.2 Hz), 4.60 (1H, dd, J=3.6, 9.9 Hz), 7.1-7.35 (11H, m), 7.45-7.65 (2H, m), 7.75-8.0 (5H, m), 8.05-8.3 (3H, m)

(+)ESI-MS (m/z): 654, 656 (M+H)⁺

### EXAMPLE 41

The following compounds were obtained according to a similar manner to that of Preparation 60 starting from the object compound of Example 27-(1).

(1) Methyl 4′-[[4-[2-[(tert-butoxycarbonyl)[(2R)-2-(3-chlorophenyl)-2-hydroxyethyl]amino]ethyl]phenyl]-sulfonyl]-1,1′-biphenyl-3-carboxylate

NMR (CDCl₃, δ): 1.34 (9H, br s) 2.60-3.00 (2H, m), 3.00-3.70 (4H, m), 3.95 (3H, s), 4.30 (1H, br s, OH), 4.85 (1H, m), 7.10-7.42 (6H, m), 7.55 (1H, t, J=8 Hz), 7.63-7.82 (3H, m), 7.90 (2H, d, J=8 Hz), 8.00 (2H, d, J=8 Hz), 8.08 (1H, d, J=8 Hz), 8.23 (1H, s)

(+)ESI-MS (m/z): 672(M+Na)⁺

(2) Methyl 4′-[[4-[2-[(tert-butoxycarbonyl)[(2R)-2-(3-chlorophenyl)-2-hydroxyethyl]amino]ethyl]phenyl]-sulfonyl]-1,1′-biphenyl-4-carboxylate

NMR (CDCl₃, δ): 1.34 (9H, br s), 2.60-3.04 (2H, m), 3.04-3.70 (4H, m) 3.95 (3H, s), 4.28 (1H, br s, OH), 4.84 (1H, m), 7.08-7.42 (6H, m), 7.61 (2H, d, J=8 Hz), 7.70 (2H, d, J=8 Hz), 7.90 (2H, d, J=8 Hz), 8.01 (2H, d, J=8 Hz), 8.12 (2H, d, J=8 Hz)

(+)ESI-MS (m/z): 672 (M+Na)⁺

### EXAMPLE 42

The following compounds were obtained according to a similar manner to that of Preparation 60 starting from the object compound of Example 27-(3).

(1) Ethyl 4′-[[4-[2-[benzyl[(2R)-2-(3-chlorophenyl)-2-hydroxyethyl]amino]ethoxy]phenyl]sulfonyl]-1,1′-biphenyl-3-carboxylate

NMR (CDCl₃, δ): 1.41 (3H, t, J=7 Hz), 2.64 (1H, dd, J=13, 10 Hz), 2.85 (1H, dd, J=13, 4 Hz), 2.86-3.20 (2H, m), 3.69 (1H, d, J=13 Hz), 3.94 (1H, d, J=13 Hz), 3.96 (br s, OH), 3.96-4.10 (2H, m), 4.41 (2H, q, J=7 Hz), 4.64 (1H, dd, J=10, 3 Hz), 6.94 (2H, d, J=8 Hz), 7.08-7.40 (9H, m), 7.53 (1H, t, J=8 Hz), 7.63-7.82 (3H, m), 7.90 (2H, d, J=8 Hz), 8.00 (2H, d, J=8 Hz), 8.08 (1H, d, J=8 Hz), 8.23 (1H, s)

(+)ESI-MS (m/z): 670 (M+H)⁺

(2) Methyl 4′-[[4-[2-[benzyl[(2R)-2-(3-chlorophenyl)-2-hydroxyethyl]amino]ethoxy]phenyl]sulfonyl]-1,1′-biphenyl-4-carboxylate

NMR (CDCl₃, δ): 2.64 (1H, dd, J=13, 10 Hz), 2.85 (1H, dd, J=13, 4 Hz), 2.86-3.20 (2H, m), 3.68 (1H, d, J=13 Hz), 3.94 (3H, s), 3.94 (1H, d, J=13 Hz), 3.96-4.10 (2H, m), 4.64 (1H, dd, J=10, 3 Hz), 6.94 (2H, d, J=8 Hz), 7.08-7.42 (9H, m), 7.63 (2H, d, J=8 Hz), 7.72 2H, d, J=8 Hz), 7.90 (2H, d, J=8 Hz), 8.00 (2H, d, J=8 Hz), 8.12 (2H, d, J=8 Hz)

(+)ESI-MS (m/z): 656 (M+H)⁺

### EXAMPLE 43

The following compounds were obtained according to a similar manner to that of Preparation 60 starting from the object compound of Example 27-(5).

(1) Ethyl 4′-[[4-[3-[benzyl[(2R)-2-(3-chlorophenyl)-2-hydroxyethyl]amino]propyl]phenyl]sulfonyl]-1,1′-biphenyl-3-carboxylate

NMR (CDCl₃, δ): 1.41 (3H, t, J=7 Hz), 1.81 (2H, quintet, J=7 Hz), 2.32-2.80 (6H, m), 3.48 (1H, d, J=13 Hz), 3.87 (1H, d, J=13 Hz), 3.90 (1H, br s, OH), 4.41 (2H, q, J=7 Hz), 4.60 (1H, dd, J=10, 4 Hz), 7.05-7.40 (11H, m), 7.53 (1H, t, J=8 Hz), 7.62-7.84 (3H, m), 7.86 (2H, d, J=8 Hz), 8.02 (2H, d, J=8 Hz), 8.08 (1H, d, J=8 Hz), 8.23 (1H, s)

(+)ESI-MS (m/z): 668 (M+H)⁺

(2) Methyl 4′-[[4-[3-[benzyl[(2R)-2-(3-chlorophenyl)-2-hydroxyethyl]amino]propyl]phenyl]sulfonyl]-1,1-biphenyl-4-carboxylate

NMR (CDCl₃, δ): 1.81 (2H, quintet, J=7 Hz), 2.35-2.80 (6H, m), 3.48 (1H, d, J=13 Hz), 3.87 (1H, d, J=13 Hz), 3.95 (3H, s), 4.60 (1H, dd, J=10, 4 Hz), 7.05-7.40 (11H, m), 7.62 (2H, d, J=8 Hz), 7.72 (2H, d, J=8 Hz), 7.86 (2H, d, J=8 Hz), 8.02 (2H, d, J=8 Hz), 8.12 (2H, d, J=8 Hz)

(+)ESI-MS (m/z): 654 (M+Na)⁺

### EXAMPLE 44

The following compound was obtained according to a similar manner to that of Preparation 60 starting from the object compound of Example 27-(2).

Ethyl 3′-[[4-[3-[benzyl[(2R)-2-(3-chlorophenyl)-2-hydroxyethyl]amino]propyl]phenyl]sulfonyl]-1,1′-biphenyl-3-carboxylate

NMR (CDCl₃, δ): 1.42 (3H, t, J=7 Hz), 1.80 (2H, quintet, J=7 Hz), 2.32-2.78 (6H, m), 3.48 (1H, d, J=13 Hz), 3.86 (1H, d, J=13 Hz), 4.43 (2H, q, J=7 Hz), 4.60 (1H, dd, J=10, 4 Hz), 7.03-8.30 (21H, m)

(+)ESI-MS (m/z): 668 (M+H)⁺

### EXAMPLE 45

The following compound was obtained according to a similar manner to that of Preparation 11 starting from the object compound of Example 27-(3).

Ethyl 4-[[4-[2-[benzyl[(2R)-2-(3-chlorophenyl)-2-hydroxyethyl]amino]ethoxy]phenyl]sulfonyl]benzoate

NMR (CDCl₃, δ): 1.39 (3H, t, J=7 Hz), 2.64 (1H, dd, J=13, 10Hz), 2.84-3.22 (2H, m), 2.85 (1H, dd, J=13, 4 Hz), 3.68 (1H, d, J=13 Hz), 3.94 (1H, d, J=13 Hz), 3.94-4.10 (2H, m), 4.39 (2H, q, J=7 Hz), 4.64 (1H, dd, J=10, 3 Hz), 6.93 (2H, d, J=8 Hz), 7.05-7.40 (9H, m), 7.87 (2H, d, J=8 Hz), 7.97 (2H, d, J=8 Hz), 8.14 (2H, d, J=8 Hz)

(+)ESI-MS (m/z): 594 (M+H)⁺

### EXAMPLE 46

The following compound was obtained according to a similar manner to that of Preparation 11 starting from the object compound of Example 27-(2).

Ethyl 3-[[4-[3-[benzyl[(2R)-2-(3-chlorophenyl)-2-hydroxyethyl]amino]propyl]phenyl]sulfonyl]benzoate

NMR (CDCl₃, δ): 1.41 (3H, t, J=7 Hz), 1.80 (2H, quintet, J=7 Hz), 2.32-2.75 (6H, m), 3.48 (1H, d, J=13 Hz), 3.87 (1H, d, J=13 Hz), 4.41 (2H, q, J=7 Hz), 4.60 (1H, dd, J=10, 4 Hz), 7.03-7.40 (11H, m), 7.59 (1H, t, J=8 Hz), 7.84 (2H, d, J=8 Hz), 8.11 (1H, d, J=8 Hz), 8.22 (1H, d, J=8 Hz), 8.59 (1H, s)

(+)ESI-MS (m/z): 592 (M+H)⁺

### EXAMPLE 47

The following compound was obtained according to a similar manner to that of Preparation 11 starting from the object compound of Example 27-(4).

Ethyl 2-[[4-[(2R)-2-[benzyl[(2R)-2-(3-chlorophenyl)-2-hydroxyethyl]amino]propyl]phenyl]sulfonyl]benzoate

NMR (CDCl₃, δ): 1.01 (3H, d, J=6 Hz), 1.38 (3H, t, J=7 Hz), 2.40-2.90 (4H, m), 2.98-3.22 (1H, m), 3.49 (1H, d, J=13 Hz), 3.57 (1H, br s, OH), 3.83 (1H, d, J=13 Hz), 4.43 (2H, q, J=7 Hz), 4.58 (1H, dd, J=10, 4 Hz), 6.85-8.20 (17H, m)

(+)ESI-MS (m/z): 592 (M+H)⁺

### EXAMPLE 48

The following compound was obtained according to a similar manner to that of Preparation 33 starting from the object compound of Example 24. 2-[3-[[4-[3-[Benzyl[(2R)-2-(3-chlorophenyl)-2-hydroxyethyl]amino]propyl]phenyl]sulfonyl]phenoxy]benzoic acid

NMR (CDCl₃, δ): 1.96 (2H, quintet, J=7 Hz), 2.35-3.00 (6H, m), 3.86 (1H, d, J=13 Hz), 3.89 (1H, d, J=13 Hz), 4.66 (1H, dd, J=10, 3 Hz), 6.80-8.10 (21H, m)

(+)ESI-MS (m/z): 656 (M+H)⁺

### EXAMPLE 49

The following compound was obtained according to a similar manner to that of Preparation 33 starting from the object compound of Example 35-(1). 2-[3-[[4-[3-[(tert-Butoxycarbonyl)[(2R)-2-(3-chlorophenyl)-2-hydroxyethyl]amino]propyl]phenyl]sulfonyl]-phenoxy]benzoic acid

NMR (DMSO-d₆, δ): 1.28 (9H, s), 1.60-1.88 (2H, m), 2.58 (2H, t, J=7 Hz), 2.98-3.44 (4H, m), 4.72 (1H, m), 5.56 (1H, br s, OH), 7.05-8.00 (16H, m)

(−)ESI-MS (m/z): 664 (M−H)⁻

### EXAMPLE 50

The following compound was obtained according to a similar manner to that of Preparation 33 starting from the object compound of Example 9-(7). 2-[3-[[4-[2-[(tert-Butoxycarbonyl)[(2R)-2-(3-chlorophenyl)-2-hydroxyethyl]amino]ethyl]phenyl]sulfonyl]-phenoxy]nicotinic acid

1.08, 1.21 (total 9H, a pair of s), 2.65-3.00 (2H, m), 3.00-3.60 (4H, m), 4.73 (1H, m) 5.59 (1H, br s, OH), 7.10-8.00 (13H, m), 8.20-8.40 (2H, m)

(−)ESI-MS (m/z): 651 (M−H)⁻

### EXAMPLE 51

The following compound was obtained according to a similar manner to that of Preparation 33 starting from the object compound of Example 35-(3). 2-[4-[[4-[2-[(tert-Butoxycarbonyl)[(2R)-2-(3-chlorophenyl)-2-hydroxyethyl]amino]ethyl]phenyl]sulfonyl]-phenoxy]nicotinic acid

NMR (DMSO-d₆, δ): 1.09, 1.21 (total 9H, a pair of s), 2.65-3.00 (2H, m), 3.00-3.55 (4H, m), 4.75 (1H, m) 5.59 (1H, br s, OH), 7.10-7.60 (9H, m), 7.89 (2H, d, J=8 Hz), 7.96 (2H, d, J=8 Hz), 8.20-8.40 (2H, m)

(−)ESI-MS (m/z): 651 (M−H)⁻

### EXAMPLE 52

The following compound was obtained according to a similar manner to that of Preparation 33 starting from the object compound of Example 35-(4). 2-[3-[(4-[3-[(tert-Butoxycarbonyl)[(2R)-2-(3-chlorophenyl)-2-hydroxyethyl]amino]propyl]phenyl]sulfonyl]-phenoxy]nicotinic acid

NMR (DMSO-d₆, δ): 1.25, 1.28 (total 9H, a pair of s), 1.74 (2H, quintet, J=7 Hz), 2.48-2.70 (2H, m), 2.95-3.55 (4H, m), 4.71 (1H, m), 5.56 (1H, br s, OH), 7.10-8.00 (13H, m), 8.15-8.40 (2H, m)

(−)ESI-MS (m/z): 665 (M−H)⁻

### EXAMPLE 53

The following compound was obtained according to a similar manner to that of Preparation 34 starting from the object compound of Example 17.

Ethyl(R)-3′-[[4-[2-[benzyl[2-(3-chlorophenyl)-2-hydroxyethyl]amino]ethyl]phenyl]sulfonyl]-1,1′-biphenyl-2-carboxylate

NMR (CDCl₃, δ): 0.85 (3H, t, J=7.1 Hz), 2.5-2.9 (6H, m), 3.55 (1H, d, J=13.4 Hz), 3.85-4.0 (3H, m), 4.62 (1H, dd, J=3.7, 9.9 Hz), 7.15-7.35 (12H, m), 7.4-7.6 (4H, m), 7.8-7.95 (5H, m)

(+)ESI-MS (m/z): 654, 656 (M+H)⁺

Preparation 78

Under nitrogen at 5° C., to a solution of 4-iodophenylacetic acid (11.6 g) in N,N-dimethylformamide (110 ml) were added (1R)-2-amino-1-(3-chlorophenyl)ethanol hydrochloride (9.19 g), 1-(3-dimethylaminopropyl)-3-ethylcarbodiimide (7.54 g) and 1-hydroxybenzotriazole (6.56 g), and the mixture was stirred at room temperature for 12 hours. The resulting mixture was poured into 1N sodium hydroxide and the aqueous mixture was extracted with a mixture of hexane and ethyl acetate (1:1). The organic layer was washed successively with water (two times) and brine, dried over anhydrous magnesium sulfate, evaporated under reduced pressure and dried in vacuo to give N-[(2R)-2-(3-chlorophenyl)-2-hudroxyethyl]-2-(4-iodophenyl)acetamide (16.7 g).

(+)ESI-MS (m/z): 438 (M+Na)⁺

Preparation 79

Under nitrogen at 5° C., to a solution of N-[(2R)-2-(3-chlorophenyl)-2-hydroxyethyl]-2-(4-iodophenyl)acetamide (16.7 g) in tetrahydrofuran (170 ml) was added dropwise a solution of borane-methyl sulfide complex (12.8 ml) in tetrahydrofuran (13 ml), and the mixture was refluxed for 1 hour. The resulting mixture was cooled to 5° C., and to this one was added 6N hydrochloric acid (82 ml) dropwise, and the mixture was stirred at room temperature for 2.5 days. The mixture was cooled to 5° C., adjusted to pH 8.7 with 3N sodium hydroxide, and to this one was added dropwise a solution of di-tert-butyl dicarbonate (9.64 g) in tetrahydrofuran (30 ml) with controlling pH. The mixture was stirred at room temperature for 2 hours. The resulting mixture was diluted with ethyl acetate. After separation, the organic layer was washed successively with water and brine, dried over anhydrous magnesium sulfate and evaporated under reduced pressure. The residue was purified by column chromatography on silica gel (hexane/ethyl acetate=7:1 to 4:1) to give tert-butyl [(2R)-2-(3-chlorophenyl)-2-hydroxyethyl][2-(4-iodophenyl)ethyl]carbamate (18.9 g).

(+)ESI-MS (m/z): 524 (M+Na)⁺

Preparation 80

The following compounds were obtained according to a similar manner to that of Preparation 56.

(1) tert-Butyl [(2R)-2-(3-chlorophenyl)-2-hydroxyethyl][2-[4-[(triisopropylsilyl)thio]phenyl]ethyl]carbamate

NMR (CDCl₃, δ): 1.05 (3H, d, J=6.3 Hz), 1.1-1.3 (3H, m), 1.48 (9H, s), 2.6-2.8 (2H, m), 3.1-3.4 (4H, m), 4.8-4.9 (1H, m), 6.9-7.1 (2H, m), 7.15-7.5 (6H, m)

(2) tert-Butyl [(2R)-2-(3-chlorophenyl)-2-hydroxyethyl]-[(1R)-1-methyl-2-[4-[(triisopropylsilyl)thio]phenyl]-ethyl]carbamate

NMR (CDCl₃, δ): 0.95-1.2 (24H, m), 1.43 (9H, br s), 2.45-2.8 (3H, m), 3.05-2.15 (1H, m), 3.3-3.55 (1H, m), 4.7-4.8 (1H, m), 6.9-7.0 (2H, m), 7.2-7.45 (6H, m)

Preparation 81

Under nitrogen at room temperature, to a solution of tert-butyl [(2R)-2-(3-chlorophenyl)-2-hydroxyethyl][2-[4-[(triisopropylsilyl)thio]phenyl]ethyl]carbamate (2.12 g) in N,N-dimethylformamide (20 ml) were added potassium carbonate (571 mg), 5-fluoro-2-nitorobenzoic acid (730 mg) and cesium fluoride (628 mg), and the mixture was stirred at 60° C. for 1.5 hours. The mixture was cooled to room temperature and to this one was added iodoethane (0.33 ml). After stirred for 3 days, the mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=3:1 to 3:1.25) to give ethyl 5-[[4-[2-[(tert-butoxycarbonyl)[(2R)-2-(3-chlorophenyl)-2-hydroxyethyl]-amino]ethyl]phenyl]thio]-2-nitrobenzoate (2.13 g).

(+)ESI-MS (m/z): 623, 625 (M+Na)⁺

Preparation 82

The following compounds were obtained according to a similar manner to that of Preparation 81.

(1) tert-Butyl [(2R)-2-(3-chlorophenyl)-2-hydroxyethyl][2-[4-[(4-formylphenyl)thio]phenyl]ethyl]carbamate

(+)ESI-MS (m/z): 534, 536 (M+Na)⁺

(2) tert-Butyl [(2R)-2-(3-chlorophenyl)-2-hydroxyethyl]-[(1R)-2-[4-[[2-formyl-4-(trifluoromethyl)phenyl]thio]-phenyl]-1-methylethyl]carbamate

(+)ESI-MS (m/z): 616, 618 (M+Na)⁺

Preparation 83

Under nitrogen at 5° C., to a solution of 2-methoxyethanol (1.0 g) in dichloromethane (10 ml) were added pyridine (1.25 g) and p-nitrobenzenesulfonyl chloride (3.2 g), and the mixture was stirred at the same temperature for 2 hours. The resulting mixture was poured into 1N hydrochloric acid and the aqueous mixture was extracted with ethyl acetate. The organic layer was washed successively with saturated aqueous sodium bicarbonate, water and brine, dried over anhydrous magnesium sulfate and evaporated under reduced pressure. The residue was purified by column chromatography on silica gel (chloroform) to give 2-methoxyethyl 4-nitrobenzenesulfonate (2.56 g).

NMR (CDCl₃, δ): 3.28 (3H, s), 3.5-3.65 (2H, m), 4.25-4.35 (2H, m), 8.05-8.2 (2H, m), 8.35-8.45 (2H, m)

Preparation 84

The following compound was obtained according to a similar manner to that of Preparation 58. 2-(2-Iodoethoxy)tetrahydro-2H-pyran

(+)ESI-MS (m/z): 279 (M+Na)⁺

Preparation 85

Under nitrogen at room temperature, to a solution of 4-[2-[(trifluoroacetyl)amino]ethyl]benzenesulfonyl chloride (2.0 g) and methyl(2-methoxyphenyl)acetate (1.37 g) in 1,2-dichloroethane (10 ml) was added aluminum chloride (2.11 g), and the mixture was refluxed for 4 days. The resulting mixture was poured into a mixture of ice-cold water and ethyl acetate, and the mixture was stirred for 10 minutes. After separation, the organic layer was washed with brine, dried over anhydrous magnesium sulfate and evaporated under reduced pressure. The residue was purified by column chromatography on silica gel (hexane/ethyl acetate=2:1 to 1:1) to give methyl [2-methoxy-5-[(4-[2-[(trifluoroacetyl)-amino]ethyl]phenyl]sulfonyl]phenyl]acetate (1.2 g).

(+)ESI-MS (m/z): 482 (M+Na)⁺

Preparation 86

The following compounds were obtained according to a similar manner to that of Preparation 85.

(1) Methyl 2-hydroxy-4-methyl-5-[[4-[(2R)-2-[(trifluoroacetyl)amino]propyl]phenyl]sulfonyl]benzoate

(+)ESI-MS (m/z): 482 (M+Na)⁺

(2) Methyl 2-hydroxy-4-methoxy-5-[[4-[(2R)-2-[(trifluoroacetyl)amino]propyl]phenyl]sulfonyl]benzoate

(+)ESI-MS (m/z): 498 (M+Na)⁺

(3) A mixture of N-[(1R)-2-[4-[(4-chloro-2-methoxyphenyl)slufonyl]phenyl]-1-methylethyl]-2,2,2-trifluoroacetamide and N-[(1R)-2-[4-[(2-chloro-4-methoxyphenyl)sulfonyl]phenyl]-1-methylethyl]-2,2,2-trifluoroacetamide

(+)ESI-MS (m/z): 458 (M+Na)⁺

Preparation 87

Under nitrogen at 5° C., to a solution of methyl [2-methoxy-5-[[4-[2-[(trifluoroacetyl)amino]ethyl]phenyl]-sulfonyl]phenyl]acetate (1.17 g) in dichloromethane (23 ml) was added boron tribromide (1M in dichloromethane, 10 ml), and the mixture was stirred at the same temperature for 30 minutes. The resulting mixture was evaporated under reduced pressure. The residue was dissolved into a mixture of water and ethyl acetate. After separation, the organic layer was washed successively with water and brine, dried over anhydrous magnesium sulfate and evaporated under reduced pressure. The residue was purified by column chromatography on silica gel (hexane/ethyl acetate=1:1 to 1:2) to give methyl [2-hydroxy-5-[[4-[2-[(trifluoroacetyl)amino]ethyl]-phenyl]sulfonyl]phenyl]acetate (787 mg).

(+)ESI-MS (m/z): 468 (M+Na)⁺

Preparation 88

The following compounds were obtained according to a similar manner to that of Preparation 87.

(1) N-[(1R)-2-[4-[(4-Chloro-2-hydroxyphenyl)sulfonyl]-phenyl]-1-methylethyl]-2,2,2-trifluoroacetamide

NMR (CDCl₃, δ): 1.23 (3H, d, J=6.7 Hz), 2.8-3.05 (2H, m), 4.15-4.4 (1H, m), 6.8-7.0 (3H, m), 7.30 (2H, d, J=8.3 Hz), 7.86 (2H, d, J=8.3 Hz), 8.15-8.20 (1H, m)

(+)ESI-MS (m/z): 444 (M+Na)⁺

(2) N-[(1R)-2-[4-[(2-Chloro-4-hydroxyphenyl)sulfonyl]-phenyl]-1-methylethyl]-2,2,2-trifluoroacetamide

NMR (CDCl₃, δ): 1.22 (3H, d, J=6.8 Hz), 2.75-3.1 (2H, m), 4.1-4.4 (1H, m), 6.9-7.05 (2H, m), 7.35 (2H, d, J=8.3 Hz), 7.57 (1H, d, J=8.5 Hz), 7.8-7.9 (2H, m)

(+)ESI-MS (m/z): 444 (M+Na)⁺

Preparation 89

Under nitrogen at room temperature, to methyl [2-hydroxy-5-[[4-[2-[(trifluoroacetyl)amino]ethyl]phenyl]-sulfonyl]phenyl]acetate (775 mg) was added 5.5N hydrogen chloride in ethanol (15 ml), and the mixture was refluxed for 19 hours. The resulting mixture was evaporated under reduced pressure. The residue was dissolved into a mixture of aqueous sodium bicarbonate and chloroform/methanol (4:1). After separation, the organic layer was dried over anhydrous magnesium sulfate, evaporated and dried in vacuo to give ethyl [5-[[4-(2-aminoethyl)phenyl]sulfonyl]-2-hydroxyphenyl]acetate (463 mg).

(+)ESI-MS (m/z): 364 (M+H)⁺

Preparation 90

The following compounds were obtained according to a similar manner to that of Preparation 89.

(1) Ethyl 2-[[4-[(2R)-2-aminopropyl]phenyl]sulfonyl]-5-chlorobenzoate

(+)ESI-MS (m/z): 382 (M+H)⁺

(2) Ethyl 4-[[4-[(2R)-2-aminopropyl]phenyl]sulfonyl]-3-chlorobenzoate

(+)ESI-MS (m/z): 382 (M+H)⁺

(3) Methyl 2-[[4-[(2R)-2-aminopropyl]phenyl]sulfonyl]-4-chlorobenzoate

(+)ESI-MS (m/z): 368 (M+H)⁺

(4) Ethyl 5-methoxy-2-[[4-[(2R)-2-[(trifluoroacetyl)amino]-propyl]phenyl]sulfonyl]benzoate

(+)ESI-MS (m/z): 404 (M+Na)⁺

(5) Methyl 2-[[4-[(2R)-2-aminopropyl]phenyl]sulfonyl]-5-methylbenzoate

(+)ESI-MS (m/z): 348 (M+H)⁺

(6) Ethyl 2-[[4-[(2R)-2-aminopropyl]phenyl]sulfonyl]-5-methoxybenzoate

(+)ESI-MS (m/z): 400 (M+Na)⁺

(7) Ethyl 2-[[4-[(2R)-2-aminopropyl]phenyl]sulfonyl]-5-phenoxybenzoate

(+)ESI-MS (m/z): 461 (M+Na)⁺

(8) Ethyl 2-[[4-[(2R)-2-aminopropyl]phenyl]sulfonyl]-5-propylbenzoate

(+)ESI-MS (m/z): 390 (M+H)⁺

(9) Ethyl 2-[[4-[(2R)-2-aminopropyl]phenyl]sulfonyl]-5-(3-methylbutyl)benzoate

(+)ESI-MS (m/z): 418 (M+H)⁺

(10) Ethyl 2-[[4-[(2R)-2-aminopropyl]phenyl]sulfonyl]-5-cyclohexylbenzoate

(+)ESI-MS (m/z): 430 (M+H)⁺

(11) Ethyl 2-[[4-[(2R)-2-aminopropyl]phenyl]sulfonyl]-4-propylbenzoate

(+)ESI-MS (m/z): 412 (M+Na)⁺

(12) Ethyl 4-[[4-[(2R)-2-aminopropyl]phenyl]sulfonyl]-3-biphenylcarboxylate

(+)ESI-MS (m/z): 424 (M+H)⁺

(13) Ethyl 5-[[4-[(2R)-2-aminopropyl]phenyl]sulfonyl]-2-hydroxy-4-methylbenzoate

(+)ESI-MS (m/z): 378 (M+H)⁺

(14) Ethyl 5-[[4-[(2R)-2-aminopropyl]phenyl]sulfonyl]-2-hydroxy-4-methoxybenzoate

(+)ESI-MS (m/z): 394 (M+H)⁺

(15) Ethyl 4-[[4-[(2R)-2-aminopropyl]phenyl]sulfonyl]-benzoate

(+)ESI-MS (m/z): 348 (M+H)⁺

(16) Ethyl 5-[[4-[(2R)-2-aminopropyl]phenyl]sulfonyl]-2-hydroxybenzoate

(+)ESI-MS (m/z): 364 (M+H)⁺

Preparation 91

Under nitrogen at room temperature, to a suspension of ethyl [5-[[4-(2-aminoethyl)phenyl]sulfonyl]-2-hydroxyphenyl]acetate (460 mg) in chloroform (10 ml) was added benzaldehyde (141 mg), and the mixture was stirred at the same temperature for 1 hour. After the resulting mixture was evaporated under reduced pressure, to a suspension of the residue in tetrahydrofuran (5 ml) was added sodium borohydride (53 mg) at 5° C. under nitrogen, followed by methanol (5 ml) dropwise, and the mixture was stirred at room temperature for 12 hours. The resulting mixture was poured into water and the aqueous mixture was extracted with ethyl acetate. The organic layer was washed with brine, dried over anhydrous magnesium sulfate and evaporated under reduced pressure. The residue was purified by column chromatography on silica gel (chloroform/methanol=20:1 to 15:1) to give ethyl [5-[[4-[2-(benzylamino)ethyl]-phenyl]sulfonyl]-2-hydroxyphenyl]acetate (385 mg).

(+)ESI-MS (m/z): 454 (M+H)⁺

Preparation 92

Under nitrogen at 5° C., to a solution of N-[(1R)-2-[4-[(4-chloro-2-hydroxyphenyl)sulfonyl]phenyl]-1-methylethyl]-2,2,2-trifluoroacetamide (1.0 g) in dichloromethane (10 ml) were added 2,6-lutidine (330 mg) and trifluoromethanesulfonic anhydride (736 mg), and the mixture was stirred at the same temperature for 1.5 hours. The resulting mixture was poured into 1N hydrochloric acid and the aqueous mixture was extracted with ethyl acetate. The organic layer was washed successively with saturated aqueous sodium bicarbonate and brine, dried over anhydrous magnesium sulfate, evaporated and dried in vacuo to give 5-chloro-2-[[4-[(2R)-2-[(trifluoroacetyl)amino]propyl]phenyl]sulfonyl]-phenyl trifluoromethanesulfonate (1.17 g).

(+)ESI-MS (m/z): 576 (M+Na)⁺

Preparation 93

The following compounds were obtained according to a similar manner to that of Preparation 92.

(1) 3-Chloro-4-[[4-[(2R)-2-[(trifluoroacetyl)amino]propyl]-phenyl]sulfonyl]phenyl trifluoromethanesulfonate

(+)ESI-MS (m/z): 576 (M+Na)⁺

(2) Methyl 4-chloro-2-[[(trifluoromethyl)sulfonyl]oxy]-benzoate

(+)ESI-MS (m/z): 341 (M+Na)⁺

(3) Methyl 5-methyl-2-[[(trifluoromethyl)sulfonyl]oxy]-benzoate

(+)ESI-MS (m/z): 321 (M+Na)⁺

Preparation 94

The following compounds were obtained according to a similar manner to that of Preparation 11.

(1) Ethyl 5-chloro-2-[[4-[(2R)-2-[(trifluoroacetyl)amino]-propyl]phenyl]sulfonyl]benzoate

(+)ESI-MS (m/z): 500 (M+Na)⁺

(2) Ethyl 3-chloro-4-[[4-[(2R)-2-[(trifluoroacetyl)amino]-propyl]phenyl]sulfonyl]benzoate

(+)ESI-MS (m/z): 500 (M+Na)⁺

Preparation 95

To a solution of 2,2,2-trifluoro-N-[(1R)-2-(4-iodophenyl)-1-methylethyl]acetamide (10 g) in 1,4-dioxane (100 ml) was added 1N sodium hydroxide (56 ml), and the mixture was stirred at 50° C. for 1 hour. 1,4-Dioxane was removed by evaporation, and the aqueous mixture was extracted with a mixture of chloroform and methanol (5:1). The organic layer was dried over anhydrous magnesium sulfate, evaporated and dried in vacuo to give (2R)-1-(4-iodophenyl)-2-propanamine (7.75 g).

(+)ESI-MS (m/z): 262 (M+H)⁺

Preparation 96

The following compound was obtained according to a similar manner to that of Example 83.

(1R)-1-(3-Chlorophenyl)-2-[[(1R)-2-(4-iodophenyl)-1-methylethyl]amino]ethanol

(+)ESI-MS (m/z): 415 (M+H)⁺

Preparation 97

To a suspension of (1R)-1-(3-chlorophenyl)-2-[[(1R)-2-(4-iodophenyl)-1-methylethyl]amino]ethanol (6.65 g) in a mixture of tetrahydrofuran (66 ml) and water (66 ml) was added dropwise a solution of di-tert-butyl dicarbonate (3.84 g) in tetrahydrofuran (10 ml) with adjusting pH to 8 by 1N sodium hydroxide, and the mixture was stirred at room temperature for 2 hours. The resulting mixture was diluted with ethyl acetate. After separation, the organic layer was washed with water, dried over anhydrous magnesium sulfate, evaporated and dried in vacuo to give tert-butyl [(2R)-2-(3-chlorophenyl)-2-hydroxyethyl][(1R)-2-(4-iodophenyl)-1-methylethyl]carbamate (8.32 g).

(+)ESI-MS (m/z): 537 (M+Na)⁺

Preparation 98

Under nitrogen at room temperature, to a solution of tert-butyl [(2R)-2-(3-chlorophenyl)-2-hydroxyethyl][(1R)-1-methyl-2-[4-[(triisopropylsilyl)thio]phenyl]ethyl]carbamate (227 mg) in N,N-dimethylformamide (5 ml) was added 2-chloro-6-fluorobenzaldehyde (69 mg) and cesium fluoride (66 mg), and the mixture was stirred at 60° C. for 1 hour. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=5:1 to 3:1) to give tert-butyl [(1R)-2-[4-[(3-chloro-2-formylphenyl)thio]phenyl]-1-methylethyl][(2R)-2-(3-chlorophenyl)-2-hydroxyethyl]carbamate (184 mg).

(+)ESI-MS (m/z): 582, 584 (M+Na)⁺

Preparation 99

A solution of 4-chloro-2-hydroxybenzoic acid (3.6 g) and concentrated sulfonic acid (3.6 ml) in methanol (36 ml) was refluxed for 3 days. The resulting mixture was poured into water and the aqueous mixture was extracted with ethyl acetate. The organic layer was washed successively with saturated aqueous sodium bicarbonate and brine, dried over anhydrous magnesium sulfate, evaporated and dried in vacuo to give methyl 4-chloro-2-hydroxybenzoate (3.59 g).

NMR (CDCl₃, δ): 3.95 (3H, s), 6.87 (1H, dd, J=2.0, 8.5 Hz), 7.01 (1H, d, J=2.0 Hz), 7.76 (1H, d, J=8.5 Hz)

Preparation 100

The following compound was obtained according to a similar manner to that of Preparation 99.

Methyl 2-hydroxy-4-methylbenzoate

NMR (CDCl₃, δ): 2.34 (3H, s), 3.92 (3H, s), 6.65-6.7 (1H, m), 6.79 (1H, m), 7.70 (1H, d, J=8.1 Hz)

Preparation 101

The following compound was obtained according to a similar manner to that of Preparation 3. 2,2,2-Trifluoro-N-[(1R)-2-(4-mercaptophenyl)-1-methylethyl]acetamide

(+)ESI-MS (m/z): 286 (M+Na)⁺

Preparation 102

Under nitrogen at room temperature, to a mixture of bis(dibenzylideneacetone)palladium(0) (328 mg) and bis(2-diphenylphosphinophenyl)ether (307 mg) was added toluene (30 ml). After being stirred at the same temperature for 10 minutes, 2,2,2-trifluoro-N-[(1R)-2-(4-mercaptophenyl)-1-methylethyl]acetamide (1.5 g), methyl 4-chloro-2-[[(trifluoromethyl)sulfonyl]oxy]benzoate (2.0 g) and potassium tert-butoxide (703 mg) were added, and the mixture was stirred at 100° C. for 1 hour. The resulting mixture was poured into water and the aqueous mixture was extracted with ethyl acetate. The organic layer was dried over anhydrous magnesium sulfate and evaporated under reduced pressure. The residue was purified by column chromatography on silica gel (hexane/ethyl acetate=5:1 to 3:1) to give methyl 4-chloro-2-[[4-[(2R)-2-[(trifluoroacetyl)amino]propyl]phenyl]-thio]benzoate (650 mg).

(+)ESI-MS (m/z): 454 (M+Na)⁺

Preparation 103

The following compound was obtained according to a similar manner to that of Preparation 102.

Methyl 5-methyl-2-[[4-[(2R)-2-[(trifluoroacetyl)amino]-propyl]phenyl]thio]benzoate

(+)ESI-MS (m/z): 434 (M+Na)⁺

Preparation 104

The following compounds were obtained according to a similar manner to that of Example 91.

(1) Methyl 4-chloro-2-[[4-[(2R)-2-[(trifluoroacetyl)amino]-propyl]phenyl]sulfonyl]benzoate

(+)ESI-MS (m/z): 486 (M+Na)⁺

(2) N-[(1R)-2-[4-[(2-Chloro-6-formylphenyl)sulfonyl]-phenyl]-1-methylethyl]-2,2,2-trifluoroacetamide

(−)ESI-MS (m/z): 432, 434 (M−H)⁻

(3) Methyl 5-methyl-2-[[4-[(2R)-2-[(trifluoroacetyl)amino]-propyl]phenyl]sulfonyl]benzoate

(+)ESI-MS (m/z): 466 (M+Na)⁺

(4) 2,2,2-Trifluoro-N-[(1R)-2-[4-[(2-formyl-4-methoxyphenyl)sulfonyl]phenyl]-1-methylethyl]acetamide

(+)ESI-MS (m/z): 452 (M+Na)⁺

(5) 2,2,2-Trifluoro-N-[(1R)-2-[4-[(4-fluoro-2-formylphenyl)sulfonyl]phenyl]-1-methylethyl]acetamide

(+)ESI-MS (m/z): 440 (M+Na)⁺

(6) 2,2,2-Trifluoro-N-[(1R)-2-[4-[(2-formyl-4-iodophenyl)sulfonyl]phenyl]-1-methylethyl]acetamide

(+)ESI-MS (m/z): 548 (M+Na)⁺

(7) 2,2,2-Trifluoro-N-[(1R)-2-[4-[(2-formyl-5-iodophenyl)sulfonyl]phenyl]-1-methylethyl]acetamide

(+)ESI-MS (m/z): 548 (M+Na)⁺

(8) 2,2,2-Trifluoro-N-[(1R)-2-[4-[(4-formylphenyl)-sulfonyl]phenyl]-1-methylethyl]acetamide

(+)ESI-MS (m/z): 422 (M+Na)⁺

Preparation 105

Under nitrogen at room temperature, to a solution of 2,2,2-trifluoro-N-[(1R)-2-(4-mercaptophenyl)-1-methylethyl]-acetamide (700 mg) in N,N-dimethylformamide (10 ml) were added 3-chloro-2-fluorobenzaldehyde (464 mg) and potassium carbonate (404 mg), and the mixture was stirred at 60° C. for 1 hour. The resulting mixture was poured into water and the aqueous mixture was extracted with ethyl acetate. The organic layer was washed successively with water and brine, dried over anhydrous magnesium sulfate, evaporated under reduced pressure. The residue was triturated with diisopropyl ether and collected by filtration followed by dryness to give N-[(1R)-2-[4-[(2-hloro-6-formylphenyl)-thio]phenyl]-1-methylethyl]-2,2,2-trifluoroacetamide (824 mg).

(+)ESI-MS (m/z): 424 (M+Na)⁺

Preparation 106

The following compounds were obtained according to a similar manner to that of Preparation 33.

(1) 3-Chloro-2-[[4-[(2R)-2-[(trifluoroacetyl)amino]propyl]-phenyl]sulfonyl]benzoic acid

(−) ESI-MS (m/z): 404 (M−COOH)⁻

(2) 5-Methoxy-2-[[4-[(2R)-2-[(trifluoroacetyl)amino]-propyl]phenyl]sulfonyl]benzoic acid

(−)ESI-MS (m/z): 400 (M−COOH)⁻

(3) 5-Phenoxy-2-[[4-[(2R)-2-[(trifluoroacetyl)amino]-propyl]phenyl]sulfonyl]benzoic acid

(−)ESI-MS (m/z): 506 (M−H)⁻

(4) 5-Iodo-2-[[4-[(2R)-2-[(trifluoroacetyl)amino]propyl]-phenyl]sulfonyl]benzoic acid

(−)ESI-MS (m/z): 540 (M−H)⁻

(5) 4-Iodo-2-[[4-[(2R)-2-[(trifluoroacetyl)amino]propyl]-phenyl]sulfonyl]benzoic acid

(−)ESI-MS (m/z): 540 (M−H)⁻

(6) 4-[[4-[(2R)-2-[(Trifluoroacetyl)amino]propyl]phenyl]-sulfonyl]benzoic acid

(−)ESI-MS (m/z): 414 (M−H)

Preparation 107

Under nitrogen at room temperature, to a solution of 3-chloro-2-[[4-[(2R)-2-[(trifluoroacetyl)amino]propyl]phenyl]-sulfonyl]benzoic acid (629 mg) in N,N-dimethylformamide (6 ml) were added iodoethane (240 mg) and potassium carbonate (213 mg), and the mixture was stirred at the same temperature for 4 hours. The resulting mixture was poured into saturated aqueous sodium bicarbonate and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=2:1 to 1:1) to give ethyl 3-chloro-2-[[4-[(2R)-2-[(trifluoroacetyl)amino]propyl]phenyl]sulfonyl]benzoate (395 mg).

(+)ESI-MS (m/z): 500 (M+Na)⁺

Preparation 108

The following compounds were obtained according to a similar manner to that of Preparation 107.

(1) Ethyl 5-methoxy-2-[[4-[(2R)-2-[(trifluoroacetyl)amino]-propyl]phenyl]sulfonyl]benzoate

(+)ESI-MS (m/z): 495 (M+Na)⁺

(2) Ethyl 5-phenoxy-2-[[4-[(2R)-2-[(trifluoroacetyl)amino]-propyl]phenyl]sulfonyl]benzoate

(+)ESI-MS (m/z): 557 (M+Na)⁺

(3) Ethyl 5-iodo-2-[[4-[(2R)-2-[(trifluoroacetyl)amino]-propyl]phenyl]sulfonyl]benzoate

(+)ESI-MS (m/z): 592 (M+Na)⁺

(4) Ethyl 4-iodo-2-[[4-[(2R)-2-[(trifluoroacetyl)amino]-propyl]phenyl]sulfonyl]benzoate

(+)ESI-MS (m/z): 592 (M+Na)⁺

(5) Ethyl 4-[[4-[(2R)-2-[(trifluoroacetyl)amino]-propyl]phenyl]sulfonyl]benzoate

(+)ESI-MS (m/z): 466 (M+Na)⁺

Preparation 109

Under nitrogen at room temperature, to a solution of tert-butyl [(2R)-2-(3-chlorophenyl)-2-hydroxyethyl][(1R)-1-methyl-2-[4-[(triisopropylsilyl)thio]phenyl]ethyl]carbamate (1.7 g) in N,N-dimethylformamide (17 ml) were added 3-fluoro-5-methoxybenzaldehyde (1.1 g) and potassium carbonate (982 mg), and the mixture was stirred at 60° C. for 1.5 hours. The resulting mixture was poured into water and the aqueous mixture was extracted with ethyl acetate. The organic layer was washed successively with water and brine, dried over anhydrous magnesium sulfate, and evaporated under reduced pressure. The residue was purified by column chromatography on silica gel (hexane/ethyl acetate=4:1 to 3:1) to give 2,2,2-trifluoro-N-[(1R)-2-[4-[(2-formyl-4-methoxyphenyl)-thio]phenyl]-1-methylethyl]acetamide (2.0 g).

(+)ESI-MS (m/z): 420 (M+Na)⁺

Preparation 110

Under nitrogen at room temperature, to a solution of tert-butyl [(2R)-2-(3-chlorophenyl)-2-hydroxyethyl][(1R)-1-methyl-2-[4-[(triisopropylsilyl)thio]phenyl]ethyl]carbamate (1.01 g) in N,N-dimethylformamide (10 ml) was added 2,5-difluorobenzaldehyde (273 mg) and cesium fluoride (292 mg), and the mixture was stirred at 60° C. for 1.5 hours. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=5:1 to 3:1) to give tert-butyl [(2R)-2-(3-chlorophnyl)-2-hydroxyethyl][(1R)-2-[4-[(4-fluoro-2-formylphenyl)thio]-phenyl]-1-methylethyl]carbamate (803 mg).

(+)ESI-MS (m/z): 566 (M+Na)⁺

Preparation 111

Under nitrogen at 5° C., to a solution of tert-butyl [(2R)-2-(3-chlorophenyl)-2-hydroxyethyl][(1R)-2-[4-[(4-fluoro-2-formylphenyl)thio]phenyl]-1-methylethyl]carbamate (790 mg) in N,N-dimethylformamide (10 ml) were added imidazole (158 mg) and tert-butyldimethylsilyl chloride (350 mg), and the mixture was stirred at room temperature for 22 hours. The resulting mixture was poured into 1N hydrochloric acid and the aqueous mixture was extracted with ethyl acetate. The organic layer was washed successively with water, saturated aqueous sodium bicarbonate and brine, dried over anhydrous magnesium sulfate and evaporated under reduced pressure. The residue was purified by column chromatography on silica gel (hexane/ethyl acetate=10:1) to give tert-butyl [(2R)-2-[[tert-butyl(dimethyl)silyl]oxy]-2-(3-chlorophenyl)ethyl][(1R)-2-[4-[(4-fluoro-2-formylphenyl)thio]phenyl]-1-methylethyl]carbamate (886 mg).

(+)ESI-MS (m/z): 680 (M+Na)⁺

Preparation 112

Under nitrogen at room temperature, to a solution of 2,2,2-trifluoro-N-[(1R)-2-(4-mercaptophenyl)-1-methylethyl]acetamide (2.0 g) in N,N-dimethylformamide (20 ml) were added 2,5-difluorobenzaldehyde (1.19 g) and potassium carbonate (1.15 g), and the mixture was stirred at 60° C. for 1 hour. The resulting mixture was poured into water and the aqueous mixture was extracted with ethyl acetate. The organic layer was washed successively with water and brine, dried over anhydrous magnesium sulfate, evaporated under reduced pressure. The residue was purified by column chromatography on silica gel (chloroform/ethyl acetate=100:1) to give 2,2,2-trifluoro-N-[(1R)-2-[4-[(4-fluoro-2-formylphenyl)thio]phenyl]-1-methylethyl]acetamide (2.28 g).

(+)ESI-MS (m/z): 408 (M+Na)⁺

Preparation 113

Under nitrogen at 5° C., to a suspension of sodium hydride (60% in oil, 32 mg) in N,N-dimethylformamide (5 ml) was added phenol (74 mg), and the mixture was stirred at room temperature for 30 minutes. To this one was added 2,2,2-trifluoro-N-[(1R)-2-[4-[(4-fluoro-2-formylphenyl)sulfonyl]phenyl]-1-methylethyl]acetamide (300 mg), and the mixture was stirred at 60° C. for 1.5 hours. The resulting mixture was poured into water and the aqueous mixture was extracted with ethyl acetate. The organic layer was washed successively with water (two times) and brine, dried over anhydrous magnesium sulfate, evaporated under reduced pressure. The residue was purified by column chromatography on silica gel (chloroform/ethyl acetate=30:1 to 20:1) to give 2,2,2-trifluoro-N-[(1R)-2-[4-[(2-formyl-4-phenoxyphenyl)sulfonyl]phenyl]-1-methylethyl]-acetamide (288 mg).

(+)ESI-MS (m/z): 514 (M+Na)⁺

Preparation 114

Under nitrogen in acetone-dry ice bath, to a solution of 2-fluoro-6-methoxybenzaldehyde (3.0 g) in dichloromethane (15 ml) was added boron tribromide (1M in dichloromethane, 21.4 ml), and the temperature of the mixture was raised to 5° C. over a period of 2 hours. The resulting mixture was poured into a mixture of ice-cold water and ethyl acetate, and stirred for 5 minutes, followed by adjusting pH to ca. 6.5 with 5N sodium hydroxide. After separation, the organic layer was washed successively with water and brine, dried over anhydrous magnesium sulfate, evaporated and dried in vacuo to give 2-fluoro-6-hydroxybenzaldehyde (2.18 g).

NMR (CDCl₃, δ): 6.55-6.65 (1H, m), 6.75-6.8 (1H, m), 7.4-7.55 (1H, m), 10.27 (1H, s)

Preparation 115

Under nitrogen at room temperature, to a solution of 2-fluoro-6-hydroxybenzaldehyde (1.04 g) in N,N-dimethylformamide (10 ml) were added chloromethyl methyl ether (1.28 g) and potassium carbonate (1.13 g), and the mixture was stirred at 45° C. for 2 hours. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=5:1 to 3:1) to give 2-fluoro-6-(methoxymethoxy)benzaldehyde (842 mg).

(+)ESI-MS (m/z): 207 (M+Na)⁺

Preparation 116

Under nitrogen at room temperature, to a solution of tert-butyl [(2R)-2-(3-chlorophenyl)-2-hydroxyethyl][(1R)-1-methyl-2-[4-[(triisopropylsilyl)thio]phenyl]ethyl]carbamate (437 mg) in N,N-dimethylformamide (4 ml) was added 2-fluoro-6-(methoxymethoxy)benzaldehyde (153 mg) and cesium fluoride (126 mg), and the mixture was stirred at 55° C. for 5 hours. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=4:1 to 3:1) to give tert-butyl [(2R)-2-(3-chlorophenyl)-2-hydroxyethyl][(1R)-2-[4-[[2-formyl-3-(methoxymethoxy)-phenyl]thio]phenyl]-1-methylethyl]carbamate (326 mg).

(+)ESI-MS (m/z): 608, 610 (M+Na)⁺

Preparation 117

Under nitrogen at room temperature, to a solution of 2,2,2-trifluoro-N-[(1R)-2-(4-mercaptophenyl)-1-methylethyl]acetamide (2.0 g) in N,N-dimethylformamide (20 ml) was added 2-fluoro-5-iodobenzaldehyde (2.09 g) and potassium carbonate (1.15 g), and the mixture was stirred at 60° C. for 2 hours. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/chloroform=1:3 to only chloroform) to give 2,2,2-trifluoro-N-[(1R)-2-[4-[(2-formyl-4-iodophenyl)thio]phenyl]-1-methylethyl]acetamide (3.06 g).

(+)ESI-MS (m/z): 516 (M+Na)⁺

Preparation 118

Under nitrogen at room temperature, to a suspension of indium (68 mg) in N,N-dimethylformamide (1 ml) was added allyl iodide (149 mg), and the mixture was stirred at room temperature for 100 minutes. Under nitrogen at room temperature, to a mixture of ethyl 5-iodo-2-[[4-[(2R)-2-[(trifluoroacetyl)amino]propyl]phenyl]sulfonyl]benzoate (336 mg), tris(dibenzylideneacetone)dipalladium(0)-chloroform adduct (27 mg), triphenylphosphine (50 mg) and lithium chloride (75 mg) was added N,N-dimethylformamide (4 ml), and the mixture was stirred at the same temperature for 15 minutes. To this one was added the allylic indium reagent which was obtained above, and the mixture was stirred at 80° C. for 1.5 hours. The resulting mixture was poured into a mixture of saturated aqueous sodium bicarbonate and ethyl acetate, and the mixture was stirred for 5 minutes. After separation, the organic layer was washed successively with water (two times) and brine, dried over anhydrous magnesium sulfate and evaporated under reduced pressure. The residue was purified by column chromatography on silica gel (chloroform/ethyl acetate=20:1 to 10:1) to give ethyl 5-allyl-2-[[4-[(2R)-2-[(trifluoroacetyl)amino]propyl]phenyl]-sulfonyl]benzoate (231 mg).

(+)ESI-MS (m/z): 506 (M+Na)⁺

Preparation 119

The following compounds were obtained according to a similar manner to that of Preparation 118.

(1) Ethyl 5-(3-methyl-2-buten-1-yl)-2-[[4-[(2R)-2-[(trifluoroacetyl)amino]propyl]phenyl]sulfonyl]benzoate

(+)ESI-MS (m/z): 534 (M+Na)⁺

(2) Ethyl 5-(2-cyclohexen-1-yl)-2-[[4-[(2R)-2-[(trifluoroacetyl)amino]propyl]phenyl]sulfonyl]benzoate

(+)ESI-MS (m/z): 546 (M+Na)⁺

(3) Ethyl 4-allyl-2-[[4-[(2R)-2-[(trifluoroacetyl)amino]-propyl]phenyl]sulfonyl]benzoate

(+)ESI-MS (m/z): 506 (M+Na)⁺

Preparation 120

A mixture of ethyl 5-allyl-2-[[4-[(2R)-2-[(trifluoroacetyl)amino]propyl]phenyl]sulfonyl]benzoate (318 mg) and 10% palladium on activated carbon (50% wet, 30 mg) in ethanol (4 ml) was stirred at room temperature in the presence of hydrogen at an atmospheric pressure for 2 hours. After filtration, the filtrate was evaporated and dried in vacuo to give ethyl 5-propyl-2-[[4-[(2R)-2-[(trifluoroacetyl)amino]propyl]phenyl]sulfonyl]benzoate (321 mg).

(+)ESI-MS (m/z): 508 (M+Na)⁺

Preparation 121

The following compounds were obtained according to a similar manner to that of Preparation 120.

(1) Ethyl 5-(3-methylbutyl)-2-[[4-[(2R)-2-[(trifluoroacetyl)amino]propyl]phenyl]sulfonyl]benzoate

(+)ESI-MS (m/z): 536 (M+Na)⁺

(2) Ethyl 4-propyl-2-[[4-[(2R)-2-[(trifluoroacetyl)amino]-propyl]phenyl]sulfonyl]benzoate

(+)ESI-MS (m/z): 508 (M+Na)⁺

Preparation 122

A mixture of ethyl 5-(2-cyclohexen-1-yl)-2-[[4-[(2R)-2-[(trifluoroacetyl)amino]propyl]phenyl]sulfonyl]benzoate (186 mg) and 10% palladium on activated carbon (50% wet, 38 mg) in ethanol (5 ml) was stirred at room temperature in the presence of hydrogen at an atmospheric pressure for 5 hours. After filtration, the filtrate was evaporated under reduced pressure. The residue was dissolved into a mixture of 1N hydrochloric acid and ethyl acetate. After separation, the organic layer was washed successively with saturated aqueous sodium bicarbonate and brine, dried over anhydrous magnesium sulfate and evaporated under reduced pressure. The residue was purified by column chromatography on silica gel (hexane/ethyl acetate=3:1 to 2:1 to give ethyl 5-cyclohexyl-2-[[4-[(2R)-2-[(trifluoroacetyl)amino]propyl]-phenyl]sulfonyl]benzoate (108 mg).

(+)ESI-MS (m/z): 548 (M+Na)⁺

Preparation 123

Under nitrogen at room temperature, to a solution of 4-bromo-2-fluorobenzaldehyde (5.0 g) in toluene (50 ml) were added ethylene glycol (4.59 g) and p-toluenesulfonic acid monohydrate (468 mg), and the mixture was refluxed for 2.5 hours to remove water by azeotropic distillation. The resulting mixture was poured into aqueous sodium bicarbonate and the aqueous mixture was extracted with toluene. The organic layer was washed successively with saturated aqueous sodium bicarbonate and brine, dried over anhydrous magnesium sulfate and evaporated under reduced pressure. The residue was purified by column chromatography on silica gel (hexane/ethyl acetate=20:1) to give 2-(4-bromo-2-fluorophenyl)-1,3-dioxolane (5.5 g).

NMR (CDCl₃, δ): 3.95-4.2 (4H, m), 6.03 (1H, s), 7.2-7.5 (3H, m)

Preparation 124

Under nitrogen in dry ice-acetone bath, to a solution of 2-(4-bromo-2-fluorophenyl)-1,3-dioxolane (5.5 g) in tetrahydrofuran (80 ml) was added butyl lithium (1.58M in hexane, 14.8 ml), and the mixture was stirred at the same temperature for 40 minutes. To this one was added a solution of iodine (17 g) in tetrahydrofuran (30 ml) in dry ice-acetone bath, and the mixture was stirred at the same temperature for 40 minutes and then at 5° C. for 1.5 hours. To the resulting mixture was added aqueous sodium thiosulfate and ethyl acetate at 5° C., and the mixture was stirred at the same temperature for 5 minutes. After separation, the organic layer was washed successively with saturated aqueous sodium bicarbonate and brine, dried over anhydrous magnesium sulfate and evaporated under reduced pressure. The residue was purified by column chromatography on silica gel (hexane/ethyl acetate=10:1) to give iodo compounds. To the solution of the iodo compounds in a mixture of tetrahydrofuran (20 ml) and methanol (20 ml) was added 6N hydrochloric acid (10 ml) at room temperature, and the mixture was stirred at the same temperature for 12 hours. The volatile solvents were removed by evaporation to give precipitates. To this one was added water, and the mixture was stirred for 1 hour. The precipitates were collected by filtration and dried in vacuo to give 2-fluoro-4-iodobenzaldehyde (1.79 g).

NMR (CDCl₃, δ): 7.5-7.75 (3H, m), 10.31 (1H, s)

Preparation 125

Under nitrogen at room temperature, to a solution of 2,2,2-trifluoro-N-[(1R)-2-(4-mercaptophenyl)-1-methylethyl]acetamide (4.0 g) in N,N-dimethylformamide (40 ml) was added 2-fluoro-4-iodobenzaldehyde (4.18 g) and potassium carbonate (2.31 g), and the mixture was stirred at 60° C. for 2 hours. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (chloroform/ethyl acetate=10:1) to give 2,2,2-trifluoro-N-[(1R)-2-[4-[(2-formyl-5-iodophenyl)thio]phenyl]-1-methylethyl]acetamide (5.02 g).

(+)ESI-MS (m/z): 516 (M+Na)⁺

Preparation 126

Under nitrogen at room temperature, to a solution of ethyl 5-iodo-2-[[4-[(2R)-2-[(trifluoroacetyl)amino]propyl]-phenyl]sulfonyl]benzoate (500 mg) and phenylboronic acid (300 mg) in 1,2-dimethoxyethane (5 ml) were added tetrakis(triphenylphosphine)palladium(0) (101 mg) and 2M sodium carbonate (1.8 ml), and the mixture was stirred at 80° C. for 7.5 hours. The resulting mixture was poured into water and the aqueous mixture was extracted with ethyl acetate. The organic layer was washed with brine, dried over anhydrous magnesium sulfate and evaporated under reduced pressure. The residue was purified by column chromatography on silica gel (hexane/ethyl acetate=2:1 to 3:2) to give ethyl 4-[[4-[(2R)-2-[(trifluoroacetyl)amino]-propyl]phenyl]sulfonyl]-3-biphenylcarboxylate (416 mg).

(+)ESI-MS (m/z): 542 (M+Na)⁺

Preparation 127

Under nitrogen at room temperature, to a solution of tert-butyl [(2R)-2-(3-chlorophenyl)-2-hydroxyethyl][(1R)-1-methyl-2-[4-[(triisopropylsilyl)thio]phenyl]ethyl]carbamate (0.92 g) in N,N-dimethylformamide (10 ml) was added 2-fluoro-5-methylbenzaldehyde (242 mg) and cesium fluoride (266 mg), and the mixture was stirred at 55° C. for 2 hours. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=6:1 to 4:1) to give tert-butyl [(2R)-2-(3-chlorophneyl)-2-hydroxyethyl][(1R)-2-[4-[(2-formyl-4-methylphenyl)thio]-phenyl]-1-methylethyl]carbamate (512 mg).

(+)ESI-MS (m/z): 562, 564 (M+Na)⁺

Preparation 128

Under nitrogen at 5° C., to methyl 2-hydroxy-4-methylbenzoate (16.1 g) was added chlorosulfonic acid (33.8 g) dropwise, and the mixture was stirred at 70° C. for 3 hours. The resulting mixture was poured into ice-cold water and the aqueous mixture was extracted with chloroform. The organic layer was dried over anhydrous magnesium sulfate and evaporated under reduced pressure. The residue was purified by column chromatography on silica gel (chloroform) to give methyl 5-(chlorosulfonyl)-2-hydroxy-4-methylbenzoate (6.96 g).

NMR (CDCl₃, δ): 2.77 (3H, s), 4.01 (3H, s), 6.99 (1H, s) 8.58 (1H, s)

Preparation 129

The following compound was obtained according to a similar manner to that of Preparation 128.

Methyl 5-(chlorosulfonyl)-2-hydroxy-4-methoxybenzoate

NMR (CDCl₃, δ) 3.98 (3H, s), 4.07 (3H, s), 6.60 (1H, s), 8.50 (1H, s)

Preparation 130

Under nitrogen at room temperature, to a solution of 2,2,2-trifluoro-N-[(1R)-2-(4-mercaptophenyl)-1-methylethyl]-acetamide (2.0 g) in N,N-dimethylformamide (20 ml) was added 4-fluorobenzaldehyde (1.04 g) and potassium carbonate (1.15 g), and the mixture was stirred at 60° C. for 2 hours. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=4:1 to 3:1) to give 2,2,2-trifluoro-N-[(1R)-2-[4-[(4-formylphenyl)-thio]phenyl]-1-methylethyl]acetamide (2.19 g).

(+)ESI-MS (m/z): 390 (M+Na)⁺

Preparation 131

Under nitrogen at room temperature, to (2-phenylethyl)amine (20 g) was added dropwise ethyl formate (49.6 g), and the mixture was stirred at 50° C. for 1.5 hours. The resulting mixture was evaporated and dried in vacuo to give (2-phenylethyl)formamide (25 mg).

(−)ESI-MS (m/z): 148 (M−H)⁻

Preparation 132

Under nitrogen at room temperature, to a mixture of (2-phenylethyl)formamide (500 mg) and methyl 5-(chlorosulfonyl)-2-hydroxybenzoate (840 mg) in nitrobenzene (5 ml) was added aluminum chloride (1.56 g), and the mixture was stirred at 100° C. for 4 hours. To the resulting mixture were added ice-cold 1N hydrochloric acid and ethyl acetate, and the mixture was stirred for 20 minutes. After separation, the organic layer was dried over anhydrous magnesium sulfate and evaporated under reduced pressure. To the residue was added hydrogen chloride methanol reagent 10 (5 ml) at room temperature under nitrogen, and the mixture was stirred at 40° C. for 2.5 hours. The resulting mixture was evaporated under reduced pressure. To the residue was added diisopropyl ether (20 ml), and the mixture was stirred for 1 hour. The precipitates were collected by filtration and dried in vacuo to give methyl 5-[[4-(2-aminoethyl)-phenyl]sulfonyl]-2-hydroxybenzoate hydrochloride (1.03 g).

(+)ESI-MS (m/z): 336 (M−HCl+H)⁺

Preparation 133

To a suspension of methyl 5-[[4-(2-aminoethyl)phenyl]-sulfonyl]-2-hydroxybenzoate hydrochloride (3.0 g) in a mixture of tetrahydrofuran (30 ml) and water (30 ml) was added dropwise a solution of di-tert-butyl dicarbonate (1.85 g) in tetrahydrofuran (5 ml) with adjusting pH to 8 by 5N sodium hydroxide, and the mixture was stirred at room temperature for 1.5 hours. The resulting mixture was diluted with ethyl acetate. After separation, the organic layer was washed with brine, dried over anhydrous magnesium sulfate, evaporated under reduced pressure. The residue was purified by column chromatography on silica gel (hexane/ethyl acetate=2:1 to 1:1) to give methyl 5-[[4-[2-[(tert-butoxycarbonyl)amino]ethyl]phenyl]sulfonyl]-2-hydroxybenzoate (3.04 g).

(+)ESI-MS (m/z): 458 (M+Na)⁺

Preparation 134

Under nitrogen at room temperature, to a solution of methyl 5-[[4-[2-[(tert-butoxycarbonyl)amino]ethyl]phenyl]-sulfonyl]-2-hydroxybenzoate (3.03 g) in N,N-dimethylformamide (30 ml) was added potassium carbonate (1.06 g) and ethyl bromoacetate (1.28 g), and the mixture was stirred at 60° C. for 1 hour. The resulting mixture was poured into water and the aqueous mixture was extracted with ethyl acetate. The organic layer was washed successively with water and brine, dried over anhydrous magnesium sulfate, evaporated and dried in vacuo to give methyl 5-[[4-[2-[(tert-butoxycarbonyl)amino]ethyl]phenyl]sulfonyl]-2-(2-ethoxy-2-oxoethoxy)benzoate (3.71 g).

(+)ESI-MS (m/z): 544 (M+Na)⁺

Preparation 135

Under nitrogen at room temperature, to methyl 5-[[4-[2-[(tert-butoxycarbonyl)amino]ethyl]phenyl]sulfonyl]-2-(2-ethoxy-2-oxoethoxy)benzoate (1.53 g) was added 4N hydrogen chloride in ethyl acetate (15 ml), and the mixture was stirred at the same temperature for 12 hours. The resulting mixture was evaporated under reduced pressure. The residue was dissolved into a mixture of saturated aqueous sodium bicarbonate and chloroform/methanol (4:1). After separation, the organic layer was dried over anhydrous magnesium sulfate, evaporated and dried in vacuo to give methyl 5-[[4-(2-aminoethyl)phenyl]sulfonyl]-2-(2-ethoxy-2-oxoethoxy)benzoate (1.04 g).

(+)ESI-MS (m/z): 422 (M+H)⁺

Preparation 136

The following compound was obtained according to a similar manner to that of Preparation 135.

Methyl 5-[[4-(2-aminoethyl)phenyl]sulfonyl]-2-(2-hydroxyethoxy)benzoate.

(+)ESI-MS (m/z): 380 (M+H)⁺

Preparation 137

Under nitrogen at 5° C., to a solution of methyl 5-[[4-[2-[(tert-butoxycarbonyl)amino]ethyl]phenyl]sulfonyl]-2-(2-ethoxy-2-oxoethoxy)benzoate (2.18 g) in tetrahydrofuran (22 ml) was added sodium borohydride (174 mg) followed by methanol (10 ml) dropwise, and the mixture was stirred at room temperature for 12 hours. The resulting mixture was poured into saturated aqueous sodium bicarbonate and the aqueous mixture was extracted with ethyl acetate. The organic layer was washed successively with water and brine, dried over anhydrous magnesium sulfate and evaporated under reduced pressure. The residue was purified by column chromatography on silica gel (hexane/ethyl acetate=1:1 to 1:5) to give methyl 5-[[4-[2-[(tert-butoxycabonyl)amino]-ethyl]phenyl]sulfonyl]-2-(2-hydroxyethoxy)benzoate (852 mg).

(+)ESI-MS (m/z): 502 (M+Na)⁺

Preparation 138

Under nitrogen at room temperature, to a suspension of methyl 5-[[4-(2-aminoethyl)phenyl]sulfonyl]-2-hydroxybenzoate hydrochloride (10 g) in methanol (300 ml) was added potassium carbonate (3.82 g), and the mixture was stirred at the same temperature for 1.5 hours. The resulting mixture was concentrated to ca. 150 ml and diluted with chloroform (150 ml). The insoluble materials were removed by filtration, and the filtrate was evaporated under reduced pressure. Under nitrogen at room temperature, to a suspension of the residue in tetrahydrofuran (300 ml) was added benzaldehyde (5.71 g), and the mixture was stirred at 50° C. for 2 hours. The resulting mixture was evaporated under reduced pressure. Under nitrogen at 5° C., to a suspension of the residue in tetrahydrofuran (300 ml) was added sodium borohydride (3.05 g) followed by methanol (300 ml) dropwise, and the mixture was stirred at room temperature for 2 hours. The resulting mixture was poured into a mixture of water and chloroform/methanol (4:1). After separation, the organic layer was washed with brine, dried over anhydrous magnesium sulfate and evaporated under reduced pressure. To the residue was added ethyl acetate, and the mixture was stirred for 12 hours. The precipitates were collected by filtration and dried in vacuo to give methyl 5-[[4-[2-(benzylamino)ethyl]phenyl]sulfonyl]-2-hydroxybenzoate (8.28 g).

(+)ESI-MS (m/z): 426 (M+H)⁺

Preparation 139

The following compound was obtained according to a similar manner to that of Preparation 9.

Methyl 2-(benzyloxy)-5-[[4-[2-[(tert-butoxycarbonyl)-amino]ethyl]phenyl]sulfonyl]benzoate

(+)ESI-MS (m/z): 548 (M+Na)⁺

Preparation 140

The following compound was obtained according to a similar manner to that of Preparation 12.

Methyl 5-[[4-[2-[benzyl(tert-butoxycarbonyl)amino]-ethyl]phenyl]sulfonyl]-2-(benzyloxy)benzoate

(+)ESI-MS (m/z): 638 (M+Na)⁺

Preparation 141

To a solution of methyl 5-[[4-[2-[benzyl(tert-butoxycarbonyl)amino]ethyl]phenyl]sulfonyl]-2-(benzyloxy)-benzoate (559 mg) in ethanol (10 ml) was added 1N sodium hydroxide (2.72 ml) at room temperature, and the mixture was stirred at 45°C for 2 hours. The resulting mixture was poured into 1N hydrochloric acid and the aqueous mixture was extracted with ethyl acetate. The organic layer was washed with brine, dried over anhydrous magnesium sulfate, evaporated and dried in vacuo to give 5-[[4-[2-[benzyl(tert-butoxycarbonyl)amino]ethyl]phenyl]sulfonyl]-2-(benzyloxy)-benzoic acid (551 mg).

(−)ESI-MS (m/z): 600 (M−H)⁻

Preparation 142

Under nitrogen at 5° C., to a solution of 5-[[4-[2-[benzyl(tert-butoxycarbonyl)amino]ethyl]phenyl]sulfonyl]-2-(benzyloxy)benzoic acid (537 mg) in N,N-dimethylformamide (6 ml) were added methylamine hydrochloride (72 mg), 1-hydroxybenzotriazole (145 mg) and 1-(3-dimethylaminopropyl)-3-ethylcarbodiimide (166 mg), and the mixture was stirred at room temperature for 3 days. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=2:1 to 1:2) to give tert-butyl benzyl[2-[4-[[4-(benzyloxy)-3-[(methylamino)carbonyl]-phenyl]sulfonyl]phenyl]ethyl]carbamate (497 mg).

(+)ESI-MS (m/z): 637 (M+Na)⁺

Preparation 143

A mixture of tert-butyl benzyl[2-[4-[[4-(benzyloxy)-3-[(methylamino)carbonyl]phenyl]sulfonyl]phenyl]ethyl]-carbamate (492 mg) and 10% palladium on activated carbon (50% wet, 25 mg) in methanol (5 ml) was stirred at room temperature in the presence of hydrogen at an atmospheric pressure for 2 hours. After filtration, the filtrate was evaporated and dried in vacuo to give tert-butyl benzyl[2-[4-[[4-hydroxy-3-[(methylamino)carbonyl]phenyl]sulfonyl]-phenyl]ethyl]carbamate (398 mg).

(+)ESI-MS (m/z): 547 (M+Na)⁺

Preparation 144

The following compound was obtained according to a similar manner to that of Preparation 135. 5-[[4-[2-(Benzylamino)ethyl]phenyl]sulfonyl]-2-hydroxy-N-methylbenzamide

(+)ESI-MS (m/z): 425 (M+H)⁺

### EXAMPLE 54

The following compound was obtained according to a similar manner to that of Preparation 6.

Ethyl 5-[[4-[2-[(tert-butoxycarbonyl)[(2R)-2-(3-chlorophenyl)-2-hydroxyethyl]amino]ethyl]phenyl]sulfonyl]-2-nitrobenzoate

(+)ESI-MS (m/z): 655 (M+Na)⁺

### EXAMPLE 55

Under nitrogen at room temperature, to a solution of ethyl 5-[[4-[2-[(tert-butoxycarbonyl)[(2R)-2-(3-chlorophenyl)-2-hydroxyethyl]amino]ethyl]phenyl]sulfonyl]-2-nitrobenzoate (1.85 g) in a mixture of ethanol (15 ml) and water (5 ml) were added reduced iron (490 mg) and ammonium chloride (78 mg), and the mixture was refluxed for 1 hour. The insoluble materials were filtered with celite and the filtrate was concentrated under reduced pressure. The residue was dissolved into a mixture of saturated aqueous sodium bicarbonate and ethyl acetate. After separation, the organic layer was washed successively with water and brine, dried over anhydrous magnesium sulfate, evaporated under reduced pressure and dried in vacuo to give ethyl 2-amino-5-[[4-[2-[(tert-butoxycarbonyl)[(2R)-2-(3-chlorophenyl)-2-hydroxyethyl]amino]ethyl]phenyl]sulfonyl]benzoate (1.6 g).

(+)ESI-MS (m/z): 625, 627 (M+Na)⁺

### EXAMPLE 56

To a solution of ethyl 2-amino-S-[[4-[2-[(tert-butoxycarbonyl)[(2R)-2-(3-chlorophenyl)-2-hydroxyethyl]-amino]ethyl]phenyl]sulfonyl]benzoate (200 mg) in ethanol (5 ml) was added 1N sodium hydroxide (0.663 ml) at room temperature, and the mixture was stirred at the same temperature for 21 hours. To the resulting mixture was added 1N hydrochloric acid (0.663 ml) and the mixture was evaporated under reduced pressure. The residue was dissolved into a mixture of water and chloroform/methanol (5:1). After separation, the organic layer was dried over anhydrous magnesium sulfate and evaporated under reduced pressure. The residue was purified by column chromatography on silica gel (chloroform/methanol=20:1 to 10:1) to give 2-amino-5-[[4-[2-[(tert-butoxycarbonyl)[(2R)-2-(3-chlorophenyl)-2-hydroxyethyl]amino]ethyl]phenyl]sulfonyl]benzoic acid (159 mg).

(−)ESI-MS (m/z): 573 (M−H)⁻

### EXAMPLE 57

Under nitrogen at room temperature, to a solution of 2-amino-5-[[4-[2-[(tert-butoxycarbonyl)[(2R)-2-(3-chlorophenyl)-2-hydroxyethyl]amino]ethyl]phenyl]sulfonyl]-benzoic acid (157 mg) in ethyl acetate (2 ml) was added 4N hydrogen chloride in ethyl acetate (2 ml), and the mixture was stirred at the same temperature for 3.5 hours. The resulting mixture was diluted with diethyl ether, and the precipitates were collected by filtration, followed by dryness to give 2-amino-5-[[4-[2-[[(2R)-2-(3-chlorophenyl)-2-hydroxyethyl]amino]ethyl]phenyl]sulfonyl]benzoic acid dihydrochloride (126 mg).

NMR (DMSO-d₆, δ): 2.9-3.3 (6H, m), 4.9-5.05 (1H, m), 6.88 (1H, d, J=8.9 Hz), 7.25-7.5 (6H, m), 7.69 (1H, dd, J=2.4, 9.0 Hz), 7.84 (2H, d, J=8.3 Hz), 8.21 (1H, d, J=2.4 Hz)

(−)ESI-MS (m/z): 473 (M−2HCl−H)⁻

### EXAMPLE 58

Under nitrogen at room temperature, to a solution of 5-[[4-[2-[(tert-butoxycarbonyl)[(2R)-2-(3-chlorophenyl)-2-hydroxyethyl]amino]ethyl]phenyl]thio]-2-nitrobenzoate (203 mg) in dichloromethane (3 ml) were added 3,4-dihydro-2H-pyran (54 mg) and pyridinium p-toluenesulfonate (8.1 mg), and the mixture was stirred at the same temperature for 4 hours. The resulting mixture was poured into water and the aqueous mixture was extracted with ethyl acetate. The organic layer was washed successively with saturated aqueous sodium bicarbonate and brine, dried over anhydrous magnesium sulfate, evaporated and dried in vacuo to give ethyl 5-[[4-[2-[(tert-butoxycarbonyl)[(2R)-2-(3-chlorophenyl)-2-(tetrahydro-2H-pyran-2-yloxy)ethyl]amino]ethyl]phenyl]-sulfonyl]-2-nitrobenzoate (266 mg).

(+)ESI-MS (m/z): 739, 741 (M+Na)⁺

### EXAMPLE 59

Under nitrogen at room temperature, to a solution of ethyl 5-[[4-[2-[(tert-butoxycarbonyl)[(2R)-2-(3-chlorophenyl)-2-(tetrahydro-2H-pyran-2-yloxy)ethyl]amino]-ethyl]phenyl]sulfonyl]-2-nitrobenzoate (261 mg) in a mixture of ethanol (6 ml) and water (2 ml) were added reduced iron (61 mg) and ammonium chloride (9.7 mg), and the mixture was refluxed for 30 minutes. The insoluble materials were removed by filtration with celite and the filtrate was concentrated under reduced pressure. The residue was dissolved into a mixture of saturated aqueous sodium bicarbonate and ethyl acetate. After separation, the organic layer was washed successively with water and brine, dried over anhydrous magnesium sulfate and evaporated under reduced pressure. The residue was purified by column chromatography on silica gel (hexane/ethyl acetate=3:1 to 2:1) to give ethyl 2-amino-5-[[4-[2-[(tert-butoxycarbonyl)-[(2R)-2-(3-chlorophenyl)-2-(tetrahydro-2H-pyran-2-yloxy)-ethyl]amino]ethyl]phenyl]sulfonyl]benzoate (168 mg).

(+)ESI-MS (m/z): 709, 711 (M+Na)⁺

### EXAMPLE 60

Under nitrogen at 5° C., to a suspension of sodium hydride (60% in oil, 13 mg) in N,N-dimethylformamide (6 ml) was added ethyl 2-amino-5-[[4-[2-[(tert-butoxycarbonyl)-[(2R)-2-(3-chlorophenyl)-2-(tetrahydro-2H-pyran-2-yloxy)ethyl]amino]ethyl]phenyl]sulfonyl]benzoate (200 mg), and the resulting mixture was stirred at the same temperature for 20 minutes. To this one was added iodomethane (45 mg) at 5° C. and the mixture was stirred at the same temperature for 80 minutes. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=3:1 to 2:1) to give ethyl 5-[[4-[2-[(tert-butoxycarbonyl)[(2R)-2-(3-chlorophenyl)-2-(tetrahydro-2H-pyran-2-yloxy)ethyl]amino]-ethyl]phenyl]sulfonyl]-2-(methylamino)benzoate (167 mg).

(+)ESI-MS (m/z): 723, 725 (M+Na)⁺

### EXAMPLE 61

The following compound was obtained according to a similar manner to that of Example 60.

Ethyl 5-[[4-[2-[(tert-butoxycarbonyl)[(2R)-2-(3-chlorophenyl)-2-(tetrahydro-2H-pyran-2-yloxy)ethyl]amino]-ethyl]phenyl]sulfonyl]-2-(ethylamino)benzoate

(+)ESI-MS (m/z): 737, 739 (M+Na)⁺

### EXAMPLE 62

Under nitrogen at room temperature, to a solution of ethyl 5-[[4-[2-[(tert-butoxycarbonyl)[(2R)-2-(3-chlorophenyl)-2-(tetrahydro-2H-pyran-2-yloxy)ethyl]amino]-ethyl]phenyl]sulfonyl]-2-(methylamino)benzoate (164 mg) in methanol (3 ml) was added a catalytic amount of p-toluenesulfonic acid monohydrate, and the mixture was stirred at the same temperature for 8.5 hours. The resulting mixture was poured into saturated aqueous sodium bicarbonate and the aqueous mixture was extracted with ethyl acetate. The organic layer was washed successively with saturated aqueous sodium bicarbonate and brine, dried over anhydrous magnesium sulfate, evaporated under reduced pressure and dried in vacuo to give ethyl 5-[[4-[2-[(tert-butoxycarbonyl)[(2R)-2-(3-chlorophenyl)-2-hydroxyethyl]-amino]ethyl]phenyl]sulfonyl]-2-(methylamino)benzoate (140 mg).

(+)ESI-MS (m/z): 639, 641 (M+Na)⁺

### EXAMPLE 63

The following compounds were obtained according to a similar manner to that of Example 62.

(1) Ethyl 5-[[4-[2-[(tert-butoxycarbonyl)[(2R)-2-(3-chlorophenyl)-2-hydroxyethyl]amino]ethyl]phenyl]-sulfonyl]-2-(ethylamino)benzoate

(+)ESI-MS (m/z): 653, 655 (M+Na)⁺

(2) Ethyl 5-[[4-[2-[(tert-butoxycarbonyl)[(2R)-2-(3-chlorophenyl)-2-hydroxyethyl]amino]ethyl]phenyl]-sulfonyl]-2-(propylamino)benzoate

(+)ESI-MS (m/z): 667, 669 (M+Na)⁺

(3) Ethyl 5-[[4-[2-[(tert-butoxycarbonyl)[(2R)-2-(3-chlorophenyl)-2-hydroxyethyl]amino]ethyl]phenyl]-sulfonyl]-2-[(2-methoxyethyl)amino]benzoate

(+)ESI-MS (m/z): 683, 685 (M+Na)⁺

(4) Ethyl 5-[[4-[2-[(tert-butoxycarbonyl)[(2R)-2-(3-chlorophenyl)-2-hydroxyethyl]amino]ethyl]phenyl]-sulfonyl]-2-[(2-hydroxyethyl)amino]benzoate

(+)ESI-MS (m/z): 669, 671 (M+Na)⁺

(5) Methyl 5-[[4-[2-[(tert-butoxycarbonyl)[(2R)-2-(3-chlorophenyl)-2-hydroxyethyl]amino]ethyl]phenyl]-sulfonyl]-2-[(methoxycarbonyl)amino]benzoate

(+)ESI-MS (m/z): 669, 671 (M+Na)⁺

(6) Ethyl 5-[[4-[2-[(tert-butoxycarbonyl)[(2R)-2-(3-chlorophenyl)-2-hydroxyethyl]amino]ethyl]phenyl]-sulfonyl]-2-[(methylsulfonyl)amino]benzoate

(−)ESI-MS (m/z): 679, 681 (M−H)⁻

### EXAMPLE 64

To a solution of ethyl 5-[[4-[2-[(tert-butoxycarbonyl)-[(2R)-2-(3-chlorophenyl)-2-hydroxyethyl]amino]ethyl]phenyl]-sulfonyl]-2-(methylamino)benzoate (137 mg) in ethanol (3 ml) was added 1N sodium hydroxide (0.444 ml) at room temperature, and the mixture was stirred at 45° C. for 4 hours. To the resulting mixture was added 1N hydrochloric acid (0.444 ml) and the mixture was evaporated under reduced pressure. The residue was dissolved into a mixture of water and chloroform/methanol (5:1). After separation, the organic layer was dried over anhydrous magnesium sulfate and evaporated under reduced pressure. The residue was purified by column chromatography on silica gel (chloroform/methanol=20:1 to 10:1) to give 5-[[4-[2-[(tert-butoxycarbonyl)-[(2R)-2-(3-chlorophenyl)-2-hydroxyethyl]amino]ethyl]phenyl]-sulfonyl]-2-(methylamino)benzoic acid (129 mg).

(−)ESI-MS (m/z): 587, 589 (M−H)⁻

### EXAMPLE 65

The following compounds were obtained according to a similar manner to that of Example 64.

(1) 5-[[4-[2-[(tert-Butoxycarbonyl)[(2R)-2-(3-chlorophenyl)-2-hydroxyethyl]amino]ethyl]phenyl]-sulfonyl]-2-(ethylamino)benzoic acid

(−)ESI-MS (m/z): 601, 603 (M−H)⁻⁽2) 5-[[4-[2-[(tert-Butoxycarbonyl)[(2R)-2-(3-chlorophenyl)-2-hydroxyethyl]amino]ethyl]phenyl]-sulfonyl]-2-(propylamino)benzoic acid

(−)ESI-MS (m/z): 615, 617 (M−H)⁻

(3) (2S)-1-[4-[[4-[2-[(tert-Butoxycarbonyl)[(2R)-2-(3-chlorophenyl)-2-hydroxyethyl]amino]ethyl]phenyl]-sulfonyl]benzyl]-2-pyrrolidinecarboxylic acid

(−)ESI-MS (m/z): 641, 643 (M−H)⁻

### EXAMPLE 66

Under nitrogen at room temperature, to a solution of 5-[[4-[2-[(tert-butoxycarbonyl)[(2R)-2-(3-chlorophenyl)-2-hydroxyethyl]amino]ethyl]phenyl]sulfonyl]-2-(methylamino)-benzoic acid (125 mg) in ethyl acetate (1 ml) was added 4N hydrogen chloride in ethyl acetate (2 ml), and the mixture was stirred at the same temperature for 2.5 hours. The resulting mixture was decanted and the residue was washed with ethyl acetate, followed by dryness to give 5-[[4-[2-[[(2R)-2-(3-chlorophenyl)-2-hydroxyethyl]amino]ethyl]-phenyl]sulfonyl]-2-(methylamino)benzoic acid hydrochloride (94 mg).

NMR (DMSO-d₆, δ): 2.88 (3H, s), 2.95-3.45 (6H, m), 4.9-5.0 (1H, m), 6.84 (1H, d, J=9.2 Hz), 7.3-7.55 (6H, m), 7.75-7.9 (3H, m), 8.26 (1H, d, J=2.4 Hz)

(−)ESI-MS (m/z): 487 (M−HCl−H)⁻

### EXAMPLE 67

Under nitrogen at room temperature, to a solution of 5-[[4-[2-[(tert-butoxycarbonyl)[(2R)-2-(3-chlorophenyl)-2-hydroxyethyl]amino]ethyl]phenyl]sulfonyl]-2-(ethylamino)-benzoic acid (82 mg) in ethyl acetate (1 ml) was added 4N hydrogen chloride in ethyl acetate (2 ml), and the mixture was stirred at the same temperature for 2 hours. The resulting mixture was evaporated under reduced pressure and dried in vacuo to give 5-[[4-[2-[[(2R)-2-(3-chlorophenyl)-2-hydroxyethyl]amino]ethyl]phenyl]sulfonyl]-2-(ethylamino)-benzoic acid hydrochloride (67 mg).

NMR (DMSO-d₆, δ): 1.18 (3H, t, J=7.1 Hz), 2.95-3.45 (8H, m), 4.9-5.0 (1H, m), 6.88 (1H, d, J=9.2 Hz), 7.3-7.5 (6H, m), 7.75-7.9 (3H, m), 8.27 (1H, d, J=2.4 Hz)

(−)ESI-MS (m/z): 501, 503 (M−HCl−H)⁻

### EXAMPLE 68

The following compounds were obtained according to a similar manner to that of Example 67.

(1) 5-[[4-[2-[[(2R)-2-(3-Chlorophenyl)-2-hydroxyethyl]-amino]ethyl]phenyl]sulfonyl]-2-(propylamino)benzoic acid hydrochloride

NMR (DMSO-d₆, δ): 0.92 (3H, t, J=7.2 Hz), 1.5-1.7 (2H, m), 2.95-3.45 (8H, m), 4.9-5.0 (1H, m), 6.89 (1H, d, J=9.2 Hz), 7.3-7.5 (6H, m), 7.75-7.9 (3H, m), 8.27 (1H, d, J=2.4 Hz)

(−)ESI-MS (m/z): 515, 517 (M−HCl−H)⁻

(2) 5-[[4-[2-[[(2R)-2-(3-Chlorophenyl)-2-hydroxyethyl]-amino]ethyl]phenyl]sulfonyl]-2-[(2-methoxyethyl)amino]-benzoic acid hydrochloride

NMR (DMSO-d₆, δ): 2.95-3.7 (13H, m), 4.9-5.0 (1H, m), 6.92 (1H, d, J=9.2 Hz), 7.3-7.5 (6H, m), 7.75-7.9 (3H, m), 8.27 (1H, d, J=2.4 Hz)

(−)ESI-MS (m/z): 531, 533 (M−HCl−H)⁻

(3) 5-[[4-[2-[[(2R)-2-(3-Chlorophenyl)-2-hydroxyethyl]-amino]ethyl]phenyl]sulfonyl]-2-[(2-hydroxyethyl)amino]-benzoic acid hydrochloride

NMR (DMSO-d₆, δ): 2.95-3.65 (10H, m), 4.9-5.0 (1H, m), 6.92 (1H, d, J=9.2 Hz), 7.3-7.55 (6H, m), 7.75-7.9 (3H, m), 8.27 (1H, d, J=2.4 Hz)

(−)ESI-MS (m/z): 517, 519 (M−HCl−H)⁻

(4) 5-[[4-[2-[[(2R)-2-(3-Chlorophenyl)-2-hydroxyethyl]-amino]ethyl]phenyl]sulfonyl]-2-(propionylamino)benzoic acid hydrochloride

NMR (DMSO-d₆, δ): 1.10 (3H, t, J=7.5 Hz), 2.95-3.6 (6H, m), 4.9-5.0 (1H, m), 7.3-7.55 (6H, m), 7.93 (2H, d, J=8.3 Hz), 8.12 (1H, dd, J=2.4, 8.9 Hz), 8.44 (1H, d, J=2.3 Hz), 8.69 (1H, d, J=8.9 Hz)

(−)ESI-MS (m/z): 529, 531 (M−HCl−H)⁻

(5) 5-[[4-[2-[[(2R)-2-(3-Chlorophenyl)-2-hydroxyethyl]-amino]ethyl]phenyl]sulfonyl]-2-(glycoloylamino)benzoic acid hydrochloride

NMR (DMSO-d₆, δ): 2.9-3.6 (6H, m), 4.0-4.05 (2H, m), 4.9-5.0 (1H, m), 7.3-7.6 (6H, m), 7.94 (2H, d, J=8.3 Hz), 8.16 (1H, dd, J=2.4, 8.9 Hz), 8.47 (1H, d, J=2.4 Hz), 8.89 (1H, d, J=9.0 Hz)

(−)ESI-MS (m/z): 531 (M−HCl−H)⁻

(6) 5-[[4-[2-[[(2R)-2-(3-Chlorophenyl)-2-hydroxyethyl]-amino]ethyl]phenyl]sulfonyl]-2-[(methoxycarbonyl)-amino]benzoic acid hydrochloride

NMR (DMSO-d₆, δ): 2.9-3.6 (6H, m), 4.9-5.0 (1H, m), 7.3-7.6 (6H, m), 7.92 (2H, d, J=8.3 Hz), 8.12 (1H, dd, J=2.3, 9.0 Hz), 8.4-8.5 (2H, m)

(−)ESI-MS (m/z): 531, 533 (M−HCl−H)⁻

(7) 5-[[4-[2-[[(2R)-2-(3-Chlorophenyl)-2-hydroxyethyl]-amino]ethyl]phenyl]sulfonyl]-2-[(methylsulfonyl)-amino]benzoic acid hydrochloride

NMR (DMSO-d₆, δ): 2.3-3.6 (9H, m), 4.9-5.0 (1H, m), 7.3-7.6 (6H, m), 7.69 (1H, d, J=8.8 Hz), 7.92 (2H, d, J=8.3 Hz), 8.07 (1H, dd, J=2.4, 8.9 Hz), 8.43 (1H, d, J=2.3 Hz)

(−)ESI-MS (m/z): 551, 553 (M−HCl−H)⁻

(8) (2S)-1-[4-[[4-[2-[[(2R)-2-(3-Chlorophenyl)-2-hydroxyethyl]amino]ethyl]phenyl]sulfonyl]benzyl]-2-pyrrolidinecarboxylic acid dihydrochloride

NMR (DMSO-d₆, δ): 1.7-2.1 (4H, m), 2.9-3.6 (8H, m), 4.15-4.6 (3H, m), 4.9-5.0 (1H, m), 7.25-7.6 (6H, m), 7.76 (2H, d, J=8.3 Hz), 7.95 (2H, d, J=8.3 Hz), 8.03 (2H, d, J=8.3 Hz)

(−)ESI-MS (m/z): 541, 543 (M−2HCl−H)⁻

(9) 2-Chloro-6-[[4-[2-[[(2R)-2-(3-chlorophenyl)-2-hydroxyethyl]amino]propyl]phenyl]sulfonyl]benzoic acid hydrochloride

NMR (DMSO-d₆, δ): 1.10 (3H, d, J=6.3 Hz), 2.7-2.9 (2H, m), 3.0-3.6 (3H, m), 5.0-5.1 (1H, m), 7.3-7.6 (6H, m), 7.67 (1H, t, J=8.0 Hz), 7.88 (1H, d, J=7.4 Hz), 7.95 (2H, d, J=6.7 Hz), 8.05-8.15 (1H, m)

(−)ESI-MS (m/z): 506, 508 (M−HCl−H)⁻

(10) (2E)-3-[2-[[4-[(2R)-2-[[(2R)-2-(3-Chlorophenyl)-2-hydroxyethyl]amino]propyl]phenyl]sulfonyl]-5-methylphenyl]acrylic acid hydrochloride

NMR (DMSO-d₆, δ): 1.06 (3H, d, J=6.3 Hz), 2-0.40 (3H, s), 2.65-2.9 (1H, m), 3.0-3.6 (4H, m), 4.9-5.05 (1H, m), 6.34 (1H, d, J=15.7 Hz), 7.3-7.6 (7H, m), 7.7-7.85 (3H, m), 8.07 (1H, d, J=8.1 Hz), 8.25 (1H, d, J=15.8 Hz)

(−)ESI-MS (m/z): 512 (M−HCl−H)⁻

(11) (2Z)-3-[2-[[4-[(2R)-2-[[(2R)-2-(3-Chlorophenyl)-2-hydroxyethyl]amino]propyl]phenyl]sulfonyl]-5-methylphenyl]acrylic acid hydrochloride

NMR (DMSO-d₆, δ): 1.05 (3H, d, J=6.3 Hz), 2.34 (3H, s) 2.6-2.9 (1H, m), 3.0-3.55 (4H, m), 4.9-5.0 (1H, m), 5.96 (1H, d, J=12.0 Hz), 7.15 (1H, s), 7.25-7.5 (8H, m), 7.76 (2H, d, J=8.2 Hz), 8.00 (1H, d, J=8.1 Hz)

(−)ESI-MS (m/z): 512 (M−HCl−H)⁻

### EXAMPLE 69

Under nitrogen at SOC, to a suspension of ethyl 2-amino-5-[[4-[2-[(tert-butoxycarbonyl)[(2R)-2-(3-chlorophenyl)-2-(tetrahydro-2H-pyran-2-yloxy)ethyl]amino]-ethyl]phenyl]sulfonyl]benzoate (134 mg) in N,N-dimethylformamide (3 ml) was added sodium hydride (60% in oil, 8.6 mg), and the resulting mixture was stirred at the same temperature for 15 minutes. To this one was added 1-bromopropane (120 mg) and potassium iodide (162 mg) at 5° C. and the mixture was stirred at room temperature for 3 days. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=3:1 to 2:1) to give ethyl 5-[[4-[2-[(tert-butoxycarbonyl)[(2R)-2-(3-chlorophenyl)-2-(tetrahydro-2H-pyran-2-yloxy)ethyl]-amino]ethyl]phenyl]sulfonyl]-2-(propylamino)benzoate (72 mg).

(+)ESI-MS (m/z): 751, 753 (M+Na)⁺

### EXAMPLE 70

Under nitrogen at 5° C., to a solution of 2-amino-5-[[4-[2-[(tert-butoxycarbonyl)[(2R)-2-(3-chlorophenyl)-2-(tetrahydro-2H-pyran-2-yloxy)ethyl]amino]ethyl]phenyl]-sulfonyl]benzoate (200 mg) in N,N-dimethylformamide (4 ml) was added sodium hydride (60% in oil, 13 mg), and the resulting mixture was stirred at the same temperature for 20 minutes. To this one was added 2-methoxyethyl 4-nitrobenzenesulfonate (228 mg) at 5° C. and the mixture was stirred at the same temperature for 6 hours. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=3:2) to give ethyl 5-[[4-[2-[(tert-butoxycarbonyl)[(2R)-2-(3-chlorophenyl)-2-(tetrahydro-2H-pyran-2-yloxy)ethyl]amino]ethyl]phenyl]-sulfonyl]-2-[(2-methoxyethyl)amino]benzoate (124 mg).

(+)ESI-MS (m/z): 767, 769 (M+Na)⁺

### EXAMPLE 71

To a solution of ethyl 5-[[4-[2-[(tert-butoxycarbonyl)-[(2R)-2-(3-chlorophenyl)-2-hydroxyethyl]amino]ethyl]phenyl]-sulfonyl]-2-[(2-methoxyethyl)amino]benzoate (42 mg) in ethanol (2 ml) was added 1N sodium hydroxide (0.19 ml) at room temperature, and the mixture was stirred at 45° C. for 2 hours. The resulting mixture was poured into 1N hydrochloric acid and the aqueous mixture was extracted with chloroform/methanol (4:1). After separation, the organic layer was dried over anhydrous magnesium sulfate and evaporated under reduced pressure. The residue was purified by column chromatography on silica gel (chloroform/methanol=20:1 to 10:1) to give 5-[[4-[2-[(tert-butoxycarbonyl)-[(2R)-2-(3-chlorophenyl)-2-hydroxyethyl]amino]ethyl]phenyl]-sulfonyl]-2-[(2-methoxyethyl)amino]benzoic acid (33 mg).

(−)ESI-MS (m/z): 631, 633 (M−H)⁻

### EXAMPLE 72

The following compounds were obtained according to a similar manner to that of Example 71.

(1) 5-[[4-[2-[(tert-Butoxycarbonyl)[(2R)-2-(3-chlorophenyl)-2-hydroxyethyl]amino]ethyl]phenyl]-sulfonyl]-2-[(2-hydroxyethyl)amino]benzoic acid

(−)ESI-MS (m/z): 617, 619 (M−H)⁻

(2) 5-[[4-[2-[(tert-Butoxycarbonyl)[(2R)-2-(3-chlorophenyl)-2-hydroxyethyl]amino]ethyl]phenyl]-sulfonyl]-2-[(methoxycarbonyl)amino]benzoic acid

(−)ESI-MS (m/z): 631, 633 (M−H)⁻

(3) 5-[[4-[2-[(tert-Butoxycarbonyl)[(2R)-2-(3-chlorophenyl)-2-hydroxyethyl]amino]ethyl]phenyl]-sulfonyl]-2-[(methylsulfonyl)amino]benzoic acid

(−)ESI-MS (m/z): 651, 653 (M−H)⁻

### EXAMPLE 73

Under nitrogen at 5° C., to a solution of ethyl 2-amino-5-[[4-[2-[(tert-butoxycarbonyl)[(2R)-2-(3-chlorophenyl)-2-(tetrahydro-2H-pyran-2-yloxy)ethyl]amino]ethyl]phenyl]-sulfonyl]benzoate (200 mg) in N,N-dimethylformamide (4 ml) was added sodium hydride (60% in oil, 13 mg), and the resulting mixture was stirred at the same temperature for 20 minutes. To this one was added 2-(2-iodoethoxy)tetrahydro-2H-pyran (224 mg) at 5° C. and the mixture was stirred at the same temperature for 5 hours. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=2:1 to 3:2) to give ethyl 5-[[4-[2-[(tert-butoxycarbonyl)[(2R)-2-(3-chorophenyl)-2-(tetrahydro-2H-pyran-2-yloxy)ethyl]amino]ethyl]phenyl]-sulfonyl]-2-[[2-(tetrahydro-2H-pyran-2-yloxy)ethyl]amino]-benzoate (177 mg).

(+)ESI-MS (m/z): 837, 839 (M+Na)⁺

### EXAMPLE 74

To a solution of ethyl 2-amino-5-[[4-[2-[(tert-butoxycarbonyl)[(2R)-2-(3-chlorophenyl)-2-(tetrahydro-2H-pyran-2-yloxy)ethyl]amino]ethyl]phenyl]sulfonyl]benzoate (200 mg) in ethanol (8 ml) was added 1N sodium hydroxide (0.58 ml) at room temperature, and the mixture was stirred at 45°C for 4 hours. To the resulting mixture was added 1N hydrochloric acid (0.58 ml) and the mixture was evaporated and dried in vacuo. To a solution of the residue in dichloromethane (3 ml) were added pyridine (230 mg) and acetyl chloride (48 mg) at 5° C. under nitrogen, and the mixture was stirred at the same temperature for 3.5 hours. The resulting mixture was poured into a mixture of 1N hydrochloric acid and ethyl acetate, and the mixture was stirred for 15 minutes. After separation, the organic layer was dried over anhydrous magnesium sulfate, evaporated under reduced pressure and dried in vacuo to give 2-(acetylamino)-5-[[4-[2-[(tert-butoxycarbonyl)[(2R)-2-(3-chlorophenyl)-2-hydroxyethyl]amino]ethyl]phenyl]sulfonyl]benzoic acid (199 mg).

(−)ESI-MS (m/z): 615, 617 (M−H)⁻

### EXAMPLE 75

Under nitrogen at room temperature, to a solution of 2-(acetylamino)-5-[[4-[2-[(tert-butoxycarbonyl)[(2R)-2-(3-chlorophenyl)-2-hydroxyethyl]amino]ethyl]phenyl]sulfonyl]-benzoic acid (197 mg) in ethyl acetate (1 ml) was added 4N hydrogen chloride in ethyl acetate (3 ml), and the mixture was stirred at the same temperature for 3 hours. The precipitates were collected by filtration, followed by dryness to give 2-(acetylamino)-5-[[4-[2-[[(2R)-2-(3-chlorophenyl)-2-hydroxyethyl]amino]ethyl]phenyl]sulfonyl]-benzoic acid hydrochlride (139 mg).

NMR (DMSO-d₆, δ): 2.17 (3H, s), 2.9-3.6 (6H, m), 4.9-5.0 (1H, m), 7.3-7.6 (6H, m), 7.93 (2H, d, J=8.2 Hz), 8.14 (1H, dd, J=2.4, 8.9 Hz), 8.43 (1H, d, J=2.3 Hz), 8.65 (1H, d, J=8.9 Hz)

(−)ESI-MS (m/z): 515, 517 (M−HCl−H)⁻

### EXAMPLE 76

The following compounds were obtained according to a similar manner to that of Example 75.

(1) 2-(Benzoylamino)-5-[[4-[2-[[(2R)-2-(3-chlorophenyl)-2-hydroxyethyl]amino]ethyl]phenyl]sulfonyl]benzoic acid hydrochloride

NMR (DMSO-d₆, δ): 2.9-3.6 (6H, m), 4.9-5.0 (1H, m), 7.3-7.7 (9H, m), 7.9-8.0 (4H, m), 8.21 (1H, dd, J=2.4, 8.9 Hz), 8.51 (1H, d, J=2.4 Hz), 8.90 (1H, d, J=8.9 Hz)

(−)ESI-MS (m/z): 577, 579 (M−HCl−H)⁻

(2) 2-[[4-[(2R)-2-[[(2R)-2-(3-Chlorophenyl)-2-hydroxyethyl]amino]propyl]phenyl]sulfonyl]-5-(methylthio)benzoic acid hydrochloride

1.05-1.15 (3H, m), 2.55 (3H, s), 2.7-3.6 (5H, m), 4.95-5.1 (1H, m), 7.3-7.6 (8H, m), 7.92 (2H, d, J=8.3 Hz), 8.00 (1H, d, J=8.5 Hz)

(−)ESI-MS (m/z): 518, 520 (M−HCl−H)⁻

(3) 2-[[4-[(2R)-2-[[(2R)-2-(3-Chlorophenyl)-2-hydroxyethyl]amino]propyl]phenyl]sulfonyl]-5-(trifluoromethyl)benzoic acid hydrochloride

NMR (DMSO-d₆, δ): 1.05-1.15 (3H, m), 2.7-2.9 (1H, m), 3.0-3.65 (4H, m), 4.95-5.1 (1H, m), 7.35-7.6 (6H, m), 7.9-8.2 (4H, m), 8.39 (1H, d, J=8.3 Hz)

(−)ESI-MS (m/z): 540, 542 (M−HCl−H)⁻

### EXAMPLE 77

To a solution of ethyl 2-amino-5-[[4-[2-[(tert-butoxycarbonyl)[(2R)-2-(3-chlorophenyl)-2-(tetrahydro-2H-pyran-2-yloxy)ethyl]amino]ethyl]phenyl]sulfonyl]benzoate (800 mg) in ethanol (10 ml) was added 1N sodium hydroxide (2.32 ml) at room temperature, and the mixture was stirred at 45° C. for 2.5 hours. To the resulting mixture was added 1N hydrochloric acid (2.32 ml) and the mixture was evaporated under reduced pressure. To the residue was added a mixture of chloroform and methanol (4:1), and the mixture was stirred for 30 minutes. The insoluble materials were removed by filtration, and the filtrate was evaporated and dried in vacuo to give 2-amino-5-[[4-[2-[(tert-butoxycarbonyl)[(2R)-2-(3-chlorophenyl)-2-(tetrahydro-2H-pyran-2-yloxy)ethyl]amino]ethyl]phenyl]sulfonyl]benzoic acid (822 mg).

(−)ESI-MS (m/z): 657, 659 (M−H)⁻

### EXAMPLE 78

Under nitrogen at 5° C., to a solution of 2-amino-S-[[4-[2-[(tert-butoxycarbonyl)[(2R)-2-(3-chlorophenyl)-2-(tetrahydro-2H-pyran-2-yloxy)ethyl]amino]ethyl]phenyl]-sulfonyl]benzoic acid (150 mg) in dichloromethane (3 ml) were added pyridine (180 mg) and propionyl chloride (51 mg), and the mixture was stirred at the same temperature for 4.5 hours. The resulting mixture was poured into a mixture of 1N hydrochloric acid and ethyl acetate, and the mixture was stirred for 15 minutes. After separation, the organic layer was dried over anhydrous magnesium sulfate and evaporated under reduced pressure. To a solution of the residue in a mixture of methanol (3 ml) and water (0.5 ml) was added a catalytic amount of p-toluenesulfonic acid monohydrate at room temperature, and the mixture was stirred at the same temperature for 43 hours. The resulting mixture was poured into 1N hydrochloric acid and the aqueous mixture was extracted with a mixture of chloroform and methanol (4:1). The organic layer was dried over anhydrous magnesium sulfate and evaporated under reduced pressure. The residue was purified by column chromatography on silica gel (chloroform/methanol=10:1 to 6:1) to give 5-[[4-[2-[(tert-butoxycarbonyl)[(2R)-2-(3-chlorophenyl)-2-hydroxyethyl]-amino]ethyl]phenyl]sulfonyl]-2-(propionylamino)benzoic acid (51 mg).

(−)ESI-MS (m/z): 629, 631 (M−H)⁻

### EXAMPLE 79

Under nitrogen at 5° C., to a solution of 2-amino-5-[[4-[2-[(tert-butoxycarbonyl)[(2R)-2-(3-chlorophenyl)-2-(tetrahydro-2H-pyran-2-yloxy)ethyl]amino]ethyl]phenyl]-sulfonyl]benzoic acid (150 mg) in dichloromethane (3 ml) were added pyridine (180 mg) and benzoyl chloride (77 mg), and the mixture was stirred at room temperature for 4 days. The resulting mixture was poured into 1N hydrochloric acid and the aqueous mixture was extracted with ethyl acetate. The organic layer was dried over anhydrous magnesium sulfate and evaporated under reduced pressure. To a solution of the residue in tetrahydrofuran (4 ml) was added 1N sodium hydroxide at room temperature, and the mixture was stirred at the same temperature for 2 hours. The resulting mixture was poured into 1N hydrochloric acid and the aqueous mixture was extracted with a mixture of chloroform and methanol (4:1). The organic layer was dried over anhydrous magnesium sulfate and evaporated under reduced pressure. To a solution of the residue in a mixture of methanol (4 ml) and water (0.5 ml) was added a catalytic amount of p-toluenesulfonic acid monohydrate at room temperature, and the mixture was stirred at the same temperature for 36 hours. The resulting mixture was poured into 1N hydrochloric acid and the aqueous mixture was extracted with a mixture of chloroform and methanol (4:1). The organic layer was dried over anhydrous magnesium sulfate and evaporated under reduced pressure. The residue was purified by column chromatography on silica gel (chloroform/methanol=10:1 to 8:1) to give 2-(benzoylamino)-5-[[4-[2-[(tert-butoxycarbonyl)[(2R)-2-(3-chlorophenyl)-2-hydroxyethyl]-amino]ethyl]phenyl]sulfonyl]benzoic acid (85 mg).

(−)ESI-MS (m/z): 677, 679 (M−H)⁻

### EXAMPLE 80

Under nitrogen at 5° C., to a solution of 2-amino-5-[[4-[2-[(tert-butoxycarbonyl)[(2R)-2-(3-chlorophenyl)-2-(tetrahydro-2H-pyran-2-yloxy)ethyl]amino]ethyl]phenyl]-sulfonyl]benzoic acid (311 mg) in dichloromethane (5 ml) were added pyridine (373 mg) and acetoxyacetyl chloride (135 mg), and the mixture was stirred at the same temperature for 100 minutes. The resulting mixture was poured into a mixture of 1N hydrochloric acid and ethyl acetate, and the mixture was stirred for 30 minutes. After separation, the organic layer was dried over anhydrous magnesium sulfate and evaporated under reduced pressure. To a solution of the residue in methanol (3 ml) was added Amberlyst 15 at room temperature, and the mixture was stirred at the same temperature for 12 hours. Amberlyst 15 was removed by filtration, and the filtrate was evaporated under reduced pressure. To a solution of the residue in tetrahydrofuran (5 ml) was added 1N sodium hydroxide (1.4 ml) at 5° C., and the mixture was stirred at the same temperature for 3 hours. The resulting mixture was poured into 1N hydrochloric acid and the aqueous mixture was extracted with a mixture of chloroform and methanol (4:1). The organic layer was dried over anhydrous magnesium sulfate and evaporated under reduced pressure. The residue was purified by column chromatography on silica gel (chloroform/methanol=10:1 to 5:1) to give 5-[[4-[2-[(tert-butoxycarbonyl)[(2R)-2-(3-chlorophenyl)-2-hydroxyethyl]amino]ethyl]phenyl]sulfonyl]-2-(glycoloylamino)benzoic acid (196 mg).

(−)ESI-MS (m/z): 631, 633 (M−H)⁻

### EXAMPLE 81

Under nitrogen at 5° C., to a solution of ethyl 2-amino-5-[[4-[2-[(tert-butoxycarbonyl)[(2R)-2-(3-chlorophenyl)-2-(tetrahydro-2H-pyran-2-yloxy)ethyl]amino]ethyl]phenyl]-sulfonyl]benzoate (300 mg) in N,N-dimethylformamide (5 ml) was added sodium hydride (60% in oil, 18 mg), and the resulting mixture was stirred at the same temperature for 20 minutes. After the mixture was cooled in dry ice-acetone bath, dimethyl carbonate (79 mg) was added, and the mixture was stirred at 5° C. for 1 hour. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=3:1 to 2:1) to give methyl 5-[[4-[2-[(tert-butoxycarbonyl)[(2R)-2-(3-chlorophenyl)-2-(tetrahydro-2H-pyran-2-yloxy)ethyl]amino]ethyl]phenyl]-sulfonyl]-2-[(methoxycarbonyl)amino]benzoate (106 mg).

(+)ESI-MS (m/z): 753, 755 (M+Na)⁺

### EXAMPLE 82

Under nitrogen at SOC, to a solution of ethyl 2-amino-5-[[4-[2-[(tert-butoxycarbonyl)[(2R)-2-(3-chlorophenyl)-2-(tetrahydro-2H-pyran-2-yloxy)ethyl]amino]ethyl]phenyl]-sulfonyl]benzoate (200 mg) in dichloromethane (4 ml) were added pyridine (230 mg) and methanesulfonyl chloride (90 mg), and the mixture was stirred at room temperature for 19.5 hours. The resulting mixture was poured into a mixture of 1N hydrochloric acid and the aqueous mixture was extracted with ethyl acetate. The organic layer was washed successively with 1N hydrochloric acid, saturated aqueous sodium bicarbonate and brine, dried over anhydrous magnesium sulfate and evaporated under reduced pressure. The residue was purified by column chromatography on silica gel (hexane/ethyl acetate=2:1 to 3:2) to give 5-[[4-[2-[(tert-butoxycarbonyl)[(2R)-2-(3-chlorophenyl)-2-(tetrahydro-2H-pyran-2-yloxy)ethyl]amino]ethyl]phenyl]sulfonyl]-2-[(methylsulfonyl)amino]benzoate (125 mg).

(−)ESI-MS (m/z): 763, 765 (M−H)⁻

### EXAMPLE 83

A solution of ethyl [5-[[4-[2-(benzylamino)ethyl]-phenyl]sulfonyl]-2-hydroxyphenyl]acetate (375 mg) and (2R)-2-(3-chlorophenyl)oxirane (141 mg) in ethanol (5 ml) was refluxed for 47.5 hours. The resulting mixture was evaporated under reduced pressure. The residue was purified by column chromatography on silica gel (hexane/ethyl acetate=2:1 to 4:3) to give ethyl[5-[[4-[2-[[(2R)-2-(3-chlorophenyl)-2-hydroxyethyl]amino]ethyl]phenyl]sulfonyl]-2-hydroxyphenyl]acetate (346 mg).

(+)ESI-MS (m/z): 608, 610 (M+H)⁺

### EXAMPLE 84

The following compounds were obtained according to a similar manner to that of Example 83.

(1) Ethyl 5-chloro-2-[[4-[(2R)-2-[[(2R)-2-(3-chlorophenyl)-2-hydroxyethyl]amino]propyl]phenyl]sulfonyl]benzoate

(+)ESI-MS (m/z): 536, 538 (M+H)⁺

(2) Ethyl 3-chloro-4-[[4-[(2R)-2-[[(2R)-2-(3-chlorophenyl)-2-hydroxyethyl]amino]propyl]phenyl]sulfonyl]benzoate

(+)ESI-MS (m/z): 536, 538 (M+H)⁺

(3) Methyl 4-chloro-2-[[4-[(2R)-2-[[(2R)-2-(3-chlorophenyl)-2-hydroxyethyl]amino]propyl]phenyl]-sulfonyl]benzoate

(+)ESI-MS (m/z): 522, 524 (M+H)⁺

(4) Ethyl 3-chloro-2-[[4-[(2R)-2-[[(2R)-2-(3-chlorophenyl)-2-hydroxyethyl]amino]propyl]phenyl]sulfonyl]benzoate

(+)ESI-MS (m/z): 535, 537 (M+H)⁺

(5) Methyl 2-[[4-[(2R)-2-[[(2R)-2-(3-chlorophenyl)-2-hydroxyethyl]amino]propyl]phenyl]sulfonyl]-5-methylbenzoate

(+)ESI-MS (m/z): 502, 504 (M+H)⁺

(6) Ethyl 2-[[4-[(2R)-2-[[(2R)-2-(3-chlorophenyl)-2-hydroxyethyl]amino]propyl]phenyl]sulfonyl]-5-methoxybenzoate

(+)ESI-MS (m/z): 532 (M+H)⁺

(7) Ethyl 2-[[4-[(2R)-2-[[(2R)-2-(3-chlorophenyl)-2-hydroxyethyl]amino]propyl]phenyl]sulfonyl]-5-phenoxybenzoate

(+)ESI-MS (m/z): 594, 596 (M+H)⁺

(8) Ethyl 2-[[4-[(2R)-2-[[(2R)-2-(3-chlorophenyl)-2-hydroxyethyl]amino]propyl]phenyl]sulfonyl]-5-propylbenzoate

(+)ESI-MS (m/z): 544, 546 (M+H)⁺

(9) Ethyl 2-[[4-[(2R)-2-[[(2R)-2-(3-chlorophenyl)-2-hydroxyethyl]amino]propyl]phenyl]sulfonyl]-5-(3-methylbutyl)benzoate

(+)ESI-MS (m/z): 572, 574 (M+H)⁺

(10) Ethyl 2-[[4-[(2R)-2-[[(2R)-2-(3-chlorophenyl)-2-hydroxyethyl]amino]propyl]phenyl]sulfonyl]-5-cyclohexylbenzoate

(+)ESI-MS (m/z): 584, 586 (M+H)⁺

(11) Ethyl 2-[[4-[(2R)-2-[[(2R)-2-(3-chlorophenyl)-2-hydroxyethyl]amino]propyl]phenyl]sulfonyl]-4-propylbenzoate

(+)ESI-MS (m/z): 544, 546 (M+H)⁺

(12) Ethyl 4-[[4-[(2R)-2-[[(2R)-2-(3-chlorophenyl)-2-hydroxyethyl]amino]propyl]phenyl]sulfonyl]-3-biphenylcarboxylate

(+)ESI-MS (m/z): 578, 580 (M+H)⁺

(13) Ethyl 5-[[4-[(2R)-2-[[(2R)-2-(3-chlorophenyl)-2-hydroxyethyl]amino]propyl]phenyl]sulfonyl]-2-hydroxy-4-methylbenzoate

(+)ESI-MS (m/z): 532, 534 (M+H)⁺

(14) Ethyl 5-[[4-[(2R)-2-[[(2R)-2-(3-chlorophenyl)-2-hydroxyethyl]amino]propyl]phenyl]sulfonyl]-2-hydroxy-4-methoxybenzoate

(+)ESI-MS (m/z): 548, 550 (M+H)⁺

(15) Methyl 5-[[4-[2-[[(2R)-2-(3-chlorophenyl)-2-hydroxyethyl]amino]ethyl]phenyl]sulfonyl]-2-(2-ethoxy-2-oxoethoxy)benzoate

(+)ESI-MS (m/z): 576 (M+H)⁺

(16) Methyl 5-[[4-[2-[[(2R)-2-(3-chlorophenyl)-2-hydroxyethyl]amino]ethyl]phenyl]sulfonyl]-2-(2-hydroxyethoxy)benzoate

(+)ESI-MS (m/z): 534, 536 (M+H)⁺

### EXAMPLE 85

To a solution of ethyl[5-[[4-[2-[[(2R)-2-(3-chlorophenyl)-2-hydroxyethyl]amino]ethyl]phenyl]sulfonyl]-2-hydroxyphenyl]acetate (343 mg) in ethanol (5 ml) was added 4N hydrogenchloride in ethyl acetate (0.5 ml), and the mixture was evaporated under reduced pressure. A mixture of the residue and 10% palladium on activated carbon (50% wet, 17 mg) in a mixture of ethanol (3 ml) and chlorobenzene (7 ml) was stirred at room temperature in the presence of hydrogen at an atmospheric pressure for 2 hours. After filtration, the filtrate was evaporated under reduced pressure. The residue was dissolved into a mixture of saturated aqueous sodium bicarbonate and ethyl acetate. After separation, the organic layer was washed with brine, dried over anhydrous magnesium sulfate and evaporated under reduced pressure. The residue was purified by column chromatography on silica gel (chloroform/methanol=20:1 to 15:1) to give ethyl[5-[[4-[2-[[(2R)-2-(3-chlorophenyl)-2-hydroxyethyl]amino]ethyl]phenyl]sulfonyl]-2-hydroxyphenyl]-acetate (258 mg)

(+)ESI-MS (m/z): 518, 520 (M+H)⁺

### EXAMPLE 86

To a solution of ethyl [5-[[4-[2-[[(2R)-2-(3-chlorophenyl)-2-hydroxyethyl]amino]ethyl]phenyl]sulfonyl]-2-hydroxyphenyl]acetate (255 mg) in methanol (5 ml) was added 1N sodium hydroxide (1.48 ml) at room temperature, and the mixture was stirred at 45° C. for 3 hours. To the resulting mixture was added 1N hydrochloric acid (2.46 ml) and the mixture was evaporated under reduced pressure. The residue was purified by reversed phase chromatography to give [5-[[4-[2-[[(2R)-2-(3-chlorophenyl)-2-hydroxyethyl]amino]-ethyl]phenyl]sulfonyl]-2-hydroxyphenyl]acetic acid hydrochloride (222 mg).

NMR (DMSO-d₆, δ): 2.95-3.4 (6H, m), 3.54 (2H, s), 4.9-5.0 (1H, m), 6.98 (1H, d, J=8.4 Hz), 7.3-7.55 (6H, m), 7.65-7.8 (2H, m), 7.86 (2H, d, J=8.2 Hz)

(−)ESI-MS (m/z): 488, 490 (M−HCl−H)⁻

### EXAMPLE 87

Under nitrogen at 5° C., to a solution of tert-butyl [(2R)-2-(3-chlorophenyl)-2-hydroxyethyl][2-[4-[(4-formylphenyl)thio]phenyl]ethyl]carbamate (1.33 g) in dichloromethane (15 ml) was added m-chloroperbenzoic acid (1.34 g), and the mixture was stirred at room temperature for 5.5 hours. The resulting mixture was poured into water which was adjusted to pH 7.2 by sodium sulfite and sodium bicarbonate and the aqueous mixture was extracted with ethyl acetate. The organic layer was washed with water, dried over anhydrous magnesium sulfate, evaporated and dried in vacuo to give tert-butyl [(2R)-2-(3-chlorophenyl)-2-hydroxyethyl][2-[4-[(4-formylphenyl)sulfonyl]phenyl]ethyl]-carbamate (1.25 g).

(+)ESI-MS (m/z): 566, 568 (M+Na)⁺

### EXAMPLE 88

Under nitrogen at 5° C., to a solution of tert-butyl [(2R)-2-(3-chlorophenyl)-2-hydroxyethyl][2-[4-[(4-formylphenyl)sulfonyl]phenyl]ethyl]carbamate (300 mg) and L-proline methyl ester (78 mg) in 1,2-dichloroethane (6 ml) was added sodium triacetoxyborohydride (292 mg), and the mixture was stirred at room temperature for 12 hours. The resulting mixture was poured into a mixture of 1N hydrochloric acid and ethyl acetate, and the mixture was stirred for 20 minutes. After separation, the organic layer was washed successively with water, saturated aqueous sodium bicarbonate and brine, dried over anhydrous magnesium sulfate and evaporated under reduced pressure. The residue was purified by column chromatography on silica gel (hexane/ethyl acetate=2:1 to 1:1) to give methyl(2S)-1-[4-[[4-[2-[(tert-butoxycarbonyl)[(2R)-2-(3-chlorophenyl)-2-hydroxyethyl]amino]ethyl]phenyl]sulfonyl]benzyl]-2-pyrrolidinecarboxylate (183 mg).

(+)ESI-MS (m/z): 679, 681 (M+Na)⁺

### EXAMPLE 89

To a solution of ethyl 5-chloro-2-[[4-[(2R)-2-[[(2R)-2-(3-chlorophenyl)-2-hydroxyethyl]amino]propyl]phenyl]-sulfonyl]benzoate (88 mg) in ethanol (2 ml) was added 1N sodium hydroxide (0.25 ml) at room temperature, and the mixture was stirred at 50° C. for 5 hours. To the resulting mixture was added 1N hydrochloric acid (0.082 ml) and the mixture was evaporated under reduced pressure. The residue was purified by reversed phase chromatography to give sodium 5-chloro-2-[[4-[(2R)-2-[[(2R)-2-(3-chlorophenyl)-2-hydroxyethyl]amino]propyl]phenyl]sulfonyl]benzoate (27 mg).

NMR (DMSO-d₆, δ): 0.94 (3H, d, J=6.0 Hz), 2.4-3.0 (5H, m), 4.55-4.7 (1H, m), 7.2-7.5 (8H, m), 7.90 (1H, d, J=8.5 Hz), 7.98 (2H, d, J=8.3 Hz)

(−)ESI-MS (m/z): 506, 508 (M−Na)⁻

### EXAMPLE 90

The following compounds were obtained according to a similar manner to that of Example 89.

(1) Sodium 3-chloro-4-[[4-[(2R)-2-[[(2R)-2-(3-chlorophenyl)-2-hydroxyethyl]amino]propyl]phenyl]-sulfonyl]benzoate

NMR (DMSO-d₆, δ): 0.93 (3H, d, J=6.0 Hz), 2.4-3.0 (5H, m), 4.55-4.7 (1H, m), 7.2-7.5 (6H, m), 7.78 (2H, d, J=8.2 Hz), 7.87 (1H, d, J=1.2 Hz), 7.99 (1H, dd, J=1.3, 8.0 Hz), 8.19 (1H, d, J=8.1 Hz)

(−)ESI-MS (m/z): 506, 508 (M−Na)⁻

(2) Sodium 4-chloro-2-[[4-[(2R)-2-[[(2R)-2-(3-chlorophenyl)-2-hydroxyethyl]amino]propyl]phenyl]-sulfonyl]benzoate

NMR (DMSO-d₆, δ): 1.00 (3H, d, J=5.9 Hz), 2.6-3.6 (5H, m), 4.9-5.1 (1H, m), 7.2-7.5 (7H, m), 7.66 (1H, dd, J=2.1, 8.2 Hz), 8.00 (1H, d, J=2.0 Hz), 8.04 (2H, d, J=8.3 Hz)

(−)ESI-MS (m/z): 506, 508 (M−Na)⁻

(3) Sodium 2-[[4-[(2R)-2-[[(2R)-2-(3-chlorophenyl)-2-hydroxyethyl]amino]propyl]phenyl]sulfonyl]-5-methylbenzoate

0.94 (3H, d, J=6.0 Hz), 2.30 (3H, s), 2.5-3.6 (5H, m), 4.65-4.85 (1H, m), 7.05-7.5 (8H, m), 7.79 (1H, d, J=8.1 Hz), 7.97 (2H, d, J=8.2 Hz)

(−)ESI-MS (m/z): 486 (M−Na)⁻

(4) Sodium 2-[[4-[(2R)-2-[[(2R)-2-(3-chlorophenyl)-2-hydroxyethyl]amino]propyl]phenyl]sulfonyl]-5-phenoxybenzoate

NMR (DMSO-d₆, δ): 0.96 (3H, d, J=5.9 Hz), 2.5-3.6 (5H, m), 4.75-4.95 (1H, m), 6.70 (1H, d, J=2.4 Hz), 6.97 (1H, dd, J=2.6, 8.7 Hz), 7.05-7.5 (11H, m), 7.9-8.0 (3H, m)

(−)ESI-MS (m/z): 564, 566 (M−Na)⁻

(5) Sodium 5-[[4-[(2R)-2-[[(2R)-2-(3-chlorophenyl)-2-hydroxyethyl]amino]propyl]phenyl]sulfonyl]-2-hydroxy-4-methylbenzoate

NMR (DMSO-d₆, δ): 1.01 (3H, d, J=6.2 Hz), 2.20 (3H, s), 2.55-2.75 (1H, m), 2.8-3.5 (4H, m), 4.7-4.85 (1H, m), 6.50 (1H, s), 7.25-7.5 (6H, m), 7.68 (2H, d, J=8.2 Hz), 8.38 (1H, s)

(−)ESI-MS (m/z): 502, 504 (M−Na)⁻

(6) Sodium 5-[[4-[(2R)-2-[[(2R)-2-(3-chlorophenyl)-2-hydroxyethyl]amino]propyl]phenyl]sulfonyl]-2-hydroxy-4-methoxybenzoate

NMR (DMSO-d₆, δ): 0.97 (3H, d, J=6.1 Hz), 2.5-3.4 (5H, m), 3.63 (3H, s), 4.6-4.8 (1H, m), 6.15 (1H, s), 7.25-7.5 (6H, m), 7.72 (2H, d, J=8.2 Hz), 8.25 (1H, s)

(−)ESI-MS (m/z): 518, 520 (M−Na)⁻

### EXAMPLE 91

Under nitrogen at 5° C., to a solution of tert-butyl [(1R)-2-[4-[(3-chloro-2-formylphenyl)thio]phenyl]-1-methylethyl][(2R)-2-(3-chlorophenyl)-2-hydroxyethyl]-carbamate (173 mg) in dichloromethane (4 ml) was added m-chloroperbenzoic acid (160 mg), and the mixture was stirred at room temperature for 2.5 hours. The resulting mixture was poured into saturated aqueous sodium bicarbonate and the aqueous mixture was extracted with ethyl acetate. The organic layer was washed successively with saturated aqueous sodium bicarbonate (two times) and brine, dried over anhydrous magnesium sulfate, evaporated and dried in vacuo to give tert-butyl [(1R)-2-[4-[(3-chloro-2-formylphenyl)-sulfonyl]phenyl]-1-methylethyl][(2R)-2-(3-chlorophenyl)-2-hydroxyethyl]carbamate (204 mg).

(+)ESI-MS (m/z): 614, 616 (M+Na)⁺

### EXAMPLE 92

The following compounds were obtained according to a similar manner to that of Preparation 33.

(1) 2-[[4-[(2R)-2-[(tert-Butoxycarbonyl)[(2R)-2-(3-chlorophenyl)-2-hydroxyethyl]amino]propyl]phenyl]-sulfonyl]-6-chlorobenzoic acid

(−)ESI-MS (m/z): 606, 608 (M−H)⁻

(2) 2-[[4-[(2R)-2-[(tert-Butoxycarbonyl)[(2R)-2-[[tert-butyl(dimethyl)silyl]oxy]-2-(3-chlorophenyl)ethyl]-amino]propyl]phenyl]sulfonyl]-5-fluorobenzoic acid

(−)ESI-MS (m/z): 704, 706 (M−H)⁻⁽3) 2-[[4-[(2R)-2-[(tert-Butoxycarbonyl)[(2R)-2-(3-chlorophenyl)-2-hydroxyethyl]amino]propyl]phenyl]-sulfonyl]-5-(trifluoromethyl)benzoic acid

(−)ESI-MS (m/z): 640, 642 (M−H)⁻

(4) 2-[[4-[(2R)-2-[(tert-Butoxycarbonyl)[(2R)-2-(3-chlorophenyl)-2-hydroxyethyl]amino]propyl]phenyl]-sulfonyl]-6-(methoxymethoxy)benzoic acid

(−)ESI-MS (m/z): 632, 634 (M−H)⁻

### EXAMPLE 93

To a solution of ethyl 3-chloro-2-[[4-[(2R)-2-[[(2R)-2-(3-chlorophenyl)-2-hydroxyethyl]amino]propyl]phenyl]-sulfonyl]benzoate (172 mg) in 1,4-dioxane (4 ml) was added 6N hydrochloric acid (6 ml) at room temperature, and the mixture was refluxed for 42 hours. The resulting mixture was evaporated under reduced pressure. The residue was purified by reversed phase chromatography to give sodium 3-chloro-2-[[4-[(2R)-2-[[(2R)-2-(3-chlorophenyl)-2-hydroxyethyl]amino]propyl]phenyl]sulfonyl]benzoate (55 mg).

NMR (DMSO-d₆, δ): 0.89 (3H, d, J=5.9 Hz), 2.45-2.9 (5H, m), 4.5-4.6 (1H, m), 7.1-7.5 (9H, m), 8.22 (2H, d, J=8.3 Hz)

### EXAMPLE 94

To a solution of ethyl 2-[[4-[(2R)-2-[[(2R)-2-(3-chlorophenyl)-2-hydroxyethyl]amino]propyl]phenyl]sulfonyl]-5-methoxybenzoate (211 mg) in ethanol (0.5 ml) was added 1N sodium hydroxide (0.79 ml) at room temperature, and the mixture was refluxed for 2.5 hours. To the resulting mixture was added 1N hydrochloric acid (0.79 ml) and the mixture was evaporated under reduced pressure. The residue was purified by column chromatography on silica gel (chloroform/methanol=10:1 to 5:1), followed by treatment of 4N hydrogen chloride in ethyl acetate to give 2-[[4-[(2R)-2-[[(2R)-2-(3-chlorophenyl)-2-hydroxyethyl]amino]-propyl]phenyl]sulfonyl]-5-methoxybenzoic acid hydrochloride (77 mg).

NMR (DMSO-d₆, δ): 1.09 (3H, d, J=6.3 Hz), 2.65-2.9 (1H, m), 3.0-3.6 (4H, m), 3.86 (3H, s), 4.95-5.1 (1H, m), 7.12 (1H, d, J=2.5 Hz), 7.23 (1H, dd, J=2.6, 8.9 Hz), 7.5-7.55 (6H, m), 7.91 (2H, d, J=8.3 Hz), 8.07 (1H, d, J=8.9 Hz)

(−)ESI-MS (m/z): 502, 504 (M−HCl−H)⁻

### EXAMPLE 95

The following compounds were obtained according to a similar manner to that of Example 91.

(1) tert-Butyl [(2R)-2-[[tert-butyl(dimethyl)silyl]oxy]-2-(3-chlorophenyl)ethyl][(1R)-2-[4-[(4-fluoro-2-formylphenyl)sulfonyl]phenyl]-1-methylethyl]carbamate

(−)ESI-MS (m/z): 704, 706 (M+H₂O−H)⁻

(2) tert-Butyl [(2R)-2-(3-chlorophenyl)-2-hydroxyethyl]-[(1R)-2-[4-[[2-formyl-4-(trifluoromethyl)]phenyl]-sulfonyl]phenyl]-1-methylethyl]carbamate

(+)ESI-MS (m/z): 648, 650 (M+Na)⁺

(3) tert-Butyl [(2R)-2-(3-chlorophenyl)-2-hydroxyethyl]-[(1R)-2-[4-[[2-formyl-3-(methoxymethoxy)phenyl]-sulfonyl]phenyl]-1-methylethyl]carbamate

(+)ESI-MS (m/z): 640, 642 (M+Na)⁺

(4) tert-Butyl [(2R)-2-(3-chlorophenyl)-2-hydroxyethyl]-[(1R)-2-[4-[(2-formyl-3-methoxyphenyl)sulfonyl]phenyl]-1-methylethyl]carbamate

(+)ESI-MS (m/z): 594, 596 (M+Na)⁺

### EXAMPLE 96

Under nitrogen at room temperature, to a solution of 2-[[4-[(2R)-2-[(tert-butoxycarbonyl)[(2R)-2-[[tert-butyl(dimethyl)silyl]oxy]-2-(3-chlorophenyl)ethyl]amino]-propyl]phenyl]sulfonyl]-5-fluorobenzoic acid (150 mg) in N,N-dimethylformamide (3 ml) was added sodium thiomethoxide (33 mg), and the mixture was stirred at 60° C. for 1.5 hours. The mixture was cooled to room temperature, and n-tetrabutylammonium fluoride (1M in tetrahydrofuran, 0.64 ml) was added. The mixture was stirred at room temperature for 1.5 hours. The resulting mixture was poured into 1N hydrochloric acid and the aqueous mixture was extracted with ethyl acetate. The organic layer was dried over anhydrous magnesium sulfate and evaporated under reduced pressure. The residue was purified by column chromatography on silica gel (chloroform/methanol=10:1 to 8:1) to give 2-[[4-[(2R)-2-[(tert-butoxycarbonyl)[(2R)-2-(3-chlorophenyl)-2-hydroxyethyl]amino]propyl]phenyl]sulfonyl]-5-(methylthio)benzoic acid (149 mg).

(−)ESI-MS (m/z): 618, 620 (M−H)⁻

### EXAMPLE 97

Under nitrogen at room temperature, to a solution of 2-[[4-[(2R)-2-[(tert-butoxycarbonyl)[(2R)-2-(3-chlorophenyl)-2-hydroxyethyl]amino]propyl]phenyl]sulfonyl]-6-(methoxymethoxy)benzoic acid (208 mg) in 1,4-dioxane (2 ml) was added 6N hydrochloric acid (2 ml), and the mixture was stirred at the same temperature for 12 hours. The resulting mixture was evaporated and dried in vacuo to give 2-[[4-[(2R)-2-[[(2R)-2-(3-chlorophenyl)-2-hydroxyethyl]amino]-propyl]phenyl]sulfonyl]-6-hydroxybenzoic acid hydrochloride (181 mg).

NMR (DMSO-d₆, δ): 1.10 (3H, d, J=6.5 Hz), 2.65-2.9 (1H, m), 3.0-3.6 (4H, m), 4.95-5.1 (1H, m), 7.18 (1H, dd, J=2.7, 6.4 Hz), 7.3-7.6 (8H, m), 7.92 (2H, d, J=8.3 Hz)

(−)ESI-MS (m/z): 488, 490 (M−HCl−H)⁻

### EXAMPLE 98

Under nitrogen at room temperature, to a solution of ethyl 2-[[4-[(2R)-2-[[(2R)-2-(3-chlorophenyl)-2-hydroxyethyl]amino]propyl]phenyl]sulfonyl]-5-propylbenzoate (179 mg) in 1,4-dioxane (2 ml) was added a solution of di-tert-butyl dicarbonate (79 mg) in 1,4-dioxane (1 ml), and the mixture was stirred at the same temperature for 7 hours. To this one was added 1N sodium hydroxide (0.99 ml) and 1,4-dioxane (2 ml), and the mixture was stirred at 50° C. for 2.5 days. The resulting mixture was poured into a mixture of 1N hydrochloric acid and chloroform/methanol (5:1), and the mixture was stirred for 20 minutes. After separation, the organic layer was dried over anhydrous magnesium sulfate and evaporated under reduced pressure. The residue was purified by column chromatography on silica gel (chloroform/methanol=20:1 to 10:1) to give 2-[[4-[(2R)-2-[(tert-butoxycarbonyl)[(2R)-2-(3-chlorophenyl)-2-hydroxyethyl]-amino]propyl]phenyl]sulfonyl]-5-propylbenzoic acid (183 mg).

(−)ESI-MS (m/z): 614, 616 (M−H)⁻

### EXAMPLE 99

Under nitrogen at room temperature, to a solution of 2-[[4-[(2R)-2-[(tert-butoxycarbonyl)[(2R)-2-(3-chlorophenyl)-2-hydroxyethyl]amino]propyl]phenyl]sulfonyl]-5-propylbenzoic acid (180 mg) in ethyl acetate (1 ml) was added 4N hydrogen chloride in ethyl acetate (2 ml), and the mixture was stirred at the same temperature for 9 hours. The resulting mixture was evaporated under reduced pressure. The residue was purified by reversed phase chromatography, followed by treatment with 1N hydrochloric acid to give 2-[[4-[(2R)-2-[[(2R)-2-(3-chlorophenyl)-2-hydroxyethyl]amino]propyl]-phenyl]sulfonyl]-5-propylbenzoic acid hydrochloride (93 mg).

NMR (DMSO-d₆, δ): 0.87 (3H, t, J=7.3 Hz), 1.10 (3H, d, J=6.3 Hz), 1.45-1.7 (2H, m), 2.55-2.9 (3H, m), 3.0-3.6 (4H, m), 4.95-5.1 (1H, m), 7.35-7.6 (8H, m), 7.93 (2H, d, J=8.3 Hz), 8.04 (1H, d, J=8.2 Hz)

(−)ESI-MS (m/z): 514, 516 (M−HCl−H)⁻

### EXAMPLE 100

To a solution of ethyl 2-[[4-[(2R)-2-[[(2R)-2-(3-chlorophenyl)-2-hydroxyethyl]amino]propyl]phenyl]sulfonyl]-5-(3-methylbutyl)benzoate (44 mg) in ethanol (2 ml) was added 1N sodium hydroxide (0.387 ml), and the mixture was stirred at 45° C. for 4.5 hours. The resulting mixture was cooled to room temperature, 1N hydrochloric acid (0.542 ml) was added, and the mixture was purified by reversed phase chromatography, followed by treatment with 1N hydrochloric acid to give 2-[[4-[(2R)-2-[[(2R)-2-(3-chlorophenyl)-2-hydroxyethyl]amino]propyl]phenyl]sulfonyl]-5-(3-methylbutyl)benzoic acid hydrochloride (41 mg).

NMR (DMSO-d₆, δ): 0.90 (6H, d, J=6.0 Hz), 1.10 (3H, d, J=6.3 Hz), 1.35-1.65 (3H, m), 2.6-2.9 (3H, m), 3.0-3.6 (4H, m), 4.95-5.1 (1H, m), 7.35-7.6 (8H, m), 7.93 (2H, d, J=8.3 Hz), 8.03 (1H, d, J=8.1 Hz)

(−)ESI-MS (m/z): 542, 544 (M−HCl−H)⁻

### EXAMPLE 101

The following compounds were obtained according to a similar manner to that of Example 100.

(1) 2-[[4-[(2R)-2-[[(2R)-2-(3-Chlorophenyl)-2-hydroxyethyl]amino]propyl]phenyl]sulfonyl]-5-cyclohexylbenzoic acid hydrochloride

NMR (DMSO-d₆, δ): 1.10 (3H, d, J=6.3 Hz), 1.15-1.9 (10H, m), 2.55-2.9 (2H, m), 3.0-3.6 (4H, m), 4.95-5.1 (1H, m), 7.35-7.6 (8H, m), 7.94 (2H, d, J=8.2 Hz), 8.04 (1H, d, J=8.3 Hz)

(−)ESI-MS (m/z): 554, 556 (M−HCl−H)⁻

(2) 2-[[4-[(2R)-2-[[(2R)-2-(3-Chlorophenyl)-2-hydroxyethyl]amino]propyl]phenyl]sulfonyl]-4-propylbenzoic acid hydrochloride

NMR (DMSO-d₆, δ): 0.89 (9H, t, J=7.3 Hz), 1.10 (3H, d, J=6.3 Hz), 1.5-1.7 (2H, m), 2.6-2.9 (3H, m), 3.0-3.6 (4H, m), 4.95-5.1 (1H, m), 7.35-7.65 (8H, m), 7.9-8.0 (3H, m)

(−)ESI-MS (m/z): 514, 516 (M−HCl−H)⁻

(3) 4-[[4-[(2R)-2-[[(2R)-2-(3-Chlorophenyl)-2-hydroxyethyl]amino]propyl]phenyl]sulfonyl]-3-biphenylcarboxylic acid hydrochloride

NMR (DMSO-d₆, δ): 1.11 (3H, d, J=6.3 Hz), 2.7-2.9 (1H, m), 3.0-3.6 (4H, m), 4.95-5.1 (1H, m), 7.3-7.6 (9H, m), 7.7-7.8 (2H, m), 7.88 (1H, d, J=1.7 Hz), 7.9-8.05 (3H, m), 8.21 (1H, d, J=8.4 Hz)

(−)ESI-MS (m/z): 548, 550 (M−HCl−H)⁻

(4) 4-[[4-[(2R)-2-[[(2R)-2-Hydroxy-2-phenylethyl]amino]-propyl]phenyl]sulfonyl]benzoic acid hydrochloride

NMR (DMSO-d₆, δ): 1.09 (3H, d, J=6.3 Hz), 2.75-2.9 (1H, m), 3.0-3.6 (4H, m), 4.9-5.0 (1H, m), 7.25-7.5 (5H, m), 7.53 (2H, d, J=8.3 Hz), 7.97 (2H, d, J=8.3 Hz), 8.0-8.2 (4H, m)

(−)ESI-MS (m/z): 438 (M−HCl−H)⁻

### EXAMPLE 102

Under nitrogen at room temperature, to a solution of tert-butyl [(2R)-2-(3-chlorophenyl)-2-hydroxyethyl][(1R)-2-[4-[(2-formyl-4-methylphenyl)sulfonyl]phenyl]-1-methylethyl]carbamate (490 mg) in tetrahydrofuran (i0 ml) was added (carboethoxymethylene)triphenylphosphorane (328 mg), and the mixture was stirred at 50° C. for 2.5 hours. The resulting mixture was poured into saturated aqueous sodium bicarbonate and the aqueous mixture was extracted with ethyl acetate. The organic layer was washed with brine, dried over anhydrous magnesium sulfate and evaporated under reduced pressure. The residue was purified by column chromatography on silica gel (chloroform/ethyl acetate=20:1 to 10:1) to give a mixture (327 mg) of ethyl(2E)-3-[2-[[4-[(2R)-2-[(tert-butoxycarbonyl)[(2R)-2-(3-chlorophenyl)-2-hydroxyethyl]amino]propyl]phenyl]sulfonyl]-5-methylphenyl]acrylate and ethyl(2Z)-3-[2-[[4-[(2R)-2-[(tert-butoxycarbonyl)[(2R)-2-(3-chlorophenyl)-2-hydroxyethyl]amino]propyl]phenyl]sulfonyl]-5-methylphenyl]-acrylate.

(+)ESI-MS (m/z): 664 (M+Na)⁺

### EXAMPLE 103

Under nitrogen at 5° C., to a solution of a mixture (225 mg) of ethyl(2E)-3-[2-[[4-[(2R)-2-[(tert-butoxycarbonyl)-[(2R)-2-(3-chlorophenyl)-2-hydroxyethyl]amino]propyl]-phenyl]sulfonyl]-5-methylphenyl]acrylate and ethyl(2Z)-3-[2-[[4-[(2R)-2-[(tert-butoxycarbonyl)[(2R)-2-(3-chlorophenyl)-2-hydroxyethyl]amino]propyl]phenyl]sulfonyl]-5-methylphenyl]acrylate, and copper(I) chloride (52 mg) in methanol (8 ml) was added sodium borohydride (133 mg) in small portions over a period of 30 minutes, and the mixture was stirred at the same temperature for 1 hour. The resulting mixture was poured into a mixture of 1N hydrochloric acid and ethyl acetate and the mixture was stirred for 5 minutes. After separation, the organic layer was washed successively with saturated aqueous sodium bicarbonate and brine, dried over anhydrous magnesium sulfate, evaporated and dried in vacuo to give ethyl 3-[2-[[4-[(2R)-2-[(terty-butoxycarbonyl)[(2R)-2-(3-chlorophenyl)-2-hydroxyethyl]amino]propyl]phenyl]sulfonyl]-5-methylphenyl]propanoate (215 mg).

(+)ESI-MS (m/z): 666 (M+Na)⁺

### EXAMPLE 104

To a solution of ethyl 3-[2-[[4-[(2R)-2-[(tert-butoxycarbonyl)[(2R)-2-(3-chlorophenyl)-2-hydroxyethyl]-amino]propyl]phenyl]sulfonyl]-5-methylphenyl]propanoate (210 mg) in ethanol (5 ml) was added 1N sodium hydroxide (0.652 ml) at room temperature, and the mixture was stirred at the same temperature for 2.5 days. The resulting mixture was poured into 1N hydrochloric acid and the aqueous mixture was extracted with a mixture of chloroform and methanol (5:1). The organic layer was dried over anhydrous magnesium sulfate and evaporated under reduced pressure. The residue was purified by column chromatography on silica gel (chloroform/methanol=30:1 to 20:1) to give 3-[2-[[4-[(2R)-2-[(tert-butoxycarbonyl)[(2R)-2-(3-chlorophenyl)-2-hydroxyethyl]amino]propyl]phenyl]sulfonyl]-5-methylphenyl]-propanoic acid (167 mg).

(−)ESI-MS (m/z): 614, 616 (M−H)⁻

### EXAMPLE 105

The following compound was obtained according to a similar manner to that of Example 57. 3-[2-[[4-[(2R)-2-[[(2R)-2-(3-Chlorophenyl)-2-hydroxyethyl]amino]propyl]phenyl]sulfonyl]-5-methylphenyl]propanoic acid hydrochloride

NMR (DMSO-d₆, δ): 1.08 (3H, d, J=6.3 Hz), 2.15-2.3 (2H, m), 2.40 (3H, s), 2.65-3.6 (7H, m), 4.95-5.1 (1H, m), 7.25-7.55 (8H, m), 7.78 (2H, d, J=8.3 Hz), 8.00 (1H, d, J=8.1 Hz)

(−)ESI-MS (m/z): 514, 516 (M−HCl−H)⁻

### EXAMPLE 106

The following compounds were obtained according to a similar manner to that of Example 104.

(1) (2E)-3-[2-[[4-[(2R)-2-[(tert-Butoxycarbonyl)[(2R)-2-(3-chlorophenyl)-2-hydroxyethyl]amino]propyl]phenyl]-sulfonyl]-5-methylphenyl]acrylic acid

(−)ESI-MS (m/z): 612, 614 (M−H)⁻

(2) (2Z)-3-[2-[[4-[(2R)-2-[(tert-Butoxycarbonyl)[(2R)-2-(3-chlorophenyl)-2-hydroxyethyl]amino]propyl]phenyl]-sulfonyl]-5-methylphenyl]acrylic acid

(−)ESI-MS (m/z): 612, 614 (M−H)⁻

### EXAMPLE 107

A solution of ethyl 4-[[4-[(2R)-2-aminopropyl]phenyl]-sulfonyl]benzoate (212 mg) and (R)-styrene oxide (73 mg) in a mixture of ethanol (10 ml) and chloroform (3 ml) was refluxed for 22 hours. The resulting mixture was evaporated under reduced pressure. The residue was purified by column chromatography on silica gel (chloroform/methanol=50:1 to 20:1) to give ethyl 4-[[4-[(2R)-2-[[(2R)-2-hydroxy-2-phenylethyl]amino]propyl]phenyl]sulfonyl]benzoate (85 mg).

(+)ESI-MS (m/z): 468 (M+H)⁺

### EXAMPLE 108

A solution of ethyl 5-[[4-[(2R)-2-aminopropyl]phenyl]-sulfonyl]-2-hydroxybenzoate (277 mg) and (R)-styrene oxide (128 mg) in a mixture of methanol (4 ml) and chloroform (4 ml) was refluxed for 47 hours. The resulting mixture was evaporated under reduced pressure. The residue was purified by column chromatography on silica gel (chloroform/methanol=50:1 to 30:1). Under nitrogen at room temperature, to a solution of the product which was obtained above, in tetrahydrofuran (2 ml) was added di-tert-butyl dicarbonate (79 mg), and the mixture was stirred at the same temperature for 12 hours. The resulting mixture was poured into water and the aqueous mixture was extracted with ethyl acetate. The organic layer was washed with brine, dried over anhydrous magnesium sulfate and evaporated under reduced pressure. The residue was purified by column chromatography on silica gel (hexane/ethyl acetate=3:1 to 2:1) to give 5-[[4-[(2R)-2-[(tert-butoxycarbonyl)[(2R)-2-hydroxy-2-phenylethyl]amino]propyl]phenyl]sulfonyl]-2-hydroxybenzoate (68 mg).

(+)ESI-MS (m/z): 606 (M+Na)⁺

### EXAMPLE 109

To a solution of 5-[[4-[(2R)-2-[(tert-butoxycarbonyl)-[(2R)-2-hydroxy-2-phenylethyl]amino]propyl]phenyl]sulfonyl]-2-hydroxybenzoate (62 mg) in ethanol (2 ml) was added 1N sodium hydroxide (0.53 ml) at room temperature, and the mixture was stirred at 45°C for 4 hours. The resulting mixture was poured into 1N hydrochloric acid and the aqueous mixture was extracted with a mixture of chloroform and methanol (4:1). After separation, the organic layer was dried over anhydrous magnesium sulfate, evaporated and dried in vacuo to give 5-[[4-[(2R)-2-[(tert-butoxycarbonyl)[(2R)-2-hydroxy-2-phenylethyl]amino]propyl]phenyl]sulfonyl]-2-hydroxybenzoic acid (65 mg).

(−)ESI-MS (m/z): 554 (M−H)⁻

### EXAMPLE 110

The following compound was obtained according to a similar manner to that of Example 66. 2-Hydroxy-5-[[4-[(2R)-2-[[(2R)-2-hydroxy-2-phenylethyl]amino]propyl]phenyl]sulfonyl]benzoic acid hydrochloride

NMR (DMSO-d₆, δ): 1.10 (3H, d, J=6.3 Hz), 2.65-3.6 (5H, m), 4.85-5.0 (1H, m), 6.85-7.0 (1H, m), 7.25-7.6 (7H, m), 7.75-7.95 (3H, m), 8.20 (1H, d, J=2.5 Hz)

(−)ESI-MS (m/z): 454 (M−HCl−H)⁻

### EXAMPLE 111

To a solution of methyl 5-[[4-[2-[[(2R)-2-(3-chlorophenyl)-2-hydroxyethyl]amino]ethyl]phenyl]sulfonyl]-2-(2-ethoxy-2-oxoethoxy)benzoate (349 mg) in ethanol (7 ml) was added 1N sodium hydroxide (1.21 ml) at room temperature, and the mixture was stirred at 60° C. for 110 minutes. The resulting mixture was evaporated under reduced pressure. The residue was triturated with ethanol, and the precipitates were collected by filtration, followed by dryness in vacuo to give disodium 5-[[4-[2-[[(2R)-2-(3-chlorophenyl)-2-hydroxyethyl]amino]ethyl]phenyl]sulfonyl]-2-(2-oxido-2-oxoethoxy)benzoate (315 mg).

NMR (DMSO-d₆, δ): 2.35-2.85 (6H, m), 4.12 (2H, s), 4.55-4.7 (1H, m), 7.0-7.1 (1H, m), 7.15-7.55 (5H, m), 7.6-7.85 (4H, m), 7.9-8.0 (1H, m)

(−)ESI-MS (m/z): 532 (M−2Na)⁻

### EXAMPLE 112

To a solution of methyl 5-[[4-[2-[[(2R)-2-(3-chlorophenyl)-2-hydroxyethyl]amino]ethyl]phenyl]sulfonyl]-2-(2-hydroxyethoxy)benzoate (207 mg) in ethanol (2 ml) was added 1N sodium hydroxide (0.388 ml) at room temperature, and the mixture was stirred at 60° C. for 2 hours. The resulting mixture was evaporated and dried in vacuo to give sodium 5-[[4-[2-[[(2R)-2-(3-chlorophenyl)-2-hydroxyethyl]-amino]ethyl]phenyl]sulfonyl]-2-(2-hydroxyethoxy)benzoate (213 mg).

NMR (DMSO-d₆, δ): 2.55-2.8 (6H, m), 3.4-3.55 (2H, m), 4.1-4.2 (2H, m), 4.55-4.65 (1H, m), 7.1-7.45 (7H, m), 7.7-7.85 (4H, m)

(−)ESI-MS (m/z): 518, 520 (M−Na)⁻

### EXAMPLE 113

The following compound was obtained according to a similar manner to that of Example 107.

Methyl 5-[[4-[2-[benzyl[(2R)-2-hydroxy-2-phenylethyl]amino]ethyl]phenyl]sulfonyl]-2-hydroxybenzoate

(+)ESI-MS (m/z): 546 (M+H)⁺

### EXAMPLE 114

To a solution of methyl 5-[[4-[2-[benzyl[(2R)-2-hydroxy-2-phenylethyl]amino]ethyl]phenyl]sulfonyl]-2-hydroxybenzoate (158 mg) in methanol (5 ml) was added hydrogen chloride methanol reagent 10 (0.5 ml), and the mixture was evaporated under reduced pressure. A mixture of the residue and 10% palladium on activated carbon (50% wet, 8 mg) in methanol (3 ml) was stirred at room temperature in the presence of hydrogen at an atmospheric pressure for 5 hours. After filtration, the filtrate was evaporated under reduced pressure. The residue was dissolved into a mixture of saturated aqueous sodium bicarbonate and chloroform/methanol (4:1). After separation, the organic layer was dried over anhydrous magnesium sulfate and evaporated under reduced pressure. Under nitrogen at room temperature, to a solution of the residue in a mixture of tetrahydrofuran (5 ml) and N,N-dimethylformamide (5 ml) was added di-tert-butyl dicarbonate (65 mg), and the mixture was stirred at the same temperature for 2.5 hours. The resulting mixture was poured into water and the aqueous mixture was extracted with ethyl acetate. The organic layer was washed successively with water (two times) and brine, dried over anhydrous magnesium sulfate and evaporated under reduced pressure. The residue was purified by column chromatography on silica gel (hexane/ethyl acetate=2:1 to 4:3) to give methyl 5-[[4-[2-[(tert-butoxycarbonyl)[(2R)-2-hydroxy-2-phenylethyl]amino]ethyl]phenyl]sulfonyl]-2-hydroxybenzoate (112 mg).

(+)ESI-MS (m/z): 578 (M+Na)⁺

### EXAMPLE 115

The following compound was obtained according to a similar manner to that of Example 109. 5-[[4-[2-[(tert-Butoxycarbonyl)[(2R)-2-hydroxy-2-phenylethyl]amino]ethyl]phenyl]sulfonyl]-2-hydroxybenzoic acid

(−)ESI-MS (m/z): 540 (M−H)⁻

### EXAMPLE 116

The following compound was obtained according to a similar manner to that of Example 75.

2-Hydroxy-5-[[4-[2-[[(2R)-2-hydroxy-2-phenylethyl]-amino]ethyl]phenyl]sulfonyl]benzoic acid hydrochloride

NMR (DMSO-d₆, δ): 2.9-3.35 (6H, m), 4.85-5.0 (1H, m), 7.05-7.15 (1H, m), 7.25-7.45 (5H, m), 7.50 (2H, d, J=8.3 Hz), 78-8.0 (3H, m), 8.25-8.3 (1H, m)

(−)ESI-MS (m/z): 440 (M−HCl−H)⁻

### EXAMPLE 117

The following compound was obtained according to a similar manner to that of Example 83. 5-[[4-[2-[Benzyl[(2R)-2-(3-chlorophenyl)-2-hydroxyethyl]amino]ethyl]phenyl]sulfonyl]-2-hydroxy-N-methylbenzamide

(+)ESI-MS (m/z): 579, 581 (M+H)⁺

### EXAMPLE 118

To a solution of 5-[[4-[2-[benzyl[(2R)-2-(3-chlorophenyl)-2-hydroxyethyl]amino]ethyl]phenyl]sulfonyl]-2-hydroxy-N-methylbenzamide (143 mg) in methanol (3 ml) was added hydrogen chloride methanol reagent 10 (0.5 ml), and the mixture was evaporated under reduced pressure. A mixture of the residue and 10% palladium on activated carbon (50% wet, 7 mg) in a mixture of chlorobenzene (2.1 ml) and methanol (0.9 ml) was stirred at room temperature in the presence of hydrogen at an atmospheric pressure for 2 hours. After filtration, the filtrate was evaporated under reduced pressure. The residue was dissolved into a mixture of saturated aqueous sodium bicarbonate and chloroform/methanol (5:1). After separation, the organic layer was dried over anhydrous magnesium sulfate and evaporated under reduced pressure. The residue was purified by column chromatography on silica gel (chloroform/methanol=20:1 to 8:1) followed by treatment of hydrogen chloride methanol reagent 10 to give 5-[[4-[2-[[(2R)-2-(3-chlorophenyl)-2-hydroxyethyl]-amino]ethyl]phenyl]sulfonyl]-2-hydroxy-N-methylbenzamide hydrochloride (71 mg).

NMR (DMSO-d₆, δ): 2.83 (3H, d, J=4.5 Hz), 2.95-3.5 (6H, m), 4.9-5.0 (1H, m), 7.10 (1H, d, J=8.7 Hz), 7.3-7.6 (6H, m), 7.85-7.95 (3H, m), 8.49 (1H, d, J=2.3 Hz)

(+)ESI-MS (m/z): 489, 491 (M−HCl+H)⁺

1. A compound of the formula [I]: 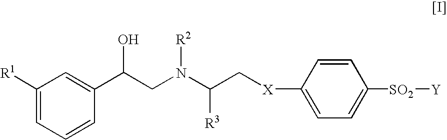
wherein
R¹ is hydrogen or halogen,
R² is hydrogen or an amino protective group,
R³ is hydrogen or lower alkyl,
X is bond, —CH₂— or —O—, and
Y is 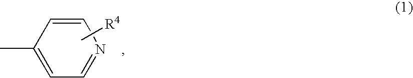
in which R⁴ is lower alkoxycarbonyl, 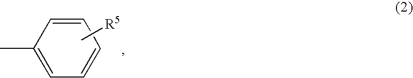
in which R⁵ is carboxy(lower)alkyl, (lower alkoxy)carbonyl(lower)alkyl, lower alkanoyl, mono(or di or tri)halo(lower)alkylsulfonyloxy, carboxyphenoxy, (lower alkoxy)carbonylphenoxy, carboxypyridyloxy, (lower alkanoyl)pyridyl, carboxypyrrolidinyl(lower)alkyl, (lower alkoxy)carbonylpyrrolidinyl(lower)alkyl, carboxyphenyl or (lower alkyl)phenyl, 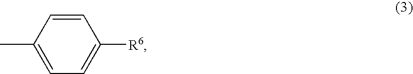
in which R⁶ is —OH, —COOH, —COOC₂H₅, 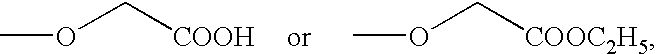
provided that
(i) when R⁶ is —OH, then X is —CH₂—,
(ii) when R⁶ is —COOH, then R¹ is —H, or
(iii) when R⁶ is —COOC₂H₅, 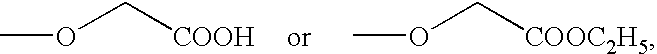
then X is —O—, 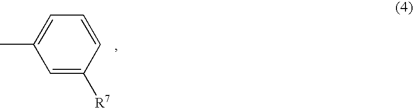
in which
R⁷ is —OH, —COOH, —COOC₂H₅, 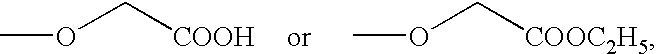
and
X is —CH₂—, 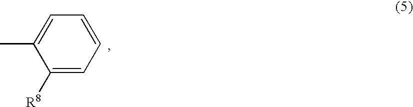
in which R⁸ is —OH, —COOH, —COOC₂H₅, 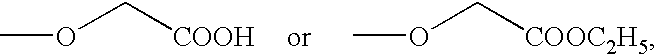
provided that when R⁸ is —OH, 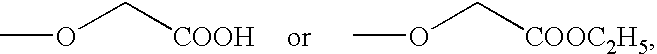
then R³ is —CH₃, 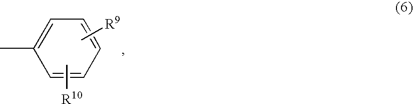
in which R⁹ is hydroxy, cyclo(lower)alkyl, mono(or di or tri)halo(lower)alkyl, hydroxy(lower)alkoxy, lower alkoxy(lower)alkoxy, carboxy(lower)alkoxy, lower alkoxycarbonyl(lower)alkoxy, phenoxy, nitro, amino, lower alkylamino, [lower alkoxy(lower)-alkyl]amino, [hydroxy(lower)alkyl]amino, [lower alkoxycarbonyl]amino, lower alkanoylamino, [hydroxy(lower)alkanoyl]amino, benzoylamino, (lower alkylsulfonyl)amino, lower alkylthio or phenyl, and
R¹⁰ is carboxy, lower alkanoyl, lower alkoxycarbonyl, carbamoyl, lower alkylcarbamoyl, carboxy(lower)alkyl, (lower alkoxycarbonyl)(lower)alkyl, carboxy(lower)-alkenyl, (lower alkoxycarbonyl)(lower)alkenyl or phenyl optionally substituted with carboxy or lower alkoxycarbonyl, 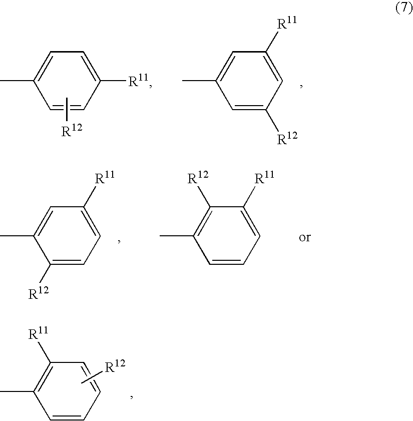
in which R¹¹ is halogen or lower alkyl, and
R¹² is carboxy, lower alkanoyl, lower alkoxycarbonyl, carbamoyl, lower alkylcarbamoyl, carboxy(lower)alkyl, (lower alkoxycarbonyl)(lower)alkyl, carboxy(lower)-alkenyl or (lower alkoxycarbonyl)(lower)alkenyl, 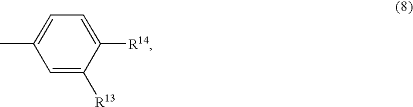
in which
R¹³ is —Cl or —CH₃,
R¹⁴ is —COOH or —COOC₂H₅, and
X is —CH₂—, 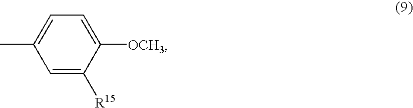
in which
R¹⁵ is —COOH or —COOC₂H₅, and
X is —CH₂—, or 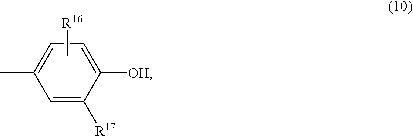
in which
R¹⁶ is lower alkyl or lower alkoxy, and
R¹⁷ is carboxy or lower alkoxycarbonyl,
or a prodrug thereof or a pharmaceutically acceptable salt thereof.

2. A compound of calim 1, wherein
R¹ is hydrogen or chloride,
R² is hydrogen or benzyl,
R³ is hydrogen or methyl,
X is bond, —CH₂— or —O—, and
Y is 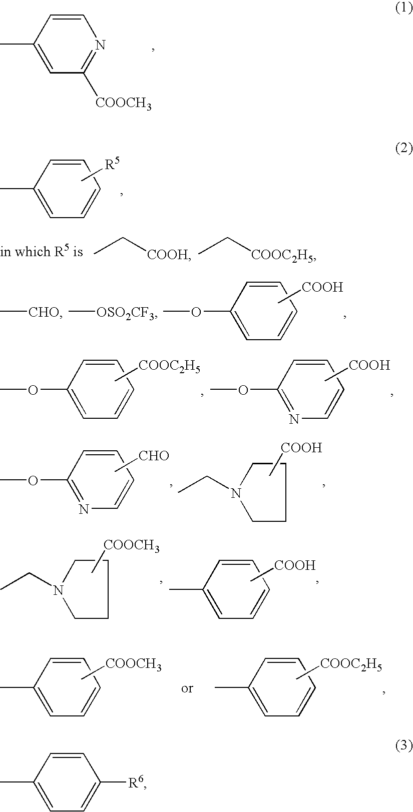
in which R⁶ is —OH, —COOH, —COOC₂H₅, 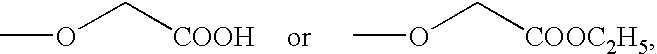
provided that
(i) when R⁶ is —OH, then X is —CH₂—,
(ii) when R⁶ is —COOH, then R¹ is —H, or
(iii) when R⁶ is —COOC₂H₅, 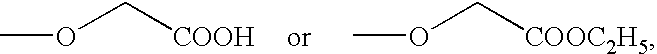
then X is —O—, 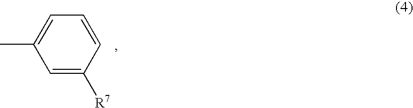
in which R⁷ is —OH, —COOH, —COOC₂H₅, 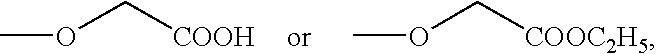
and X is —CH₂—, 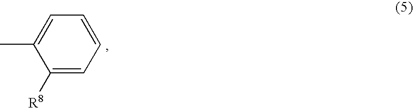
in which R⁸ is —OH, —COOH, —COOC₂H₅, 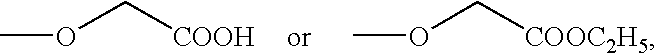
provided that
when R⁸ is —OH, 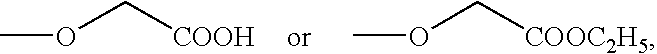
then R³ is —CH₃, 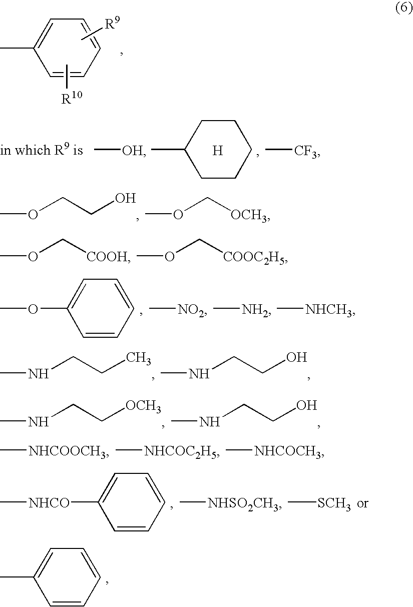
and
R¹⁰ is —COOH, —CHO, —COOCH₃, —COOC₂H₅, —CONH₂, —CONHCH₃. —CONHC₂H₅, 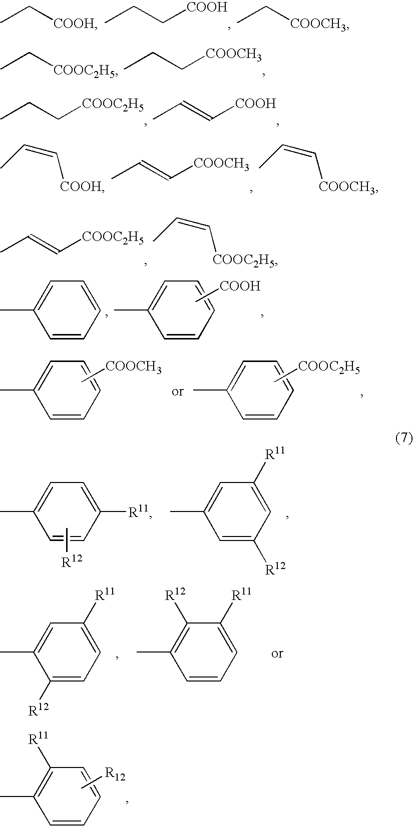
in which
R¹¹ is —F, —Cl or —CH₃, and
R¹² is —COOH, —CHO, —COOCH₃, —COOC₂H₅, —CONH₂₁—CONHCH₃, —CONHC₂H₅, 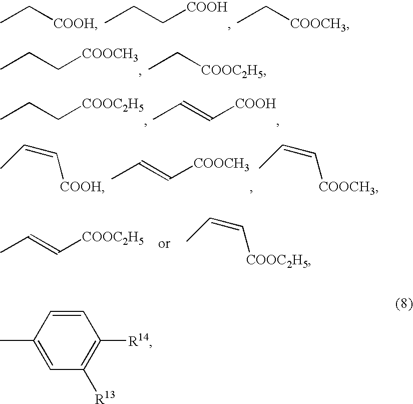
in which
R¹³ is —Cl or —CH₃,
R¹⁴ is —COOH or —COOC₂H₅, and
X is —CH₂—, 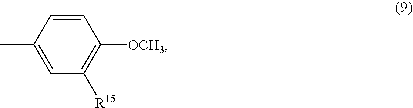
in which
R¹⁵ is —COOH or —COOC₂H₅, and
X is —CH₂—, or 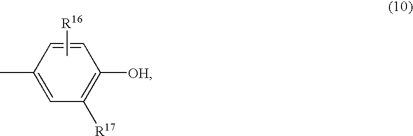
in which
R¹⁶ is —CH₃ or —OCH₃, and
R¹⁷ is —COOH, —COOCH₃ or —COOC₂H₅.

3. A compound of claim 2, wherein
Y is 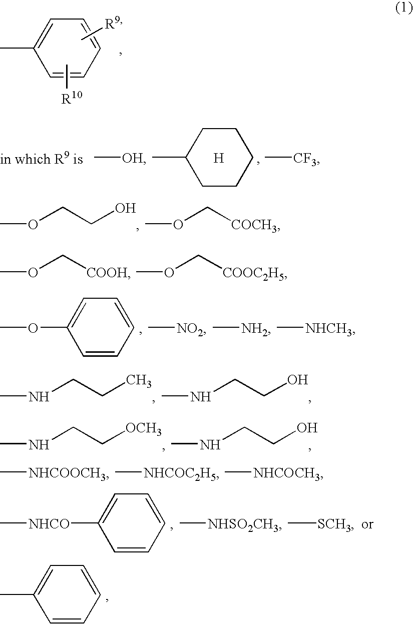
and
R¹⁰ is —COOH, —CHO, —COOCH₃, —COOC₂H₅, —CONH₂, —CONHCH₃, —CONHC₂H₅, 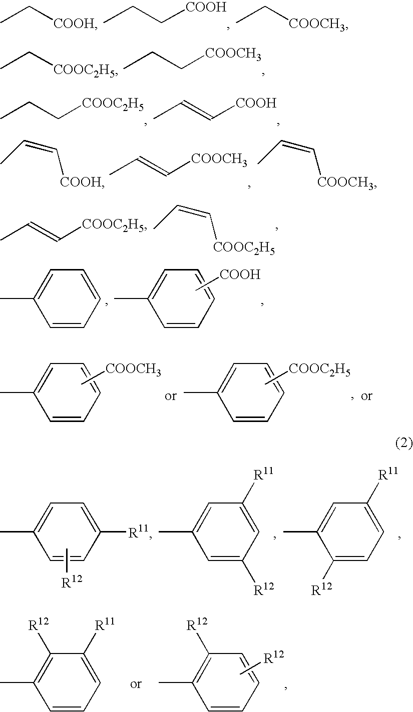
in which
R¹¹ is —F, —Cl or —CH₃, and
R¹² is —COOH, —CHO, —COOCH₃. —COOC₂H₅, —CONH₂, —CONHCH₃, —CONHC₂H₅,

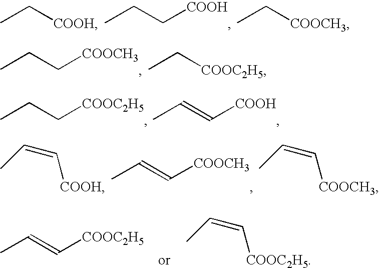

4. A compound of claim 3, wherein
Y is 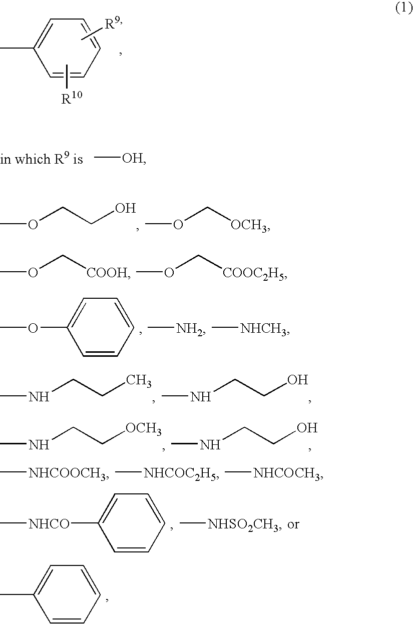
and
R¹⁰ is —COOH, —COOCH₃, —COOC₂H₅, —CONH₂, —CONHCH₃, —CONHC₂H₅, 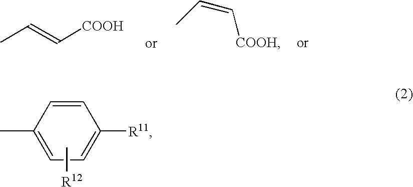
in which
R¹¹ is —CH₃, and
R¹² is —COOH, —COOCH₃, —COOC₂H₅, —CONH₂, —CONHCH₃, —CONHC₂H₅,

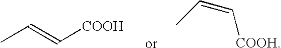

5. A compound of claim 4, which is selected from a group of
(1) 2-Amino-5-[[4-[2-[[(2R)-2-(3-chlorophenyl)-2-hydroxyethyl]amino]ethyl]phenyl]sulfonyl]benzoic acid,
(2) 5-[[4-[2-[[(2R)-2-(3-Chlorophenyl)-2-hydroxyethyl]amino]ethyl]phenyl]sulfonyl]-2-(methylamino)benzoic acid,
(3) 5-[[4-[2-[[(2R)-2-(3-Chlorophenyl)-2-hydroxyethyl]amino]ethyl]phenyl]sulfonyl]-2-[(methylsulfonyl)amino]benzoic acid,
(4) 5-[[4-[2-[[(2R)-2-(3-Chlorophenyl)-2-hydroxyethyl]amino]ethyl]phenyl]sulfonyl]-2-(propionylamino)benzoic acid,
(5) 5-[[4-[2-[[(2R)-2-(3-Chlorophenyl)-2-hydroxyethyl]amino]ethyl]phenyl]sulfonyl]-2-[(2-hydroxyethyl)amino]benzoic acid,
(6) 5-[[4-[2-[[(2R)-2-(3-Chlorophenyl)-2-hydroxyethyl]amino]ethyl]phenyl]sulfonyl]-2-hydroxy-N-methylbenzamide,
(7) [4-[[4-[3-[[(2R)-2-(3-Chlorophenyl)-2-hydroxyethyl]amino]propyl]phenyl]sulfonyl]-phenoxy]acetic acid,
(8) 2-Hydroxy-5-[[4-[2-[[(2R)-2-hydroxy-2-phenylethyl]amino]ethyl]phenyl]sulfonyl]benzoic acid,
(9) 5-[[4-[3-[[(2R)-2-(3-chlorophenyl)-2-hydroxyethyl]amino]propyl]phenyl]sulfonyl]-2-hydroxybenzoic acid,
(10) 2-[[4-[(2R)-2-[[(2R)-2-(3-Chlorophenyl)-2-hydroxyethyl]amino]propyl]phenyl]sulfonyl]-5-propylbenzoic acid,
(11) 4-[[4-[(2R)-2-[[(2R)-2-(3-Chlorophenyl)-2-hydroxyethyl]amino]propyl]phenyl]sulfonyl]-3-biphenylcarboxylic acid, and
(12) (2Z)-3-[2-[[4-[(2R)-2-[[(2R)-2-(3-Chlorophenyl)-2-hydroxyethyl]amino]propyl]phenyl]sulfonyl]-5-methylphenyl]acrylic acid,
or a pharmaceutically acceptable salt thereof.

6. A process for preparing a compound of claim 1, or a salt thereof,
which comprises,
(i) reacting a compound [II] of the formula: 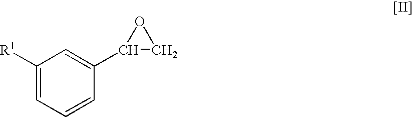
wherein R¹ is defined in claim 1,
with a compound [III] of the formula: 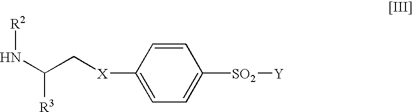
wherein R², R³, X and Y are each as defined in claim 1,
or a salt thereof, to give a compound [I] of the formula: 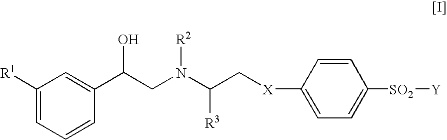
wherein R¹, R², R³, X and Y are each as defined in claim 1,
or a salt thereof,
(ii) subjecting a compound [Ia] of the formula: 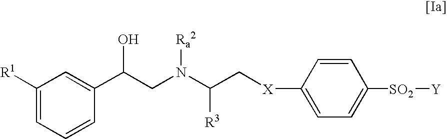
wherein
R¹, R³, X and Y are each as defined in claim 1, and
Rₐ² is an amino protective group,
or a salt thereof, to elimination reaction of the amino protective group, to give a compound [Ib] of the formula: 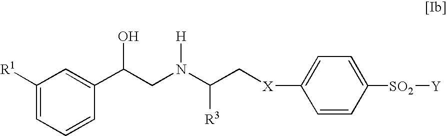
wherein R¹, R³, X and Y are each as defined in claim 1,
or a salt thereof, and
(iii) reacting a compound [Ic] of the formula: 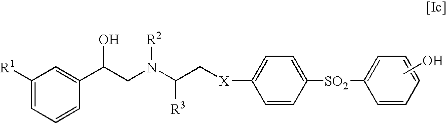
wherein R¹, R², R³ and X are each as defined in claim 1,
or a salt thereof with a compound [IV] of the formula:
Z-Rₐ⁵[IV]
wherein
Rₐ⁵ is lower alkyl optionally substituted with carboxy or lower alkoxycarbonyl; phenyl substituted with lower alkanoyl, carboxy or lower alkoxycarbonyl; or pyridyl optionally substituted with lower alkanoyl, carboxy or lower alkoxycarbonyl, and
Z is halogen,
or a salt thereof to give a compound [Id] of the formula: 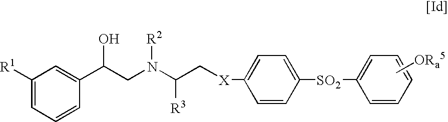
wherein
R¹, R², R³ and X are each as defined in claim 1, and
Rₐ⁵ is as defined above,
or a salt thereof.

7. A pharmaceutical composition which comprises, as an active ingredient, a compound of claim 1 or a pharmaceutically acceptable salt thereof in admixture with pharmaceutically acceptable carriers or excipients.

8. Use of a compound of claim 1 or a pharmaceutically acceptable salt thereof for the manufacture of a medicament.

9. A compound of claim 1 or a pharmaceutically acceptable salt thereof for use as a medicament.

10. A compound of claim 1 or a pharmaceutically acceptable salt thereof for use as selective β₃ adrenergic receptor agonists.

11. A method for the prophylactic and/or the therapeutic treatment of pollakiuria or urinary incontinence which comprises administering a compound of claim 1 or a pharmaceutically acceptable salt thereof to a human being or an animal.
